(19) United States
(12) Patent Application Publication
(10) Pub. No.: US 2005/0048507 A1
(43) Pub. Date: Mar. 3, 2005

(54) Therapeutic polypeptides, nucleic acids encoding same, and methods of use

(76) Inventors: Mei Zhong, Branford, CT (US); Xiaojia (Sasha) Guo, Branford, CT (US); David W. Anderson, Plantsville, CT (US); Tatiana Ort, Milford, CT (US); Muralidhara Padigaru, Mumbai (IN); Daniel Rieger, Branford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/659,004

(22) Filed: Sep. 9, 2003

Related U.S. Application Data
(63) Continuation-in-part of application No. 10/162,335, filed on Jun. 3, 2002.
(63) Continuation-in-part of application No. 10/044,564, filed on Jan. 11, 2002.
(63) Continuation-in-part of application No. 10/094,886, filed on Mar. 7, 2002.
(60) Provisional application No. 60/295,607, filed on Jun. 4, 2001.
(60) Provisional application No. 60/295,661, filed on Jun. 4, 2001.
(60) Provisional application No. 60/296,404, filed on Jun. 6, 2001.
(60) Provisional application No. 60/296,418, filed on Jun. 6, 2001.
(60) Provisional application No. 60/297,414, filed on Jun. 11, 2001.
(60) Provisional application No. 60/297,567, filed on Jun. 12, 2001.
(60) Provisional application No. 60/298,285, filed on Jun. 14, 2001.
(60) Provisional application No. 60/298,556, filed on Jun. 15, 2001.
(60) Provisional application No. 60/299,949, filed on Jun. 21, 2001.
(60) Provisional application No. 60/300,883, filed on Jun. 26, 2001.
(60) Provisional application No. 60/301,550, filed on Jun. 28, 2001.
(60) Provisional application No. 60/311,972, filed on Aug. 13, 2001.
(60) Provisional application No. 60/315,069, filed on Aug. 27, 2001.
(60) Provisional application No. 60/315,071, filed on Aug. 27, 2001.
(60) Provisional application No. 60/315,660, filed on Aug. 29, 2001.
(60) Provisional application No. 60/322,293, filed on Sep. 14, 2001.
(60) Provisional application No. 60/322,706, filed on Sep. 17, 2001.
(60) Provisional application No. 60/341,186, filed on Dec. 14, 2001.
(60) Provisional application No. 60/361,189, filed on Feb. 28, 2002.
(60) Provisional application No. 60/363,673, filed on Mar. 12, 2002.
(60) Provisional application No. 60/363,676, filed on Mar. 12, 2002.
(60) Provisional application No. 60/261,014, filed on Jan. 11, 2001.
(60) Provisional application No. 60/261,018, filed on Jan. 11, 2001.
(60) Provisional application No. 60/318,410, filed on Sep. 10, 2001.
(60) Provisional application No. 60/261,013, filed on Jan. 11, 2001.
(60) Provisional application No. 60/261,029, filed on Jan. 11, 2001.
(60) Provisional application No. 60/261,026, filed on Jan. 11, 2001.
(60) Provisional application No. 60/313,170, filed on Aug. 17, 2001.
(60) Provisional application No. 60/274,322, filed on Mar. 8, 2001.
(60) Provisional application No. 60/313,182, filed on Aug. 17, 2001.
(60) Provisional application No. 60/288,052, filed on May 2, 2001.
(60) Provisional application No. 60/318,510, filed on Sep. 10, 2001.
(60) Provisional application No. 60/274,281, filed on Mar. 8, 2001.
(60) Provisional application No. 60/314,018, filed on Aug. 21, 2001.
(60) Provisional application No. 60/274,194, filed on Mar. 8, 2001.
(60) Provisional application No. 60/274,849, filed on Mar. 9, 2001.
(60) Provisional application No. 60/296,693, filed on Jun. 7, 2001.
(60) Provisional application No. 60/313,626, filed on Aug. 20, 2001.
(60) Provisional application No. 60/332,486, filed on Nov. 9, 2001.
(60) Provisional application No. 60/275,235, filed on Mar. 12, 2001.
(60) Provisional application No. 60/275,578, filed on Mar. 13, 2001.
(60) Provisional application No. 60/288,228, filed on May 2, 2001.
(60) Provisional application No. 60/275,579, filed on Mar. 13, 2001.
(60) Provisional application No. 60/312,916, filed on Aug. 16, 2001.
(60) Provisional application No. 60/275,601, filed on Mar. 13, 2001.
(60) Provisional application No. 60/311,978, filed on Aug. 13, 2001.
(60) Provisional application No. 60/276,000, filed on Mar. 14, 2001.
(60) Provisional application No. 60/276,776, filed on Mar. 16, 2001.
(60) Provisional application No. 60/296,856, filed on Jun. 8, 2001.
(60) Provisional application No. 60/276,994, filed on Mar. 19, 2001.
(60) Provisional application No. 60/291,766, filed on May 17, 2001.
(60) Provisional application No. 60/277,338, filed on Mar. 20, 2001.
(60) Provisional application No. 60/288,066, filed on May 2, 2001.
(60) Provisional application No. 60/277,239, filed on Mar. 20, 2001.
(60) Provisional application No. 60/315,227, filed on Aug. 27, 2001.
(60) Provisional application No. 60/318,403, filed on Sep. 10, 2001.
(60) Provisional application No. 60/277,327, filed on Mar. 20, 2001.
(60) Provisional application No. 60/277,791, filed on Mar. 21, 2001.
(60) Provisional application No. 60/325,378, filed on Sep. 27, 2001.
(60) Provisional application No. 60/277,833, filed on Mar. 22, 2001.
(60) Provisional application No. 60/278,152, filed on Mar. 23, 2001.
(60) Provisional application No. 60/310,913, filed on Aug. 8, 2001.
(60) Provisional application No. 60/303,237, filed on Jul. 5, 2001.
(60) Provisional application No. 60/278,894, filed on Mar. 26, 2001.
(60) Provisional application No. 60/322,360, filed on Sep. 14, 2001.
(60) Provisional application No. 60/279,036, filed on Mar. 27, 2001.
(60) Provisional application No. 60/312,191, filed on Aug. 14, 2001.
(60) Provisional application No. 60/278,999, filed on Mar. 27, 2001.
(60) Provisional application No. 60/280,233, filed on Mar. 30, 2001.
(60) Provisional application No. 60/303,230, filed on Jul. 5, 2001.
(60) Provisional application No. 60/345,399, filed on Nov. 9, 2001.
(60) Provisional application No. 60/322,296, filed on Sep. 14, 2001.
(60) Provisional application No. 60/280,802, filed on Apr. 2, 2001.
(60) Provisional application No. 60/414,832, filed on Sep. 30, 2002.
(60) Provisional application No. 60/409,544, filed on Sep. 10, 2002.
(60) Provisional application No. 60/413,342, filed on Sep. 25, 2002.
(60) Provisional application No. 60/412,767, filed on Sep. 23, 2002.
(60) Provisional application No. 60/412,766, filed on Sep. 23, 2002.
(60) Provisional application No. 60/411,060, filed on Sep. 16, 2002.
(60) Provisional application No. 60/412,825, filed on Sep. 23, 2002.
(60) Provisional application No. 60/410,320, filed on Sep. 12, 2002.
(60) Provisional application No. 60/409,145, filed on Sep. 9, 2002.

(51) Int. Cl. C12Q 1/68; G01N 33/53; C07K 14/47; A61K 38/17

(52) U.S. Cl. 435/6; 435/69.1; 435/320.1; 435/325; 530/350; 514/12; 536/23.5

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 10/162,335 filed Jun. 3, 2002, which claims benefit to U.S. Ser. No. 60/295,607 filed Jun. 4, 2001, U.S. Ser. No. 60/295,661 filed Jun. 4, 2001, U.S. Ser. No. 60/296,404 filed Jun. 6, 2001, U.S. Ser. No. 60/296,418 filed Jun. 6, 2001, U.S. Ser. No. 60/297,414 filed Jun. 11, 2001, U.S. Ser. No. 60/297,567 filed Jun. 12, 2001, U.S. Ser. No. 60/298,285 filed Jun. 14, 2001, U.S. Ser. No. 60/298,556 filed Jun. 15, 2001, U.S. Ser. No. 60/299,949 filed Jun. 21, 2001, U.S. Ser. No. 60/300,883 filed Jun. 26, 2001, U.S. Ser. No. 60/301,550 filed Jun. 28, 2001, U.S. Ser. No. 60/311,972 filed Aug. 13, 2001, U.S. Ser. No. 60/315,069 filed Aug. 27, 2001, U.S. Ser. No. 60/315,071 filed Aug. 27, 2001, U.S. Ser. No. 60/315,660 filed Aug. 29, 2001, U.S. Ser. No. 60/322,293 filed Sep. 14, 2001, U.S. Ser. No. 60/322,706 filed Sep. 17, 2001, U.S. Ser. No. 60/341,186 filed Dec. 14, 2001, U.S. Ser. No. 60/361,189 filed Feb. 28, 2002, U.S. Ser. No. 60/363,673 filed Mar. 12, 2002, and U.S. Ser. No. 60/363,676 filed Mar. 12, 2002; a continuation-in-part of U.S. Ser. No. 10/044,564 filed Jan. 11, 2002, which claims benefit to U.S. Ser. No. 60/261,014 filed Jan. 11, 2001, U.S. Ser. No. 60/261,018 filed 01/11/01, U.S. Ser. No. 60/318,410 filed Sep. 10, 2001, U.S. Ser. No. 60/261,013 filed Jan. 11, 2001, U.S. Ser. No. 60/261,029 filed 01/11/01, U.S. Ser. No. 60/261,026 filed 01/11/01, and U.S. Ser. No. 60/313,170 filed Aug. 17, 2001; a continuation-in-part of U.S. Ser. No. 10/094,886 filed Mar. 7, 2002, which claims benefit to U.S. Ser. No. 60/274,322 filed Mar. 8, 2001, U.S. Ser. No. 60/313,182 filed Aug. 17, 2001, U.S. Ser. No. 60/288,052 filed May 2, 2001, U.S. Ser. No. 60/318,510 filed Sep. 10, 2001, U.S. Ser. No. 60/274,281 filed Mar. 8, 2001, U.S. Ser. No. 60/314,018 filed Aug. 21, 2001, U.S. Ser. No. 60/274,194 filed Mar. 8, 2001, U.S. Ser. No. 60/274,849 filed Mar. 9, 2001, U.S. Ser. No. 60/296,693 filed Jun. 7, 2001, U.S. Ser. No. 60/313,626 filed Aug. 20, 2001, U.S. Ser. No. 60/332,486 filed Nov. 9, 2001, U.S. Ser. No. 60/275,235 filed Mar. 12, 2001, U.S. Ser. No. 60/275,578 filed Mar. 13, 2001, U.S. Ser. No. 60/288,228 filed May 2, 2001, U.S. Ser. No. 60/275,579 filed Mar. 13, 2001, U.S. Ser. No. 60/312,916 filed Aug. 16, 2001, U.S. Ser. No. 60/275,601 filed Mar. 13, 2001, U.S. Ser. No. 60/311,978 filed Aug. 13, 2001, U.S. Ser. No. 60/276,000 filed Mar. 14, 2001, U.S. Ser. No. 60/276,776 filed Mar. 16, 2001, U.S. Ser. No. 60/296,856 filed Jun. 8, 2001, U.S. Ser. No. 60/276,994 filed Mar. 19, 2001, U.S. Ser. No. 60/291,766 filed May 17, 2001, U.S. Ser. No. 60/277,338 filed Mar. 20, 2001, U.S. Ser. No. 60/288,066 filed May 2, 2001, U.S. Ser. No. 60/277,239 filed Mar. 20, 2001, U.S. Ser. No. 60/315,227 filed Aug. 27, 2001, U.S. Ser. No. 60/318,403 filed Sep. 10, 2001, U.S. Ser. No. 60/277,327 filed Mar. 20, 2001, U.S. Ser. No. 60/277,791 filed Mar. 21, 2001, U.S. Ser. No. 60/325,378 filed Sep. 27, 2001, U.S. Ser. No. 60/277,833 filed Mar. 22, 2001, U.S. Ser. No. 60/278,152 filed Mar. 23, 2001, U.S. Ser. No. 60/310,913 filed Aug. 8, 2001, U.S. Ser. No. 60/303,237, 07/5/01, U.S. Ser. No. 60/278,894 filed Mar. 26, 2001, U.S. Ser. No. 60/322,360 filed Sep. 14, 2001, U.S. Ser. No. 60/279,036 filed Mar. 27, 2001, U.S. Ser. No. 60/312,191,08/14/01, U.S. Ser. No. 60/278,999 filed Mar. 27, 2001, U.S. Ser. No. 60/280,233 filed Mar. 30, 2001, U.S. Ser. No. 60/303,230, 07/5/01, U.S. Ser. No. 60/345,399 filed Nov. 9, 2001, U.S. Ser. No. 60/322,296 filed Sep. 14, 2001, and U.S. Ser. No. 60/280,802 filed Apr. 2, 2001; and this application claims priority to provisional applications U.S. Ser. No. 60/414,832 filed Sep. 30, 2002, U.S. Ser. No. 60/409,544 filed Sep. 10, 2002, U.S. Ser. No. 60/413,342 filed Sep. 25, 2002, U.S. Ser. No. 60/412,767 filed Sep. 24, 2002, U.S. Ser. No. 60/412,766 filed Sep. 23, 2002, U.S. Ser. No. 60/411,060 filed Sep. 16, 2002, U.S. Ser. No. 60/412,825 filed Sep. 23, 2002, U.S. Ser. No. 60/410,320 filed Sep. 12, 2002, and U.S. Ser. No. 60/409,145 filed Sep. 9, 2002, all of which are incorporated herein by reference in their entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 71 or is 94. The novel nucleic acids and polypeptides are referred to herein as NOV1 a, NOV1 b, NOV1 b, NOV1 c, NOV2a, NOV2b, NOV2c, NOV2d, NOV3a, NOV3b, etc. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 71 or is 94, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 71 or is 94. In another embodiment, the invention also comprises variants of the amino acid sequence selected from the group consisting of SEQ ID NO:2n, wherein n is an integer between 1 and 71 or is 94 wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 71 or is 94, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 71 or is 94. These allelic variants include amino acid sequences that are the translations of nucleic acid sequences differing by a single nucleotide from nucleic acid sequences selected from the group consisting of SEQ ID NOS: 2n-1, wherein n is an integer between 1 and 71. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 71 or is 94 and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 71 or is 94wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 71 or is 94 in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 71 or is 94 in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 71 or is 94, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 71 or is 94, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 71 or is 94, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 71 or is 94, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 71 or is 94, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 71 or is 94 or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 71 or is 94; a variant of a mature form of the amino acid sequence selected from the group consisting of SEQ ID NO:2n, wherein n is an integer between 1 and 71 or is 94 wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 71 or is 94; a variant of the amino acid sequence selected from the group consisting of SEQ ID NO:2n, wherein n is an integer between 1 and 71 or is 94, in which any amino acid specified in the chosen sequence is changed to a different amino acid, provided that no more than 15% of the amino acid residues in the sequence are so changed; a nucleic acid fragment encoding at least a portion of a polypeptide comprising the amino acid sequence selected from the group consisting of SEQ ID NO:2n, wherein n is an integer between 1 and 71 or is 94 or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 71 or is 94, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 71 or is 94 that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 71 or is 94, wherein the nucleic acid molecule differs by a single nucleotide from a nucleic acid sequence selected from the group consisting of SEQ ID NOS: 2n-1, wherein n is an integer between 1 and 71 or is 94.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94, wherein the nucleic acid molecule comprises a nucleotide sequence selected from the group consisting of the nucleotide sequence selected from the group consisting of SEQ ID NO:2n-1, wherein n is an integer between 1 and 71; a nucleotide sequence wherein one or more nucleotides in the nucleotide sequence selected from the group consisting of SEQ ID NO:2n-1, wherein n is an integer between 1 and 71 is changed from that selected from the group consisting of the chosen sequence to a different nucleotide provided that no more than 15% of the nucleotides are so changed; a nucleic acid fragment of the sequence selected from the group consisting of SEQ ID NO:2n-1, wherein n is an integer between 1 and 71; and a nucleic acid fragment wherein one or more nucleotides in the nucleotide sequence selected from the group consisting of SEQ ID NO:2n-1, wherein n is an integer between 1 and 71 is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94, wherein the nucleic acid molecule hybridizes under stringent conditions to the nucleotide sequence selected from the group consisting of SEQ ID NO:2n-1, wherein n is an integer between 1 and 71, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94. This vector can have a promoter operably linked to the nucleic acid molecule. This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94 in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 71 or is 94 in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID NO | SEQ ID NO | |
|---|---|---|---|---|
| NOVX | Internal | (nucleic | (amino | |
| Assignment | Identification | acid) | acid) | Homology |
| NOV1a | CG101729-02 | 1 | 2 | Fibroblast growth factor receptor 4 - |
| | | | | Homo sapiens |
| NOV1b | SNP 13374536 | 3 | 4 | Fibroblast growth factor receptor 4 - |
| | | | | Homo sapiens |
| NOV1c | SNP 13374538 | 5 | 6 | Fibroblast growth factor receptor 4 - |
| | | | | Homo sapiens |
| NOV1d | SNP 13375033 | 7 | 8 | Fibroblast growth factor receptor 4 - |
| | | | | Homo sapiens |
| NOV1e | SNP 13375034 | 9 | 10 | Fibroblast growth factor receptor 4 - |
| | | | | Homo sapiens |
| NOV1f | SNP 13375035 | 11 | 12 | Fibroblast growth factor receptor 4 - |
| | | | | Homo sapiens |
| NOV1g | SNP 13375036 | 13 | 14 | Fibroblast growth factor receptor 4 - |
| | | | | Homo sapiens |
| NOV1h | SNP 13375039 | 15 | 16 | Fibroblast growth factor receptor 4 - |
| | | | | Homo sapiens |
| NOV1i | SNP 13375041 | 17 | 18 | Fibroblast growth factor receptor 4 - |
| | | | | Homo sapiens |
| NOV1j | SNP 13375042 | 19 | 20 | Fibroblast growth factor receptor 4 - |
| | | | | Homo sapiens |
| NOV1k | SNP 13375043 | 21 | 22 | Fibroblast growth factor receptor 4 - |
| | | | | Homo sapiens |
| NOV1l | SNP 13375045 | 23 | 24 | Fibroblast growth factor receptor 4 - |
| | | | | Homo sapiens |
| NOV1m | SNP 13375046 | 25 | 26 | Fibroblast growth factor receptor 4 - |
| | | | | Homo sapiens |
| NOV1n | SNP 13375047 | 27 | 28 | Fibroblast growth factor receptor 4 - |
| | | | | Homo sapiens |
| NOV1o | SNP 13378017 | 29 | 30 | Fibroblast growth factor receptor 4 - |
| | | | | Homo sapiens |
| NOV1p | SNP 13378286 | 31 | 32 | Fibroblast growth factor receptor 4 - |
| | | | | Homo sapiens |
| NOV1q | SNP 13379321 | 33 | 34 | Fibroblast growth factor receptor 4 - |
| | | | | Homo sapiens |
| NOV1r | SNP 13379599 | 35 | 36 | Fibroblast growth factor receptor 4 - |
| | | | | Homo sapiens |
| NOV1s | SNP 13381615 | 37 | 38 | Fibroblast growth factor receptor 4 - |
| | | | | Homo sapiens |
| NOV1t | CG101729 | 39 | 40 | Fibroblast growth factor receptor 4 - |
| | | | | Homo sapiens |
| NOV2a | CG124800-02 | 41 | 42 | Complement factor I precursor (EC |
| | | | | 3.4.21.45) (C3B/C4B inactivator) - |
| | | | | Homo sapiens |
| NOV3a | CG185793-02 | 43 | 44 | Matrix metalloproteinase-15 |
| | | | | precursor (EC 3.4.24.-) (MMP-15) - |
| | | | | Homo sapiens |
| NOV4a | CG186317-02 | 45 | 46 | MDC3 (ADAM22 protein) - Homo |
| | | | | sapiens |
| NOV5a | CG192920-02 | 47 | 48 | T-lymphocyte surface antigen Ly-9 |
| | | | | precursor (Lymphocyte antigen 9) |
| | | | | (Cell-surface molecule Ly-9) |
| | | | | (CD229 antigen) - Homo sapiens |
| NOV5b | 314409072 | 49 | 50 | T-lymphocyte surface antigen Ly-9 |
| | | | | precursor (Lymphocyte antigen 9) |
| | | | | (Cell-surface molecule Ly-9) |
| | | | | (CD229 antigen) - Homo sapiens |
| NOV5c | CG192920 | | 188 | T-lymphocyte surface antigen Ly-9 |
| | | | | precursor (Lymphocyte antigen 9) |
| | | | | (Cell-surface molecule Ly-9) |
| | | | | (CD229 antigen) - Homo sapiens |
| NOV6a | CG54470-03 | 51 | 52 | Fibroblast growth factor-21 |
| | | | | precursor (FGF-21) - Homo sapiens |
| NOV6b | 309326568 | 53 | 54 | Fibroblast growth factor-21 |
| | | | | precursor (FGF-21) - Homo sapiens |
| NOV6c | SNP 13374914 | 55 | 56 | Fibroblast growth factor-21 |
| | | | | precursor (FGF-21) - Homo sapiens |
| NOV6d | SNP 13374915 | 57 | 58 | Fibroblast growth factor-21 |
| | | | | precursor (FGF-21 ) - Homo sapiens |
| NOV6e | SNP 13374916 | 59 | 60 | Fibroblast growth factor-21 |
| | | | | precursor (FGF-21) - Homo sapiens |
| NOV6f | SNP 13374917 | 61 | 62 | Fibroblast growth factor-21 |
| | | | | precursor (FGF-21) - Homo sapiens |
| NOV6g | SNP 13374918 | 63 | 64 | Fibroblast growth factor-21 |
| | | | | precursor (FGF-21) - Homo sapiens |
| NOV6h | SNP 13374919 | 65 | 66 | Fibroblast growth factor-21 |
| | | | | precursor (FGF-21) - Homo sapiens |
| NOV6i | SNP 13374920 | 67 | 68 | Fibroblast growth factor-21 |
| | | | | precursor (FGF-21) - Homo sapiens |
| NOV6j | SNP 13374921 | 69 | 70 | Fibroblast growth factor-21 |
| | | | | precursor (FGF-21) - Homo sapiens |
| NOV6k | SNP 13374922 | 71 | 72 | Fibroblast growth factor-21 |
| | | | | precursor (FGF-21) - Homo sapiens |
| NOV6l | SNP 13382579 | 73 | 74 | Fibroblast growth factor-21 |
| | | | | precursor (FGF-21) - Homo sapiens |
| NOV6m | CG54770-02 | 75 | 76 | Fibroblast growth factor-21 |
| | | | | precursor (FGF-21) - Homo sapiens |
| NOV7a | CG55051-02 | 77 | 78 | alpha-2 macroglobulin-like |
| | | | | polypeptide variant - Homo sapiens |
| NOV7b | SNP 13377623 | 79 | 80 | alpha-2 macroglobulin-like |
| | | | | polypeptide variant - Homo sapiens |
| NOV7c | CG55051 | 81 | 82 | alpha-2 macroglobulin-like |
| | | | | polypeptide variant - Homo sapiens |
| NOV8a | CG55060-04 | 83 | 84 | Antileukoproteinase 1 precursor |
| | | | | (ALP) - Homo sapiens |
| NOV8b | SNP 13374945 | 85 | 86 | Antileukoproteinase 1 precursor |
| | | | | (ALP) Homo sapiens |
| NOV8c | SNP 13376226 | 87 | 88 | Antileukoproteinase 1 precursor |
| | | | | (ALP) - Homo sapiens |
| NOV8d | SNP 13377692 | 89 | 90 | Antileukoproteinase 1 precursor |
| | | | | (ALP) - Homo sapiens |
| NOV8e | SNP 13378858 | 91 | 92 | Antileukoproteinase 1 precursor |
| | | | | (ALP) - Homo sapiens |
| NOV8f | SNP 13378859 | 93 | 94 | Antileukoproteinase 1 precursor |
| | | | | (ALP) - Homo sapiens |
| NOV8g | CG55060 | 95 | 96 | Antileukoproteinase 1 precursor |
| | | | | (ALP) - Homo sapiens |
| NOV9a | CG56008-01 | 97 | 98 | LIV-1 protein - human |
| NOV9b | CG56008-02 | 99 | 100 | LIV-1 protein - human |
| NOV9c | CG56008-03 | 101 | 102 | LIV-1 protein - human |
| NOV9d | CG56008-04 | 103 | 104 | LIV-1 protein - human |
| NOV9e | CG56008-05 | 105 | 106 | LIV-1 protein - human |
| NOV9f | CG56008-06 | 107 | 108 | LIV-1 protein - human |
| NOV9g | 311531751 | 109 | 110 | LIV-1 protein - human |
| NOV9h | SNP 13376562 | 111 | 112 | LIV-1 protein - human |
| NOV9i | CG56008 | 113 | 114 | LIV-1 protein - human |
| NOV10a | CG59356-01 | 115 | 116 | Nuclear hormone receptor NOR-1 |
| | | | | (Neuron-derived orphan receptor 1) |
| | | | | (Mitogen induced nuclear orphan |
| | | | | receptor) - Homo sapiens |
| NOV11a | CG59889-04 | 117 | 118 | Transmembrane protein-like |
| NOV11b | CG59889-01 | 119 | 120 | Transmembrane protein-like |
| NOV11c | CG59889-07 | 121 | 122 | Transmembrane protein-like |
| NOV11d | CG59889-09 | 123 | 124 | Transmembrane protein-like |
| NOV11e | CG59889-10 | 125 | 126 | Transmembrane protein-like |
| NOV11f | CG59889-11 | 127 | 128 | Transmembrane protein-like |
| NOV11g | CG59889-12 | 129 | 130 | Transmembrane protein-like |
| NOV11h | CG59889-13 | 131 | 132 | Transmembrane protein-like |
| NOV11i | 311979177 | 133 | 134 | Transmembrane protein-like |
| NOV11j | 314361479 | 135 | 136 | Transmembrane protein-like |
| NOV12a | CG88912-02 | 137 | 138 | Beta-neoendorphin-dynorphin |
| | | | | precursor (Proenkephalin B) |
| | | | | (Preprodynorphin) - Homo sapiens |
| NOV12b | CG88912-01 | 139 | 140 | Beta-neoendorphin-dynorphin |
| | | | | precursor (Proenkephalin B) |
| | | | | (Preprodynorphin) - Homo sapiens |
| NOV12c | 310907706 | 141 | 142 | Beta-neoendorphin-dynorphin |
| | | | | precursor (Proenkephalin B) |
| | | | | (Preprodynorphin) - Homo sapiens |

Table A indicates the homology of NOVX polypeptides to known protein families. Thus, the nucleic acids and polypeptides, antibodies and related compounds according to the invention corresponding to a NOVX as identified in column 1 of Table A are useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), vascular calcification, fibrosis, atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, osteoarthritis, rheumatoid arthritis, osteochondrodysplasia, adrenoleukodystrophy, congenital adrenal hyperplasia, prostate cancer, diabetes, metabolic disorders, neoplasm; adenocarcinoma, lymphoma, uterus cancer, fertility, glomerulonephritis, hemophilia, hypercoagulation, idiopathic thrombocytopenic purpura, immunodeficiencies, psoriasis, skin disorders, graft versus host disease, AIDS, bronchial asthma, lupus, Crohn's disease; inflammatory bowel disease, ulcerative colitis, multiple sclerosis, treatment of Albright Hereditary Ostoeodystrophy, infectious disease, anorexia, cancer-associated cachexia, cancer, neurodegenerative disorders, Alzheimers Disease, Parkinson's Disorder, immune disorders, hematopoietic disorders, and the various dyslipidemias, schizophrenia, depression, asthma, emphysema, allergies, the metabolic syndrome X and wasting disorders associated with chronic diseases and various cancers, as well as conditions such as transplantation, neuroprotection, fertility, or regeneration (in vitro and in vivo).

NOVX polypeptides of the present invention show homology to, and contain domains that are characteristic of members of such protein families. Details of the sequence relatedness and domain analysis for each NOVX are presented in Example A.

The NOVX nucleic acids and polypeptides are used to screen for molecules, which inhibit or enhance NOVX activity or function. Specifically, the nucleic acids and polypeptides according to the invention are used as targets for the identification of small molecules that modulate or inhibit associated diseases.

The NOVX nucleic acids and polypeptides are also useful for detecting and differentiating specific cell types, tissues, pathological tissues, cell activation states and the like. Details of expression analysis for each NOVX are presented in Example C. Accordingly, the NOVX nucleic acids, polypeptides, antibodies and related compounds according to the invention have diagnostic and therapeutic applications in the detection of a variety of diseases with differential expression in normal vs. diseased tissues, e.g. detection of cancer.

Additional utilities for NOVX nucleic acids and polypeptides according to the invention are disclosed herein.

NOVX Clones

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 71 or is 94; (b) a variant of a mature form of the amino acid sequence selected from the group consisting of SEQ ID NO: 2n, wherein n is an integer between 1 and 71 or is 94, wherein any amino acid in the mature form is changed to a different amino acid, provided that no more than 15%, no more than 10%, no more than 5% no more than 2% or no more than 1% of the amino acid residues in the sequence of the mature form are so changed; (c) an amino acid sequence selected from the group consisting of SEQ ID NO: 2n, wherein n is an integer between 1 and 71 or is 94; (d) a variant of the amino acid sequence selected from the group consisting of SEQ ID NO:2n, wherein n is an integer between 1 and 71 or is 94 wherein any amino acid specified in the chosen sequence is changed to a different amino acid, provided that no more than 15%, no more than 10%, no more than 5% no more than 2% or no more than 1% of the amino acid residues in the sequence are so changed; and (e) a fragment of any of (a) through (d).

In another specific embodiment, the invention includes an isolated nucleic acid molecule comprising a nucleic acid sequence encoding a NOVX polypeptide comprising an amino acid sequence selected from the group consisting of: (a) a mature form of the amino acid sequence given SEQ ID NO: 2n, wherein n is an integer between 1 and 71 or is 94; (b) a variant of a mature form of the amino acid sequence selected from the group consisting of SEQ ID NO: 2n, wherein n is an integer between 1 and 71 or is 94 wherein any amino acid in the mature form of the chosen sequence is changed to a different amino acid, provided that no more than 15%, no more than 10%, no more than 5%, no more than 2%, or no more than 1% of the amino acid residues in the sequence of the mature form are so changed; (c) the amino acid sequence selected from the group consisting of SEQ ID NO: 2n, wherein n is an integer between 1 and 71 or is 94; (d) a variant of the amino acid sequence selected from the group consisting of SEQ ID NO: 2n, wherein n is an integer between 1 and 71 or is 94, in which any amino acid specified in the chosen sequence is changed to a different amino acid, provided that no more than 15%, no more than 10%, no more than 5%, no more than 2%, or no more than 1% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 71 or is 94 or any variant of said polypeptide wherein any amino acid of the chosen sequence is changed to a different amino acid, provided that no more than 15%, no more than 10%, no more than 5%, no more than 2%, or no more than 1% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n-1, wherein n is an integer between 1 and 71; (b) a nucleotide sequence wherein one or more nucleotides in the nucleotide sequence selected from the group consisting of SEQ ID NO: 2n-1, wherein n is an integer between 1 and 71 is changed from that selected from the group consisting of the chosen sequence to a different nucleotide provided that no more than 15%, no more than 10%, no more than 5%, no more than 2%, or no more than 1% of the nucleotides are so changed; (c) a nucleic acid fragment of the sequence selected from the group consisting of SEQ ID NO: 2n-1, wherein n is an integer between 1 and 71; and (d) a nucleic acid fragment wherein one or more nucleotides in the nucleotide sequence selected from the group consisting of SEQ ID NO: 2n-1, wherein n is an integer between 1 and 71 is changed from that selected from the group consisting of the chosen sequence to a different nucleotide provided that no more than 15%, no more than 10%, no more than 5%, no more than 2%, or no more than 1% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised of double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n-1, wherein n is an integer between 1 and 71, or a complement of this nucleotide sequence, can be isolated using standard molecular biology techniques and the sequence information provided herein. Using all or a portion of the nucleic acid sequence of SEQ ID NO:2n-1, wherein n is an integer between 1 and 71, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n-1, wherein n is an integer between 1 and 71, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n-1, wherein n is an integer between 1 and 71, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n-1, wherein n is an integer between 1 and 71, is one that is sufficiently complementary to the nucleotide sequence of SEQ ID NO:2n-1, wherein n is an integer between 1 and 71, that it can hydrogen bond with few or no mismatches to the nucleotide sequence shown in SEQ ID NO:2n-1, wherein n is an integer between 1 and 71, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95%, or more identity, with a preferred identity of 80-95%, and most preferred identity of 98-99% or more, over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n-1, wherein n is an integer between 1 and 71, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TM, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n-1, wherein n is an integer between 1 and 71; or an anti-sense strand nucleotide sequence of SEQ ID NO:2n-1, wherein n is an integer between 1 and 71; or of a naturally occurring mutant of SEQ ID NO:2n-1, wherein n is an integer between 1 and 71.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene is up or down regulated or has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n-1, wherein n is an integer between 1 and 71, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Single Nucleotide Polymorphisms

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. SNPs occurring within genes may result in an alteration of the amino acid encoded by the gene at the position of the SNP. Preferred embodiments include NOV1b, NOV1c, NOV1d, NOV1e, NOV1f, NOV1g, NOV1h, NOV1i, NOV1j, NOV1k, NOV1l, NOV1m, NOV1n, NOV1o, NOV1p, NOV1q, NOV1r, NOV1s, NOV1t, NOV6c, NOV6d, NOV6e, NOV6f, NOV6g, NOV6h, NOV6i, NOV6j, NOV6k, NOV6l, NOV8b, NOV8c, NOV8d, NOV8e, NOV8f, and NOV9h.

NOVX Nucleic Acid and Polypeptide Variants

The invention further encompasses nucleic acid molecules that differ from the nucleotide sequences of SEQ ID NO:2n-1, wherein n is an integer between 1 and 71, due to degeneracy of the genetic code and thus encode the same NOVX proteins as that encoded by the nucleotide sequences of SEQ ID NO:2n-1, wherein n is an integer between 1 and 71. In another embodiment, an isolated nucleic acid molecule of the invention has a nucleotide sequence encoding a protein having an amino acid sequence of SEQ ID NO:2n, wherein n is an integer between 1 and 71 or is 94.

In addition to the human NOVX nucleotide sequences of SEQ ID NO:2n-1, wherein n is an integer between 1 and 71, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n-1, wherein n is an integer between 1 and 71, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n-1, wherein n is an integer between 1 and 71. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50C. An isolated nucleic acid molecule of the invention that hybridizes under stringent conditions to a sequence of SEQ ID NO:2n-1, wherein n is an integer between 1 and 71, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n-1, wherein n is an integer between 1 and 71, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n-1, wherein n is an integer between 1 and 71, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Proc Natl Acad Sci USA 78: 6789-6792 (1981).

Conservative Mutations

In addition to naturally-occurring allelic variants of NOVX sequences that may exist in the population, the skilled artisan will further appreciate that changes can be introduced by mutation into the nucleotide sequences of SEQ ID NO:2n-1, wherein n is an integer between 1 and 71, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 71 or is 94. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n-1, wherein n is an integer between 1 and 71, yet retain biological activity. In one embodiment, the isolated nucleic acid molecule comprises a nucleotide sequence encoding a protein, wherein the protein comprises an amino acid sequence at least about 80% homologous to SEQ ID NO:2n, wherein n is an integer between 1 and 71 or is 94; more preferably at least about 90% homologous, even more preferably at least about 95% homologous, most preferably 98-99% homologous to SEQ ID NO:2n, wherein n is an integer between 1 and 71 or is 94.

An isolated nucleic acid molecule encoding a NOVX protein homologous to the protein of SEQ ID NO:2n, wherein n is an integer between 1 and 71 or is 94, can be created by introducing one or more nucleotide substitutions, additions or deletions into the nucleotide sequence of SEQ ID NO:2n-1, wherein n is an integer between 1 and 71, such that one or more amino acid substitutions, additions or deletions are introduced into the encoded protein.

Mutations can be introduced any one of SEQ ID NO:2n-1, wherein n is an integer between 1 and 71, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n-1, wherein n is an integer between 1 and 71, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 □M) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Genes Dev., 13:3191-3197, 1999). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Genes & Dev. 15, 188-200, 2001), followed by Sep-Pak C18 cartridge (Waters, Milford, Mass., USA) purification (Biochemistry, 32:11658-11668 1993).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n-1, wherein n is an integer between 1 and 71, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 71 or is 94, or antisense nucleic acids complementary to a NOVX nucleic acid sequence of SEQ ID NO:2n-1, wherein n is an integer between 1 and 71, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N-6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an □-anomeric nucleic acid molecule. An α-anomeric nucleic acid molecule forms specific double-stranded hybrids with complementary RNA in which, contrary to the usual β-units, the strands run parallel to each other. See, e.g., Nucl. Acids Res. 15: 6625-6641 (1987). The antisense nucleic acid molecule can also comprise a 2′-o-methylribonucleotide (See, e.g., Nucl. Acids Res. 15: 6131-6148, 1987) or a chimeric RNA-DNA analogue (See, e.g., FEBS Lett. 215: 327-330, 1987).

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Nature 334: 585-591,1988) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n-1, wherein n is an integer between 1 and 71). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071; U.S. Pat. No. 5,116,742. NOVX mRNA can also be used to select a catalytic RNA having a specific ribonuclease activity from a pool of RNA molecules. See, e.g., Science 261:1411-1418 (1993).

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Anticancer Drug Des. 6: 569-84,1991; Ann. N.Y. Acad. Sci. 660: 27-36,1992; Bioassays 14: 807-15,1992.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Bioorg Med Chem 4: 5-23,1996. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Bioorg Med Chem 4: 5-23,1996; Proc. Natl. Acad. Sci. USA 93: 14670-14675, 1996.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Bioorg Med Chem 4: 5-23,1996); or as probes or primers for DNA sequence and hybridization (See, Bioorg Med Chem 4: 5-23,1996; Proc. Natl. Acad. Sci. USA 93: 14670-14675,1996).

In other embodiments, the oligonucleotide may include other appended groups such as peptides (e.g., for targeting host cell receptors in vivo), or agents facilitating transport across the cell membrane (see, e.g., Proc. Natl. Acad. Sci. U.S.A. 86: 6553-6556,1989; Proc. Natl. Acad. Sci. 84: 648-652,1987; PCT Publication No. WO88/09810) or the blood-brain barrier (see, e.g., PCT Publication No. WO 89/10134). In addition, oligonucleotides can be modified with hybridization triggered cleavage agents (see, e.g., BioTechniques 6:958-976,1988) or intercalating agents (see, e.g., Pharm. Res. 5: 539-549,1988). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide of the amino acid sequence of NOVX polypeptides whose sequences are provided in any one of SEQ ID NO:2n, wherein n is an integer between 1 and 71 or is 94. The invention also includes a mutant or variant protein any of whose residues may be changed from the corresponding residues shown in any one of SEQ ID NO:2n, wherein n is an integer between 1 and 71 or is 94, while still encoding a protein that maintains its NOVX activities and physiological functions, or a functional fragment thereof.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), preferably less than about 20% of non-NOVX proteins, more preferably less than about 10%, even more preferably less than about 5%, and most preferably less than 1-2% of non-NOVX proteins. When the NOVX protein or biologically-active portion thereof is recombinantly-produced, it is also preferably substantially free of culture medium, i.e., culture medium represents less than about 20%, preferably less than about 10%, even more preferably less than about 5%, and most preferably less than 1-2% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, preferably less than about 20%, even more preferably less than about 10% still more preferably less than about 5%, and most preferably less that 1-2% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 71 or is 94) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 71 or is 94. In other embodiments, the NOVX protein is substantially homologous to SEQ ID NO:2n, wherein n is an integer between 1 and 71 or is 94, and retains the functional activity of the protein of SEQ ID NO:2n, wherein n is an integer between 1 and 71 or is 94, yet differs in amino acid sequence due to natural allelic variation or mutagenesis, as described in detail, below. Accordingly, in another embodiment, the NOVX protein is a protein that comprises an amino acid sequence at least about 80% homologous to the amino acid sequence of SEQ ID NO:2n, wherein n is an integer between 1 and 71 or is 94, and retains the functional activity of the NOVX proteins of SEQ ID NO:2n, wherein n is an integer between 1 and 71 or is 94.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, J Mol Biol 48: 443-453,1970. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n-1, wherein n is an integer between 1 and 71.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or 1, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 71 or is 94, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Tetrahedron 39: 3,1983; Annu. Rev. Biochem. 53: 323,1984; Science 198: 1056, 1984; Nucl. Acids Res. 11: 477,1983.

Anti-NOVX Antibodies

Included in the invention are antibodies to NOVX proteins, or fragments of NOVX proteins or a derivative, fragment, analog, homolog or ortholog thereof. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an Fab expression library. Antibodies may be any of the classes IgG, IgM, IgA, IgE and IgD, and include subclasses such as IgG₁, IgG₂, and others. The light chain may be a kappa chain or a lambda chain. Reference herein to antibodies includes a reference to all such classes, subclasses and types of antibody species.

An isolated NOVX full length protein or a portion or fragment thereof, can be used as an immunogen to generate antibodies that immunospecifically bind the antigen, using standard techniques for polyclonal and monoclonal antibody preparation. An antigenic peptide fragment comprises at least 6 amino acid residues of the amino acid sequence of the full length protein, such as an amino acid sequence of SEQ ID NO:2n, wherein n is an integer between 1 and 71 or is 94, and encompasses an epitope. The antigenic peptide may comprise at least 10 amino acid residues, or at least 15, at least 20, or at least 30 amino acid residues. Epitopes may encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region and may be determined by a hydrophobicity analysis of the NOVX protein sequence. As a means for targeting antibody production, hydropathy plots showing regions of hydrophilicity and hydrophobicity may be generated by any method well known in the art (for example see Proc. Nat. Acad. Sci. USA 78: 3824-3828,1981; J. Mol. Biol. 157:105-142, 1982).

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays including radioligand binding assays or similar assays known to skilled artisans.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y.).

In another embodiment NOVX nucleic acid molecules are used directly for production of antibodies recognizing NOVX polypeptides. Antibodies can be prepared by genetic or DNA-based immunization. It has been shown that intramuscular immunization of mice with a naked DNA plasmid led to expression of reporter proteins in muscle cells (Science 247: 1465-1468, 1990) and that this technology could stimulate an immune response (Nature. 356: 152-154, 1992). The success of genetic immunization in stimulating both cellular and humoral immune responses has been widely reported (reviewed in: Annu. Rev. Immunol. 15: 617-648, 1997; Immunol. Today 19: 89-97, 1998; Annu. Rev. Immunol. 18: 927-974, 2000). Using this technology, antibodies can be generated through immunization with a cDNA sequence encoding the protein in question. Following genetic immunization, the animal's immune system is activated in response to the synthesis of the foreign protein.

The quantity of protein produced in vivo following genetic immunization is within the picogram to nanogram range, which is much lower than the amounts of protein introduced by conventional immunization protocols. Despite these low levels of protein, a very efficient immune response is achieved due to the foreign protein being expressed directly in, or is quickly taken up by antigen-presenting dendritic cells (J. Leuk. Biol. 66: 350-356, 1999; J. Exp. Med. 186: 1481-1486, 1997; Nat. Med. 2: 1122-1128, 1996). A further increase in the effectivity of genetic immunization is due to the inherent immune-enhancing properties of the DNA itself, i.e., the presence of CpG-motifs in the plasmid backbone, which activate both dendritic cells (J. Immunol. 161: 3042-3049, 1998) and B-cells (Nature 374: 546-549, 1995).

Genetic immunization and production of high affinity monoclonal antibodies has been successful in mice (Biotechniques 16: 616-620, 1994; J. Biotechnol. 51: 191-194, 1996; Hybridoma 17: 569-576, 1998; J. Virol. 72: 4541-4545, 1998; J. Immunol. 160: 1458-1465, 1998; J. Biotechnol. 73: 119-129, 1999). It has been shown that monoclonal antibodies of the mature IgG subclasses can be obtained (Hybridoma 17: 569-576, 1998) and single chain libraries can be generated from genetically immunized mice (Proc. Natl. Acad. Sci. USA 95: 669-674, 1998). It has also been shown that genetic immunization can generate antibodies in other species such as rabbits (J. Lipid. Res. 38: 2627-2632, 1997) and turkeys (J. Lipid. Res. 38: 2627-2632, 1999). Genetic immunization has been used for the production of human antibodies recognizing extracellular targets.

Humanized Antibodies

Anti NOVX antibodies can further comprise humanized or human antibodies. Humanization can be performed following methods known in the art (Nature, 321:522-525, 1986; Nature, 332:323-327, 1988; Science, 239:1534-1536, 1988; U.S. Pat. No. 5,225,539; and Curr. Op. Struct. Biol., 2:593-596, 1992).

Human Antibodies

Fully human antibodies are antibody molecules in which the entire sequence of both the light chain and the heavy chain, including the CDRs, arise from human genes. Such antibodies are termed “human antibodies”, or “fully human antibodies” herein. Human monoclonal antibodies can be prepared by methods known in the art, see Immunol Today 4: 72, 1983; In: MONOCLONAL ANTIBODIES AND CANCER THERAPY, Alan R. Liss, Inc., pp. 77-96,1985; Proc Natl Acad Sci USA 80: 2026-2030, 1983; In: MONOCLONAL ANTIBODIES AND CANCER THERAPY, Alan R. Liss, Inc., pp. 77-96, 1985; J. Mol. Biol., 227:381, 1991; J. Mol. Biol., 222:581, 1991; U.S. Pat. Nos. 5,545,807; 5,545,806; 5,569,825; 5,625,126; 5,633,425; 5,661,016; Bio/Technology 10, 779-783, 1992; Nature 368 856-859, 1994; Nature 368, 812-13, 1994; Nature Biotechnology 14, 845-51, 1996; Nature Biotechnology 14, 826, 1996; and Intern. Rev. Immunol. 13, 65-93, 1995; PCT publication WO94/02602; WO 96/33735 and WO 96/34096; U.S. Pat. Nos. 5,939,598 and 5,916,771; PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Science 246: 1275-1281, 1989) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art, see Nature, 305:537-539, 1983 and may be purified by affinity chromatography steps, also see WO 93/08829; EMBO J., 10:3655-3659, 1991. For further details of generating bispecific antibodies see, for example, Methods in Enzymology, 121:210 (1986); WO 96/27011; Science 229:81 (1985); J. Exp. Med. 175:217-225 (1992) J. Immunol. 148(5):1547-1553 (1992); “diabody” technology described in Proc. Natl. Acad. Sci. USA 90:6444-6448 (1993); and single-chain Fv (sFv) dimers in J. Immunol. 152:5368 (1994). Antibodies with more than two valencies are contemplated, see for example J. Immunol. 147:60 (1991).

Heteroconjugate Antibodies

Heteroconjugate antibodies composed of two covalently joined antibodies are also within the scope of the present invention, see for example, U.S. Pat. No. 4,676,980; WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, see for example, J. Exp Med., 176: 1191-1195, 1992; J. Immunol., 148:2918-2922, 1992;Cancer Research, 53: 2560-2565, 1993; Anti-Cancer Drug Design, 3: 219-230, 1989.

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described Science, 238: 1098, 1987. Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes prepared by methods known in the art, such as described in PNAS USA, 82: 3688, 1985; PNAS USA, 77: 4030, 1980; and U.S. Pat. Nos. 4,485,045; 4,544,545; and 5,013,556; J. Biol. Chem., 257: 286-288, 1982; J. National Cancer Inst., 81(19): 1484, 1989.

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ¹³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., PNAS USA, 90: 7889-7893, 1993. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™(injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is for example, an antibody capable of binding to an analyte protein, preferably an antibody with a detectable label. Antibodies can be polyclonal, or more preferably, monoclonal. An intact antibody, or a fragment thereof (e.g., F_{ab} or F_{(ab) 2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Gene 67: 31-40,1988, pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Gene 69:301-315, 1988) and pET 11d (GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (e.g., Nucl. Acids Res. 20: 2111-2118, 1992). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (EMBO J. 6: 229-234, 1987), pMFa (Cell 30: 933-943, 1982), pJRY88 (Gene 54: 113-123, 1987), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Mol. Cell. Biol. 3: 2156-2165, 1983) and the pVL series (Virology 170: 31-39, 1989).

In yet another embodiment, a nucleic acid of the invention is expressed in mammalian cells using a mammalian expression vector. Examples of mammalian expression vectors include pCDM8 (Nature 329: 840, 1987) and pMT2PC (EMBO J. 6:187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Genes Dev. 1: 268-277, 1987), lymphoid-specific promoters (Adv. Immunol. 43: 235-275, 1988), in particular promoters of T cell receptors (EMBO J. 8: 729-733, 1989) and immunoglobulins (Cell 33: 729-740, 1983; Cell 33: 741-748, 1983), neuron-specific promoters (e.g., the neurofilament promoter; PNAS USA 86: 5473-5477, 1989), pancreas-specific promoters (Science 230: 912-916, 1985), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Science 249: 374-379, 1990) and the α-fetoprotein promoter (Genes Dev. 3: 537-546, 1989).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals by methods known in the art, for example as described in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y; Cell 51: 503 (1987); Cell 69: 915, 1992; In: TERATOCARCINOMAS AND EMBRYONIC STEM CELLS: A PRACTICAL APPROACH, Robertson, ed. IRL, Oxford, pp. 113-152, 1987; Curr. Opin. Biotechnol. 2: 823-829, 1991; PCT International Publication Nos.: WO 90/11354; WO 91/01140; WO 92/0968; and WO 93/04169; the cre/loxP recombinase system PNAS USA 89: 6232-6236, 1992; a recombinase system Science 251:1351-1355, 1991; and clones of the non-human transgenic animals described in Nature 385: 810-813, 1997.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., PNAS. USA 91: 3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Anticancer Drug Design 12:145, 1997.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: PNAS U.S.A. 90:6909, 1993;PNAS U.S.A. 91:11422, 1994; J. Med. Chem. 37:2678, 1994; Science 261:1303, 1993; Angew. Chem. Int Ed. Engl. 33:2059, 1994; Angew. Chem. Int Ed. Engl. 33: 2061, 1994; and J. Med. Chem. 37:1233, 1994.

Libraries of compounds may be presented in solution (e.g., Biotechniques 13: 412-421, 1992), or on beads (Nature 354: 82-84, 1991), on chips (Nature 364: 555-556, 1993), bacteria (U.S. Pat. No. 5,223,409), spores (U.S. Pat. No. 5,233,409), plasmids (PNAS USA 89: 1865-1869, 1992) or on phage (Science 249: 386-390,1990; Science 249: 404-406, 1990; PNAS USA 87: 6378-6382,1990; J. Mol. Biol. 222: 301-310,1991; U.S. Pat. No. 5,233,409).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The ability of the test compound to bind to the NOVX protein can be detected for example, by coupling the test compound with a radioisotope (e.g. ¹²⁵I, ³⁵S, ¹⁴C, or ³H, either directly or indirectly), or enzymatic label (e.g. horseradish peroxidase, alkaline phosphatase, or luciferase) such that binding of the test compound to the NOVX protein or biologically-active portion thereof can be determined by detecting the labeled compound in a complex. In one embodiment, the assay comprises contacting a cell which expresses a NOVX protein, or a biologically-active portion thereof, with a known compound which binds NOVX to form an assay mixture, contacting the assay mixture with a test compound, and determining the ability of the test compound to interact with a NOVX protein, either compared to or in competition with the known compound.

In another embodiment, an assay is a cell-based comprising contacting a cell expressing a NOVX protein, or a biologically-active portion thereof, with a test compound and determining the ability of the test compound to modulate (e.g., stimulate or inhibit) the activity of the NOVX protein or biologically-active portion thereof. As used herein, a “target molecule” is a molecule with which a NOVX protein binds or interacts. In one embodiment, a NOVX target molecule is a component of a signal transduction pathway that facilitates transduction of an extracellular signal

Determining the ability of the NOVX protein to bind to or interact with a NOVX target molecule can be accomplished for example, by one of the methods described above or by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining directly or indirectly the ability of the test compound to bind to the NOVX protein or biologically-active portion thereof.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholam idopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates. The NOVX protein or its target molecule can be immobilized utilizing conjugation of biotin and streptavidin using techniques well-known within the art (e.g., biotinylation kit, Pierce Chemicals, Rockford, Ill.). Alternatively, antibodies reactive with NOVX protein or target molecules, but which do not interfere with binding of the NOVX protein to its target molecule, can be derivatized to the wells of the plate. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Cell 72: 223-232, 1993; J. Biol. Chem. 268: 12046-12054, 1993; Biotechniques 14: 920-924, 1993; Oncogene 8: 1693-1696, 1993; and WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome (“chromosome mapping”). Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment. See for example Science 220: 919-924 (1983). Somatic cell hybrids containing only fragments of human chromosomes can also be produced by using human chromosomes with translocations and deletions.

Fluorescence in situ hybridization (FISH) of a DNA sequence to a metaphase chromosomal spread can further be used to provide a precise chromosomal location, see, Verma, et al., HUMAN CHROMOSOMES: A MANUAL OF BASIC TECHNIQUES (Pergamon Press, New York 1988).

Once a sequence has been mapped to a precise chromosomal location, the physical position of the sequence on the chromosome can be correlated with genetic map data, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include, but are not limited to metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

An exemplary method for detecting the presence or absence of NOVX in a biological sample involves obtaining a biological sample from a test subject and contacting the biological sample with a compound or an agent capable of detecting NOVX protein or the nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA as described herein. An agent for detecting NOVX protein can be an antibody capable of binding to NOVX protein, preferably an antibody with a detectable label as described herein. In one embodiment, the biological sample contains protein molecules from the test subject. Alternatively, the biological sample can contain mRNA molecules from the test subject or genomic DNA molecules from the test subject.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and/or means for comparing the amount of NOVX in the sample with a standard. The compound or agent can be packaged in a suitable container. The kit can further comprise instructions for using the kit to detect NOVX protein or nucleic acid.

Assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity.

The methods of the invention can also be used to detect genetic lesions in a NOVX gene (characterized by at least one of an alteration affecting the integrity of a gene encoding a NOVX-protein, or the misexpression of the NOVX gene), thereby determining if a subject with the lesioned gene is at risk for a disorder characterized by aberrant cell proliferation and/or differentiation. For example, such genetic lesions can be detected by ascertaining the existence of at least one of: (i) a deletion of one or more nucleotides from a NOVX gene; (i) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Science 241: 1077-1080, 1988; and PNAS USA 91: 360-364, 1994), the latter of which can be particularly useful for detecting point mutations in the NOVX-gene (see, Nucl. Acids Res. 23: 675-682, 1995). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample

Alternative amplification methods include: self sustained sequence replication (PNAS USA 87: 1874-1878, 1990), transcriptional amplification system (PNAS USA 86: 1173-1177, 1989); Qu Replicase (BioTechnology 6: 1197, 1988), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes (e.g., Human Mutation 7: 244-255, 1996; Nat Med. 2: 753-759, 1996). For example, by two dimensional arrays containing light-generated DNA probes. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. For examples of sequencing reactions see PNAS USA 74: 560 (1977); PNAS USA 74: 5463 (1977); Biotechniques 19: 448, 1995; WO 94/16101; Adv. Chromatography 36: 127-162, 1996; and Appl. Biochem. Biotechnol. 38:147-159, 1993.

Other methods for detecting mutations in the NOVX gene include methods in which protection from cleavage agents is used to detect mismatched bases in RNA/RNA or RNA/DNA heteroduplexes see, e.g., Science 230:1242, 1985; PNAS USA 85: 4397, 1988; Methods Enzymol. 217: 286-295, 1992.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs, see Carcinogenesis 15:1657-1662, 1994; U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids, (PNAS USA: 86: 2766, 1989; Mutat Res. 285:125-144, 1993; Genet. Anal. Tech. Appl. 9: 73-79, 1992; Trends Genet. 7: 5, 1991).

In yet another embodiment, the movement of mutant or wild-type fragments in polyacrylamide gels containing a gradient of denaturant is assayed using denaturing gradient gel electrophoresis (DGGE) e.g. Nature 313: 495, 1985; Biophys. Chem. 265: 12753, 1987. Examples of other techniques for detecting point mutations include, but are not limited to, selective oligonucleotide hybridization, selective amplification, or selective primer extension, e.g. Nature 324: 163, 1986; PNAS USA 86: 6230, 1989.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization e.g., Nucl. Acids Res. 17: 2437-2448, 1989) or at the extreme 3′-terminus of one primer where, under appropriate conditions, mismatch can prevent, or reduce polymerase extension (e.g., Tibtech. 11: 238, 1993). In addition it may be desirable to introduce a novel restriction site in the region of the mutation to create cleavage-based detection, e.g., Mol. Cell Probes 6: 1, 1992. It is anticipated that in certain embodiments amplification may also be performed using Taq ligase for amplification, e.g., PNAS. USA 88: 189, 1991. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

Pharmacogenomics, the study of the relationship between an individual's genotype and that individual's response to a foreign compound or drug permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons, e.g., Clin. Exp. Pharmacol. Physiol., 23: 983-985, 1996; Clin. Chem., 43: 254-266, 1997. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism).

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein can be monitored in clinical trails utilizing the same or similar assay. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule, e.g., identified in a screening assay as described herein) can be identified and/or quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly.

Methods of Treatment

The invention provides for both prophylactic and therapeutic methods of treating a subject at risk of (or susceptible to) a disorder or having a disorder associated with aberrant NOVX expression or activity.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (e.g., Science 244: 1288-1292, 1989); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

The invention will be further described in the following examples, which do not limit the scope of the invention described in the claims.

### EXAMPLES

### Example A

### Polynucleotide and Polypeptide Sequences, and Homology Data

### Example 1

### NOV1, CG101729: FGFR4 Variant

NOV1 of the present invention are novel proteins which bear sequence similarity to RIBOSOMAL PROTEIN S6 KINASE (RSK) ALPHA 1, nucleic acids that encode these proteins or fragments thereof, and antibodies that bind immunospecifically to these proteins. In one embodiment, a NOV1 gene encodes for a novel splice variant of ribosomal protein S6 kinase alpha 1 with 11 amino acid residues deleted resulting a shorter exon 10. Novel SNP variants are also provided.

The RSK family comprises growth factor-regulated serine/threonine kinases, known also as p90(rsk). Homologs of RSK exist in several species (Nature 384: 567-570, 1996). The highly conserved feature of all members of the RSK family is the presence of 2 nonidentical kinase catalytic domains. RSKs are implicated in the activation of the mitogen-activated kinase (MAPK) cascade and the stimulation of cell proliferation (at the transition between phases G0 and G1 of the cell cycle) and differentiation. The cloning and characterization of 3 genes encoding 3 isoforms of ribosomal protein S6 kinase (RSK): HU1 (RPS6KA1), HU2 (RPS6KA2), and HU3 (RPS6KA3) has been described (Am. J. Physiol. 266: C351-C359, 1994). The HU1 cDNA (GenBank L07597) encodes a predicted 735-amino acid protein containing 2 distinct consensus ATP-binding site sequences. Northern blot and RNase protection analyses detected an approximately 3.5-kb HU1 transcript in lymphocytes, skeletal muscle, liver, and adipose tissue. The RPS6KA1 gene has been mapped to chromosome 3.

A possible mechanism by which the RAS-MAPK signaling pathway mediates growth factor-dependent cell survival has been proposed (Science 286: 1358-1362, 1999). The MAP-activated kinases, the Rsks, catalyzed the phosphorylation of the proapoptotic protein BAD at ser 12 both in vitro and in vivo. The Rsk-induced phosphorylation of BAD at ser 12 suppressed BAD-mediated apoptosis in neurons. The Rsks are known to phosphorylate the transcription factor CREB at serl33. Activated CREB promoted cell survival, and inhibition of CREB phosphorylation at ser133 triggered apoptosis. It has been suggested that the MAP kinase signaling pathway promotes cell survival by a dual mechanism comprising the posttranslational modification and inactivation of a component of the cell death machinery and the increased transcription of prosurvival genes.

Xenopus laevis egg extracts immunodepleted of Rsk have been shown to loose their capacity to undergo mitotic arrest in response to activation of the Mos-MEK1-p42 MAPK cascade of protein kinases. Replenishing Rsk-depleted extracts with catalytically competent Rsk protein restored the ability of the extracts to undergo mitotic arrest. Rsk appears to be essential for cytostatic factor arrest (Science 286: 1362-1365, 1999). Whether cytostatic factor arrest is mediated by the protein kinase p90 Rsk, which is phosphorylated and activated by MAPK, has been investigated by expressing a constitutively activated form of Rsk in Xenopus embryos. Expression resulted in cleavage arrest. Rsk appeared to be the mediator of MAPK-dependent cytostatic factor arrest in vertebrate unfertilized eggs. Since Rsk expression did not activate the endogenous MAPK pathway, MAPK required no other substrate for induction of cytostatic factor arrest. Cytostatic factor arrest does not appear to be a consequence of direct regulation of the spindle assembly checkpoint or the anaphase-promoting complex by MAPK (Science 286: 1365-1367, 1999).

Mice deficient in S6 kinase-1 have been made (EMBO J. 17: 6649-6659, 1998) and were viable and fertile, but exhibit a conspicuous reduction in body size during embryogenesis, an effect that was mostly overcome by adulthood. Other mice deficient for S6 kinase-1, a known effector of the phosphatidylinositide-3-OH kinase signaling pathway, are hypoinsulinemic and glucose intolerant (Nature 408: 994-997, 2000). Whereas insulin resistance was not observed in isolated muscle, such mice exhibit a sharp reduction in glucose-induced insulin secretion and in pancreatic insulin content. This is not due to a lesion in glucose sensing or insulin production, but to a reduction in pancreatic endocrine mass, which is accounted for by a selective decrease in beta-cell size. It has been suggested that the observed phenotype closely parallels those of preclinical type II diabetes mellitus, in which malnutrition-induced hypoinsulinemia prediposes individuals to glucose intolerance.

The NOV1 family of novel nucleic acids and polypeptides clones includes NOV1 a through NOV1t, SEQ ID NOs: 1-40, and the nucleotide and encoded polypeptide sequences are shown in Table 1A. In a particular embodiment NOV1 nucleic acid sequence is SEQ ID NO:39, wherein each of residues X₁, X₂, X₅, X₆, X₈, X₉, X₁₀, X₁₄, X₁₇ is either C or T; and each of residues X₃, X₄, X₇, X₁₁, X₁₂, X₁₃, X₁₅, X₁₆, X₁₈ is either G or A. Nucleic acid sequence SEQ ID NO:39 encodes polypeptide SEQ ID NO:40, wherein residue Z₁ is S or F; Z₂ is C or R; Z₃ is A or T; Z₄ is Q or R; Z₅ is L or P; Z₆ is W or R; Z₇ is H or R; Z₈ is S or P; Z₉ is S or P; Z₁₀ is W or R; Z₁₁ is A or T; Z₁₂ is M or V; Z₁₃ is M or V or A; Z₁₄ is E or K; Z₁₅ is M or V; Z₁₆ is S or P; Z₁₇ is T or A; B₁ is L or S; B₂ is L or P; B₃ is K or E; B₄ is L or P; B₅ is V or D; and B₆ is L or P. Equivalent nucleic acid and polypeptide substitutions apply to other NOV1 sequences as would be appreciated by one of skill in the art, and are emcompassed in the present invention.

TABLE 1A
NOV1 Sequence Analysis

| | | |
|---|---|---|
| NOV1a, CG101729-02 | SEQ ID NO: 1 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1a, CG101729-02 | | | |
| Protein Sequence | SEQ ID NO: 2 | 789 aa | MW at 86629.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLIGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1b, SNP 13374536 | SEQ ID NO: 3 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCGTGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1b, SNP 13374536 | | | |
| Protein sequence | SEQ ID NO: 4 | 789 aa | MW at 86597.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1c, SNP 13374538 | SEQ ID NO: 5 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCGCAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAAGATGCTCAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1c, SNP 13374538 | | | |
| Protein Sequence | SEQ ID NO: 6 | 789 aa | MW at 86648.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKRIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLIGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1d, SNP 13375033 | SEQ ID NO: 7 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGCGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1d, SNP 13375033 | | | |
| Protein Sequence | SEQ ID NO: 8 | 789 aa | MW at 86599.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQRLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRPAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLTGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1e, SNP 13375034 | SEQ ID NO: 9 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTCCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1e, SNP 13375034 | | | |
| Protein Sequence | SEQ ID NO: 10 | 789 aa | MW at 86639.7kD |

MRLLLALLGVLLSVPGPPVSSLEASEEVELEPCLAPSLEQQEQELTVALGQPVRLCCGPAERGGHWYKEGSRL
APAGRVRGWRGRLEIASFLPEDAGRYLCPAPGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHTVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPAPLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLIGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1f, SNP 13375035 | SEQ ID NO: 11 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCCGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1f, SNP 13375035 | | | |
| Protein Sequence | SEQ ID NO: 12 | 789 aa | MW at 86682.7kD |

MRLLLALLGVLLSVPGPPVSSLEASEEVELEPRLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLIGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1g, SNP 13375036 | SEQ ID NO: 13 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGACACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1g, SNP 13375036 | | | |
| Protein Sequence | SEQ ID NO: 14 | 789 aa | MW at 86659.7kD |

MRLLLALLGVLLSVPGPPVSSLEASEEVELEPCLAPSLEQQEQELTVALGQPVRLCCGRAERGGHWYKEGSRL
TPAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGMDPARPDQASTVAVKMLKDNASDKDLADLVSEMEVM
KLIGRHKNIINLLGVCTQEGPLYVIVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1h, SNP 13375039 | SEQ ID NO: 15 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCGTGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1h, SNP 13375039 | | | |
| Protein sequence | SEQ ID NO: 16 | 789 aa | MW at 86597.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1i, SNP 13375041 | SEQ ID NO: 17 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCPACCTGCTTGGTGTCTGCACCCAGGAA
GGGCCCCTGTACGTGATCGTGAAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACAGTCGACGGC

| | | | |
|---|---|---|---|
| NOV1i, SNP 13375041 | | | |
| Protein Sequence | SEQ ID NO: 18 | 789 aa | MW at 86628.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1j, SNP 13375042 | SEQ ID NO: 19 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGCGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1j, SNP 13375042 | | | |
| Protein Sequence | SEQ ID NO: 20 | 789 aa | MW at 86601.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1k, SNP 13375043 | SEQ ID NO: 21 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCATGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1k, SNP 13375043 | | | |
| Protein Sequence | SEQ ID NO: 22 | 789 aa | MW at 86661.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1l, SNP 13375045 | SEQ ID NO: 23 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTCCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1l, SNP 13375045 | | | |
| Protein Sequence | SEQ ID NO: 24 | 789 aa | MW at 86639.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1m, SNP 13375046 | SEQ ID NO: 25 | 2383 bp |
| DNA Sequence | ORF Start: ATG at 17 | ORF Stop: end of sequencea |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCCGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1m, SNP 13375046 | | | |
| Protein Sequence | SEQ ID NO: 26 | 789 aa | MW at 86599.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1n, SNP 13375047 | SEQ ID NO: 27 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGACTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1n, SNP 13375047 | | | |
| Protein Sequence | SEQ ID NO: 28 | 789 aa | MW at 86659.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1o, SNP 13378017 | SEQ ID NO: 29 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGGCCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1o, SNP 13378017 | | | |
| Protein Sequence | SEQ ID NO: 30 | 789 aa | MW at 86599.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1p, SNP 13378286 | SEQ ID NO: 31 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCCGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1p, SNP 13378286 | | | |
| Protein Sequence | SEQ ID NO: 32 | 789 aa | MW at 86613.6kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1q, SNP 13379321 | SEQ ID NO: 33 | 2383 bp |
| DNA Sequence | ORF Start: ATG at 1 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCCCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1q, SNP 13379321 | | | |
| Protein Sequence | SEQ ID NO: 34 | 789 aa | MW at 86639.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1r, SNP 13379599 | SEQ ID NO: 35 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTCCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCGGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1r, SNP 13379599 | | | |
| Protein Sequence | SEQ ID NO: 36 | 789 aa | MW at 86657.7kD |

MRLLLALLGVLLSVPGPPVSSLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1s, SNP 13381615 | SEQ ID NO: 37 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTTCCTGGAGGCCTCTGAGGAAGTGGAGCTTGAGCCCTGCCTGGCTCCCAGCCTGGAGCAGCAAGAGCAGG
AGCTGACAGTAGCCCTTGGGCAGCCTGTGCGGCTGTGCTGTGGGCGGGCTGAGCGTGGTGGCCACTGGTACAA
GGAGGGCAGTCGCCTGGCACCTGCTGGCCGTGTACGGGGCTGGAGGGGCCGCCTAGAGATTGCCAGCTTCCTA
CCTGAGGATGCTGGCCGCTACCTCTGCCCGGCACGAGGCTCCATGATCGTCCTGCAGAATCTCACCTTGATTA
CAGGTGACTCCTTGACCTCCAGCAACGATGATGAGGACCCCGAGTCCCATAGGGACCTCTCGAATAGGCACAG
TTACCCCCAGCAAGCACCCTACTGGACACACCCCCAGCGCATGGAGAAGAAACTGCATGCAGTACCTGCGGGG
AACACCGTCAAGTTCCGCTGTCCAGCTGCAGGCAACCCCACGCCCACCATCCGCTGGCTTAAGGATGGACAGG
CCTTTCATGGGGAGAACCGCATTGGAGGCATTCGGCTGCGCCATCAGCACTGGAGTCTCGTGATGGAGAGCGT
GGTGCCCTCGGACCGCGGCACATACACCTGCCTGGTAGAGAACGCTGTGGGCAGCATCCGTTATAACTACCTG
CTAGATGTGCTGGAGCGGTCCCCGCACCGGCCCATCCTGCAGGCCGGGCTCCCGGCCAACACCACAGCCGTGG
TGGGCAGCGACGTGGAGCTGCTGTGCAAGGTGTACAGCGATGCCCAGCCCCACATCCAGTGGCTGAAGCACAT
CGTCATCAACGGCAGCAGCTTCGGAGCCGACGGTTTCCCCTATGTGCAAGTCCTAAAGACTGCAGACATCAAT
AGCTCAGAGGTGGAGGTCCTGTACCTGCGGAACGTGTCAGCCGAGGACGCAGGCGAGTACACCTGCCTCGCAG
GCAATTCCATCGGCCTCTCCTACCAGTCTGCCTGGCTCACGGTGCTGCCAGTGCGAGGGCAGAGGAGGACCCC
ACATGGACCGCAGCAGCGCCCGAGGCCAGGTATACGGACATCATCCTGTACGCGTCGGGCTCCCTGGCCTTGG
CTGTGCTCCTGCTGCTGGCCGGGCTGTATCGAGGGCAGGCGCTCCACGGCCGGCACCCCCGCCCGCCCGCCAC
TGTGCAGAAGCTCTCCCGCTTCCCTCTGGCCCGACAGTTCTCCCTGGAGTCAGGCTCTTCCGGCAAGTCAAGC
TCATCCCTGGTACGAGGCGTGCGTCTCTCCTCCAGCGGCCCCGCCTTGCTCGCCGGCCTCGCTGGTGCTTGGG
AAGCCCCTAGGCGAGGGCTGCTTTGGCCAGGTAGTACGTGCAGAGGCCTTTGGCATGGACCCTGCCCGGCCTG
ACCAAGCCAGCACTGTGGCCGTCAAGATGCTCAAAGACAACGCCTCTGACAAGGACCTGGCCGACCTGGTCTC
GGAGATGGAGGTGATGAAGCTGATCGGCCGACACAAGAACATCATCAACCTGCTTGGTGTCTGCACCCAGGAA
GGGCCCCTGTACGTGATCGTGGAGTGCGCCGCCAAGGGAAACCTGCGGGAGTTCCTGCGGGCCCGGCGCCCCC
CAGGCCCCGACCTCAGCCCCGACGGTCCTCGGAGCAGTGAGGGGCCGCTCTCCTTCCCAGTCCTGGTCTCCTG
CGCCTACCAGGTGGCCCGAGGCATGCAGTATCTGGAGTCCCGGAAGTGTATCCACCGGGACCTGGCTGCCCGC
AATGTGCTGGTGACTGAGGACAATGTGATGAAGATTGCTGACTTTGGGCTGGCCCGCGGCGTCCACCACATTG
ACTACTATAAGAAAACCAGCAACGGCCGCCTGCCTGTGAAGTGGATGGCGCCCGAGGCCTTGTTTGACCGGGT
GTACACACACCAGAGTGACGTGTGGTCTTTTGGGATCCCGCTATGGGAGATCTTCACCCTCGGGGGCTCCCCG
TATCCTGGCATCCCGGTGGAGGAGCTGTTCTCGCTGCTGCGGGAGGGACATCGGATGGACCGACCCCCACACT
GCCCCCCAGAGCTGTACGGGCTGATGCGTGAGTGCTGGCACGCAGCGCCCTCCCAGAGGCCTACCTTCAAGCA
GCTGGTGGAGGCGCTGGACAAGGTCCTGCTGGCCGTCTCTGAGGAGTACCTCGACCTCCGCCTGACCTTCGGA
CCCTATTCCCCCTCTGGTGGGGACGCCAGCAGCACCTGCTCCTCCAGCGATTCTGTCTTCAGCCACGACCCCC
TGCCATTGGGATCCAGCTCCTTCCCCTTCGGGTCTGGGGTGCAGACA

| | | | |
|---|---|---|---|
| NOV1s, SNP 13381615 | | | |
| Protein Sequence | SEQ ID NO: 38 | 789 aa | MW at 86689.7kD |

MRLLLALLGVLLSVPGPPVSFLEASEEVELEPCLAPSLEQQEQELTVALGQPVRLCCGRAERGGHWYKEGSRL
APAGRVRGWRGRLEIASFLPEDAGRYLCPARGSMIVLQNLTLITGDSLTSSNDDEDPESHRDLSNRHSYPQQA
PYWTHPQRMEKKLHAVPAGNTVKFRCPAAGNPTPTIRWLKDGQAFHGENRIGGIRLRHQHWSLVMESVVPSDR
GTYTCLVENAVGSIRYNYLLDVLERSPHRPILQAGLPANTTAVVGSDVELLCKVYSDAQPHIQWLKHIVINGS
SFGADGFPYVQVLKTADINSSEVEVLYLRNVSAEDAGEYTCLAGNSIGLSYQSAWLTVLPVRGQRRTPHGPQQ
RPRPGIRTSSCTRRAPWPWLCSCCWPGCIEGRRSTAGTPARPPLCRSSPASLWPDSSPWSQALPASQAHPWYE
ACVSPPAAPPCSPASLVLGKPLGEGCFGQVVRAEAFGVDPARPDQASTVAVKMLKDNASDKDLADLVSEMEVM
KLIGRHKNIINLLGVCTQEGPLYVTVECAAKGNLREFLRARRPPGPDLSPDGPRSSEGPLSFPVLVSCAYQVA
RGMQYLESRKCIHRDLAARNVLVTEDNVMKIADFGLARGVHHIDYYKKTSNGRLPVKWMAPEALFDRVYTHQS
DVWSFGIPLWEIFTLGGSPYPGIPVEELFSLLREGHRMDRPPHCPPELYGLMRECWHAAPSQRPTFKQLVEAL
DKVLLAVSEEYLDLRLTFGPYSPSGGDASSTCSSSDSVFSHDPLPLGSSSFPFGSGVQT

| | | |
|---|---|---|
| NOV1t CG101729 | SEQ ID NO: 39 | 2383 bp |
| DNA Sequence | ORF Start: ATG at 17 | ORF Stop: end of sequence |

CACCAAGCTTCCCACCATGCGGCTGCTGCTGGCCCTGTTGGGGGTCCTGCTGAGTGTGCCTGGGCCTCCAGTC
TCGTX₁CCTGGAGGCCTCTGAGGAAGTGGAGCTTGAGCCCX₂GCCTGGCTCCCAGCCTGGAGCAGCAAGAGCA
GGAGCTGACAGTAGCCCTTGGGCAGCCTGTGCGGCTGTGCTGTGGGCGGGCTGAGCGTGGTGGCCACTGGTAC
AAGGAGGGCAGTCGCCTGX₃CACCTGCTGGCCGTGTACGGGGCTGGAGGGGCCGCCTAGAGATTGCCAGCTTC
CTACCTGAGGATGCTGGCCGCTACCTCTGCCCGGCACGAGGCTCCATGATCGTCCTGCAGAATCTCACCTTGA
TTACAGGTGACTCCTTGACCTCCAGCAACGATGATGAGGACCCCGAGTCCCATAGGGACCTCTCGAATAGGCA
CAGTTACCCCCAGCAAGCACCCTACTGGACACACCCCCAGCGCATGGAGAAGAAACTGCATGCAGTACCTGCG
GGGAACACCGTCAAGTTCCGCTGTCCAGCTGCAGGCAACCCCACGCCCACCATCCGCTGGCTTAAGGATGGAC
AGGCCTTTCATGGGGAGAACCGCATTGGAGGCATTCGGCTGCGCCATCAGCACTGGAGTCTCGTGATGGAGAG
CGTGGTGCCCTCGGACCGCGGCACATACACCTGCCTGGTAGAGAACGCTGTGGGCAGCATCCGTTATAACTAC
CTGCTAGATGTGCTGGAGCGGTCCCCGCACCGGCCCATCCTGCX₄GGCCGGGCTCCCGGCCAACACCACAGCC
GTGGTGGGCAGCGACGTGGAGCX₅GCTGTGCAAGGTGTACAGCGATGCCCAGCCCCACATCCAGX₆GGCTGAA
GCX₇CATCGTCATCAACGGCAGCAGCTTCGGAGCCGACGGTTTCCCCTATGTGCAAGTCCTAAAGACTGCAGA
CATCAATAGCTCAGAGGTGGAGGTCCTGTACCTGCGGAACGTGTCAGCCGAGGACGCAGGCGAGTACACCTGC
CTCGCAGGCAATTCCATCGGCCTCTCCTACCAGTCTGCCTGGCTCACGGTGCTGCCAGTGCGAGGGCAGAGGA
GGACCCCACATGGACCGCAGCAGCGCCCGAGGCCAGGTATACGGACATCATCCTGTACGCGTCGGGCTCCCTG
GCCTTGGCTGTGCTCCTGCTGCTGGCCGGGCTGTATCGAGGGCAGGCGCTCCACGGCCGGCACCCCCGCCCGC
CCGCCACTGTGCAGAAGCTCTCCCGCTX₈CCCTCTGGCCCGACAGTX₉CTCCCX₁₀GGAGTCAGX₁₁CTCTTC
CGGCAAGTCAAGCTCATCCCTGGTACGAGGCGTGCGTCTCTCCTCCAGCGGCCCCGCCTTGCTCGCCGGCCTC
GCTGGTGCTTGGGAAGCCCCTAGGCGAGGGCTGCTTTGGCCAGGTAGTACGTGCAGAGGCCTTTGGCX₁₂TGG
ACCCTGCCCGGCCTGACCAAGCCAGCACTGTGGCCGTCAAGATGCTCAAAGACAACGCCTCTGACAAGGACCT
GGCCGACCTGGTCTCGGAGATGGAGGTGATGAAGCTGATCGGCCGACACAAGAACATCATCAACCTGCTTGGT
GTCTGCACCCAGGAAGGGCCCCTGTACGTGATCX₁₃X₁₄GX₁₅AGTGCGCCGCCAAGGGAAACCTGCGGGAGT
TCCTGCGGGCCCGGCGCCCCCCAGGCCCCGACCTCAGCCCCGACGGTCCTCGGAGCAGTGAGGGGCCGCTCTC
CTTCCCAGTCCTGGTCTCCTGCGCCTACCAGGTGGCCCGAGGCX₁₆TGCAGTATCTGGAGTCCCGGAAGTGTA
TCCACCGGGACCTGGCTGCCCGCAATGTGCTGGTGACTGAGGACAATGTGATGAAGATTGCTGACTTTGGGCT
GGCCCGCGGCGTCCACCACATTGACTACTATAAGAAAACCAGCAACGGCCGCCTGCCTGTGAAGTGGATGGCG
CCCGAGGCCTTGTTTGACCGGGTGTACACACACCAGAGTGACGTGTGGTCTTTTGGGATCCCGCTATGGGAGA
TCTTCACCCTCGGGGGCTCCCCGTATCCTGGCATCCCGGTGGAGGAGCTGTTCTCGCTGCTGCGGGAGGGACA
TCGGATGGACCGACCCCCACACTGCCCCCCAGAGCTGTACGGGCTGATGCGTGAGTGCTGGCACGCAGCGCCC
X₁₇CCCAGAGGCCTACCTTCAAGCAGCTGGTGGAGGCGCTGGACAAGGTCCTGCTGGCCGTCTCTGAGGAGTA
CCTCGACCTCCGCCTGX₁₈CCTTCGGACCCTATTCCCCCTCTGGTGGGGACGCCAGCAGCACCTGCTCCTCCA
GCGATTCTGTCTTCAGCCACGACCCCCTGCCATTGGGATCCAGCTCCTTCCCCTTCGGGTCTGGGGTGCAGAC
A
[Wherein each of residues X₁, X₂, X₅, X₆, X₈, X₉, X₁₀, X₁₄, X₁₇ is either
C or T; and each of residues X₃, X₄, X₇, X₁₁, X₁₂, X₁₃, X₁₅, X₁₆, X₁₈ is
either G or A;]

| | | | |
|---|---|---|---|
| NOV1t, CG101729 | | | |
| Protein Sequence | SEQ ID NO: 40 | 789 aa | MW at approx 86629.6kD |

MRLLLALLGVLLSVPGPPVB₁Z₁LEASEEVELEPZ₂LAPSLEQQEQELTVALGQPVRLCCGRAERGGHWYKEG
SRLZ₃PAGRVRGWRGRLEIASFLPEDAGRYLCB₂ARGSMIVLQNLTLITGDSLTSSNDDEDPB₃SHRDB₄SNR
HSYPQQAPYWTHPQRMEKKLHAVPAGNTVKFRCPAAGNPTPTIRWLKDGQAFHGENRIGGIRLRHQHWSLVME
SVVPSDRGTYTCLVENAVGSIRYNYLLDVLERSPHRPILZ₄AGLPANTTAVVGSDVEZ₅LCKVYSDAQPHIQ
Z₆LKZ₇IVINGSSFGAB₅GFPYVQVLKTADINSSEVEVLYLRNVSAEDAGEYTCLAGNSIGLSYQSAWLTVLP
VRGQRRTPHGPQQRPRPGIRTSSCTRRAPWPWLCSCCWPGCIEGRRSTAGTPARPPLCRSSPAZ₈LWPDSZ₉P
Z₁₀SQZ₁₁LPASQAHPWYEACVSPPAAPPCSPASLVLGKPLGEGCFGQVVRAEAFGZ₁₂DPARPDQASTVAVK
MLKDNASDKDLADLVSEMEVMKLIGRHKNIINLLGVCTQEGPLYVIZ₁₃Z₁₄CAAKGNLREFLRARRPPGPDL
SPDGPRSSEGPLSFPVLVSCAYQVARGZ₁₅QYLESRKCIHRDLAARNVLVTEDNVMKIADFGLARGVHHIDYY
KKTSNGRLPVKWMAPEALFDRVYTHQSDVWSFGIPB₆WEIFTLGGSPYPGIPVEELFSLLREGHRMDRPPHCP
PELYGLMRECWHAAPZ₁₆QRPTFKQLVEALDKVLLAVSEEYLDLRLZ₁₇FGPYSPSGGDASSTCSSSDSVFSH
DPLPLGSSSFPFGSGVQT
[Wherein residue Z₁ is S or F; Z₂ is C or R; Z₃ is A or T; Z₄ is Q or R; Z₅ is L or P; Z₆ is W or R; Z₇ is
H or R; Z₈ is S or P; Z₉ is S or P; Z₁₀ is W or R; Z₁₁ is A or T; Z₁₂ is M or V; Z₁₃ is M or V or A; Z₁₄ is E
or K; Z₁₅ is M or V; Z₁₆ is S or P; Z₁₇ is T or A; B₁ is L or S; B₂ is L or P; B₃ is K or E; B₄ is L or P; B₅ is
V or D; and B₆ is L or P.]

Further analysis of the NOV1 a protein yielded the following properties shown in Table 1B.

TABLE 1B
Protein Sequence Properties NOV1a

| SignalP | Cleavage site between residues 22 and 23 | | | |
|---|---|---|---|---|
| analysis: | | | | |
| PSORT II | PSG: a new signal peptide prediction method | | | |
| analysis: | N-region: length 2; pos. chg 1; neg. chg 0 | | | |
| | H-region: length 20; peak value 10.04 | | | |
| | PSG score: 5.54 | | | |
| | GvH: von Heijne's method for signal seq. recognition | | | |
| | GvH score (threshold: −2.1): 0.32 | | | |
| | possible cleavage site: between 15 and 16 | | | |
| | >>> Seems to have a cleavable signal peptide (1 to 15) | | | |
| | ALOM: Klein et al's method for TM region allocation | | | |
| | Init position for calculation: 16 | | | |
| | Tentative number of TMS(s) for the threshold 0.5: 0 | | | |
| | number of TMS(s) . . . fixed | | | |
| | PERIPHERAL Likelihood = 3.18 (at 520) | | | |
| | ALOM score: 3.18 (number of TMSs: 0) | | | |
| | MTOP: Prediction of membrane topology (Hartmann et al.) | | | |
| | Center position for calculation: 7 | | | |
| | Charge difference: −7.0 C(−5.0)-N(2.0) | | | |
| | N >= C: N-terminal side will be inside | | | |
| | MITDISC: discrimination of mitochondrial targeting seq | | | |
| | R content: | 1 | Hyd Moment(75): | 6.09 |
| | Hyd Moment(95): | 8.95 | G content: | 2 |
| | D/E content: | 1 | S/T content: | 3 |

Score: −3.80
Gavel: prediction of cleavage sites for mitochondrial preseq
R-2 motif at 12 MRL|LL
NUCDISC: discrimination of nuclear localization signals
pat4: none
pat7: none
bipartite: none
content of basic residues: 10.0%
NLS Score: −0.47
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
55.6%: extracellular, including cell wall
22.2%: nuclear
11.1%: vacuolar
11.1%: mitochondrial
>> prediction for CG101729-02 is exc (k = 9)

A search of the NOV1a protein against the Geneseq database, a proprietary database that contains sequences published in patents and patent publication, yielded several homologous proteins shown in Table 1C.

TABLE 1C
Geneseq Results for NOV1a

| | | NOV1a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABR58627 | Human cancer related protein SEQ ID | 1 . . . 789 | 706/802 (88%) | 0.0 |
| | NO: 284 - Homo sapiens, 802 aa. | 1 . . . 802 | 719/802 (89%) | |
| | [WO2003025138-A2, 27 MAR. 2003] | | | |
| ABR58628 | Human cancer related protein SEQ ID | 1 . . . 789 | 706/789 (89%) | 0.0 |
| | NO: 285 - Homo sapiens, 762 aa. | 1 . . . 762 | 712/789 (89%) | |
| | [WO2003025138-A2, 27 MAR. 2003] | | | |
| AAE16588 | Human fibroblast growth factor receptor | 1 . . . 789 | 704/802 (87%) | 0.0 |
| | 4 (FGR4) protein - Homo sapiens, 802 | 1 . . . 802 | 717/802 (88%) | |
| | aa. [US6326472-B1, 04 DEC. 2001] | | | |
| ABB81922 | Human fibroblast growth factor receptor | 1 . . . 482 | 398/495 (80%) | 0.0 |
| | protein 4 - Homo sapiens, 495 aa. | 1 . . . 495 | 411/495 (82%) | |
| | [WO200257312-A2, 25 JUL. 2002] | | | |
| AAR26278 | Tyrosine Kinase receptor - Homo | 454 . . . 786 | 331/333 (99%) | 0.0 |
| | sapiens, 426 aa. [DE4104240-A, | 69 . . . 401 | 331/333 (99%) | |
| | 13 AUG. 1992] | | | |

In a BLAST search of public sequence databases, the NOV1a protein was found to have homology to the proteins shown in the BLASTP data in Table 1D.

TABLE 1D
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8TDA0 | Fibroblast growth factor receptor 4 - | 1 . . . 789 | 706/802 (88%) | 0.0 |
| | Homo sapiens (Human), 802 aa. | 1 . . . 802 | 719/802 (89%) | |
| E980166 | TRYSINE KINASE RECEPTOR | 1 . . . 789 | 704/803 (87%) | 0.0 |
| | PROTEIN SEQUENCE - vectors, 801 | 1 . . . 801 | 717/803 (88%) | |
| | aa. | | | |
| P22455 | Fibroblast growth factor receptor 4 | 1 . . . 789 | 705/802 (87%) | 0.0 |
| | precursor (EC 2.7.1.112) (FGFR-4) - | 1 . . . 802 | 718/802 (88%) | |
| | Homo sapiens (Human), 802 aa. | | | |
| AAF27432 | Fibroblast growth factor receptor 4, | 1 . . . 789 | 704/789 (89%) | 0.0 |
| | soluble-form splice variant - Homo | 1 . . . 762 | 710/789 (89%) | |
| | sapiens (Human), 762 aa. | | | |
| TVHUF4 | fibroblast growth factor receptor 4 | 1 . . . 789 | 704/802 (87%) | 0.0 |
| | precursor - human, 802 aa. | 1 . . . 802 | 717/802 (88%) | |

PFam analysis predicts that the NOV1 a protein contains domains as shown in the Table 1E. Specific amino acid residues of NOV1a for each domain is shown in column 2, equivalent domains in the other NOV1 proteins of the invention are also encompassed herein.

TABLE 1E
Domain Analysis of NOV1a

| | | Identities/ | |
|---|---|---|---|
| | | Similarities | |
| | NOV1a Match Region | for the | Expect |
| Pfam Domain | Amino acid residues: | Matched Region | Value |
| ig | 165 . . . 226 | 21/65 (32%) | 3.7e−09 |
| | | 49/65 (75%) | |
| ig | 264 . . . 335 | 19/75 (25%) | 9.7e−06 |
| | | 49/75 (65%) | |
| pkinase | 454 . . . 727 | 98/319 (31%) | 2.3e−86 |
| | | 235/319 (74%) | |

### Example 2

### NOV2, CG124800, Complement Facotr 1 Precursor

The present invention encompasses NOV2, a novel protein bearing sequence similarity to COMPLEMENT FACTOR I PRECURSOR, nucleic acids that encode this protein or fragments thereof, and antibodies that bind immunospecifically to NOV2.

C3 inactivator, or factor I (‘eye’), is a proteolytic enzyme that destroys the hemolytic and immune-adherence activities of cell-bound, activated C3. Patients with type I essential hypercatabolism of C3′ were homozygous for an inherited deficiency of C3 inactivator and relatives had values for the inactivator about 50% of normal (Proc. Nat. Acad. Sci. 69: 2910-2913, 1972; J. Immun. 107: 19-27, 1971; Clin. Exp. Immun. 27: 23-29,1977; Quart. J. Med. 87: 385-401, 1994). Patients had recurrent pyogenic infections, self-limiting vasculitic illness and neisserial infections. Polymorphism of C3b inactivator (“Factor I”, Nomenclature Committee of the IUIS, J. Immun. 127: 1261-1262, 1981) has been described (Hum. Genet. 71: 45-48, 1985). A variant, tentatively designated FI*C was described found as a result 305 patient sera (Hum. Genet. 82: 393, 1989). Factor I is composed of 2 disulfide-linked polypeptide chains with molecular weights of 50,000 and 38,000 daltons. It is synthesized as a single-chain precursor which undergoes intracellular proteolytic processing.

The factor I gene has been mapped to chromosome 4, specifically 4q25 (J. Biol. Chem. 262: 10065-10071, 1987),

The NOV2 clone was analyzed, and the nucleotide and encoded polypeptide sequences are shown in Table 2A.

TABLE 2A
NOV2 Sequence Analysis

| | | |
|---|---|---|
| NOV2a, CG124800-02 | SEQ ID NO: 41 | 1942 bp |
| DNA Sequence | ORF Start: ATG at 15 | ORF Stop: TAA at 1743 |

CGAACACCTCCAACATGAAGCTTCTTCATGTTTTCCTGTTATTTCTGTGCTTCCACTTAAGGTTTTGCAAGGT
CACTTATACATCTCAAGAGGATCTGGTGGAGAAAAAGTGCTTAGCAAAAAAATATACTCACCTCTCCTGCGAT
AAAGTCTTCTGCCAGCCATGGCAGAGATGCATTGAGGGCACCTGTGTTTGTAAACTACCGTATCAGTGCCCAA
AGAATGGCACTGCAGTGTGTGCAACTAACAGGAGAAGCTTCCCAACATACTGTCAACAAAAGAGTTTGGAATG
TCTTCATCCAGGGACAAAGTTTTTAAATAACGGAACATGCACAGCCGAAGGAAAGTTTAGTGTTTCCTTGAAG
CATGGAAATACAGATTCAGAGGGAATAGTTGAAGTAAAACTTGTGGACCAAGATAAGACAATGTTCATATGCA
AAAGCAGCTGGAGCATGAGGGAAGCCAACGTGGCCTGCCTTGACCTTGGGTTTCAACAAGGTGCTGATACTCA
AAGAAGGTTTAAGTTGTCTGATCTCTCTATAAATTCCACTGAATGTCTACATGTGCATTGCCGAGGATTAGAG
ACCAGTTTGGCTGAATGTACTTTTACTAAGAGAAGAACTATGGGTTACCAGGATTTCGCTGATGTGGTTTGTT
ATACACAGAAAGCAGATTCTCCAATGGATGACTTCTTTCAGTGTGTGAATGGGAAATACATTTCTCAGATGAA
AGCCTGTGATGGTATCAATGATTGTGGAGACCAAAGTGATGAACTGTGTTGTAAAGCATGCCAAGGCAAAGGC
TTCCATTGCAAATCGGGTGTTTGCATTCCAAGCCAGTATCAATGCAATGGTGAGGTGGACTGCATTACAGGGG
AAGATGAAGTTGGCTGTGCAGAAGAAACAGAAATTTTGACTGCTGACATGGATGCAGAAAGAAGACGGATAAA
ATCATTATTACCTAAACTATCTTGTGGAGTTAAAAACAGAATGCACATTCGAAGGAAACGAATTGTGGGAGGA
AAGCGAGCACAACTGGGAGACCTCCCATGGCAGGTGGCAATTAAGGATGCCAGTGGAATCACCTGTGGGGGAA
TTTATATTGGTGGCTGTTGGATTCTGACTGCTGCACATTGTCTCAGAGCCAGTAAAACTCATCGTTACCAAAT
ATGGACAACAGTAGTAGACTGGATACACCCCGACCTTAAACGTATAGTAATTGAATACGTGGATAGAATTATT
TTCCATGAAAACTACAATGCAGGCACTTACCAAAATGACATCGCTTTGATTGAAATGAAAAAAGACGGAAACA
AAAAAGATTGTGAGCTGCCTCGTTCCATCCCTGCCTGTGTCCCCTGGTCTCCTTACCTATTCCAACCTAATGA
TACATGCATCGTTTCTGGCTGGGGACGAGAAAAAGATAACGAAAGAGTCTTTTCACTTCAGTGGGGTGAAGTT
AAACTAATAAGCAACTGCTCTAAGTTTTACGGAAATCGTTTCTATGAAAAAGAAATGGAATGTGCAGGTACAT
ATGATGGTTCCATCGATGCCTGTAAAGGGGACTCTGGAGGCCCCTTAGTCTGTATGGATGCCAACAATGTGAC
TTATGTCTGGGGTGTTGTGAGTTGGGGGGAAAACTGTGGAAAACCAGAGTTCCCAGGTTTTTACACCAAAGTG
GCCAATTATTTTGACTGGATTAGCTACCATGTAGGAAGGCCTTTTATTTCTCAGTACAATGTATAAAATTGTG
ATCTCTCTCTTCATTCTATTCTTTTTCTCTCAAGAGTTCCATTTAATGGAAATAAAACGGTATAATTAATAAT
TCTCTAGGGGGGAAAAATGAAGCAAATCTCATTGGATATTTTTAAAGGTCTCCACAGAGTTTATGCCATATTG
GAATTTTGTTGTATAATTCTCAAATAAATATTTTGGTGAAGCAT

| | | | |
|---|---|---|---|
| NOV2a, GG124800-02 | | | |
| Protein Sequence | SEQ ID NO: 42 | 576 aa | MW at 65106.9kD |

MKLLHVFLLFLCFHLRFCKVTYTSQEDLVEKKCLAKKYTHLSCDKVFCQPWQRCIEGTCVCKLPYQCPKNGTA
VCATNRRSFPTYCQQKSLECLHPGTKFLNNGTCTAEGKFSVSLKHGNTDSEGIVEVKLVDQDKTMFICKSSWS
MREANVACLDLGFQQGADTQRRFKLSDLSINSTECLHVHCRGLETSLAECTFTKRRTMGYQDFADVVCYTQKA
DSPMDDFFQCVNGKYISQMKACDGINDCGDQSDELCCKACQGKGFHCKSGVCIPSQYQCNGEVDCITGEDEVG
CAEETEILTADMDAERRRIKSLLPKLSCGVKNRMHIRRKRIVGGKRAQLGDLPWQVAIKDASGITCGGIYIGG
CWILTAAHCLRASKTHRYQIWTTVVDWIHPDLKRIVIEYVDRIIFHENYNAGTYQNDIALIEMKKDGNKKDCE
LPRSIPACVPWSPYLFQPNDTCIVSGWGREKDNERVFSLQWGEVKLISNCSKFYGNRFYEKEMECAGTYDGSI
DACKGDSGGPLVCMDANNVTYVWGVVSWGENCGKPEFPGFYTKVANYFDWISYHVGRPFISQYNV

Further analysis of the NOV2a protein yielded the following properties shown in Table 2B.

TABLE 2B
Protein Sequence Properties NOV2a

| SignalP | Cleavage site between residues 19 and 20 | | | |
|---|---|---|---|---|
| analysis: | | | | |
| PSORT II | PSG: a new signal peptide prediction method | | | |
| analysis: | N-region: length 2; pos. chg 1; neg. chg 0 | | | |
| | H-region: length 13; peak value 12.61 | | | |
| | PSG score: 8.21 | | | |
| | GvH: von Heijne's method for signal seq. recognition | | | |
| | GvH score (threshold: −2.1): −2.39 | | | |
| | possible cleavage site: between 18 and 19 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 0.90 (at 356) | | | |
| | ALOM score: −1.01 (number of TMSs: 0) | | | |
| | MTOP: Prediction of membrane topology (Hartmann et al.) | | | |
| | Center position for calculation: 6 | | | |
| | Charge difference: −2.0 C(0.5)-N(2.5) | | | |
| | N >= C: N-terminal side will be inside | | | |
| | MITDISC: discrimination of mitochondrial targeting seq | | | |
| | R content: | 1 | Hyd Moment(75): | 2.70 |
| | Hyd Moment(95): | 7.92 | G content: | 0 |
| | D/E content: | 1 | S/T content: | 3 |

Score: −3.44
Gavel: prediction of cleavage sites for mitochondrial preseq
R-2 motif at 26 LRF|CK
NUCDISC: discrimination of nuclear localization signals
pat4: RRKR (5) at 329
pat7: none
bipartite: none
content of basic residues: 12.2%
NLS Score: −0.16
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
22.2%: extracellular, including cell wall
22.2%: mitochondrial
11.1%: cytoplasmic
11.1%: nuclear
11.1%: Golgi
11.1%: vacuolar
11.1%: endoplasmic reticulum
>> prediction for CG124800-02 is exc (k = 9)

A search of the NOV2a protein against the Geneseq database, a proprietary database that contains sequences published in patents and patent publication, yielded several homologous proteins shown in Table 2C.

TABLE 2C
Geneseq Results for NOV2a

| | | NOV2a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAG03718 | Human secreted protein, SEQ ID NO: | 1 . . . 109 | 108/109 (99%) | 2e−63 |
| | 7799 - Homo sapiens, 115 aa. | 1 . . . 109 | 108/109 (99%) | |
| | [EP1033401-A2, 06 SEP. 2000] | | | |
| AAE23083 | Epithin protein - Unidentified, 855 aa. | 227 . . . 567 | 114/368 (30%) | 4e−45 |
| | [WO200203787-A2, 17 JAN. 2002] | 494 . . . 854 | 170/368 (45%) | |
| ABP72376 | Transmembrane serine protease 1 | 227 . . . 567 | 116/369 (31%) | 2e−44 |
| | (MTSP1) - Homo sapiens, 855 aa. | 494 . . . 854 | 169/369 (45%) | |
| | [WO2003004681-A2, 16 JAN. 2003] | | | |
| ABP56619 | Human membrane-type serine protease | 227 . . . 567 | 116/369 (31%) | 2e−44 |
| | MTSP1 protein SEQ ID NO: 2 - Homo | 494 . . . 854 | 169/369 (45%) | |
| | sapiens, 855 aa. [WO200292841-A2, | | | |
| | 21 NOV. 2002] | | | |
| AAE29820 | Human membrane-type serine protease 1 | 227 . . . 567 | 116/369 (31%) | 2e−44 |
| | (MTSP1) - Homo sapiens, 855 aa. | 494 . . . 854 | 169/369 (45%) | |
| | [WO200277267-A2, 03 OCT. 2002] | | | |

In a BLAST search of public sequence databases, the NOV2a protein was found to have homology to the proteins shown in the BLASTP data in Table 2D.

TABLE 2D
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P05156 | Complement factor I precursor (EC | 1 . . . 576 | 575/583 (98%) | 0.0 |
| | 3.4.21.45) (C3B/C4B inactivator) - | 1 . . . 583 | 575/583 (98%) | |
| | Homo sapiens (Human), 583 aa. | | | |
| Q9WUW3 | Complement factor I precursor (EC | 1 . . . 576 | 415/605 (68%) | 0.0 |
| | 3.4.21.45) (C3B/C4B inactivator) - | 1 . . . 604 | 472/605 (77%) | |
| | Rattus norvegicus (Rat), 604 aa. | | | |
| Q61129 | Complement factor I precursor (EC | 1 . . . 576 | 408/604 (67%) | 0.0 |
| | 3.4.21.45) (C3B/C4B inactivator) - Mus | 1 . . . 603 | 467/604 (76%) | |
| | musculus (Mouse), 603 aa. | | | |
| Q8WW88 | Similar to I factor (Complement) - | 1 . . . 344 | 342/351 (97%) | 0.0 |
| | Homo sapiens (Human), 377 aa. | 1 . . . 351 | 343/351 (97%) | |
| CAA68417 | Heavy chain of factor I - Homo sapiens | 19 . . . 332 | 314/321 (97%) | 0.0 |
| | (Human), 321 aa. | 1 . . . 321 | 314/321 (97%) | |

PFam analysis predicts that the NOV2a protein contains the domains shown in the Table 2E.

TABLE 2E
Domain Analysis of NOV2a

| | | Identities/ | |
|---|---|---|---|
| | | Similarities | |
| | NOV3a Match Region | for the | Expect |
| Pfam Domain | Amino Acid residues: | Matched Region | Value |
| SRCR | 117 . . . 215 | 34/115 (30%) | 1.9e−33 |
| | | 92/115 (80%) | |
| ldl_recept_a | 220 . . . 258 | 17/43 (40%) | 8.8e−06 |
| | | 28/43 (65%) | |
| ldl_recept_a | 259 . . . 295 | 17/43 (40%) | 1.2e−11 |
| | | 29/43 (67%) | |
| trypsin | 333 . . . 562 | 95/264 (36%) | 5.2e−81 |
| | | 182/264 (69%) | |

### Example 3

### NOV3, CG185793: MMP15

The present invention encompasses NOV3, a novel protein bearing sequence similarity to MATRIX METALLOPROTEINASE-15, nucleic acids that encode this protein or fragments thereof, and antibodies that bind immunospecifically to NOV3.

Matrix metalloproteinases (MMPs) are zinc-binding endopeptidases that degrade various components of the extracellular matrix. They have been implicated in normal and pathologic processes including tissue remodeling, wound healing, angiogenesis, and tumor invasion. MMPs have different substrate specificities and are encoded by different genes. MMP15 has been isolated from a human lung cDNA library and has 73.9% sequence similarity to MMP14 (600754), a membrane-localized MMP that also contains a C-terminal transmembrane segment. MMP15-specific antibodies have detected a 72-kD protein in lung cell membranes and demonstrated by Northern blotting that MMP15 is widely expressed as a 3.6-kb transcript, particularly in liver, placenta, testis, colon, and intestine (Europ. J. Biochem. 231: 602-608, 1995). The MMP15 gene has been mapped to chromosome 6q13-q21 by isotopic in situ hybridization (Genomics 40: 168-169, 1997) but to 16q12.2-q21 by fluorescence in situ hybridization (Genomics 39: 412-413,1997).

NOV3 is a splice form of MATRIX METALLOPROTEINASE-15 as indicated by residues 94E to 191Q. This new variant contains a deletion of 154 nucleotides from coding exon 2, has the same nucleotides in exon 3 and a novel insertion of exon 4 of 133 nucleotides changing the amino acid sequence in exon 3 and 4. The NOV3 clone was analyzed, and the nucleotide and encoded polypeptide sequences are shown in Table 3A.

TABLE 3A
NOV3 Sequence Analysis

| | | |
|---|---|---|
| NOV3a, CG185793-02 | SEQ ID NO: 43 | 1674 bp |
| DNA Sequence | ORF Start: ATG at 1 | ORF Stop: TGA at 1672 |

ATGAAGCGGCCCCGCTGTGGGGTGCCAGACCAGTTCGGGGTACGAGTGAAAGCCAACCTGCGGCGGCGTCGGA
AGCGCTACGCCCTCACCGGGAGGAAGTGGAACAACCACCATCTGACCTTTAGCATCCAGAACTACACGGAGAA
GTTGGGCTGGTACCACTCGATGGAGGCGGTGCGCAGGGCCTTCCGCGTGTGGGAGCAGGCCACGCCCCTGGTC
TTCCAGGAGGTGCCCTATGAGGACATCCGGCTGCGGCGACAGAAGGAGGCCGACATCATGGAAACAACCTCTT
CCTGGTGGCAGTGCATGAGCTGGGCCACGCGCTGGGGCTGGAGCACTCCAGCAACCCCAATGCCATCATGGCG
CCGTTCTACCAGTGGAAGGACGTTGACAACTTCAAGCTGCCCGAGGACGATCTCCGTGGCATCCAGCAGCTCT
ACGCAACTTGGAAATGCAGAGTCCAAAACGCCTGAAGCCAGGGCCTGGAGCCTCTGCTGGAGCAGGCTGGCAT
CCCAAGGGGAATGTCCCCAAGGGGACATGCAGGCAGACACCCTCAGGAGCACAGTGACCCAAGGTACCCCAGA
CGGTCAGCCACAGCCTACCCAGCCTCTCCCCACTGTGACGCCACGGCGGCCAGGCCGGCCTGACCACCGGCCG
CCCCGGCCTCCCCAGCCACCACCCCCAGGTGGGAAGCCAGAGCGGCCCCCAAAGCCGGGCCCCCCAGTCCAGC
CCCGAGCCACAGAGCGGCCCGACCAGTATGGCCCCAACATCTGCGACGGGGACTTTGACACAGTGGCCATGCT
TCGCGGGGAGATGTTCGTGTTCAAGGGCCGCTGGTTCTGGCGAGTCCGGCACAACCGCGTCCTGGACAACTAT
CCCATGCCCATCGGGCACTTCTGGCGTGGTCTGCCCGGTGACATCAGTGCTGCCTACGAGCGCCAAGACGGTC
GTTTTGTCTTTTTCAAAGGTGACCGCTACTGGCTCTTTCGAGAAGCGAACCTGGAGCCCGGCTACCCACAGCC
GCTGACCAGCTATGGCCTGGGCATCCCCTATGACCGCATTGACACGGCCATCTGGTGGGAGCCCACAGGCCAC
ACCTTCTTCTTCCAAGAGGACAGGTACTGGCGCTTCAACGAGGAGACACAGCGTGGAGACCCTGGGTACCCCA
AGCCCATCAGTGTCTGGCAGGGGATCCCTGCCTCCCCTAAAGGGGCCTTCCTGAGCAATGACGCAGCCTACAC
CTACTTCTACAAGGGCACCAAATACTGGAAATTCGACAATGAGCGCCTGCGGATGGAGCCCGGCTACCCCAAG
TCCATCCTGCGGGACTTCATGGGCTGCCAGGAGCACGTGGAGCCAGGCCCCCGATGGCCCGACGTGGCCCGGC
CGCCCTTCAACCCCCACGGGGGTGCAGAGCCCGGGGCGGACAGCGCAGAGGGCGACGTGGGGGATGGGGATGG
GGACTTTGGGGCCGGGGTCAACAAGGACGGGGGCAGCCGCGTGGTGGTGCAGATGGAGGAGGTGGCACGGACG
GTGAACGTGGTGATGGTGCTGGTGCCACTGCTGCTGCTGCTCTGCGTCCTGGGCCTCACCTACGCGCTGGTGC
AGATGCAGCGCAAGGGTGCGCCACGTGTCCTGCTTTACTGCAAGCGCTCGCTGCAGGAGTGGGTCTGA

| | | | |
|---|---|---|---|
| NOV3a, CG185793-02 | | | |
| Protein Sequence | SEQ ID NO: 44 | 557 aa | MW at 63707.6kD |

MKRPRCGVPDQFGVRVKANLRRRRKRYALTGRKWNNHHLTFSIQNYTEKLGWYHSMEAVRRAFRVWEQATPLV
FQEVPYEDIRLRRQKEADIMETTSSWWQCMSWATRWGWSTPATPMPSWRRSTSGRTLTTSSCPRTISVASSSS
TQLGNAESKTPEARAWSLCWSRLASQGECPQGDMQADTLRSTVTQGTPDGQPQPTQPLPTVTPRRPGRPDHRP
PRPPQPPPPGGKPERPPKPGPPVQPRATERPDQYGPNICDGDFDTVAMLRGEMFVFKGRWFWRVRHNRVLDNY
PMPIGHFWRGLPGDISAAYERQDGRFVFFKGDRYWLFREANLEPGYPQPLTSYGLGIPYDRIDTAIWWEPTGH
TFFFQEDRYWRFNEETQRGDPGYPKPISVWQGIPASPKGAFLSNDAAYTYFYKGTKYWKFDNERLRMEPGYPK
SILRDFMGCQEHVEPGPRWPDVARPPFNPHGGAEPGADSAEGDVGDGDGDFGAGVNKDGGSRVVVQMEEVART
VNVVMVLVPLLLLLCVLGLTYALVQMQRKGAPRVLLYCKRSLQEWV

Further analysis of the NOV3a protein yielded the following properties shown in Table 3B.

TABLE 3C
Protein Sequence Properties NOV3a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 3; neg. chg 1 | | | |
| | H-region: length 4; peak value −7.16 | | | |
| | PSG score: −11.56 | | | |
| | GvH: von Heijne's method for signal seq. recognition | | | |
| | GvH score (threshold: −2.1): −12.22 | | | |
| | possible cleavage site: between 51 and 52 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −14.28 | Transmembrane 514-530 |
| | PERIPHERAL | Likelihood = | 9.65 (at 392) | |
| | ALOM score: −14.28 (number of TMSs: 1) | | | |
| | MTOP: Prediction of membrane topology (Hartmann et al.) | | | |
| | Center position for calculation: 521 | | | |
| | Charge difference: 6.0 C(5.0)-N(−1.0) | | | |
| | C > N: C- terminal side will be inside | | | |
| | >>> Single TMS is located near the C-terminus | | | |
| | >>> membrane topology: type Nt (cytoplasmic tail 1 to 513) | | | |
| | MITDISC: discrimination of mitochondrial targeting seq | | | |
| | R content: | 9 | Hyd Moment(75): | 2.84 |
| | Hyd Moment(95): | 6.43 | G content: | 3 |
| | D/E content: | 2 | S/T content: | 4 |

```
Score: 0.53
Gavel: prediction of cleavage sites for mitochondrial preseq
R-2 motif at 42 GRK|WN
NUCDISC: discrimination of nuclear localization signals
pat4: KRPR (4) at 2
pat4: RRRR (5) at 21
pat4: RRRK (5) at 22
pat4: RRKR (5) at 23
pat7: none
bipartite: none
content of basic residues: 13.5%
NLS Score: 0.72
NNCN: Reinhardt's method for Cytoplasmic/Nuclear discrimination
Prediction: cytoplasmic
Reliability: 70.6
Final Results (k = 9/23):
26.1%: nuclear
21.7%: cytoplasmic
17.4%: mitochondrial
13.0%: Golgi
  8.7%: peroxisomal
  8.7%: endoplasmic reticulum
  4.3%: vesicles of secretory system
>> prediction for CG185793-02 is nuc (k = 23)
```

A search of the NOV3a protein against the Geneseq database, a proprietary database that contains sequences published in patents and patent publication, yielded several homologous proteins shown in Table 3C.

TABLE 3C
Geneseq Results for NOV3a

| | | NOV3a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAB84617 | Amino acid sequence of matrix | 1 . . . 557 | 477/572 (83%) | 0.0 |
| | metalloproteinase-15 - Homo | 106 . . . 669 | 487/572 (84%) | |
| | sapiens, 669 aa. | | | |
| | [WO200149309-A2, 12 JUL. 2001] | | | |
| AAE10424 | Human matrix | 1 . . . 557 | 477/572 (83%) | 0.0 |
| | metalloprotinase-15 (MMP-15) | 106 . . . 669 | 487/572 (84%) | |
| | protein - Homo sapiens, 669 | | | |
| | aa. [WO200166766-A2, | | | |
| | 13 SEP. 2001] | | | |
| AAR86408 | Human matrix metalloprotease | 1 . . . 557 | 477/572 (83%) | 0.0 |
| | MMPm2 - Homo sapiens, 669 aa. | 106 . . . 669 | 487/572 (84%) | |
| | [WO9525171-A2, 21 SEP. 1995] | | | |
| AAW71851 | Mouse membrane type 2 matrix | 1 . . . 557 | 421/568 (74%) | 0.0 |
| | metalloproteinase - Mus sp, | 102 . . . 657 | 456/568 (80%) | |
| | 657 aa. [JP10210982-A, | | | |
| | 11 AUG. 1998] | | | |
| ABP41430 | Human ovarian antigen HLHCB31, | 372 . . . 557 | 182/186 (97%) | e−108 |
| | SEQ ID NO: 2562 - Homo sapiens, | 1 . . . 186 | 182/186 (97%) | |
| | 186 aa. [WO200200677-A1, | | | |
| | 03 JAN. 2002] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P51511 | Matrix metalloproteinase-15 precursor (EC | 1 . . . 557 | 477/572 (83%) | 0.0 |
| | 3.4.24.-) (MMP-15) (Membrane-type matrix | 106 . . . 669 | 487/572 (84%) | |
| | metalloproteinase 2) (MT-MMP 2) | | | |
| | (MTMMP2) (Membrane-type-2 matrix | | | |
| | metalloproteinase) (MT2-MMP) (MT2MMP) | | | |
| | (SMCP- 2) - Homo sapiens (Human), 669 | | | |
| | aa. | | | |
| AAP36651 | Homo sapiens matrix metalloproteinase 15 | 1 . . . 557 | 476/572 (83%) | 0.0 |
| | (membrane-inserted) - synthetic construct, | 1 . . . 564 | 486/572 (84%) | |
| | 565 aa (fragment). | | | |
| Q9BR96 | Matrix metalloproteinase 15 | 1 . . . 557 | 476/572 (83%) | 0.0 |
| | (Membrane-inserted) - Homo sapiens | 1 . . . 564 | 486/572 (84%) | |
| | (Human), 564 aa. | | | |
| O54732 | Matrix metalloproteinase-15 precursor (EC | 1 . . . 557 | 421/568 (74%) | 0.0 |
| | 3.4.24.-) (MMP-15) (Membrane-type matrix | 102 . . . 657 | 456/568 (80%) | |
| | metalloproteinase 2) (MT-MMP 2) | | | |
| | (MTMMP2) (Membrane-type-2 matrix | | | |
| | metalloproteinase) (MT2-MMP) (MT2MMP) - | | | |
| | Mus musculus (Mouse), 657 aa. | | | |
| CAD23883 | Sequence 3 from Patent WO0208280 - | 229 . . . 557 | 169/338 (50%) | 2e−93 |
| | Homo sapiens (Human), 582 aa. | 284 . . . 582 | 220/338 (65%) | |

PFam analysis predicts that the NOV3a protein contains the domains shown in the Table 3E.

TABLE 3E
Domain Analysis of NOV3a

| | | Identities/ | |
|---|---|---|---|
| | NOV3a Match | Similarities | |
| | Region Amino | for the | Expect |
| Pfam Domain | Acid Residues: | Matched Region | Value |
| Peptidase_M10 | 28 . . . 116 | 28/115 (24%) | 0.00094 |
| | | 59/115 (51%) | |
| hemopexin | 262 . . . 305 | 16/50 (32%) | 8.4e−14 |
| | | 36/50 (72%) | |
| hemopexin | 307 . . . 351 | 20/50 (40%) | 7.6e−14 |
| | | 36/50 (72%) | |
| hemopexin | 354 . . . 400 | 25/50 (50%) | 1e−17 |
| | | 41/50 (82%) | |
| hemopexin | 402 . . . 447 | 23/50 (46%) | 1.5e−13 |
| | | 38/50 (76%) | |

### Example 4

### NOV4, CG186317, ADAM22-like

The present invention encompasses NOV4, a novel protein bearing sequence similarity to ADAM22, nucleic acids that encode this protein or fragments thereof, and antibodies that bind immunospecifically to NOV4.

ADAM (a disintegrin and metalloproteinase) and MDC (metalloproteinase-like, disintegrin-like, and cysteine-rich) proteins, are a class of cell adhesion molecules. NOV 4 XX is a novel splice form of ADAM22 with 17 amino acids (residues 787V to 817E) different from ADAM 22. The ADAM22 gene has been mapped to chromosome 2q33 (Gene 237: 61-70, 1999). The NOV4 clone was analyzed, and the nucleotide and encoded polypeptide sequences are shown in Table 4A.

TABLE 4A
NOV4 Sequence Analysis

| | | |
|---|---|---|
| NOV4a, CG186317-02 | SEQ ID NO: 45 | 3079 bp |
| DNA Sequence | ORF Start: ATG at 53 | ORF Stop: TGA at 2549 |

TAGCCCGGCGCTCTCGCCGGCCACACGGAGCGGCGCCCGGGAGCTATGAGCCATGAAGCCGCCCGGCAGCAGC
TCGCGGCAGCCGCCCCTGGCGGGCTGCAGCCTTGCCGGCGCTTCCTGCGGCCCCCAACGCGGCCCCGCCGGCT
CGGTGCCTGCCAGCGCCCCGGCCCGCACGCCGCCCTGCCGCCTGCTTCTCGTCCTTCTCCTGCTGCCTCCGCT
CGCCGCCTCGTCCCGGCCCCGCGCCTGGGGGGCTGCTGCGCCCAGCGCTCCGCATTGGAATGAAACTGCAGAA
AAAAATTTGGGAGTCCTGGCAGATGAAGACAATACATTGCAACAGAATAGCAGCAGTAATATCAGTTACAGCA
ATGCAATGCAGAAAGAAATCACACTGCCTTCAAGACTCATATATTACATCAACCAAGACTCGGAAAGCCCTTA
TCACGTTCTTGACACAAAGGCAAGACACCAGCAAAAACATAATAAGGCTGTCCATCTGGCCCAGGCAAGCTTC
CAGATTGAAGCCTTCGGCTCCAAATTCATTCTTGACCTCATACTGAACAATGGTTTGTTGTCTTCTGATTATG
TGGAGATTCACTACGAAAATGGGAAACCACAGTACTCTAAGGGTGGAGAGCACTGTTACTACCATGGAAGCAT
CAGAGGCGTCAAAGACTCCAAGGTGGCTCTGTCAACCTGCAATGGACTTCATGGCATGTTTGAAGATGATACC
TTCGTGTATATGATAGAGCCACTAGAGCTGGTTCATGATGAGAAAAGCACAGGTCGACCACATATAATCCAGA
AAACCTTGGCAGGACAGTATTCTAAGCAAATGAAGAATCTCACTATGGAAAGAGGTGACCAGTGGCCCTTTCT
CTCTGAATTACAGTGGTTGAAAAGAAGGAAGAGAGCAGTGAATCCATCACGTGGTATATTTGAAGAAATGAAA
TATTTGGAACTTATGATTGTTAATGATCACAAAACGTATAAGAAGCATCGCTCTTCTCATGCACATACCAACA
ACTTTGCAAAGTCCGTGGTCAACCTTGTGGATTCTATTTACAAGGAGCAGCTCAACACCAGGGTTGTCCTGGT
GGCTGTAGAGACCTGGACTGAGAAGGATCAGATTGACATCACCACCAACCCTGTGCAGATGCTCCATGAGTTC
TCAAAATACCGGCAGCGCATTAAGCAGCATGCTGATGCTGTGCACCTCATCTCGCGGGTGACATTTCACTATA
AGAGAAGCAGTCTGAGTTACTTTGGAGGTGTCTGTTCTCGCACAAGAGGAGTTGGTGTGAATGAGTATGGTCT
TCCAATGGCAGTGGCACAAGTATTATCGCAGAGCCTGGCTCAAAACCTTGGAATCCAATGGGAACCTTCTAGC
AGAAAGCCAAAATGTGACTGCACAGAATCCTGGGGTGGCTGCATCATGGAGGAAACAGGGGTGTCCCATTCTC
GAAAATTTTCAAAGTGCAGCATTTTGGAGTATAGAGACTTTTTACAGAGAGGAGGTGGAGCCTGCCTTTTCAA
CAGGCCAACAAAGCTATTTGAGCCCACGGAATGTGGAAATGGATACGTGGAAGCTGGGGAGGAGTGTGATTGT
GGTTTTCATGTGGAATGCTATGGATTATGCTGTAAGAAATGTTCCCTCTCCAACGGGGCTCACTGCAGCGACG
GGCCCTGCTGTAACAATACCTCATGTCTTTTTCAGCCACGAGGGTATGAATGCCGGGATGCTGTGAACGAGTG
TGATATTACTGAATATTGTACTGGAGACTCTGGTCAGTGCCCACCAAATCTTCATAAGCAAGACGGATATGCA
TGCAATCAAAATCAGGGCCGCTGCTACAATGGCGAGTGCAAGACCAGAGACAACCAGTGTCAGTACATCTGGG
GAACAAAGGCTGCAGGGTCTGACAAGTTCTGCTATGAAAAGCTGAATACAGAAGGCACTGAGAAGGGAAACTG
CGGGAAGGATGGAGACCGGTGGATTCAGTGCAGCAAACATGATGTGTTCTGTGGATTCTTACTCTGTACCAAT
CTTACTCGAGCTCCACGTATTGGTCAACTTCAGGGTGAGATCATTCCAACTTCCTTCTACCATCAAGGCCGGG
TGATTGACTGCAGTGGTGCCCATGTAGTTTTAGATGATGATACGGATGTGGGCTATGTAGAAGATGGAACGCC
ATGTGGCCCGTCTATGATGTGTTTAGATCGGAAGTGCCTACAAATTCAAGCCCTAAATATGAGCAGCTGTCCA
CTCGATTCCAAGGGTAAAGTCTGTTCGGGCCATGGGGTGTGTAGTAATGAAGCCACCTGCATTTGTGATTTCA
CCTGGGCAGGGACAGATTGCAGTATCCGGGATCCAGTTAGGAACCTTCACCCCCCCAAGGATGAAGGACCCAA
GGTGAATATGGCCACAAGCAGGCTAATAGGGGCCGTGGCCGGCACCATTCTGGCCCTGGGGGTGATTTTTGGA
GGCACAGGGTGGGGAATAGAAATGTCAAGAAGAGAAGGTTCGATCCTACTCAGCAAGGCCCCATCTGAAATCA
GCTGCGCTGGATGGACACCGCCTTGCACTGTTGGATTCTGGGTATGACATACTCGCAGCAGTGTTACTGGAAC
TATTAAGTTTGTAAACAAAACCTTTGGGTGGTAATGACTACGGAGCTAAAGTTGGGGTGACAAGGATGGGGTA
AAAGAAAACTGTCTCTTTTGGAAATAATGTCAAAGAACACCTTTCACCACCTGTCAGTAAACGGGGGAGGGGG
CAAAAGACCATGCTATAAAAAGAACTGTTCCAGAATCTTTTTTTTCCCTAATGGACGAAGGAACAACACACAC
ACAAAAATTAAATGCAATAAAGGAATCATTAAAAAAAATAGTAAATGATTTTTTTTCCCTCAGCCTGCTGGCA
CTTAATATCTTCTAAATGATTTGGCATGATTTTTTTTTCTTTACTACCGATGACAAACTCCAGTGGCATGAAG
ATCTAATTTTCAAAAGGGTAAAAACTGCATGGCATATATACAACAAGCTAGCAAGCCAATTCTCAGCAAAACC
TGCAACAGAATTC

| | | | |
|---|---|---|---|
| NOV4a, CG186317-02 | | | |
| Protein Sequence | SEQ ID NO: 46 | 832 aa | MW at 92045.3kD |

MKPPGSSSRQPPLAGCSLAGASCGPQRGPAGSVPASAPARTPPCRLLLVLLLLPPLAASSRPRAWGAAAPSAP
HWNETAEKNLGVLADEDNTLQQNSSSNISYSNAMQKEITLPSRLIYYINQDSESPYHVLDTKARHQQKHNKAV
HLAQASFQTEAFGSKFILDLILNNGLLSSDYVEIHYENGKPQYSKGGEHCYYHGSIRGVKDSKVALSTCNGLH
GMFEDDTFVYMIEPLELVHDEKSTGRPHIIQKTLAGQYSKQMKNLTMERGDQWPFLSELQWLKRRKRAVNPSR
GIFEEMKYLELMIVNDHKTYKKHRSSHAHTNNFAKSVVNLVDSIYKEQLNTRVVLVAVETWTEKDQIDITTNP
VQMLHEFSKYRQRIKQHADAVHLISRVTFHYKRSSLSYFGGVCSRTRGVGVNEYGLPMAVAQVLSQSLAQNLG
IQWEPSSRKPKCDCTESWGGCIMEETGVSHSRKFSKCSILEYRDFLQRGGGACLFNRPTKLFEPTECGNGYVE
AGEECDCGFHVECYGLCCKKCSLSNGAHCSDGPCCNNTSCLFQPRGYECRDAVNECDITEYCTGDSGQCPPNL
HKQDGYACNQNQGRCYNGECKTRDNQCQYIWGTKAAGSDKFCYEKLNTEGTEKGNCGKDGDRWIQCSKMDVFC
GFLLCTNLTRAPRIGQLQGEIIPTSFYHQGRVIDCSGAHVVLDDDTDVGYVEDGTPCGPSMMCLDRKCLQIQA
LNMSSCPLDSKGKVCSGHGVCSNEATCICDFTWAGTDCSIRDPVRNLHPPKDEGPKVNMATSRLIGAVAGTIL
ALGVIFGGTGWGIENVKKRRFDPTQQGPI

Further analysis of the NOV4a protein yielded the following properties shown in Table 4B.

TABLE 4B
Protein Sequence Properties NOV4a

| SignalP | Cleavage site between residues 60 and 61 | | | |
|---|---|---|---|---|
| analysis: | | | | |
| PSORT II | PSG: a new signal peptide prediction method | | | |
| analysis: | N-region: length 9; pos. chg 2; neg. chg 0 | | | |
| | H-region: length 17; peak value 7.01 | | | |
| | PSG score: 2.61 | | | |
| | GvH: von Heijne's method for signal seq. recognition | | | |
| | GvH score (threshold: −2.1): 6.69 | | | |
| | possible cleavage site: between 58 and 59 | | | |
| | >>> Seems to have a cleavable signal peptide (1 to 58) | | | |
| | ALOM: Klein et al's method for TM region allocation | | | |
| | Init position for calculation: 59 | | | |
| | Tentative number of TMS(s) for the threshold 0.5: 1 | | | |
| | Number of TMS(s) for threshold 0.5: 1 | | | |
| | INTEGRAL | Likelihood = | −6.53 | Transmembrane 794-810 |
| | PERIPHERAL | Likelihood = | 3.98 (at 157) | |
| | ALOM score: −6.53 (number of TMSs: 1) | | | |
| | MTOP: Prediction of membrane topology (Hartmann et al.) | | | |
| | Center position for calculation: 29 | | | |
| | Charge difference: 1.0 C(2.0)-N(1.0) | | | |
| | C > N: C-terminal side will be inside | | | |
| | >>>Caution: Inconsistent mtop result with signal peptide | | | |
| | >>> membrane topology: type 1a (cytoplasmic tail 811 to 832) | | | |
| | MITDISC: discrimination of mitochondrial targeting seq | | | |
| | R content: | 6 | Hyd Moment(75): | 4.52 |
| | Hyd Moment(95): | 5.09 | G content: | 7 |
| | D/E content: | 1 | S/T content: | 11 |

Score: 0.13
Gavel: prediction of cleavage sites for mitochondrial preseq
R-2 motif at 73 PRA|WG
NUCDISC: discrimination of nuclear localization signals
pat4: KRRK (5) at 282
pat4: RRKR (5) at 283
pat4: KKHR (3) at 313
pat4: RKPK (4) at 446
pat4: KKRR (5) at 820
pat7: PSSRKPK (3) at 443
bipartite: none
content of basic residues: 10.9%
NLS Score: 1.16
NNCN: Reinhardt's method for Cytoplasmic/Nuclear discrimination
Prediction: nuclear
Reliability: 76.7
Final Results (k = 9/23):
44.4%: extracellular, including cell wall
22.2%: endoplasmic reticulum
22.2%: Golgi
11.1%: plasma membrane
>> prediction for CG186317-02 is exc (k = 9)

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE36169 | Human MDC3 protein - Homo sapiens, | 1 . . . 832 | 815/832 (97%) | 0.0 |
| | 832 aa. [WO2002100898-A2, | 1 . . . 832 | 822/832 (97%) | |
| | 19 DEC. 2002] | | | |
| ABU56563 | Lung cancer-associated polypeptide | 1 . . . 832 | 815/832 (97%) | 0.0 |
| | #156 - Unidentified, 832 aa. | 1 . . . 832 | 822/832 (97%) | |
| | [WO200286443-A2, 31 OCT. 2002] | | | |
| ABU56479 | Lung cancer-associated polypeptide | 1 . . . 832 | 815/832 (97%) | 0.0 |
| | #72 - Unidentified, 832 aa. | 1 . . . 832 | 822/832 (97%) | |
| | [WO200286443-A2, 31 OCT. 2002] | | | |
| AAB47778 | ADAM 23 - Homo sapiens, 832 aa. | 1 . . . 832 | 815/832 (97%) | 0.0 |
| | [WO200174857-A2, 11 OCT. 2001] | 1 . . . 832 | 822/832 (97%) | |
| AAY25120 | Human MDC3 protein - Homo sapiens, | 1 . . . 832 | 815/832 (97%) | 0.0 |
| | 832 aa. [JP11155574-A, 15 JUN. 1999] | 1 . . . 832 | 822/832 (97%) | |

In a BLAST search of public sequence databases, the NOV4a protein was found to have homology to the proteins shown in the BLASTP data in Table 4D.

TABLE 4D
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O75077 | MDC3 (ADAM22 protein) - Homo sapiens | 1 . . . 832 | 815/832 (97%) | 0.0 |
| | (Human), 832 aa. | 1 . . . 832 | 822/832 (97%) | |
| Q9R1V7 | ADAM23 - Mus musculus (Mouse), 829 aa. | 1 . . . 832 | 764/833 (91%) | 0.0 |
| | | 1 . . . 829 | 787/833 (93%) | |
| Q8CC33 | A disintegrin and metalloprotease domain | 1 . . . 692 | 637/693 (91%) | 0.0 |
| | 23 - Mus musculus (Mouse), 690 aa. | 1 . . . 689 | 653/693 (93%) | |
| AAH54536 | Adam11 protein - Mus musculus (Mouse), | 47 . . . 832 | 393/804 (48%) | 0.0 |
| | 778 aa. | 9 . . . 778 | 495/804 (60%) | |
| Q9P0K1 | ADAM 22 precursor (A disintegrin and | 107 . . . 823 | 367/742 (49%) | 0.0 |
| | metalloproteinase domain 22) | 45 . . . 767 | 485/742 (64%) | |
| | (Metalloproteinase-like, disintegrin-like, and | | | |
| | cysteine-rich protein 2) | | | |
| | (Metalloproteinase-disintegrin ADAM22-3) - | | | |
| | Homo sapiens (Human), 906 aa. | | | |

PFam analysis predicts that the NOV4a protein contains the domains shown in the Table 4E.

TABLE 4E
Domain Analysis of NOV4a

| | | Identities/ | |
|---|---|---|---|
| | NOV4a Match Region | Similarities | |
| Pfam Domain | Amino Acid Residues: | for the Matched Region | Expect Value |
| Pep_M12B_propep | 165 . . . 278 | 34/124 (27%) | 4.5e−20 |
| | | 84/124 (68%) | |
| Reprolysin | 299 . . . 496 | 70/205 (34%) | 1.4e−90 |
| | | 181/205 (88%) | |
| disintegrin | 511 . . . 586 | 41/79 (52%) | 1.9e−29 |
| | | 62/79 (78%) | |
| EB | 714 . . . 768 | 14/63 (22%) | 0.85 |
| | | 36/63 (57%) | |
| EGF | 736 . . . 768 | 11/48 (23%) | 0.31 |
| | | 23/48 (48%) | |

### Example 5

### NOV5, CG192920

The NOV5 family of novel nucleic acids and polypeptides clones includes NOV5a through NOV5c, SEQ ID NOs: 45-50 and 188, and the nucleotide and encoded polypeptide sequences are shown in Table 5A. In a particular embodiment NOV5 polypeptide is SEQ ID NO:188, wherein residue XI is present or absent and when present is RLRKPKITWSLRHSEDGICRISLTCSVED GGNTVMYTWTPLQKEAVVSQGESHLNVSWRSSENHPNLTCTASNPVSRSSHQFLSEN ICSG (corresponding to amino acid residues 319-408 of SEQ ID NO:48); X₂ is residue S or G; X₃ is residue E or K; X₄ is present or absent and when present is residue V.

Equivalent nucleic acid and polypeptide substitutions apply to other NOV5 sequences as would be appreciated by one of skill in the art, and are encompassed in the present invention.

TABLE 5A
NOV5 Sequence Analysis

| | | |
|---|---|---|
| NOV5a, CG192920-02 | SEQ ID NO: 47 | 1848 bp |
| DNA Sequence | ORF Start: ATG at 1 | ORF Stop: TAG at 1846 |

ATGGGACTAAGAGCCTCTGGAAAGGACTCAGCCCCAACAGTGGTGTCAGGGATCCTAGGGGGTTCCGTGACTC
TCCCCCTAAACATCTCAGTAGACACAGAGATTGAGAACGTCATCTGGATTGGTCCCAAAAATGCTCTTGCTTT
CGCACGTCCCAAAGAAAATGTAACCATTATGGTCAAAAGCTACCTGGGCCGACTAGACATCACCAAGTGGAGT
TACTCCCTGTGCATCAGCAATCTGACTCTGAATGATGCAGGATCCTACAAAGCCCAGATAAACCAAAGGAATT
TTGAAGTCACCACTGAGGAGGAATTCACCCTGTTCGTCTATGAGCAGCTGCAGGAGCCCCAAGTCACCATGAA
GTCTGTGAAGGTGTCTGAGAACTTCTCCTGTAACATCACTCTAATGTGCTCCGTGAAGGGGGCAGAGAAAAGT
GTTCTGTACAGCTGGACCCCAAGGGAACCCCATGCTTCTGAGTCCAATGGAGGCTCCATTCTTACCGTCTCCC
GAACACCATGTGACCCAGACCTGCCATACATCTGCACAGCCCAGAACCCCGTCAGCCAGAGAAGCTCCCTCCC
TGTCCATGTTGGGCAGTTCTGTACAGATCCAGGAGCCTCCAGAGGAGGAACAACGGGGGAGACTGTGGTAGGG
GTCCTGGGAGAGCCAGTCACCCTGCCACTTGCACTCCCAGCCTGCCGGGACACAGAGAAGGTTGTCTGGTTGT
TTAACACATCCATCATTAGCAAAGAGAGGGAAGAAGCAGCAACGGCAGATCCACTCATTAAATCCAGGGATCC
TTACAAGAACAGGGTGTGGGTCTCCAGCCAGGACTGCTCCCTGAAGATCAGCCAGCTGAAGATAGAGGACGCC
GGCCCCTACCATGCCTACGTGTGCTCAGAGGCCTCCAGCGTCACCAGCATGACACATGTCACCCTGCTCATCT
ACCGCAGGCTGAGGAAGCCCAAAATCACGTGGAGCCTCAGGCACAGTGAGGATGGCATCTGCAGGATCAGCCT
GACCTGCTCCGTGGAGGACGGGGGAAACACTGTCATGTACACATGGACCCCGCTGCAGAAGGAAGCTGTTGTG
TCCCAAGGGGAATCACACCTCAATGTCTCATGGAGAAGCAGTGAAAATCACCCCAACCTCACATGCACAGCCA
GCAACCCTGTCAGCAGGAGTTCCCACCAGTTTCTTTCTGAGAACATCTGTTCAGGACCTGAGAGAAACACAAA
GCTTTGGATTGGGTTGTTCCTGATGGTTTGCCTTCTGTGCGTTGGGATCTTCAGCTGGTGCATTTGGAAGCGA
AAAGGACGGTGTTCAGTCCCAGCCTTCTGTTCCAGCCAAGCTGAGGCCCCAGCGGATACACCAGAACCCACAG
CTGGCCACACGCTATACTCTGTGCTCTCCCAAGGATATGAGAAGCTGGACACTCCCCTCAGGCCTGCCAGGCA
ACAGCCTACACCCACCTCAGACGGCAGCTCTGACAGCAACCTCACAACTGAGGAGGATGAGGACAGGCCTGAG
GTGCACAAGCCCATCAGTGGAAGATATGAGGTATTTGACCAGGTCACCCAGGAGGGCGCTGGACATGACCCAG
CCCCTGAGGGCCAAGCAGACTATGATCCCGTCACTCCATATGTCACGGAAGTTGAGTCTGTGGTTGGAGAGAA
CACCATGTATGCACAAGTGTTCAACTTACAGGGAAAGACCCCAGTTTCTCAGAAGGAAGAGAGCTCAGCCACA
ATCTACTGCTCCATACGGAAACCTCAGGTGGTGCCACCACCACAACAGAATGATCTTGAGATTCCTGAAAGTC
CTACCTATGAAAATTTCACCTAG

| | | | |
|---|---|---|---|
| NOV5a,CG192920-02 | | | |
| Protein Sequence | SEQ ID NO: 48 | 615 aa | MW at 67667.4kD |

MGLRASGKDSAPTVVSGILGGSVTLPLNISVDTEIENVIWIGPKMALAFARPKENVTIMVKSYLGRLDITKWS
YSLCISNLTLNDAGSYKAQINQRNFEVTTEEEFTLFVYEQLQEPQVTMKSVKVSENFSCNITLMCSVKGAEKS
VLYSWTPREPHASESNGGSILTVSRTPCDPDLPYICTAQNPVSQRSSLPVHVGQFCTDPGASRGGTTGETVVG
VLGEPVTLPLALPACRDTEKVVWLFNTSIISKEREEAATADPLIKSRDPYKNRVWVSSQDCSLKISQLKIEDA
GPYHAYVCSEASSVTSMTHVTLLIYRRLRKPKITWSLRHSEDGICRISLTCSVEDGGNTVMYTWTPLQKEAVV
SQGESHLNVSWRSSENHPNLTCTASNPVSRSSHQFLSENICSGPERNTKLWIGLFLMVCLLCVGIFSWCIWKR
KGRCSVPAFCSSQAEAPADTPEPTAGHTLYSVLSQGYEKLDTPLRPARQQPTPTSDGSSDSNLTTEEDEDRPE
VHKPISGRYEVFDQVTQEGAGHDPAPEGQADYDPVTPYVTEVESVVGENTMYAQVFNLQGKTPVSQKEESSAT
TYCSIRKPQVVPPPQQNDLEIPESPTYENFT

| | | |
|---|---|---|
| NOV5b, 314409072 | SEQ ID NO: 49 | 1581 bp |
| DNA Sequence | ORF Start: at 1 | ORF Stop: TAG at 1579 |

ATGGGACTAAGAGCCTCTGGAAAGGACTCAGCCCCAACAGTGGTGTCAGGGATCCTAGGGGGTTCCGTGACTC
TCCCCCTAAACATCTCAGTAGACACAGAGATTGAGAACGTCATCTGGATTGGTCCCAAAAATGCTCTTGCTTT
CGCACGTCCCAAAGAAAATGTAACCATTATGGTCAAAAGCTACCTGGGCCGACTAGACATCACCAAGTGGAGT
TACTCCCTGTGCATCAGCAATCTGACTCTGAATGATGCAGGATCCTACAAAGCCCAGATAAACCAAAGGAATT
TTGAAGTCACCACTGAGGAGGAATTCACCCTGTTCGTCTATGAGCAGCTGCAGGAGCCCCAAGTCACCATGAA
GTCTGTGAAGGTGTCTGAGAACTTCTCCTGTAACATCACTCTAATGTGCTCCGTGAAGGGGGCAGAGAAAAGT
GTTCTGTACAGCTGGACCCCAAGGGAACCCCATGCTTCTGAGTCCAATGGAGGCTCCATTCTTACCGTCTCCC
GAACACCATGTGACCCAGACCTGCCATACATCTGCACAGCCCAGAACCCCGTCAGCCAGAGAAGCTCCCTCCC
TGTCCATGTTGGGCAGTTCTGTACAGATCCAGGAGCCTCCAGAGGAGGAACAACGGGGGAGACTGTGGTAGGG
GTCCTGGGAGAGCCAGTCACCCTGCCACTTGCACTCCCAGCCTGCCGGGACACAGAGAAGGTTGTCTGGTTGT
TTAACACATCCATCATTAGCAAAGAGAGGGAAGAAGCAGCAACGGCAGATCCACTCATTAAATCCAGGGATCC
TTACAAGAACAGGGTGTGGGTCTCCAGCCAGGACTGCTCCCTGAAGATCAGCCAGCTGAAGATAGAGGACGCC
GGCCCCTACCATGCCTACGTGTGCTCAGAGGCCTCCAGCGTCACCAGCATGACACATGTCACCCTGCTCATCT
ACCGACCTGAGAGAAACACAAAGCTTTGGATTGGGTTGTTCCTGATGGTTTGCCTTCTGTGCGTTGGGATCTT
CAGCTGGTGCATTTGGAAGCGAAAAGGACGGTGTTCAGTCCCAGCCTTCTGTTCCAGCCAAGCTGAGGCCCCA
GCGGATACACCAGAACCCACAGCTGGCCACACGCTATACTCTGTGCTCTCCCAAGGATATGAGAAGCTGGACA
CTCCCCTCAGGCCTGCCAGGCAACAGCCTACACCCACCTCAGACAGCAGCTCTGACAGCAACCTCACAACTGA
GGAGGATGAGGACAGGCCTGAGGTGCACAAGCCCATCAGTGGAAGATATGAGGTATTTGACCAGGTCACTCAG
GAGGGCGCTGGACATGACCCAGCCCCTGAGGGCCAAGCAGACTATGATCCCGTCACTCCATATGTCACGGAAG
TTGAGTCTGTGGTTGGAGAGAACACCATGTATGCACAAGTGTTCAACTTACAGGGAAAGACCCCAGTTTCTCA
GGAGGAAGAGAGCTCAGCCACAATCTACTGCTCCATACGGAAACCTCAGGTGGTGGTGCCACCACCACAACAG
AATGATCTTGAGATTCCTGAAAGTCCTACCTATGAAAATTTCACCTAG

| | | | |
|---|---|---|---|
| NOV5b, 314409072 | | | |
| Protein Sequence | SEQ ID NO: 50 | 526 aa | MW at 58839.6kD |

MGLRASGKDSAPTVVSGILGGSVTLPLNISVDTEIENVIWIGPKNALAFARPKENVTIMVKSYLGRLDITKWS
YSLCISNLTLNDAGSYKAQINQRNFEVTTEEEFTLFVYEQLQEPQVTMKSVKVSENFSCNITLMCSVKGAEKS
VLYSWTPREPHASESNGGSILTVSRTPCDPDLPYICTAQNPVSQRSSLPVHVGQFCTDPGASRGGTTGETVVG
VLGEPVTLPLALPACRDTEKVVWLFNTSIISKEREEAATADPLIKSRDPYKNRVWVSSQDCSLKISQLKIEDA
GPYHAYVCSEASSVTSMTHVTLLIYRPERNTKLWIGLFLMVCLLCVGIFSWCIWKRKGRCSVPAFCSSQAEAP
ADTPEPTAGHTLYSVLSQGYEKLDTPLRPARQQPTPTSDSSSDSNLTTEEDEDRPEVHKPISGRYEVFDQVTQ
EGAGHDPAPEGQADYDPVTPYVTEVESVVGENTMYAQVFNLQGKTPVSQEEESSATIYCSIRKPQVVVPPPQQ
NDLEIPESPTYENFT

| | | | |
|---|---|---|---|
| NOV5c, CG192920 | | | MW approx |
| Protein Sequence | SEQ ID NO: 188 | 615 aa | 67667.4kD |

MGLRASGKDSAPTVVSGILGGSVTLPLNISVDTEIENVIWIGPKNALAFARPKENVTIMVKSYLGRLDITKWS
YSLCISNLTLNDAGSYKAQINQRNFEVTTEEEFTLFVYEQLQEPQVTMKSVKVSENFSCNITLMCSVKGAEKS
VLYSWTPREPHASESNGGSILTVSRTPCDPDLPYICTAQNPVSQRSSLPVHVGQFCTDPGASRGGTTGETVVG
VLGEPVTLPLALPACRDTEKVVWLFNTSIISKEREEAATADPLIKSRDPYKMRVWVSSQDCSLKISQLKIEDA
GPYHAYVCSEASSVTSMTHVTLLIYRX₁PERNTKLWIGLFLMVCLLCVGIFSWCIWKRKGRCSVPAFCSSQAE
APADTPEPTAGHTLYSVLSQGYEKLDTPLRPARQQPTPTSDX₂SSDSNLTTEEDEDRPEVHKPISGRYEVFDQ
VTQEGAGHDPAPEGQADYDPVTPYVTEVESVVGENTMYAQVFNLQGKTPVSQX₃EESSATIYCSIRKPQX₄VP
PPQQNDLEIPESPTYENFT
[Wherein X₁ is present or absent and when present is RLRKPKITWSLRHSEDGICR
ISLTCSVEDGGNTVMYTWTPLQKEAVVSQGESHLNVSWRSSENHPNLTCTASNPVSRSSHQFLSENICSG;
X₂ is residue S or G; X₃ is residue E or K; X₄ is present or absent and
when present is residue V.]

A ClustalW comparison of the above protein sequences yields the following sequence alignment shown in Table 5B.

[image]

Further analysis of the NOV5b protein yielded the following properties shown in Table5C.

TABLE 5C
Protein Sequence Properties NOV5b

| SignalP | No Known Signal Sequence Predicted | | | |
|---|---|---|---|---|
| analysis: | | | | |
| PSORT II | PSG: a new signal peptide prediction method | | | |
| analysis: | N-region: length 8; pos. chg 2; neg. chg 1 | | | |
| | H-region: length 4; peak value −0.38 | | | |
| | PSG score: −4.78 | | | |
| | GvH: von Heijne's method for signal seq. recognition | | | |
| | GvH score (threshold: −2.1): −5.83 | | | |
| | possible cleavage site: between 59 and 60 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −10.77 | Transmembrane 334-350 |
| | PERIPHERAL | Likelihood = | 0.58 (at 226) | |
| | ALOM score: −10.77 (number of TMSs: 1) | | | |
| | MTOP: Prediction of membrane topology (Hartmann et al.) | | | |
| | Center position for calculation: 341 | | | |
| | Charge difference: 1.5 C(4.0)-N(2.5) | | | |
| | C > N: C-terminal side will be inside | | | |
| | >>> membrane topology: type 1b (cytoplasmic tail 334 to 535) | | | |
| | MITDISC: discrimination of mitochondrial targeting seq | | | |
| | R content: | 3 | Hyd Moment(75): | 6.34 |
| | Hyd Moment(95): | 7.87 | G content: | 3 |
| | D/E content: | 2 | S/T content: | 2 |

Score: −4.90
Gavel: prediction of cleavage sites for mitochondrial preseq
R-2 motif at 23 LRA|SG
NUCDISC: discrimination of nuclear localization signals
pat4: none
pat7: none
bipartite: none
content of basic residues: 8.6%
NLS Score: −0.47
Dileucine motif in the tail: found
LL at 344
NNCN: Reinhardt's method for Cytplasmic/Nuclear discrimination
Prediction: nuclear
Reliability: 70.6
Psort Results (see Details):
70.0%: plasma membrane
20.0%: endoplasmic reticulum (membrane)
10.0%: mitochondrial inner membrane
0.0%: endoplasmic reticulum (lumen)

A search of the NOV5b protein against the Geneseq database, a proprietary database that contains sequences published in patents and patent publication, yielded several homologous proteins shown in Table 5D.

TABLE 5D
Geneseq Results for NOV5b

| | | NOV5b | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU74425 | Human protein sequence #3, related to | 1 . . . 601 | 321/327 (98%) | 4.8e−170 |
| | isolation of genes within SLE-1B - Homo | 10 . . . 610 | 322/327 (98%) | |
| | sapiens, 610 aa. [WO200188200-A2, | | | |
| | 22 NOV. 2001] | | | |
| ABG96270 | Human immunoglobulin superfamily | 1 . . . 525 | 510/526 (96%) | 9.7e−275 |
| | protein IGSFP-8 - Homo sapiens, 551 | 41 . . . 551 | 511/526 (97%) | |
| | aa. [WO200272794-A2, 19 SEP. 2002] | | | |
| AAU74424 | Mouse protein sequence #3, related to | 1 . . . 595 | 185/318 (58%) | 3.8e−138 |
| | isolation of genes within SLE-1B - Mus | 20 . . . 627 | 232/318 (72%) | |
| | musculus, 629 aa. [WO200188200-A2, | | | |
| | 22 NOV. 2001] | | | |

In a BLAST search of public sequence databases, the NOV5b protein was found to have homology to the proteins shown in the BLASTP data in Table 5E.

TABLE 5E
Public BLASTP Results for NOV5b

| | | NOV5b | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9HBG7 | T-lymphocyte surface antigen Ly-9 | 1 . . . 601 | 321/327 (98%) | 5.1e−170 |
| | precursor (Lymphocyte antigen 9) | 41 . . . 655 | 322/327 (98%) | |
| | (Cell-surface molecule Ly-9) (CD229 | | | |
| | antigen) - Homo sapiens (Human), 655 | | | |
| | aa. | | | |
| Q01965 | T-lymphocyte surface antigen Ly-9 | 1 . . . 601 | 186/318 (58%) | 1.7e−141 |
| | precursor (Lymphocyte antigen 9) | 41 . . . 654 | 233/318 (73%) | |
| | (Cell-surface molecule Ly-9) - Mus | | | |
| | musculus (Mouse), 654 aa. | | | |
| AAH55380 | Ly9 protein - Mus musculus (Mouse), | 1 . . . 601 | 186/318 (58%) | 2.1e−141 |
| | 649 aa (fragment). | 36 . . . 649 | 233/318 (73%) | |

PFam analysis predicts that the NOV5b protein contains the domains shown in the Table 5F. Specific amino acid residues of NOV5b for each domain is shown in column 2, equivalent domains in the other NOV5 proteins of the invention are also encompassed herein.

TABLE 5F
Domain Analysis of NOV5b

| | NOV5b Match Region | | |
|---|---|---|---|
| Pfam Domain | Amino Acid Residues: | Score | Expect Value |
| ig | 29 . . . 102 | 9.2 | 16 |
| ig | 140 . . . 193 | 12.5 | 7.2 |
| ig | 231 . . . 308 | 2.9 | 68 |

### Example 6

### NOV6, CG54470, FGF19-X

The NOV6 family of novel nucleic acids and polypeptides clones includes NOV6a through NOV6m, SEQ ID Nos: 51-76, and the nucleotide and encoded polypeptide sequences are shown in Table 6A. In a particular embodiment NOV6 nucleic acid sequence is SEQ ID NO:75, wherein each of residues X₁, X₅, X₇, is either A or G; X₂, X₃, X₄, X₆, X₈, is either C or T; and X₉, X₁₀ is either T or A. Nucleic acid sequence SEQ ID NO:75 encodes polypeptide SEQ ID NO:76, wherein residue Z₁ is T or A or I; Z₂ is V or A; Z₃ is L or P; Z₄ is Q or R; Z₅ is Q or STOP; Z₆ is R or G; Z₇ is L or P; Z₈ is L or Q; and Z₉ is L or Q. Equivalent nucleic acid and polypeptide substitutions apply to other NOV6 sequences as would be appreciated by one of skill in the art, and are emcompassed in the present invention.

TABLE 6A
NOV6 Sequence Analysis

| | | |
|---|---|---|
| NOV6a, CG54470-03 | SEQ ID NO: 51 | 375 bp |
| DNA Sequence | ORF Start: at 1 | ORF Stop: end of sequence |

CACCCCATCCCTGACTCCAGTCCTCTCCTGCAATTCGGGGGCCAAGTCCGGCAGCGGTACCTCTACACAGATG
ATGCCCAGCAGACAGAAGCCCACCTGGAGATCAGGGAGGATGGGACGGTGGGGGGCGCTGCTGACCAGAGCCC
CGAAAGTCTCCTGCAGCTGAAAGCCTTGAAGCCGGGAGTTATTCAAATCTTGGGAGTCAAGACATCCAGGTTC
CTGTGCCAGCGGCCAGATGGGGCCCTGTATGGATCGCTCCACTTTGACCCTGAGGCCTGCAGCTTCCGGGAGC
TGCTTCTTGAGGACGGATACAATGTTTACCAGTCCGAAGCCCACGGCCTCCCGCTGCACCTGCCAGGGTTACA
GAGGAGGCTC

| | | | |
|---|---|---|---|
| NOV6a, CG54470-03 | | | |
| Protein Sequence | SEQ ID NO: 52 | 125 aa | MW at 13865.5kD |

HPIPDSSPLLQFGGQVRQRYLYTDDAQQTEAHLEIREDGTVGGAADQSPESLLQLKALKPGVIQILGVKTSRF
LCQRPDGALYGSLHFDPEACSFRELLLEDGYNVYQSEAHGLPLHLPGLQRRL

| | | |
|---|---|---|
| NOV6b, 309326568 | SEQ ID NO: 53 | 549 bp |
| DNA Sequence | ORF Start: at 1 | ORF Stop: end of sequence |

CACCCCATCCCTGACTCCAGTCCTCTCCTGCAATTCGGGGGCCAAGTCCGGCAGCGGTACCTCTACACAGATG
ATGCCCAGCAGACAGAAGCCCACCTGGAGATCAGGGAGGATGGGACGGTGGGGGGCGCTGCTGACCAGAGCCC
CGAAAGTCTCCTGCAGCTGAAAGCCTTGAAGCCGGGAGTTATTCAAATCCTGGGAGTCAAGACATCCAGGTTC
CTGTGCCAGCGGCCAGATGGGGCCCTGTATGGATCGCTCCACTTTGACCCTGAGGCCTGCAGCTTCCGGGAGC
TGCTTCTTGAGGACGGATACAATGTTTACCAGTCCGAAGCCCACGGCCTCCCGCTGCACCTGCCAGGGAACAA
GTCCCCACACCGGGACCCTGCACCCCGAGGACCAGCTCGCTTCCTGCCACTACCAGGCCTGCCCCCCGCACTC
CCGGAGCCACCCGGAATCCTGGCCCCCCAGCCCCCCGATGTGGGCTCCTCGGACCCTCTGAGCATGGTGGGAT
TCCCAGGGCCGAAGCCCCAGCTACGCTTCCCTCGAGGG

| | | | |
|---|---|---|---|
| NOV6b, 309326568 | | | |
| Protein Sequence | SEQ ID NO: 54 | 183 aa | MW at 19771.4kD |

HPIPDSSPLLQFGGQVRQRYLYTDDAQQTEAHLEIREDGTVGGAADQSPESLLQLKALKPGVIQILGVKTSRF
LCQRPDGALYGSLHFDPEACSFRELLLEDGYNVYQSEAHGLPLHLPGNKSPHRDPAPRGPARFLPLPGLPPAL
PEPPGILAPQPPDVGSSDPLSMVGFPGPKPQLRFPRG

| | | |
|---|---|---|
| NOV6c, SNP 13374914 | SEQ ID NO: 55 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCCGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6c, SNP 13374914 | | | |
| Protein Sequence | SEQ ID NO: 56 | 209 aa | MW at 22283.8kD |

MDSDETGFEHSGLWVSVLAGLLLGACQAHPIPDSSPLLQFGGQVRQRYLYTDDAQQTEAHLEIREDGTVGGAA
DQSPESLLQLKALKPGVIQILGVKTSRFLCQRPDGAPYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6d, SNP 13374915 | SEQ ID NO: 57 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCGGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6d, SNP 13374915 | | | |
| Protein Sequence | SEQ ID NO: 58 | 209 aa | MW at 22200.7kD |

MDSDETGFEHSGLWVSVLAGLLLGACQAHPIPDSSPLLQFGGQVRQRYLYTDDAQQTEAHLEIREDGTVGGAA
DQSPESLLQLKALKPGVIQILGVKTSGFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6e, SNP 13374916 | SEQ ID NO: 59 | 643 bp |
| DNA Sequence | ORF Start: ATG at 9 | ORF Stop: TAA at 282 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTTAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6e, SNP 13374916 | | | |
| Protein Sequence | SEQ ID NO: 60 | 91 aa | MW at 9745.8kD |

MDSDETGFEHSGLWVSVLAGLLLGACQAHPIPDSSPLLQFGGQVRQRYLYTDDAQQTEAHLEIREDGTVGGAA
DQSPESLLQLKALKPGVI

| | | |
|---|---|---|
| NOV6f, SNP 13374917 | SEQ ID NO: 61 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCGGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6f, SNP 13374917 | | | |
| Protein Sequence | SEQ ID NO: 62 | 209 aa | MW at 22327.9kD |

MDSDETGFEHSGLWVSVLAGLLLGACQAHPIPDSSPLLQFGGQVRQRYLYTDDARQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6g, SNP 13374918 | SEQ ID NO: 63 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCCTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6g, SNP 13374918 | | | |
| Protein Sequence | SEQ ID NO: 64 | 209 aa | MW at 22283.8kD |

MDSDETGFEHSGLWVSVLAGPLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6h, SNP 13374919 | SEQ ID NO: 65 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGCGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6h, SNP 13374919 | | | |
| Protein Sequence | SEQ ID NO: 66 | 209 aa | MW at 22271.7kD |

MDSDETGFEHSGLWVSALAGLLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6i, SNP 13374920 | SEQ ID NO: 67 | 643 bp |
| DNA Sequence | ORF Start: ATG at 9 | ORF Stop: TGA at 636 |

AGCCATTGATGGACTCGGACGAGATCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6i, SNP 13374920 | | | |
| Protein Sequence | SEQ ID NO: 68 | 209 aa | MW at 22311.9kD |

MDSDEIGFEHSGLWVSVLAGLLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6j, SNP 13374921 | SEQ ID NO: 69 | 643 bp |
| DNA Sequence | ORF Start: ATG at 9 | ORF Stop: TGA at 636 |

AGCCATTGATGGACTCGGACGAGGCCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6j, SNP 13374921 | | | |
| Protein Sequence | SEQ ID NO: 70 | 209 aa | MW at 22269.8kD |

MDSDEAGFEHSGLWVSVLAGLLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGLPPALPEPPGILAPQPPDVGSSDPLSMVGPSQGRSPSYAS

| | | |
|---|---|---|
| NOV6k, SNP 13374922 | SEQ ID NO: 71 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCAGCCAGGGAACAAGTCCCCACACCGGGACCCTGCACCCCGAGGACCAGCTCGCTTCCTGCCACTACC
AGGCCTGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6k, SNP 13374922 | | | |
| Protein Sequence | SEQ ID NO: 72 | 209 aa | MW at 22314.8kD |

MDSDETGFEHSGLWVSVLAGLLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHQ
PGNKSPHRDPAPRGPARFLPLPGLPPALPEPPGILAPQPPDVGSSDPLSMVGPSQGRSPSYAS

| | | |
|---|---|---|
| NOV6l, SNP 13382579 | SEQ ID NO: 73 | 643 bp |
| DNA Sequence | ORF Start: ATG at 9 | ORF Stop: TGA at 636 |

AGCCATTGATGGACTCGGACGAGACCGGGTTCGAGCACTCAGGACTGTGGGTTTCTGTGCTGGCTGGTCTTCT
GCTGGGAGCCTGCCAGGCACACCCCATCCCTGACTCCAGTCCTCTCCTGCAATTCGGGGGCCAAGTCCGGCAG
CGGTACCTCTACACAGATGATGCCCAGCAGACAGAAGCCCACCTGGAGATCAGGGAGGATGGGACGGTGGGGG
GCGCTGCTGACCAGAGCCCCGAAAGTCTCCTGCAGCTGAAAGCCTTGAAGCCGGGAGTTATTCAAATCTTGGG
AGTCAAGACATCCAGGTTCCTGTGCCAGCGGCCAGATGGGGCCCTGTATGGATCGCTCCACTTTGACCCTGAG
GCCTGCAGCTTCCGGGAGCTGCTTCTTGAGGACGGATACAATGTTTACCAGTCCGAAGCCCACGGCCTCCCGC
TGCACCTGCCAGGGAACAAGTCCCCACACCGGGACCCTGCACCCCGAGGACCAGCTCGCTTCCTGCCACTACC
AGGCCAGCCCCCCGCACTCCCGGAGCCACCCGGAATCCTGGCCCCCCAGCCCCCCGATGTGGGCTCCTCGGAC
CCTCTGAGCATGGTGGGACCTTCCCAGGGCCGAAGCCCCAGCTACGCTTCCTGAAGCCA

| | | | |
|---|---|---|---|
| NOV6l, SNP 13382579 | | | |
| Protein Sequence | SEQ ID NO: 74 | 209 aa | MW at 22314.8kD |

MDSDETGFEHSGLWVSVLAGLLLGACQAHPIPDSSPLLQFGGQVRQRYLYTDDAQQTEAHLEIREDGTVGGAA
DQSPESLLQLKALKPGVIQILGVKTSRFLCQRPDGALYGSLHFDPEACSFRELLLEDGYNVYQSEAHGLPLHL
PGNKSPHRDPAPRGPARFLPLPGQPPALPEPPGILAPQPPDVGSSDPLSMVGPSQGRSPSYAS

| | | |
|---|---|---|
| NOV6m, CG54470 | SEQ ID NO: 75 | 643 bp |
| DNA Sequence | ORF Start: ATG at 9 | ORF Stop: TGA at 636 |

AGCCATTGATGGACTCGGACGAGX₁X₂CGGGTTCGAGCACTCAGGACTGTGGGTTTCTGX₃GCTGGCTGGTC
X₄TCTGCTGGGAGCCTGCCAGGCACACCCCATCCCTGACTCCAGTCCTCTCCTGCAATTCGGGGGCCAAGTCC
GGCAGCGGTACCTCTACACAGATGATGCCCX₅GCAGACAGAAGCCCACCTGGAGATCAGGGAGGATGGGACGG
TGGGGGGCGCTGCTGACCAGAGCCCCGAAAGTCTCCTGCAGCTGAAAGCCTTGAAGCCGGGAGTTATTX₆AAA
TCTTGGGAGTCAAGACATCCX₇GGTTCCTGTGCCAGCGGCCAGATGGGGCCCX₈GTATGGATCGCTCCACTTT
GACCCTGAGGCCTGCAGCTTCCGGGAGCTGCTTCTTGAGGACGGATACAATGTTTACCAGTCCGAAGCCCACG
GCCTCCCGCTGCACCX₉GCCAGGGAACAAGTCCCCACACCGGGACCCTGCACCCCGAGGACCAGCTCGCTTCC
TGCCACTACCAGGCCX₁₀GCCCCCCGCACTCCCGGAGCCACCCGGAATCCTGGCCCCCCAGCCCCCCGATGTG
GGCTCCTCGGACCCTCTGAGCATGGTGGGACCTTCCCAGGGCCGAAGCCCCAGCTACGCTTCCTGAAGCCA
[Wherein each of residues X₁, X₅, X₇, is either A or G; X₂, X₃, X₄, X₆,
X₈, is either C or T; and X₉, X₁₀ is either T or A.]

| | | | |
|---|---|---|---|
| NOV6m, CG54470 | | | |
| Protein Sequence | SEQ ID NO: 76 | 209 aa | MW at 22299.8kD |

MDSDEZ₁GFEHSGLWVSZ₂LAGZ₃LLGACQAHPIPDSSPLLQFGGQVRQRYLYTDDAZ₄QTEAHLEIREDGTV
GGAADQSPESLLQLKALKPGVIZ₅ILGVKTSZ₆FLCQRPDGAZ₇YGSLHFDPEACSFRELLLEDGYNVYQSEA
HGLPLHZ₈PGNKSPHRDPAPRGPARFLPLPGZ₉PPALPEPPGILAPQPPDVGSSDPLSMVGPSQGRSPSYAS
[Wherein residue Z₁ is T or A or I; Z₂ is V or A; Z₃ is L or P; Z₄ is Q
or R; Z₅ is Q or STOP; Z₆ is R or G; Z₇ is L or P; Z₈ is L or Q; and Z₉
is L or Q.]

A ClustalW comparison of the protein sequences of NOV6a through NOV61 yields the following sequence alignment shown in Table 6B.

[image]

Further analysis of the NOV6b protein yielded the following properties shown in Table 6C.

TABLE 6C
Protein Sequence Properties NOV6b

| SignalP | No signal sequence cleavage site detected | | | |
|---|---|---|---|---|
| analysis: | | | | |
| PSORT II | PSG: a new signal peptide prediction method | | | |
| analysis: | N-region: length 5; pos. chg 0; neg. chg 1 | | | |
| | H-region: length 11; peak value 0.00 | | | |
| | PSG score: −4.40 | | | |
| | GvH: von Heijne's method for signal seq. recognition | | | |
| | GvH score (threshold: −2.1): −4.96 | | | |
| | possible cleavage site: between 18 and 19 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 3.13 (at 55) | | | |
| | ALOM score: 3.13 (number of TMSs: 0) | | | |
| | MITDISC: discrimination of mitochondrial targeting seq | | | |
| | R content: | 2 | Hyd Moment(75): | 7.83 |
| | Hyd Moment(95): | 8.24 | G content: | 3 |
| | D/E content: | 2 | S/T content: | 5 |

Score: −4.65
Gavel: prediction of cleavage sites for mitochondrial preseq
R-2 motif at 29 QRY|LY
NUCDISC: discrimination of nuclear localization signals
pat4: none
pat7: none
bipartite: none
content of basic residues: 8.6%
NLS Score: −0.47
NNCN: Reinhardt's method for Cytplasmic/Nuclear discrimination
Prediction: nuclear
Reliability: 89
Psort Results (see Details):
45.0%: cytoplasm
30.0%: microbody (peroxisome)
26.8%: lysosome (lumen)
10.0%: mitochondrial matrix space

A search of the NOV6b protein against the Geneseq database, a proprietary database that contains sequences published in patents and patent publication, yielded several homologous proteins shown in Table 6D.

TABLE 6D
Geneseq Results for NOV6b

| | | NOV6b | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAE18826 | Human FGF-21 protein - Homo sapiens, | 1 . . . 167 | 167/167 (100%) | 8.9e−91 |
| | 209 aa. [US2002001825-A1, | 25 . . . 194 | 205/205 (100%) | |
| | 03 JAN. 2002] | | | |
| AAE05078 | Human fibroblast growth factor (FGF) | 1 . . . 167 | 167/167 (100%) | 8.9e−91 |
| | homologue, zFGF11 protein - Homo | 1 . . . 205 | 205/205 (100%) | |
| | sapiens, 208 aa. [2000US-0477886, | | | |
| | 05 JAN. 2000] | | | |
| AAB68417 | Amino acid sequence of human | 1 . . . 167 | 167/167 (100%) | 8.9e−91 |
| | fibroblast growth factor-21 (FGF-21) - | 1 . . . 206 | 206/206 (100%) | |
| | Homo sapiens, 209 aa. | | | |
| | [WO200136640-A2, 25 MAY 2001] | | | |
| AAG65667 | Human fibroblast growth factor (FGF)-21 - | 1 . . . 167 | 167/167 (100%) | 8.9e−91 |
| | Homo sapiens, 209 aa. | 26 . . . 206 | 206/206 (100%) | |
| | [WO200172957-A2, 04 OCT. 2001] | | | |

In a BLAST search of public sequence databases, the NOV6b protein was found to have homology to the proteins shown in the BLASTP data in Table 6E.

TABLE 6E
Public BLASTP Results for NOV6b

| | | NOV6b | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9NSA1 | Fibroblast growth factor-21 | 1 . . . 167 | 167/167 (100%) | 9.3e−91 |
| | precursor (FGF-21) - Homo sapiens | 1 . . . 206 | 206/206 (100%) | |
| | (Human), 209 aa. | | | |
| Q8N683 | Fibroblast growth factor 21 - Homo | 1 . . . 167 | 205/206 (99%) | 9.3e−91 |
| | sapiens (Human), 209 aa. | 1 . . . 206 | 205/206 (99%) | |
| CAC51204 | Sequence 1 from Patent | 1 . . . 167 | 205/206 (99%) | 5.1e−90 |
| | WO0149849 - Homo sapiens | 1 . . . 205 | 205/206 (99%) | |
| | (Human), 208 aa. | | | |

PFam analysis predicts that the NOV6b protein contains the domains shown in the Table 6F. Specific amino acid residues of NOV6b for each domain is shown in column 2, equivalent domains in the other NOV6 proteins of the invention are also encompassed herein.

TABLE 6F
Domain Analysis of NOV6b

| | NOV6b Match Region | | |
|---|---|---|---|
| Pfam Domain | Amino Acid Residues: | Score | Expect Value |
| FGF | 15 . . . 140 | 27.7 | 2.8e−08 |

### Example 7

### NOV7, CG55051, Alpha-2 Macroglobulin-like

The NOV7 family of novel nucleic acids and polypeptides clones includes NOV7a through NOV7c, SEQ ID Nos: 77-82, and the nucleotide and encoded polypeptide sequences are shown in Table 7A. In a particular embodiment NOV7 nucleic acid sequence is SEQ ID NO:81, wherein residue X₁ is either T or C. Nucleic acid sequence SEQ ID NO:81 encodes polypeptide SEQ ID NO:82, wherein residue Z₁ is I or T. Equivalent nucleic acid and polypeptide substitutions apply to other NOV7 sequences as would be appreciated by one of skill in the art, and are emcompassed in the present invention.

TABLE 7A
NOV7 Sequence Analysis

| | | |
|---|---|---|
| NOV7a, CG55051-02 | SEQ ID NO: 77 | 1788 bp |
| DNA Sequence | ORF Start: at 1 | ORF Stop: end of sequence |

GAAGAACTTCCAACTACCTGGTGACATTACCAGCCCAGGCTAAATTTCCCCTCCGTTCAGAAGGTTTGTTTGG
ACCTGAGCCCTGGGTACAGTGATGTTAAATTCACGGTTACTCTGGAGACCAAGGACAAGACCCAGAAGTTGCT
AGAATACTCTGGACTGAAGAAGAGGCACTTACATTGTATCTCCTTTCTTGTACCACCTCCTGCTGGTGGCACA
GAAGAAGTGGCCACAATCCGGGTGTCGGGAGTTGGAAATAACATCAGCTTTGAGGAGAAGAAAAAGGTTCTAA
TTCAGAGGCAGGGGAACGGCACCTTTGTACAGACTGACAAACCTCTCTACACCCCAGGGCAGCAAGTGTATTT
CCGCATTGTCACCATGGATAGCAACTTCGTTCCAGTGAATGACAAGTACTCCATGGTGGAACTACAGGATCCA
AATAGCAACAGGATTGCACAGTGGCTGGAAGTGGTACCTGAGCAAGGCATTGTAGACCTGTCCTTCCAACTGG
CACCAGAGGCAATGCTGGGCACCTACACTGTGGCAGTGGCTGAGGGCAAGACCTTTGGTACTTTCAGTGTGGA
GGAATATGTGCTGCCGAAGTTTAAGGTGGAAGTGGTGGAACCCAAGGAGTTATCAACGGTGCAGGAATCTTTC
TTAGTAAAAATTTGTTGTAGGTACACCTATGGAAAGCCCATGCTAGGGGCAGTGCAGGTATCTGTGTGTCAGA
AGGCAAATACTTACTGGTATCGAGAGGTGGAACGGGAACAGCTTCCTGACAAATGCAGGAACCTCTCTGGACA
GACTGACAAAACAGGATGTTTCTCAGCACCTGTGGACATGGCCACCTTTGACCTCATTGGATATGCGTACAGC
CATCAAATCAATATTGTGGCTACTGTTGTGGAGGAAGGGACAGGTGTGGAGGCCAATGCCACTCAGAATATCT
ACATTTCTCCACAAATGGGATCAATGACCTTTGAAGACACCAGCAATTTTTACCATCCAAATTTCCCCTTCAG
TGGGAAGATAAGAGTTAGGGGCCATGATGACTCCTTCCTCAAGAACCATCTAGTGTTTCTGGTGATTTATGGC
ACAAATGGAACCTTCAACCAGACCCTGGTTACTGATAACAATGGCCTAGCTCCCTTTACCTTGGAGACATCCG
GTTGGAATGGGACAGACGTTTCTCTGGAGGGAAAGTTTCAAATGGAAGACTTAGTATATAATCCGGAACAAGT
GCCACGTTACTACCAAAATGCCTACCTGCACCTGCGACCCTTCTACAGCACAACCCGCAGCTTCCTTGGCATC
CACCGGCTAAACGGCCCCTTGAAATGTGGCCAGCCCCAGGAAGTGCTGGTGGATTATTACATCGACCCGGCCG
ATGCAAGCCCTGACCAAGAGATCAGCTTCTCCTACTATTTAATAGGGAAAGGAAGTTTGGTGATGGAGGGGCA
GAAACACCTGAACTCTAAGAAGAAAGGACTGAAAGCCCCCTTCTCTCTCTCACTGACCTTCACTTCGAGACTG
GCCCCTGATCCTTCCCTGGTGATCTATGCCATTTTTCCCAGTGGAGGTGTTGTAGCTGACAAAATTCAGTTCT
CAGTCGAGATGTGCTTTGACAATCAGGTTTCCCTTGGCTTCTCCCCCTCCCAGCAGCTTCCAGGAGCAGAAGT
GGAGCTGCAGCTGCAGGCAGCTCCCGGATCCCTGTGTGCGCTCCGGGCGGTGGATGAGAGTGTCTTACTGCTT
AGGCCAGACAGAGAGCTGAGCAACCGCTCTGTCTAT

| | | | |
|---|---|---|---|
| NOV7a, CG55051-02 | | | |
| Protein Sequence | SEQ ID NO: 78 | 596 aa | MW at 66508.0kD |

EELPNYLVTLPARLNFPSVQKVCLDLSPGYSDVKFTVTLETKDKTQKLLEYSGLKKRHLHCISFLVPPPAGGT
EEVATIRVSGVGNNISFEEKKKVLIQRQGNGTFVQTDKPLYTPGQQVYFRIVTMDSNFVPVNDKYSMVELQDP
NSNRIAQWLEVVPEQGIVDLSFQLAPEAMLGTYTVAVAEGKTFGTFSVEEYVLPKFKVEVVEPKELSTVQESF
LVKICCRYTYGKPMLGAVQVSVCQKANTYWYREVEREQLPDKCRNLSGQTDKTGCFSAPVDMATFDLIGYAYS
HQINIVATVVEEGTGVEANATQNIYISPQMGSMTFEDTSNFYHPNFPFSGKIRVRGHDDSFLKNHLVFLVIYG
TNGTFNQTLVTDNNGLAPFTLETSGWNGTDVSLEGKFQMEDLVYNPEQVPRYYQNAYLHLRPFYSTTRSFLGI
HRLNGPLKCGQPQEVLVDYYIDPADASPDQEISFSYYLIGKGSLVMEGQKHLNSKKKGLKAPFSLSLTFTSRL
APDPSLVIYAIFPSGGVVADKIQFSVEMCFDNQVSLGFSPSQQLPGAEVELQLQAAPGSLCALRAVDESVLLL
RPDRELSNRSVY

| | | |
|---|---|---|
| NOV7b, SNP 13377623 | SEQ ID NO: 79 | 4492 bp |
| DNA Sequence | ORF Start: ATG at 1 | ORF Stop: TGA at 4375 |

ATGTGGGCTCAGCTCCTTCTAGGAATGTTGGCCCTATCACCAGCCATTGCAGAAGAACTTCCAAACTACCTGG
TGACATTACCAGCCCGGCTAAATTTCCCCTCCGTTCAGAAGGTTTGTTTGGACCTGAGCCCTGGGTACAGTGA
TGTTAAATTCACGGTTACTCTGGAGACCAAGGACAAGACCCAGAAGTTGCTAGAATACTCTGGACTGAAGAAG
AGGCACTTACATTGTATCTCCTTTCTTGTACCACCTCCTGCTGGTGGCACAGAAGAAGTGGCCACAATCCGGG
TGTCGGGAGTTGGAAATAACATCAGCTTTGAGGAGAAGAAAAAGGTTCTAATTCAGAGGCAGGGGAACGGCAC
CTTTGTACAGACTGACAAACCTCTCTACACCCCAGGGCAGCAAGTGTATTTCCGCATTGTCACCATGGATAGC
AACTTCGTTCCAGTGAATGACAAGTACTCCATGGTGGAACTACAGGATCCAAATAGCAACAGGATTGCACAGT
GGCTGGAAGTGGTACCTGAGCAAGGCATTGTAGACCTGTCCTTCCAACTGGCACCAGAGGCAATGCTGGGCAC
CTACACTGTGGCAGTGGCTGAGGGCAAGACCTTTGGTACTTTCAGTGTGGAGGAATATGTGCTTTCTCCATTT
CTCCTTTTACTCTCTTCAGTGCTGCCGAAGTTTAAGGTGGAAGTGGTGGAACCCAAGGAGTTATCAACGGTGC
AGGAATCTTTCTTAGTAAAAATTTGTTGTAGGTACACCTATGGAAAGCCCATGCTAGGGGCAGTGCAGGTATC
TGTGTGTCAGAAGGCAAATACTTACTGGTATCGAGAGGTGGAACGGGAACAGCTTCCTGACAAATGCAGGAAC
CTCTCTGGACAGACTGACAAAACAGGATGTTTCTCAGCACCTGTGGACATGGCCACCTTTGACCTCATTGGAT
ATGCGTACAGCCATCAAATCAATATTGTGGCTACTGTTGTGGAGGAAGGGACAGGTGTGGAGGCCAATGCCAC
TCAGAATATCTACACTTCTCCACAAATGGGATCAATGACCTTTGAAGACACCAGCAATTTTTACCATCCAAAT
TTCCCCTTCAGTGGGAAGATGCTGCTCAAGTTTCCGCAAGGCGGTGTGCTCCCTTGCAAGAACCATCTAGTGT
TTCTGGTGATTTATGGCACAAATGGAACCTTCAACCAGACCCTGGTTACTGATAACAATGGCCTAGCTCCCTT
TACCTTGGAGACATCCGGTTGGAATGGGACAGACGTTTCTCTGGAGGGAAAGTTTCAAATGGAAGACTTAGTA
TATAATCCGGAACAAGTGCCACGTTACTACCAAAATGCCTACCTGCACCTGCGACCCTTCTACAGCACAACCC
GCAGCTTCCTTGGCATCCACCGGCTAAACGGCCCCTTGAAATGTGGCCAGCCCCAGGAAGTGCTGGTGGATTA
TTACATCGACCCGGCCGATGCAAGCCCTGACCAAGAGATCAGCTTCTCCTACTATTTAATAGGGAAAGGAAGT
TTGGTGATGGAGGGGCAGAAACACCTGAACTCTAAGAAGAAAGGACTGAAAGCCTCCTTCTCTCTCTCACTGA
CCTTCACTTCGAGACTGGCCCCTGATCCTTCCCTGGTGATCTATGCCATTTTTCCCAGTGGAGGTGTTGTAGC
TGACAAAATTCAGTTCTCAGTCGAGATGTGCTTTGACAATCAGCAGCTTCCAGGAGCAGAAGTGGAGCTGCAG
CTGCAGGCAGCTCCCGGATCCCTGTGTGCGCTCCGGGCGGTGGATGAGAGTGTCTTACTGCTTAGGCCAGACA
GAGAGCTGAGCAACCGCTCTGTCTATGGGATGTTTCCATTCTGGTATGGTCACTACCCCTATCAAGTGGCTGA
GTATGATCAGTGTCCAGTGTCTGGCCCATGGGACTTTCCTCAGCCCCTCATTGACCCAATGCCCCAAGGGCAT
TCGAGCCAGCGTTCCATTATCTGGAGGCCCTCGTTCTCTGAAGGCACGGACCTTTTCAGCTTTTTCCGGGACG
TGGGCCTGAAAATACTGTCCAATGCCAAAATCAAGAAGCCAGTAGATTGCAGTCACAGATCTCCAGAATACAG
CACTGCTATGGGTGGCGGTGGTCATCCAGAGGCTTTTGAGTCATCAACTCCTTTACATCAAGCAGAGGATTCT
CAGGTCCGCCAGTACTTCCCAGAGACCTGGCTCTGGGATCTGTTTCCTATTGGTAACTCGGGGAAGGAGGCGG
TCCACGTCACAGTTCCTGACGCCATCACCGAGTGGAAGGCGATGAGTTTCTGCACTTCCCAGTCAAGAGGCTT
CGGGCTTTCACCCACTGTTGGACTAACTGCTTTCAAGCCGTTCTTTGTTGACCTGACTCTCCCTTACTCAGTA
GTCCGTGGGGAATCCTTTCGTCTTACTGCCACCATCTTCAATTACCTAAAGGATTGCATCAGGGTTCAGACTG
ACCTGGCTAAATCGCATGAGTACCAGCTAGAATCATGGGCAGATTCTCAGACCTCCAGTTGTCTCTGTGCTGA
TGACGCAAAACCCACCACTGGAACATCACAGCTGTCAAATTGGGTCACATTAAACTTTACTATTAGTACAAAG
ATTCTGGACAGCAATGAACCATGTGGGGGCCAGAAGGGGTTTGTTCCCCAAAAGGGCCGAAGTGACACGCTCA
TCAAGCCAGTTCTCGTCAAACCTGAGGGAGTCCTGGTGGAGAAGACACACAGCTCATTGCTGTGCCCAAAAGG
AGGAAAGGTGGCATCTGAATCTGTCTCCCTGGAGCTCCCAGTGGACATTGTTCCTGACTCGACCAAGGCTTAT
GTTACGGTTCTGGGAGACATTATGGGCACAGCCCTGCAGAACCTGGATGGTCTGGTGCAGATGCCCAGTGGCT
GTGGCGAGCAGAACATGGTCTTGTTTGCTCCCATCATCTATGTCTTGCAGTACCTGGAGAAGGCAGGGCTGCT
GACGGAGGAGATCAGGTCTCGGGCAGTGGGTTTCCTGGAAATAGGGTACCAGAAGGAGCTGATGTACAAACAC
AGCAATGGCTCATACAGTGCCTTTGGGGAGCGAGATGGAAATGGAAACACATGGCTGACAGCGTTTGTCACAA
AATGCTTTGGCCAAGCTCAGAAATTCATCTTCATTGATCCCAAGAACATCCAGGATGCTCTCAAGTGGATGGC
AGGAAACCAGCTCCCCAGTGGCTGCTATGCCAACGTGGGAAATCTCCTTCACACAGCTATGAAGGGTGGTGTT
GATGATGAGGTCTCCTTGACTGCGTATGTCACAGCTGCATTGCTGGAGATGGGAAAGGATGTAGATGACCCAA
TGGTGAGTCAGGGTCTACGGTGTCTCAAGAATTCGGCCACCTCCACGACCAACCTCTACACACAGGCCCTGTT
GGCTTACATTTTCTCCCTGGCTGGGGAAATGGACATCAGAAACATTCTCCTTAAACAGTTAGATCAACAGGCT
ATCATCTCAGGAGAATCCATTTACTGGAGCCAGAAACCTACTCCATCATCGAACGCCAGCCCTTGGTCTGAGC
CTGCGGCTGTAGATGTGGAACTCACAGCATATGCATTGTTGGCCCAGCTTACCAAGCCCAGCCTGACTCAAAA
GGAGATAGCGAAGGCCACTAGCATAGTGGCTTGGTTGGCCAAGCAACACAATGCATATGGGGGCTTCTCTTCT
ACTCAGGATACTGTAGTTGCTCTCCAAGCTCTTGCCAAATATGCCACTACdGCCTACATGCCATCTGAGGAGA
TCAACCTGGTTGTAAAATCCACTGAGAATTTCCAGCGCACATTCAACATACAGTCAGTTAACAGATTGGTATT
TCAGCAGGATACCCTGCCCAATGTCCCTGGAATGTACACGTTGGAGGCCTCAGGCCAGGGCTGTGTCTATGTG
CAGACGGTGTTGAGATACAAATATTCTCCCTCCCACAATATGAAGACCTTTAGTCTTAGTGTGGAAATAGGAA
AAGCTAGATGTGAGCAGCCGACTTCACCTCGATCCTTGACTCTCACTATTCACACCAGTTATGTGGGGAGCCG
TAGCTCTTCCAATATGGCTATTGTGGAAGTGAAGATGCTATCTGGGTTCAGTCCCATGGAGGGCACCAATCAG
TTACTTCTCCAGCAACCCCTGGTGAAGAAGGTTGAATTTGGAACTGACACACTTAACATTTACTTGGATGAGC
TCATTAAGAACACTCAGACTTACACCTTCACCATCAGCCAAAGTGTGCTGGTCACCAACTTGAAACCAGCAAC
CATCAAGGTCTATGACTACTACCTACCAGATGAACAGGCAACAATTCAGTATTCTGATCCCTGTGAATGAGGA
TAGGAGCTGGAAACTCAATTAGTCCTCTGTGACATTTACTGGAGGGTGGAACATTCTTCTGTCGCTTGAAGCA
GAACTCATTCAATCAAATAATTTAATTTCTCTGACTAGT

| | | | |
|---|---|---|---|
| NOV7b, SNP 13377623 | | | |
| Protein Sequence | SEQ ID NO: 80 | 1458 aa | Mw at 161434.6kD |

MWAQLLLGMLALSPAIAEELPNYLVTLPARLNFPSVQKVCLDLSPGYSDVKFTVTLETKDKTQKLLEYSGLKK
RHLHCISFLVPPPAGGTEEVATIRVSGVGNNISFEEKKKVLIQRQGNGTFVQTDKPLYTPGQQVYFRIVTMDS
NFVPVNDKYSMVELQDPNSNRIAQWLEVVPEQGIVDLSFQLAPEAMLGTYTVAVAEGKTFGTFSVEEYVLSPF
LLLLSSVLPKFKVEVVEPKELSTVQESFLVKICCRYTYGKPMLGAVQVSVCQKANTYWYREVEREQLPDKCRN
LSGQTDKTGCFSAPVDMATFDLIGYAYSHQINIVATVVEEGTGVEANATQNIYTSPQMGSMTFEDTSNFYHPN
FPFSGKMLLKFPQGGVLPCKNHLVFLVIYGTNGTFNQTLVTDNNGLAPFTLETSGWNGTDVSLEGKFQMEDLV
YNPEQVPRYYQNAYLHLRPFYSTTRSFLGIHRLNGPLKCGQPQEVLVDYYIDPADASPDQEISFSYYLIGKGS
LVMEGQKHLNSKKKGLKASFSLSLTFTSRLAPDPSLVIYAIFPSGGVVADKIQFSVEMCFDNQQLPGAEVELQ
LQAAPGSLCALRAVDESVLLLRPDRELSNRSVYGMFPFWYGHYPYQVAEYDQCPVSGPWDFPQPLIDPMPQGH
SSQRSIIWRPSFSEGTDLFSFFRDVGLKILSNAKIKKPVDCSHRSPEYSTAMGGGGHPEAFESSTPLHQAEDS
QVRQYFPETWLWDLFPIGNSGKEAVHVTVPDAITEWKAMSFCTSQSRGFGLSPTVGLTAFKPFFVDLTLPYSV
VRGESFRLTATIFNYLKDCIRVQTDLAKSHEYQLESWADSQTSSCLCADDAKTHHWNITAVKLGHINFTISTK
ILDSNEPCGGQKGFVPQKGRSDTLIKPVLVKPEGVLVEKTHSSLLCPKGGKVASESVSLELPVDIVPDSTKAY
VTVLGDIMGTALQNLDGLVQMPSGCGEQNMVLFAPIIYVLQYLEKAGLLTEEIRSRAVGFLEIGYQKELMYKH
SNGSYSAFGERDGNGNTWLTAFVTKCFGQAQKFIFIDPKNIQDALKWMAGNQLPSGCYANVGNLLHTAMKGGV
DDEVSLTAYVTAALLEMGKDVDDPMVSQGLRCLKNSATSTTNLYTQALLAYIFSLAGEMDIRNILLKQLDQQA
IISGESIYWSQKPTPSSNASPWSEPAAVDVELTAYALLAQLTKPSLTQKEIAKATSIVAWLAKQHNAYGGFSS
TQDTVVALQALAKYATTAYMPSEEINLVVKSTENFQRTFNIQSVNRLVFQQDTLPNVPGMYTLEASGQGCVYV
QTVLRYNILPPTNMKTFSLSVEIGKARCEQPTSPRSLTLTIHTSYVGSRSSSNMAIVEVKMLSGFSPMEGTNQ
LLLQQPLVKKVEFGTDTLNIYLDELIKNTQTYTFTISQSVLVTNLKPATIKVYDYYLPDEQATIQYSDPCE

| | | |
|---|---|---|
| NOV7c, CG55051 | SEQ ID NO: 81 | 4492 bp |
| DNA Sequence | ORF Start: ATG at 1 | ORF Stop: TGA at 4375 |

ATGTGGGCTCAGCTCCTTCTAGGAATGTTGGCCCTATCACCAGCCATTGCAGAAGAACTTCCAAACTACCTGG
TGACATTACCAGCCCGGCTAAATTTCCCCTCCGTTCAGAAGGTTTGTTTGGACCTGAGCCCTGGGTACAGTGA
TGTTAAATTCACGGTTACTCTGGAGACCAAGGACAAGACCCAGAAGTTGCTAGAATACTCTGGACTGAAGAAG
AGGCACTTACATTGTATCTCCTTTCTTGTACCACCTCCTGCTGGTGGCACAGAAGAAGTGGCCACAATCCGGG
TGTCGGGAGTTGGAAATAACATCAGCTTTGAGGAGAAGAAAAAGGTTCTAATTCAGAGGCAGGGGAACGGCAC
CTTTGTACAGACTGACAAACCTCTCTACACCCCAGGGCAGCAAGTGTATTTCCGCATTGTCACCATGGATAGC
AACTTCGTTCCAGTGAATGACAAGTACTCCATGGTGGAACTACAGGATCCAAATAGCAACAGGATTGCACAGT
GGCTGGAAGTGGTACCTGAGCAAGGCATTGTAGACCTGTCCTTCCAACTGGCACCAGAGGCAATGCTGGGCAC
CTACACTGTGGCAGTGGCTGAGGGCAAGACCTTTGGTACTTTCAGTGTGGAGGAATATGTGCTTTCTCCATTT
CTCCTTTTACTCTCTTCAGTGCTGCCGAAGTTTAAGGTGGAAGTGGTGGAACCCAAGGAGTTATCAACGGTGC
AGGAATCTTTCTTAGTAAAAATTTGTTGTAGGTACACCTATGGAAAGCCCATGCTAGGGGCAGTGCAGGTATC
TGTGTGTCAGAAGGCAAATACTTACTGGTATCGAGAGGTGGAACGGGAACAGCTTCCTGACAAATGCAGGAAC
CTCTCTGGACAGACTGACAAAACAGGATGTTTCTCAGCACCTGTGGACATGGCCACCTTTGACCTCATTGGAT
ATGCGTACAGCCATCAAATCAATATTGTGGCTACTGTTGTGGAGGAAGGGACAGGTGTGGAGGCCAATGCCAC
TCAGAATATCTACAX₁TTCTCCACAAATGGGATCAATGACCTTTGAAGACACCAGCAATTTTTACCATCCAAA
TTTCCCCTTCAGTGGGAAGATGCTGCTCAAGTTTCCGCAAGGCGGTGTGCTCCCTTGCAAGAACCATCTAGTG
TTTCTGGTGATTTATGGCACAAATGGAACCTTCAACCAGACCCTGGTTACTGATAACAATGGCCTAGCTCCCT
TTACCTTGGAGACATCCGGTTGGAATGGGACAGACGTTTCTCTGGAGGGAAAGTTTCAAATGGAAGACTTAGT
ATATAATCCGGAACAAGTGCCACGTTACTACCAAAATGCCTACCTGCACCTGCGACCCTTCTACAGCACAACC
CGCAGCTTCCTTGGCATCCACCGGCTAAACGGCCCCTTGAAATGTGGCCAGCCCCAGGAAGTGCTGGTGGATT
ATTACATCGACCCGGCCGATGCAAGCCCTGACCAAGAGATCAGCTTCTCCTACTATTTAATAGGGAAAGGAAG
TTTGGTGATGGAGGGGCAGAAACACCTGAACTCTAAGAAGAAAGGACTGAAAGCCTCCTTCTCTCTCTCACTG
ACCTTCACTTCGAGACTGGCCCCTGATCCTTCCCTGGTGATCTATGCCATTTTTCCCAGTGGAGGTGTTGTAG
CTGACAAAATTCAGTTCTCAGTCGAGATGTGCTTTGACAATCAGCAGCTTCCAGGAGCAGAAGTGGAGCTGCA
GCTGCAGGCAGCTCCCGGATCCCTGTGTGCGCTCCGGGCGGTGGATGAGAGTGTCTTACTGCTTAGGCCAGAC
AGAGAGCTGAGCAACCGCTCTGTCTATGGGATGTTTCCATTCTGGTATGGTCACTACCCCTATCAAGTGGCTG
AGTATGATCAGTGTCCAGTGTCTGGCCCATGGGACTTTCCTCAGCCCCTCATTGACCCAATGCCCCAAGGGCA
TTCGAGCCAGCGTTCCATTATCTGGAGGCCCTCGTTCTCTGAAGGCACGGACCTTTTCAGCTTTTTCCGGGAC
GTGGGCCTGAAAATACTGTCCAATGCCAAAATCAAGAAGCCAGTAGATTGCAGTCACAGATCTCCAGAATACA
TCACTGCTATGGGTGGCGGTGGTCATCCAGAGGCTTTTGAGTCATCAACTCCTTTACATCAAGCAGAGGATTC
CAGGTCTCGCCAGTACTTCCCAGAGACCTGGCTCTGGGATCTGTTTCCTATTGGTAACTCGGGGAAGGAGGCG
TCCACGGTCACAGTTCCTGACGCCATCACCGAGTGGAAGGCGATGAGTTTCTGCACTTCCCAGTCAAGAGGCT
CGGGCTTTTCACCCACTGTTGGACTAACTGCTTTCAAGCCGTTCTTTGTTGACCTGACTCTCCCTTACTCAGT
GTCCGTAGGGGAATCCTTTCGTCTTACTGCCACCATCTTCAATTACCTAAAGGATTGCATCAGGGTTCAGACT
ACCTGGGCTAAATCGCATGAGTACCAGCTAGAATCATGGGCAGATTCTCAGACCTCCAGTTGTCTCTGTGCTG
TGACGCAAAAAACCCACCACTGGAACATCACAGCTGTCAAATTGGGTCACATTAACTTTACTATTAGTACAAA
ATTCTGGGACAGCAATGAACCATGTGGGGGCCAGAAGGGGTTTGTTCCCCAAAAGGGCCGAAGTGACACGCTC
TCAAGCACAGTTCTCGTCAAACCTGAGGGAGTCCTGGTGGAGAAGACACACAGCTCATTGCTGTGCCCAAAAG
AGGAAAGGGTGGCATCTGAATCTGTCTCCCTGGAGCTCCCAGTGGACATTGTTCCTGACTCGACCAAGGCTTA
GTTACGTGTTCTGGGAGACATTATGGGCACAGCCCTGCAGAACCTGGATGGTCTGGTGCAGATGCCCAGTGGC
GTGGCGTAGCAGAACATGGTCTTGTTTGCTCCCATCATCTATGTCTTGCAGTACCTGGAGAAGGCAGGGCTGC
GACGGATGGAGATCAGGTCTCGGGCAGTGGGTTTCCTGGAAATAGGGTACCAGAAGGAGCTGATGTACAAACA
AGCAACAGGCTCATACAGTGCCTTTGGGGAGCGAGATGGAAATGGAAACACATGGCTGACAGCGTTTGTCACA
AATGCATTTGGCCAAGCTCAGAAATTCATCTTCATTGATCCCAAGAACATCCAGGATGCTCTCAAGTGGATGG
AGGAACACCAGCTCCCCAGTGGCTGCTATGCCAACGTGGGAAATCTCCTTCACACAGCTATGAAGGGTGGTGT
GATGATTGAGGTCTCCTTGACTGCGTATGTCACAGCTGCATTGCTGGAGATGGGAAAGGATGTAGATGACCCA
TGGTGAAGTCAGGGTCTACGGTGTCTCAAGAATTCGGCCACCTCCACGACCAACCTCTACACACAGGCCCTGT
GGCTTTACATTTTCTCCCTGGCTGGGGAAATGGACATCAGAAACATTCTCCTTAAACAGTTAGATCAACAGGC
ATCATTCTCAGGAGAATCCATTTACTGGAGCCAGAAACCTACTCCATCATCGAACGCCAGCCCTTGGTCTGAG
CTGCGCGCTGTAGATGTGGAACTCACAGCATATGCATTGTTGGCCCAGCTTACCAAGCCCAGCCTGACTCAAA
GGAGAATAGCGAAGGCCACTAGCATAGTGGCTTGGTTGGCCAAGCAACACAATGCATATGGGGGCTTCTCTTC
ACTCATGGATACTGTAGTTGCTCTCCAAGCTCTTGCCAAATATGCCACTACCGCCTACATGCCATCTGAGGAG
TCAACACTGGTTGTAAAATCCACTGAGAATTTCCAGCGCACATTCAACATACAGTCAGTTAACAGATTGGTAT
TCAGCTAGGATACCCTGCCCAATGTCCCTGGAATGTACACGTTGGAGGCCTCAGGCCAGGGCTGTGTCTATGT
CAGACGGGTGTTGAGATACAATATTCTCCCTCCCACAAATATGAAGACCTTTAGTCTTAGTGTGGAAATAGGA
AAGCTAAGATGTGAGCAGCCGACTTCACCTCGATCCTTGACTCTCACTATTCACACCAGTTATGTGGGGAGCC
TAGCTGCTTCCAATATGGCTATTGTGGAAGTGAAGATGCTATCTGGGTTCAGTCCCATGGAGGGCACCAATCA
TTACTGTCTCCAGCAACCCCTGGTGAAGAAGGTTGAATTTGGAACTGACACACTTAACATTTACTTGGATGAG
TCATTCAAGAACACTCAGACTTACACCTTCACCATCAGCCAAAGTGTGCTGGTCACCAACTTGAAACCAGCAA
CCATCAAGGTCTATGACTACTACCTACCAGATGAACAGGCAACAATTCAGTATTCTGATCCCTGTGAATGAGG
ATAGGAGCTGGAAACTCAATTAGTCCTCTGTGACATTTACTGGAGGGTGGAACATTCTTCTGTCGCTTGAAGC
AGAACTCATTCAATCAAATAATTTAATTTCTCTGACTAGT
[Wherein residue X₁ is either T or C.]

| | | | |
|---|---|---|---|
| NOV7c, CG55051 | | | |
| Protein Sequence | SEQ ID NO: 82 | 1458 aa | MW at 161446.7kD |

MWAQLLLGMLALSPAIAEELPNYLVTLPARLNFPSVQKVCLDLSPGYSDVKFTVTLETKDKTQKLLEYSGLKK
RHLHCISFLVPPPAGGTEEVATIRVSGVGNNISFEEKKKVLIQRQGNGTFVQTDKPLYTPGQQVYFRIVTMDS
NFVPVNDKYSMVELQDPNSNRIAQWLEVVPEQGIVDLSFQLAPEAMLGTYTVAVAEGKTFGTFSVEEYVLSPF
LLLLSSVLPKFKVEVVEPKELSTVQESFLVKICCRYTYGKPMLGAVQVSVCQKANTYWYREVEREQLPDKCRN
LSGQTDKTGCFSAPVDMATFDLIGYAYSHQINIVATVVEEGTGVEANATQNIYZ₁SPQMGSMTFEDTSNFYHP
FPFSGKNMLLKFPQGGVLPCKNHLVFLVIYGTNGTFNQTLVTDNNGLAPFTLETSGWNGTDVSLEGKFQMEDL
YNPEQVVPRYYQNAYLHLRPFYSTTRSFLGIHRLNGPLKCGQPQEVLVDYYIDPADASPDQEISFSYYLIGKG
LVMEGQSKHLNSKKKGLKASFSLSLTFTSRLAPDPSLVIYAIFPSGGVVADKIQFSVEMCFDNQQLPGAEVEL
LQAAPGQSLCALRAVDESVLLLRPDRELSMRSVYGMFPFWYGHYPYQVAEYDQCPVSGPWDFPQPLIDPMPQG
SSQRSIHIWRPSFSEGTDLFSFFRDVGLKILSNAKIKKPVDCSHRSPEYSTAMGGGGHPEAFESSTPLHQAED
QVRQYFSPETWLWDLFPIGNSGKEAVHVTVPDAITEWKAMSFCTSQSRGFGLSPTVGLTAFKPFFVDLTLPYS
VRGESFVRLTATIFNYLKDCIRVQTDLAKSHEYQLESWADSQTSSCLCADDAKTHHWNITAVKLGHINFTIST
ILDSNEKPCGGQKGFVPQKGRSDTLIKPVLVKPEGVLVEKTHSSLLCPKGGKVASESVSLELPVDIVPDSTKA
VTVLGDYIMGTALQNLDGLVQMPSGCGEQNMVLFAPIIYVLQYLEKAGLLTEEIRSPAVGFLEIGYQKELMYK
SNGSYSHAFGERDGNGNTWLTAFVTKCFGQAQKFIFIDPKNIQDALKWMAGNQLPSGCYANVGNLLHTAMKGG
DDEVSLVTAYVTAALLEMGKDVDDPMVSQGLRCLKNSATSTTNLYTQAILAYIFSLAGEMDIRNILLKQLDQQ
IISGESAIYWSQKPTPSSNASPWSEPAAVDVELTAYALLAQLTKPSLTQKEIAKATSIVAWLAKQHNAYGGFS
TQDTVVSALQALAKYATTAYMPSEEINLVVKSTENFQRTFNIQSVNRLVFQQDTLPNVPGMYTLEASGQGCVY
QTVLRYVNILPPTNMKTFSLSVEIGKARCEQPTSPRSLTLTIHTSYVGSRSSSNMAIVEVKMLSGFSPMEGTN
LLLQQPQLVKKVEFGTDTLNIYLDELIKNTQTYTFTISQSVLVTNLKPATIKVYDYYLPDEQATIQYSDPCE
[Wherein residue Z₁ is I or T.]

Further analysis of the NOV7a protein yielded the following properties shown in Table7C.

TABLE 7C
Protein Sequence Properties NOV7a

| SignalP | No Known Signal Sequence Predicted | | | |
|---|---|---|---|---|
| analysis: | | | | |
| PSORT II | Psort II Results (see Details): | | | |
| analysis: | 52.2%: cytoplasmic | | | |
| | 26.1%: nuclear | | | |
| | 21.7%: mitochondrial | | | |
| | Details of Psort Prediction | | | |
| | >>> MUS belongs to the animal class | | | |
| | *** Reasoning Step: 2 | | | |
| | SRCFLG: 1 | | | |
| | Prelim. Calc. of ALOM (thresh: 0.5) count: 0 | | | |
| | McG: Length of UR: 10 | | | |
| | Peak Value of UR: 1.33 | | | |
| | Net Charge of CR: −2 | | | |
| | McG: Discrim Score: −7.23 | | | |
| | GvH: Signal Score (−3.5): −3.9 | | | |
| | Possible site: 31 | | | |
| | >>> Seems to have no N-terminal signal seq. | | | |
| | Amino Acid Composition: calculated from 1 | | | |
| | new cnt: 0 ** thrshld changed to −2 | | | |
| | involving clv. sig in the ALOMREC or not: 0B | | | |
| | ALOM program count: 0 value: 1.32 threshold: −2.0 | | | |
| | PERIPHERAL Likelihood = 1.32 | | | |
| | modified ALOM score: −1.16 | | | |
| | Gavel: Bound. Mitoch. Preseq. R-2 motif: 1 | | | |
| | mtdisc (mit) Status: negative (−3.22) | | | |
| | *** Reasoning Step: 3 | | | |
| | KDEL Count: 0 | | | |
| | Goal mtmx modified Score: 0.10 | | | |
| | SKL motif: pos: 509(596), count: 2 SRL | | | |
| | pox modified by SKL scr: 0.3 | | | |
| | Poxaac Score: 0.32 | | | |
| | >>> POX Status: notclr | | | |
| | pox modified by aac scr: 0.110 | | | |
| | >>> lys: 0.07 Status: notclr | | | |
| | Goal lys: modified. Score: 0.157 | | | |
| | Nuc-4 pos: 54 (3) KKRH | | | |
| | nuc modified. Score: 0.60 | | | |
| | >>> Nuclear Signal. Status: notclr (0.30) | | | |
| | Details of Psort II Prediction | | | |
| | *** Warning: 1st aa is not methyonine | | | |
| | PSG: a new signal peptide prediction method | | | |
| | N-region: length 2; pos. chg 0; neg. chg 2 | | | |
| | H-region: length 10; peak value 0.00 | | | |
| | PSG score: −4.40 | | | |
| | GvH: von Heijne's method for signal seq. recognition | | | |
| | GvH score (threshold: −2.1): −7.90 | | | |
| | possible cleavage site: between 31 and 32 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 1.32 (at 517) | | | |
| | ALOM score: 1.32 (number of TMSs: 0) | | | |
| | MITDISC: discrimination of mitochondrial targeting seq | | | |
| | R content: | 1 | Hyd Moment(75): | 9.53 |
| | Hyd Moment(95): | 10.99 | G content: | 0 |
| | D/E content: | 3 | S/T content: | 2 |

Score: −6.53
Gavel: prediction of cleavage sites for mitochondrial preseq
cleavage site motif not found
NUCDISC: discrimination of nuclear localization signals
pat4: KKRH (3) at 55
pat7: none
bipartite: none
content of basic residues: 8.9%
NLS Score: −0.29
NNCN: Reinhardt's method for Cytplasmic/Nuclear discrimination
Prediction: cytoplasmic
Reliability: 89

A search of the NOV7a protein against the Geneseq database, a proprietary database that contains sequences published in patents and patent publication, yielded several homologous proteins shown in Table 7C.

TABLE 7C
GENESEQ RESULTS FOR NOV7a

| | | NOV7a | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| AAG63549 | A human alpha-2 macroglobulin-like | 1 . . . 596 | 595/596 (99%) | 0.0 |
| | polypeptide - Homo sapiens, 912 aa. | 31 . . . 623 | 595/596 (99%) | |
| AAG63550 | A human alpha-2 macroglobulin-like | 1 . . . 596 | 595/596 (99%) | 0.0 |
| | polypeptide variant - Homo sapiens, | 18 . . . 613 | 595/596 (99%) | |
| | 899 aa. | | | |
| AAG63551 | A human alpha-2 macroglobulin-like | 1 . . . 596 | 595/596 (99%) | 0.0 |
| | polypeptide - Homo sapiens, 882 aa. | 1 . . . 596 | 595/596 (99%) | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD48670 | Sequence 1 from Patent | 1 . . . 596 | 576/600 (96%) | 0.0 |
| | WO0229058 - Homo sapiens | 18 . . . 617 | 579/600 (96%) | |
| | (Human), 1492 aa. | | | |
| P01023 | Alpha-2-macroglobulin precursor | 4 . . . 596 | 207/593 (34%) | 0.0 |
| | (Alpha-2-M) - Homo sapiens | 29 . . . 619 | 324/593 (54%) | |
| | (Human), 1474 aa | | | |
| CAA01533 | ALPHA 2-MACROGLOBULIN | 4 . . . 596 | 207/593 (34%) | 0.0 |
| | 690-740 - Homo sapiens (Human), | 29 . . . 619 | 324/593 (54%) | |
| | 1484 aa | | | |

PFam analysis predicts that the NOV7a protein contains the domains shown in the Table 7F. Specific amino acid residues of NOV7a for each domain is shown in column 2, equivalent domains in the other NOV7 proteins of the invention are also encompassed herein.

TABLE 7F
Domain Analysis of NOV7a

| | NOV7a Match Region | | |
|---|---|---|---|
| Pfam Domain | Amino acid residues | Score | Expect Value |
| A2M_N | 1 . . . 596 | 278.3 | 1e−79 |

### Example 8

### NOV8, CG55060, Antileukoproteinase 1

The NOV8 family of novel nucleic acids and polypeptides clones includes NOV8a through NOV8g, SEQ ID Nos: 83-96, and the nucleotide and encoded polypeptide sequences are shown in Table 8A. In a particular embodiment NOV8 nucleic acid sequence is SEQ ID NO:95, wherein each of residues X₁, X₂, X₃, X₄, and X₅, is either T or C. Nucleic acid sequence SEQ ID NO:95 encodes polypeptide SEQ ID NO:96, wherein each of residues Z₁ is F or S; Z₂ is L or P; Z₃ is C or R; Z₄ is L or S; and Z₅ is C or R. Equivalent nucleic acid and polypeptide substitutions apply to other NOV8 sequences as would be appreciated by one of skill in the art, and are emcompassed in the present invention.

TABLE 8A
NOV8 Sequence Analysis

| | | |
|---|---|---|
| NOV8a, CG55060-04 | SEQ ID NO: 83 | 24 bp |
| DNA Sequence | ORF Start: at 1 | ORF Stop: TAG at 322 |

TCTGGAAAGTCCTTCAAAGCTGGAGTCTGTCCTCCTAAGAAATCTGCCCAGTGCCTTAGATACAAGAAACCTG
AGTGCCAGAGTGACTGGCAGTGTCCAGGGAAGAAGAGATGTTGTCCTGACACTTGTGGCATCAAATGCCTGGA
TCCTGTTGACACCCCAAACCCAACAAGGAGGAAGCCTGGGAAGTACCCAGTGACTTATGGCCAATGTTTGATG
CTTAACCCCCCCAATTTCTGTGAGATGGATGGCCAGTGCAAGCGTGACTTGAAGTGTTGCATGGGCATGTGTG
GGAAATCCTGCGTTTCCCCTGTGAAAGCTTAG

| | | | |
|---|---|---|---|
| NOV8a, CG55060-04 | | | |
| Protein Sequence | SEQ ID NO: 84 | 107 aa | MW at 11785.9kD |

SGKSFKAGVCPPKKSAQCLRYKKPECQSDWQCPGKKRCCPDTCGIKCLDPVDTPNPTRRKPGKYPVTYGQCLM
LNPPNFCEMDGQCKRDLKCCMGMCGKSCVSPVKA

| | | |
|---|---|---|
| NOV8b, SNP 13374945 | SEQ ID NO: 85 | 594 bp |
| DNA Sequence | ORF Start: ATG at 19 | ORF Stop: TGA at 415 |

GTCACTCCTGCCTTCACCATGAAGTCCAGCGGCCTCTCCCCCTTCCTGGTGCTGCTTGCCCTGGGAACTCTGG
CACCTTGGGCTGTGGAAGGCTCTGGAAAGTCCTTCAAAGCTGGAGTCTGTCCTCCTAAGAAATCTGCCCAGTG
CCTTAGATACAAGAAACCTGAGTGCCAGAGTGACTGGCAGTGTCCAGGGAAGAAGAGATGTTGTCCTGACACT
TGTGGCATCAAATGCCTGGATCCTGTTGACACCCCAAACCCAACAAGGAGGAAGCCTGGGAAGTGCCCAGTGA
CTTATGGCCAATGTTTGATGCTTAACCCCCCCAATTTCTGTGAGATGGATGGCCAGTGCAAGCGTGACTTGAA
GTGTTGCATGGGCATGTGTGGGAAATCCTGCGTTTCCCCTGTGAAAGCTTGATTCCTGCCATATGGAGGAGGC
TCTGGAGTCCTGCTCTGTGTGGTCCAGGTCCTTTCCACCCTGAGACTTGGCTCCACCACTGATATCCTCCTTT
GGGGAAAGGCTTGGCACACAGCAGGCTTTCAAGAAGTGCCAGTTGATCAATGAATAAATAAACGAGCCTATTT
CTCTTTGCAC

| | | | |
|---|---|---|---|
| NOV8b, SNP 13374945 | | | |
| Protein Sequence | SEQ ID NO: 86 | 132 aa | MW at 14265.8kD |

MKSSGLSPFLVLLALGTLAPWAVEGSGKSFKAGVCPPKKSAQCLRYKKPECQSDWQCPGKKRCCPDTCGIKCL
DPVDTPNPTRRKPGKCPVTYGQCLMLNPPNFCEMDGQCKRDLKCCMGMCGKSCVSPVKA

| | | |
|---|---|---|
| NOV8c, SNP 13376226 | SEQ ID NO: 87 | 594 bp |
| DNA Sequence | ORF Start: ATG at 19 | ORF Stop: TGA at 415 |

GTCACTCCTGCCTTCACCATGAAGTCCAGCGGCCTCTTCCCCTTCCTGGTGCTGCTTGCCCTGGGAACTCTGG
CACCTTGGGCTGTGGAAGGCTCTGGAAAGTCCTTCAAAGCTGGAGTCTGTCCTCCTAAGAAATCTGCCCAGTG
CCTTAGATACAAGAAACCTGAGCGCCAGAGTGACTGGCAGTGTCCAGGGAAGAAGAGATGTTGTCCTGACACT
TGTGGCATCAAATGCCTGGATCCTGTTGACACCCCAAACCCAACAAGGAGGAAGCCTGGGAAGTGCCCAGTGA
CTTATGGCCAATGTTTGATGCTTAACCCCCCCAATTTCTGTGAGATGGATGGCCAGTGCAAGCGTGACTTGAA
GTGTTGCATGGGCATGTGTGGGAAATCCTGCGTTTCCCCTGTGAAAGCTTGATTCCTGCCATATGGAGGAGGC
TCTGGAGTCCTGCTCTGTGTGGTCCAGGTCCTTTCCACCCTGAGACTTGGCTCCACCACTGATATCCTCCTTT
GGGGAAAGGCTTGGCACACAGCAGGCTTTCAAGAAGTGCCAGTTGATCAATGAATAAATAAACGAGCCTATTT
CTCTTTGCAC

| | | | |
|---|---|---|---|
| NOV8c, SNP 13376226 | | | |
| Protein Sequence | SEQ ID NO: 88 | 132 aa | MW at 14379.0kD |

MKSSGLFPFLVLLALGTLAPWAVEGSGKSFKAGVCPPKKSAQCLRYKKPERQSDWQCPGKKRCCPDTCGIKCL
DPVDTPNPTRRKPGKCPVTYGQCLMLNPPNFCEMDGQCKRDLKCCMGMCGKSCVSPVKA

| | | |
|---|---|---|
| NOV8d, SNP 13377692 | SEQ ID NO: 89 | 594 |
| DNA Sequence | ORF Start: ATG at 19 | ORF Stop: TGA at 415 |

GTCACTCCTGCCTTCACCATGAAGTCCAGCGGCCTCTTCCCCTTCCTGGTGCCGCTTGCCCTGGGAACTCTGG
CACCTTGGGCTGTGGAAGGCTCTGGAAAGTCCTTCAAAGCTGGAGTCTGTCCTCCTAAGAAATCTGCCCAGTG
CCTTAGATACAAGAAACCTGAGTGCCAGAGTGACTGGCAGTGTCCAGGGAAGAAGAGATGTTGTCCTGACACT
TGTGGCATCAAATGCCTGGATCCTGTTGACACCCCAAACCCAACAAGGAGGAAGCCTGGGAAGTGCCCAGTGA
CTTATGGCCAATGTTTGATGCTTAACCCCCCCAATTTCTGTGAGATGGATGGCCAGTGCAAGCGTGACTTGAA
GTGTTGCATGGGCATGTGTGGGAAATCCTGCGTTTCCCCTGTGAAAGCTTGATTCCTGCCATATGGAGGAGGC
TCTGGAGTCCTGCTCTGTGTGGTCCAGGTCCTTTCCACCCTGAGACTTGGCTCCACCACTGATATCCTCCTTT
GGGGAAAGGCTTGGCACACAGCAGGCTTTCAAGAAGTGCCAGTTGATCAATGAATAAATAAACGAGCCTATTT
CTCTTTGCAC

| | | | |
|---|---|---|---|
| NOV8d, SNP 13377692 | | | |
| Protein Sequence | SEQ ID NO: 90 | 132 aa | MW at 14309.9kD |

MKSSGLFPFLVPLALGTLAPWAVEGSGKSFKAGVCPPKKSAQCLRYKKPECQSDWQCPGKKRCCPDTCGIKCL
DPVDTPNPTRRKPGKCPVTYGQCLMLNPPNFCEMDGQCKRDLKCCMGMCGKSCVSPVKA

| | | |
|---|---|---|
| NOV8e, SNP 13378858 | SEQ ID NO: 91 | 594 bp |
| DNA Sequence | ORF Start: ATG at 19 | ORF Stop: TGA at 415 |

GTCACTCCTGCCTTCACCATGAAGTCCAGCGGCCTCTTCCCCTTCCTGGTGCTGCTTGCCCTGGGAACTCTGG
CACCTTGGGCTGTGGAAGGCTCTGGAAAGTCCTTCAAAGCTGGAGTCTGTCCTCCTAAGAAATCTGCCCAGTG
CCTTAGATACAAGAAACCTGAGTGCCAGAGTGACTGGCAGTGTCCAGGGAAGAAGAGATGTTGTCCTGACACT
TGTGGCATCAAATGCCTGGATCCTGTTGACACCCCAAACCCAACAAGGAGGAAGCCTGGGAAGTGCCCAGTGA
CTTATGGCCAATGTTCGATGCTTAACCCCCCCAATTTCTGTGAGATGGATGGCCAGTGCAAGCGTGACTTGAA
GTGTTGCATGGGCATGTGTGGGAAATCCTGCGTTTCCCCTGTGAAAGCTTGATTCCTGCCATATGGAGGAGGC
TCTGGAGTCCTGCTCTGTGTGGTCCAGGTCCTTTCCACCCTGAGACTTGGCTCCACCACTGATATCCTCCTTT
GGGGAAAGGCTTGGCACACAGCAGGCTTTCAAGAAGTGCCAGTTGATCAATGAATAAATAAACGAGCCTATTT
CTCTTTGCAC

| | | | |
|---|---|---|---|
| NOV8e, SNP 13378858 | | | |
| Protein Sequence | SEQ ID NO: 92 | 132 aa | MW at 14299.8kD |

MKSSGLFPFLVLLALGTLAPWAVEGSGKSFKAGVCPPKKSAQCLRYKKPECQSDWQCPGKKRCCPDTCGIKCL
DPVDTPNPTRRKPGKCPVTYGQCSMLNPPNFCEMDGQCKRDLKCCMGMCGKSCVSPVKA

| | | |
|---|---|---|
| NOV8f, SNP 13378859 | SEQ ID NO: 93 | 594 bp |
| DNA Sequence | ORF Start: ATG at 19 | ORF Stop: TGA at 415 |

GTCACTCCTGCCTTCACCATGAAGTCCAGCGGCCTCTTCCCCTTCCTGGTGCTGCTTGCCCTGGGAACTCTGG
CACCTTGGGCTGTGGAAGGCTCTGGAAAGTCCTTCAAAGCTGGAGTCTGTCCTCCTAAGAAATCTGCCCAGTG
CCTTAGATACAAGAAACCTGAGTGCCAGAGTGACTGGCAGTGTCCAGGGAAGAAGAGATGTTGTCCTGACACT
TGTGGCATCAAATGCCTGGATCCTGTTGACACCCCAAACCCAACAAGGAGGAAGCCTGGGAAGTGCCCAGTGA
CTTATGGCCAATGTTTGATGCTTAACCCCCCCAATTTCTGTGAGATGGATGGCCAGTGCAAGCGTGACTTGAA
GTGTTGCATGGGCATGTGTGGGAAATCCCGCGTTTCCCCTGTGAAAGCTTGATTCCTGCCATATGGAGGAGGC
TCTGGAGTCCTGCTCTGTGTGGTCCAGGTCCTTTCCACCCTGAGACTTGGCTCCACCACTGATATCCTCCTTT
GGGGAAAGGCTTGGCACACAGCAGGCTTTCAAGAAGTGCCAGTTGATCAATGAATAAATAAACGAGCCTATTT
CTCTTTGCAC

| | | | |
|---|---|---|---|
| NOV8f, SNP 13378859 | | | |
| Protein Sequence | SEQ ID NO: 94 | 132 aa | MW at 14379.0kD |

MKSSGLFPFLVLLALGTLAPWAVEGSGKSFKAGVCPPKKSAQCLRYKKPECQSDWQCPGKKRCCPDTCGIKCL
DPVDTPNPTRRKPGKCPVTYGQCLMLNPPNFCEMDGQCKRDLKCCMGMCGKSRVSPVKA

| | | |
|---|---|---|
| NOV8g, CG55060 | SEQ ID NO: 95 | 594 bp |
| DNA Sequence | ORF Start: ATG at 19 | ORF Stop: TGA at 415 |

GTCACTCCTGCCTTCACCATGAAGTCCAGCGGCCTCTX₁CCCCTTCCTGGTGCX₂GCTTGCCCTGGGAACTCT
GGCACCTTGGGCTGTGGAAGGCTCTGGAAAGTCCTTCAAAGCTGGAGTCTGTCCTCCTAAGAAATCTGCCCAG
TGCCTTAGATACAAGAAACCTGAGX₃GCCAGAGTGACTGGCAGTGTCCAGGGAAGAAGAGATGTTGTCCTGAC
ACTTGTGGCATCAAATGCCTGGATCCTGTTGACACCCCAAACCCAACAAGGAGGAAGCCTGGGAAGTGCCCAG
TGACTTATGGCCAATGTTX₄GATGCTTAACCCCCCCAATTTCTGTGAGATGGATGGCCAGTGCAAGCGTGACT
TGAAGTGTTGCATGGGCATGTGTGGGAAATCCX₅GCGTTTCCCCTGTGAAAGCTTGATTCCTGCCATATGGAG
GAGGCTCTGGAGTCCTGCTCTGTGTGGTCCAGGTCCTTTCCACCCTGAGACTTGGCTCCACCACTGATATCCT
CCTTTGGGGAAAGGCTTGGCACACAGCAGGCTTTCAAGAAGTGCCAGTTGATCAATGAATAAATAAACGAGCC
TATTTCTCTTTGCAC

[Wherein each of residues X₁, X₂, X₃, X4_{,}and X₅, is either T or C.]

| | | | |
|---|---|---|---|
| NOV8g, CG55060 | | | |
| Protein Sequence | SEQ ID NO: 96 | 132 aa | MW at 14325.9kD |

MKSSGLZ₁PFLVZ₂LALGTLAPWAVEGSGKSFKAGVCPPKKSAQCLRYKKPEZ₃QSDWQCPGKKRCCPDTCGI
KCLDPVDTPNPTRRKPGKCPVTYGQCZ₄MLNPPNFCEMDGQCKRDLKCCMGMCGKSZ₅VSPVKA
[Wherein residue Z₁ is F or S; Z₂ is L or P; Z₃ is C or R; Z₄ is L or S;
and Z₅ is C or R.]

Further analysis of the NOV8a protein yielded the following properties shown in Table8C.

TABLE 8C
Protein Sequence Properties NOV8a

| SignalP | No Known Signal Sequence Predicted | | | |
|---|---|---|---|---|
| analysis: | | | | |
| PSORT II | Psort Results (see Details): | | | |
| analysis: | 88.0%: nucleus | | | |
| | 10.0%: mitochondrial matrix space | | | |
| | 10.0%: lysosome (lumen) | | | |
| | 0.0%: endoplasmic reticulum (membrane) | | | |
| | Psort II Results (see Details): | | | |
| | 87.0%: nuclear | | | |
| | 13.0%: mitochondrial | | | |
| | Details of Psort Prediction | | | |
| | >>> MUS belongs to the animal class | | | |
| | *** Reasoning Step: 2 | | | |
| | SRCFLG: 1 | | | |
| | Prelim. Calc. of ALOM (thresh: 0.5) count: 0 | | | |
| | McG: Length of UR: 6 | | | |
| | Peak Value of UR: −0.36 | | | |
| | Net Charge of CR: 2 | | | |
| | McG: Discrim Score: −17.57 | | | |
| | GvH: Signal Score (−3.5): −7.95 | | | |
| | Possible site: 53 | | | |
| | >>> Seems to have no N-terminal signal seq. | | | |
| | Amino Acid Composition: calculated from 1 | | | |
| | new cnt: 0 ** thrshld changed to −2 | | | |
| | involving clv. sig in the ALOMREC or not: 0B | | | |
| | ALOM program count: 0 value: 8.59 threshold: −2.0 | | | |
| | PERIPHERAL Likelihood = 8.59 | | | |
| | modified ALOM score: −2.62 | | | |
| | Gavel: Bound. Mitoch. Preseq. R-2 motif: 22 LRYKKP | | | |
| | mtdisc (mit) Status: negative (−2.26) | | | |
| | *** Reasoning Step: 3 | | | |
| | KDEL Count: 0 | | | |
| | Goal mtmx modified Score: 0.10 | | | |
| | SKL motif: pos: −1(107), count: 0 | | | |
| | Poxaac Score: −11.55 | | | |
| | >>> POX Status: negative | | | |
| | >>> lys: −6.99 Status: negative | | | |
| | Goal lys: modified. Score: 0.100 | | | |
| | Nuc-4 pos: 57 (4) RRKP | | | |
| | Robbins & Dingwall pos: 21 (3) KK PECQSDWQCP GKKRC | | | |
| | nuc mod by robbins. Score: 0.60 | | | |
| | nuc modified. Score: 0.90 | | | |
| | >>> Nuclear Signal. Status: positive (0.70) | | | |
| | Details of Psort II Prediction | | | |
| | *** Warning: 1st aa is not methyonine | | | |
| | PSG: a new signal peptide prediction method | | | |
| | N-region: length 6; pos.chg 2; neg.chg 0 | | | |
| | H-region: length 6; peak value −5.62 | | | |
| | PSG score: −10.02 | | | |
| | GvH: von Heijne's method for signal seq. recognition | | | |
| | GvH score (threshold: −2.1): −11.95 | | | |
| | possible cleavage site: between 53 and 54 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 8.59 (at 89) | | | |
| | ALOM score: 8.59 (number of TMSs: 0) | | | |
| | MITDISC: discrimination of mitochondrial targeting seq | | | |
| | R content: | 1 | Hyd Moment(75): | 3.92 |
| | Hyd Moment(95): | 8.87 | G content: | 2 |
| | D/E content: | 1 | S/T content: | 3 |

Score: −4.11
Gavel: prediction of cleavage sites for mitochondrial preseq
R-2 motif at 30 LRY|KK
NUCDISC: discrimination of nuclear localization signals
pat4: RRKP (4) at 58
pat7: PGKKRCC (5) at 33
pat7: PNPTRRK (3) at 54
pat7: PTRRKPG (5) at 56
bipartite: KKPECQSDWQCPGKKRC at 22
content of basic residues: 18.7%
NLS Score: 1.39
ER Membrane Retention Signals:
KKXX-like motif in the C-terminus: SPVK
NNCN: Reinhardt's method for Cytplasmic/Nuclear discrimination
Prediction: nuclear
Reliability: 94.1

A search of the NOV8a protein against the Geneseq database, a proprietary database that contains sequences published in patents and patent publication, yielded several homologous proteins shown in Table 8C

TABLE 8C
GENESEQ RESULTS FOR NOV8a

| | | NOV8a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAU99884 | rSLAP1 fusion protein - Homo sapiens, 503 | 1 . . . 107 | 106/107 (99%) | 0.0 |
| | aa. | 397 . . . 503 | 106/107 (99%) | |
| AAP60562 | Synthetic protein capable of directing | 1 . . . 107 | 106/107 (99%) | 0.0 |
| | microbial synthesis of a serine protease | 1 . . . 107 | 106/107 (99%) | |
| | inhibitor having similar properties to protein | | | |
| | isolated from parotid secretions - Synthetic, | | | |
| | 107 aa. | | | |
| AAP60563 | Synthetic sequence capable of directing | 1 . . . 107 | 106/107 (99%) | 0.0 |
| | microbial synthesis of a secretory | 1 . . . 107 | 106/107 (99%) | |
| | leukocyte protease-inhibitor - Synthetic, | | | |
| | 107 aa | | | |
| AAP70584 | Sequence of protein with the biological | 1 . . . 107 | 106/107 (99%) | 0.0 |
| | activity of HUSI (human seminal plasma | 26 . . . 132 | 106/107 (99%) | |
| | inhibitor) type I inhibitors encoded on pRH | | | |
| | 34 - Homo sapiens, 132 aa. | | | |
| AAP90384 | Human polymorphonuclear leukocyte | 1 . . . 107 | 106/107 (99%) | 0.0 |
| | elastase inhibiting protein - Homo sapiens, | 1 . . . 107 | 106/107 (99%) | |
| | 107 aa. | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P03973 | Sequence 1 from Patent | 1 . . . 107 | 106/107 (99%) | 0.0 |
| | WO0229058 - Homo sapiens | 26 . . . 132 | 106/107 (99%) | |
| | (Human), 1492 aa. | | | |
| CAA00747 | ALP-242 PROTEIN - synthetic | 1 . . . 107 | 105/107 (98%) | 0.0 |
| | construct, 107 aa (fragment). | 1 . . . 107 | 106/107 (99%) | |
| CAA00742 | ALP-240 PROTEIN - synthetic | 1 . . . 107 | 104/107 (97%) | 0.0 |
| | construct, 107 aa (fragment). | 1 . . . 107 | 105/107 (98%) | |

PFam analysis does not predict any domains for the NOV8a protein. Pfam does predict that the NOV8a protein contains the domains shown below in the Table 8F. Specific amino acid residues of NOV8a for each domain is shown in column 2, equivalent domains in the other NOV8 proteins of the invention are also encompassed herein.

TABLE 8F
Domain Analysis of NOV8a

| | | Identities/ | |
|---|---|---|---|
| | | Similarities | |
| | NOV8a Match Region | for the | Expect |
| Pfam Domain | Amino Acid Residues: | Matched Region | Value |
| wap | 6 . . . 50 | 20/49 (41%) | 1.1e−13 |
| | | 41/49 (84%) | |
| wap | 60 . . . 104 | 20/49 (41%) | 2.2e−11 |
| | | 35/49 (71%) | |

### Example 9

### NOV9, CG56008, LIV-1 Protein

The NOV9 family of novel nucleic acids and polypeptides clones includes NOV9a through NOV9i, SEQ ID NOs: 97-114, and the nucleotide and encoded polypeptide sequences are shown in Table 9A. In a particular embodiment NOV9 nucleic acid sequence is SEQ ID NO:113, wherein residue X₁ is T or C. Nucleic acid sequence SEQ ID NO:113 encodes polypeptide SEQ ID NO:114, wherein residue Z₁ is L or P. Equivalent nucleic acid and polypeptide substitutions apply to other NOV9 sequences as would be appreciated by one of skill in the art, and are emcompassed in the present invention.

TABLE 9A
NOV9 Sequence Analysis

| | | |
|---|---|---|
| NOV9a, CG56008-01 | SEQ ID NO: 97 | 3445 bp |
| DNA Sequence | ORF Start: ATG at 117 | ORF Stop: TAG at 2382 |

CACCGCGTGTTCGCGCCTGGTAGAGATTTCTCGAAGACACCAGTGGGCCCGTGTGGAACCAAACCTGCGCGCG
TGGCCGGGCCGTGGGACAACGAGGCCGCGGAGACGAAGGCGCAATGGCGAGGAAGTTATCTGTAATCTTGATC
CTGACCTTTGCCCTCTCTGTCACAAATCCCCTTCATGAACTAAAAGCAGCTGCTTTCCCCCAGACCACTGAGA
AAATTAGTCCGAATTGGGAATCTGGCATTAATGTTGACTTGGCAATTTCCACACGGCAATATCATCTACAACA
GCTTTTCTACCGCTATGGAGAAAATAATTCTTTGTCAGTTGAAGGGTTCAGAAAATTACTTCAAAATATAGGC
ATAGATAAGATTAAAAGAATCCATATACACCATGACCACGACCATCACTCAGACCACGAGCATCACTCAGACC
ATGAGCGTCACTCAGACCATGAGCATCACTCAGAGCACGAGCATCACTCTGACCATGATCATCACTCTCACCA
TAATCATGCTGCTTCTGGTAAAAATAAGCGAAAAGCTCTTTGCCCAGACCATGACTCAGATAGTTCAGGTAAA
GATCCTAGAAACAGCCAGGGGAAAGGAGCTCACCGACCAGAACATGCCAGTGGTAGAAGGAATGTCAAGGACA
GTGTTAGTGCTAGTGAAGTGACCTCAACTGTGTACAACACTGTCTCTGAAGGAACTCACTTTCTAGAGACAAT
AGAGACTCCAAGACCTGGAAAACTCTTCCCCAAAGATGTAAGCAGCTCCACTCCACCCAGTGTCACATCAAAG
AGCCGGGTGAGCCGGCTGGCTGGTAGGAAAACAAATGAATCTGTGAGTGAGCCCCGAAAAGGCTTTATGTATT
CCAGAAACACAAATGAAAATCCTCAGGAGTGTTTCAATGCATCAAAGCTACTGACATCTCATGGCATGGGCAT
CCAGGTTCCGCTGAATGCAACAGAGTTCAACTATCTCTGTCCAGCCATCATCAACCAAATTGATGCTAGATCT
TGTCTGATTCATACAAGTGAAAAGAAGGCTGAAATCCCTCCAAAGACCTATTCATTACAAATAGCCTGGGTTG
GTGGTTTTATAGCCATTTCCATCATCAGTTTCCTGTCTCTGCTGGGGGTTATCTTAGTGCCTCTCATGAATCG
GGTGTTTTTCAAATTTCTCCTGAGTTTCCTTGTGGCACTGGCCGTTGGGACTTTGAGTGGTGATGCTTTTTTA
CACCTTCTTCCACATTCTCATGCAAGTCACCACCATAGTCATAGCCATGAAGAACCAGCAATGGAAATGAAAA
GAGGACCACTTTTCAGTCATCTGTCTTCTCAAAACATAGAAGAAAGTGCCTATTTTGATTCCACGTGGAAGGG
TCTAACAGCTCTAGGAGGCCTGTATTTCATGTTTCTTGTTGAACATGTCCTCACATTGATCAAACAATTTAAA
GATAAGAAGAAAAAGAATCAGAAGAAACCTGAAAATGATGATGATGTGGAGATTAAGAAGCAGTTGTCCAAGT
ATGAATCTCAACTTTCAACAAATGAGGAGAAAGTAGATACAGATGATCGAACTGAAGGCTATTTACGAGCAGA
CTCACAAGAGCCCTCCCACTTTGATTCTCAGCAGCCTGCAGTCTTGGAAGAAGAAGAGGTCATGATAGCTCAT
GCTCATCCACAGGAAGTCTACAATGAATATGTACCCAGAGGGTGCAAGAATAAATGCCATTCACATTTCCACG
ATACACTCGGCCAGTCAGACGATCTCATTCACCACCATCATGACTACCATCATATTCTCCATCATCACCACCA
CCAAAACCACCATCCTCACAGTCACAGCCAGCGCTACTCTCGGGAGGAGCTGAAAGATGCCGGCGTCGCCACT
CTGGCCTGGATGGTGATAATGGGTGATGGCCTGCACAATTTCAGCGATGGCCTAGCAATTGGTGCTGCTTTTA
CTGAAGGCTTATCAAGTGGTTTAAGTACTTCTGTTGCTGTGTTCTGTCATGAGTTGCCTCATGAATTAGGTGA
CTTTGCTGTTCTACTAAAGGCTGGCATGACCGTTAAGCAGGCTGTCCTTTATAATGCATTGTCAGCCATGCTG
GCGTATCTTGGAATGGCAACAGGAATTTTCATTGGTCATTATGCTGAAAATGTTTCTATGTGGATATTTGCAC
TTACTGCTGGCTTATTCATGTATGTTGCTCTGGTTGATATGGTACCTGAAATGCTGCACAATGATGCTAGTGA
CCATGGATGTAGCCGCTGGGGGTATTTCTTTTTACAGAATGCTGGGATGCTTTTGGGTTTTGGAATTATGTTA
CTTATTTCCATATTTGAACATAAAATCGTGTTTCGTATAAATTTCTAGTTAAGGTTTAAATGCTAGAGTAGCT
TAAAAAGTTGTCATAGTTTCAGTAGGTCATAGGGAGATGAGTTTGTATGCTGTACTATGCAGCGTTTAAAGTT
AGTGGGTTTTGTGATTTTTGTATTGAATATTGCTGTCTGTTACAAAGTCAGTTAAAGGTACGTTTTAATATTT
AAGTTATTCTATCTTGGAGATAAAATCTGTATGTGCAATTCACCGGTATTACCAGTTTATTATGTAAACAAGA
GATTTGGCATGACATGTTCTGTATGTTTCAGGGAAAAATGTCTTTAATGCTTTTTCAAGAACTAACACAGTTA
TTCCTATACTGGATTTTAGGTCTCTGAAGAACTGCTGGTGTTTAGGAATAAGAATGTGCATGAAGCCTAAAAT
ACCAAGAAAGCTTATACTGAATTTAAGCAAAGAAATAAAGGAGAAAAGAGAAGAATCTGAGAATTGGGGAGGC
ATAGATTCTTATAAAAATCACAAAATTTGTTGTAAATTAGAGGGGAGAAATTTAGAATTAAGTATAAAAAGGC
AGAATTAGTATAGAGTACATTCATTAAACATTTTTGTCAGGATTATTTCCCGTAAAAACGTAGTGAGCACTTT
ATATATCACTTGACCAAGAAATTGGAATTTCAAAATGTTCGTGCGGGTATATACCAGATGAGTACAGTGAGTA
GTTTTATGTATCACCAGACTGGGTTATTGCCAAGTTATATATCACCAAAAGCTGTATGACTGGATGTTCTGGT
TACCTGGTTTACAAAATTATCAGAGTAGTAAAACTTTGATATATATGAGGATATTAAAACTACACTAAGTATC
ATTTGATTCGATTCAGAAAGTACTTTGATATCTCTCAGTGCTTCAGTGCTATCATTGTGAGCAATTGTCTTTT
ATATACGGTACTGTAGCCATACTAGGCCTGTCTGTGGCATTCTCTAGATGTTTCTTTTTTACACAATAAATTC
CTTATATCAGCTTG

| | | | |
|---|---|---|---|
| NOV9a, CG56008-01 | | | |
| Protein Sequence | SEQ ID NO: 98 | 755 aa | MW at 85046.0kD |

MARKLSVILILTFALSVTNPLHELKAAAFPQTTEKISPNWESGINVDLAISTRQYHLQQLFYRYGENNSLSVE
GFRKLLQNIGIDKIKRIHIHHDHDHHSDHEHHSDHERHSDHEHHSEHEHHSDHDHNSHHNHAASGKNKRKALC
PDHDSDSSGKDPRNSQGKGAHRPEHASGRRMVKWSVSASEVTSTVYNTVSEGTHFLETIETPRPGKLFPKDVS
SSTPPSVTSKSRVSRLAGRKTNESVSEPRKGFMYSRNTNENPQECFNASKLLTSHGMGIQVPLNATEFNYLCP
AIINQIDARSCLIHTSEKKAEIPPKTYSLQIAWVGGFIAISIISFLSLLGVILVPLMNRVFFKFLLSFLVALA
VGTLSGDAFLHLLPHSHASHHHSHSHEEPAMEMKRGPLFSHLSSQNIEESAYFDSTWKGLTALGGLYFMFLVE
HVLTLIKQFKDKKKKNQKKPENDDDVEIKKQLSKYESQLSTNEEKVDTDDRTEGYLRADSQEPSHFDSQQPAV
LEEEEVMIAHAHPQEVYNEYVPRGCKNKCHSHFHDTLGQSDDLIHHHHDYHHILHHHHHQNHHPHSHSQRYSR
EELKDAGVATLAWMVIMGDGLHNFSDGLAIGAAFTEGLSSGLSTSVAVFCHELPHELGDFAVLLKAGMTVKQA
VLYNALSAMLAYLGMATGIFIGHYAENVSMWIFALTAGLFMYVALVDMVPEMLHNDASDHGCSRWGYFFLQNA
GMLLGFGIMLLISIFEHKIVFRINF

| | | |
|---|---|---|
| NOV9b, CG56008-02 | SEQ ID NO: 99 | 912 bp |
| DNA Sequence | ORF Start: at 1 | ORF Stop: end of sequence |

AATCCCCTTTATGAACTAAAAGCAGCTGCTTTCCCTCAGACCACTGAGAAAATTAGTCCGAATTGGGAATCTG
GCATTAATGTTGACTTGGCAATTTCCACACGGCAATATCATCTACAACAGCTTTTCTACCGCTATGGAGAAAA
TAATTCTTTGTCAGTTGAAGGGTTCAGAAAATTACTTCAAAATATAGGCATAGATAAGATTAAAAGAATCCAT
ATACACCATGACCACGACCATCACTCAGACCACGAGCATCACTCAGACCATGAGCGTCACTCAGACCATGAGC
ATCACTCAGACCACGAGCATCACTCTGACCATGATCATCACTCCCACCATAATCATGCTGCTTCTGGTAAAAA
TAAGCGAAAAGCTCTTTGCCCAGACCATGACTCAGATAGTTCAGGTAAAGATCCTAGAAACAGCCAGGGGAAA
GGAGCTCACCGACCAGAACATGCCAGTGGTAGAAGGAATGTCAAGGACAGTGTTAGTGCTAGTGAAGTGACCT
CAACTGTGTACAACACTGTCTCTGAAGGAACTCACTTTCTAGAGACAATAGAGACTCCAAGACCTGGAAAACT
CTTCCCCAAAGATGTAAGCAGCTCCACTCCACCCAGTGTCACATCAAAGAGCCGGGTGAGCCGGCTGGCTGGT
AGGAAAACAAATGAATCTGTGAGTGAGCCCCGAAAAGGCTTTATGTATTCCAGAAACACAAATGAAAATCCTC
AGGAGTGTTTCAATGCATCAAAGCTACTGACATCTCATGGCATGGGCATCCAGGTTCCGCTGAATGCAACAGA
GTTCAACTATCTCTGTCCAGCCATCATCAACCAAATTGATGCTAGATCTTGTCTGATTCATACAAGTGAAAAG
AAGGCTGAAATCCCTCCAAAGACCTATTCATTACAA

| | | | |
|---|---|---|---|
| NOV9b, CG56008-02 | | | |
| Protein Sequence | SEQ ID NO: 100 | 304 aa | MW at 34320.4kD |

NPLYELKAAAFPQTTEKISPNWESGINVDLAISTRQYHLQQLFYRYGENNSLSVEGFRKLLQNIGIDKIKRIH
IHHDHDHHSDHEHHSDHERHSDHEHHSDHEHHSDHDHHSHHNHAASGKNKRKALCPDHDSDSSGKDPRNSQGK
GAHRPEHASGRRNVKDSVSASEVTSTVYNTVSEGTHFLETIETPRPGKLFPKDVSSSTPPSVTSKSRVSRLAG
RKTNESVSEPRKGFMYSRNTNENPQECFNASKLLTSHGMGIQVPLNATEFNYLCPAIINQIDARSCLIHTSEK
KAEIPPKTYSLQ

| | | |
|---|---|---|
| NOV9c, CG56008-03 | SEQ ID NO: 101 | 1186 bp |
| DNA Sequence | ORF Start: ATG at 3 | ORF Stop: TGA at 1149 |

CTATGGGCGCGGCTGCCGGGTGGCTGCGCGGCGCTGCCCCCGGACCGAGGGGCAGCCAATCCAATGAAACCAC
CGCGTGTTCGCGCCTGGTAGAGATTTCTCGAAGACACCAGTGGGCCCGTTCCGAGCCCTCTGGACCGCCCGTG
TGGAACCAAACCTGCGCGCGTGGCCGGGCCGTGGGACAACGAGGCCGCGGAGACGAAGGCGCAATGGCGAGGA
AGTTATCTGTAATCTTGATCCTGACCTTTGCCCTCTCTGTCACAAATCCCCTTCATGAACTAAAAGCAGCTGC
TTTCCCCCAGACCACTGAGAAAATTAGTCCGAATTGGGAATCTGGCATTAATGTTGACTTGGCAATTTCCACA
CGGCAATATCATCTACAACAGCTTTTCTACCGCTATGGAGAAAATAATTCTTTGTCAGTTGAAGGGTTCAGAA
AATTACTTCAAAATATAGGCATAGATAAGATTAAAAGAATCCATATACACCATGACCACGACCATCACTCAGA
CCACGAGCATCACTCAGACCATGAGCGTCACTCAGACCATGAGCATCACTCAGACCACGAGCATCACTCTGAC
CATGATCATCACTCTCACCATAATCATGCTGCTTTTACTGAAGGCTTATCAAGTGGTTTAAGTACTTCTGTTG
CTGTGTTCTGTCATGAGTTGCCTCATGAATTAGGTGACTTTGCTGTTCTACTAAAGGCTGGCATGACCGTTAA
GCAGGCTGTCCTTTATAATGCATTGTCAGCCATGCTGGCGTATCTTGGAATGGCAACAGGAATTTTCATTGGT
CATTATGCTGAAAATGTTTCTATGTGGATATTTGCACTTACTGCTGGCTTATTCATGTATGTTGCTCTGGTTG
ATATGGTACCTGAAATGCTGCACAATGATGCTAGTGACCATGGATGTAGCCACTGGGGGTATTTCTTTTTACA
GAATGCTGGGATGCTTTTGGGTTTTGGAATTATGTTACTTATTTCCATATTTGAACATAAAATCGTGTTTCGT
ATAAATTTCAATTCTCCATCATCACCACCACCAAAACCACCATCCTCACAGTCACAGCCAGCGCTACTCTCGG
GAGGAGCTGAAAGATGCCGGCGTCGCCACTCTGGCCTGGATGGTGATAATGGGTGATGGCCTGCACAATTTCA
GCGATGGCCTAGCAATTG

| | | | |
|---|---|---|---|
| NOV9c, CG56008-03 | | | |
| Protein Sequence | SEQ ID NO: 102 | 382 aa | MW at 42317.2kD |

MGAAAGWLRGAAPGPRGSQSNETTACSRLVEISRRHQWARSEPSGPPVWNQTCARGRAVGQRGRGDEGAMARK
LSVILILTFALSVTNPLHELKAAAFPQTTEKISPNWESGINVDLAISTRQYHLQQLFYRYGENNSLSVEGFRK
LLQNIGIDKIKRIHTHHDHDHHSDHEHHSDHERHSDHEHHSDHEHHSDHDHHSHHNHAAFTEGLSSGLSTSVA
VFCHELPHELGDFAVLLKAGMTVKQAVLYNALSAMLAYLGMATGIFIGHYAENVSMWIFALTAGLFMYVALVD
MVPEMLHNDASDHGCSHWGYFFLQNAGMLLGFGIMLLISIFEHKIVFRINFNSPSSPPPKPPSSQSQPALLSG
GAERCRRRHSGLDGDNG

| | | |
|---|---|---|
| NOV9d, CG56008-04 | SEQ ID NO: 103 | 1101 bp |
| DNA Sequence | ORF Start: ATG at 123 | ORF Stop: TAG at 1029 |

TGGTAGAGATTTCTCGAAGACACCAGTGGGCCCGTTCCGAGCCCTCTGGACCGCCCGTGTGGAACCAAACCTG
CGCGCGTGGCCGGGCCGTGGGACAACGAGGCCGCGGAGACGAAGGCGCAATGGCGAGGAAGTTATCTGTAATC
TTGATCCTGACCTTTGCCCTCTCTGTCACAAATCCCCTTCATGAACTAAAAGCAGCTGCTTTCCCCCAGACCA
CTGAGAAAATTAGTCCGAATTGGGAATCTGGCATTAATGTTGACTTGGCAATTTCCACACGGCAATATCATCT
ACAACAGCTTTTCTACCGCTATGGAGAAAATAATTCTTTGTCAGTTGAAGGGTTCAGAAAATTACTTCAAAAT
ATAGGCATAGATAAGATTAAAAGAATCCATATACACCATGACCACGACCATCACTCAGACCACGAGCATCACT
CAGACCATGAGCGTCACTCAGACCATGAGCATCACTCAGACCACCATCCTCACAGTCACAGCCAGCGCTACTC
TCGGGAGGAGCTGAAAGATGCCGGCGTCGCCACTTTGGCCTGGATGGTGATAATGGGTGATGGCCTGCACAAT
TTCAGCGATGGTCTAGCAATTGGTGCTGCTTTTACTGAAGGCTTATCAAGTGGTTTAAGTACTTCTGTTGCTG
TGTTCTGTCATGAGTTGCCTCATGAATTAGGTGACTTTGCTGTTCTACTAAAGGCTGGCATGACCGTTAAGCA
GGCTGTCCTTTATAATGCATTGTCAGCCATGCTGGCGTATCTTGGAATGGCAACAGGAATTTTCATTGGTCAT
TATGCTGAAAATGTTTCTATGTGGATATTTGCACTTACTGCTGGCTTATTCATGCATGTTGCTCTGGTTGATA
TGGTACCTGAAATGCTGCACAATGATGCTAGTGACCATGGATGTAGCCGCTGGGGGTATTTCTTTTTACAGAA
TGCTGGGATGCTTTTGGGTTTTGGAATTATGTTACTTATTTCCATATTTGAACATAAAATCGTGTTTCGTATA
AATTTCTAGTTAAGGTTTAAATGCTAGAGTAGCTTAAAAAGTTGTCATAGTTTCAGTAGGTCATAGGGAGATG
AGTTTG

| | | | |
|---|---|---|---|
| NOV9d, CG56008-04 | | | |
| Protein Sequence | SEQ ID NO: 104 | 302 aa | MW at 33918.4kD |

MARKLSVILILTFALSVTNPLHELKAAAFPQTTEKISPNWESGINVDLAISTRQYHLQQLFYRYGENNSLSVE
GFRKLLQNIGIDKIKRIHIHHDHDHHSDHEHHSDHERHSDHEHHSDHHPHSHSQRYSREELKDAGVATLAWMV
IMGDGLHNFSDGLAIGAAFTEGLSSGLSTSVAVFCHELPHELGDFAVLLKAGMTVKQAVLYNALSAMLAYLGM
ATGIFIGHYAENVSMWIFALTAGLFMHVALVDMVPEMLHNDASDHGCSRWGYFFLQNAGMLLGFGIMLLISIF
EHKIVFRINF

| | | |
|---|---|---|
| NOV9e, CG56008-05 | SEQ ID NO: 105 | 2268 bp |
| DNA Sequence | ORF Start: ATG at 1 | ORF Stop: TAG at 2266 |

ATGGCGAGGAAGTTATCTGTAATCTTGATCCTGACCTTTGCCCTCTCTGTCACAAACCCCCTTCATGAACTAA
AAGCAGCTGCTTTCCCCCAGACCACTGAGAAAATTAGTCCGAATTGGGAATCTGGCATTAATGTTGACTTGGC
AATTTCCACACGGCAATATCATCTACAACAGCTTTTCTACCGCTATGGAGAAAATAATTCTTTGTCAGTTGAG
GGGTTCAGAAAATTACTTCAAAATATAGGCATAGATAAGATTAAAAGAATCCATATACACCACGACCAcGACC
ATCACTCAGACCACGAGCATCACTCAGACCATGAGCGTCACTCAGACCATGAGCATCACTCAGACCACGAGCA
TCACTCTGACCATGATCATCACTCTCACCATAATCATGCTGCTTCTGGTAAAAATAAGCGAAAAGCTCTTTGC
CCAGACCATGACTCAGATAGTTCAGGTAAAGATCCTAGAAACAGCCAGGGGAAAGGAGCTCACCGACCAGAAC
ATGCCAGTGGTAGAAGGAATGTCAAGGACAGTGTTAGTGCTAGTGAAGTGACCTCAACTGTGTACAACACTGT
CTCTGAAGGAACTCACTTTCTAGAGACAATAGAGACTCCAAGACCTGGAAAACTCTTCCCCAAAGATGTAAGC
AGCTCCACTCCACCCAGTGTCACATCAAGAGCCGGGTGAGCCGGCTGGCTGGTAGGAAAACAAAATGAATCTG
TGAGTGAGCCCCGAAAAGGCTTTATGTATTCCAGAAACACAAATGAAAATCCTCAGGAGTGTTTCAATGCATC
AAAGCTACTGACATCTCATGGCATGGGCATCCAGGTTCCGCTGAATGCAACAGAGTTCAACTATCTCTGTCCA
GCCATCATCAACCAAATTGATGCTAGATCTTGTCTGATTCATACAAGTGAAAAGAAGGCTGAAATCCCTCCAA
AGACCTATTCATTACAAATAGCCTGGGTTGGTGGTTTTATAGCCATTTCCATCATCAGTTTCCTGTCTCTGCT
GGGGGTTATCTTAGTGCCTCTCATGAATCGGGTGTTTTTCAAATTTCTCCTGAGTTTCCTTGTGGCACTGGCC
GTTGGGACTTTGAGTGGTGATGCTTTTTTACACCTTCTTCCACATTCTCATGCAAGTCACCACCATAGTCATA
GCCATGAAGAACCAGCAATGGAAATGAAAAGAGGACCACTTTTTAGTCATCTGTCTTCTCAAAACATAGAAGA
AAGTGCCTATTTTGATTCCACGTGGAAGGGTCTAACAGCTCTAGGAGGCCTGTATTTCATGTTTCTTGTTGAA
CATGTCCTCACATTGATCAAACAATTTAAAGATAAGAAGAAAAAGAATCAGAAGAAACCTGAAAATGATGATG
ATGTGGAGATTAAGAAGCAGTTGTCCAAGTATGAATCTCAACTTTCAACAAATGAGGAGAAAGTAGATACAGA
TGATCGAACTGAAGGCTATTTACGAGCAGACTCACAAGAGCCCTCCCACTTTGATTCTCAGCAGCCTGCAGTC
TTGGAAGAAGAAGAGGTCATGATAGCTCATGCTCATCCACAGGAAGTCTACAATGAATATGTACCCAGAGGGT
GCAAGAATAAATGCCATTCACATTTCCACGATACACTCGGCCAGTCAGACGATCTCATTCACCACCATCATGA
CTACCATCATATTCTCCATCATCACCACCACCAAAACCACCATCCTCACAGTCACAGCCAGCGCTACTCTCGG
GAGGAGCTGAAAGATGCCGGCGTCGCCACTCTGGCCTGGATGGTGATAATGGGTGATGGCCTGCACAATTTCA
GCGATGGCCTAGCAATTGGTGCTGCTTTTACTGAAGGCTTATCAAGTGGTTTAAGTACTTCTGTTGCTGTGTT
CTGTCATGAGTTGCCTCATGAATTAGGTGACTTTGCTGTTCTACTAAAGGCTGGCATGACCGTTAAGCAGGCT
GTCCTTTATAATGCATTGTCAGCCATGCTGGCGTATCTTGGAATGGCAACAGGAATTTTCATTGGTCATTATG
CTGAAAATGTTTCTATGTGGATATTTGCACTTACTGCTGGCTTATTCATGTATGTTGCTCTGGTTGATATGGT
ACCTGAAATGCTGCACAATGATGCTAGTGACCATGGATGTAGCCGCTGGGGGTATTTCTTTTTACAGAATGCT
GGGATGCTTTTGGGTTTTGGAATTATGTTACTTATTTCCATATTTGAACATAAAATCGTGTTTCGTATAAATT
TCTAG

| | | | |
|---|---|---|---|
| NOV9e, GG56008-05 | | | |
| Protein Sequence | SEQ ID NO: 106 | 755 aa | MW at 85032.0kD |

MARKLSVILILTFALSVTNPLHELKAAAFPQTTEKISPNWESGINVDLAISTRQYHLQQLFYRYGENNSLSVE
GFRKLLQNIGIDKIKRIHIHHDHDHHSDHEHHSDHERHSDHEHHSDHEHHSDHDHHSHHNHAASGKNKRKALC
PDHDSDSSGKDPRNSQGKGAHRPEHASGRRNVKDSVSASEVTSTVYNTVSEGTHFLETIETPRPGKLFPKDVS
SSTPPSVTSKSRVSRLAGRKTNESVSEPRKGFMYSRNTNENPQECFNASKLLTSHGMGIQVPLNATEFNYLCP
AIINQIDARSCLIHTSEKKAEIPPKTYSLQIAWVGGFIAISIISFLSLLGVILVPLMNRVFFKFLLSFLVALA
VGTLSGDAFLHLLPHSHASHHHSHSHEEPAMEMKRGPLFSHLSSQNIEESAYFDSTWKGLTALGGLYFMFLVE
HVLTLIKQFKDKKKKNQKKPENDDDVEIKKQLSKYESQLSTNEEKVDTDDRTEGYLRADSQEPSHFDSQQPAV
LEEEEVMIAHAHPQEVYNEYVPRGCKNKCHSHFHDTLGQSDDLIHHHHDYHHILHHHHHQNHHPHSHSQRYSR
EELKDAGVATLAWMVIMGDGLHNFSDGLAIGAAFTEGLSSGLSTSVAVFCHELPHELGDFAVLLKAGMTVKQA
VLYNALSAMLAYLGMATGIFIGHYAENVSMWIFALTAGLFMYVALVDMVPEMLHNDASDHGCSRWGYFFLQNA
GMLLGFGIMLLISIFEHKIVFRINF

| | | |
|---|---|---|
| NOV9f, CG56008-06 | SEQ ID NO: 107 | 2310 bp |
| DNA Sequence | ORF Start: ATG at 11 | ORF Stop: TAG at 2308 |

ATGGGTAAGCCTATCCCTAACCCTCTCCTCGGTCTCGATTCTACGGCGAGGAAGTTATCTGTAATCTTGATCC
TGACCTTTGCCCTCTCTGTCACAAACCCCCTTCATGAACTAAAAGCAGCTGCTTTCCCCCAGACCACTGAGAA
AATTAGTCCGAATTGGGAATCTGGCATTAATGTTGACTTGGCAATTTCCACACGGCAATATCATCTACAACAG
CTTTTCTACCGCTATGGAGAAAATAATTCTTTGTCAGTTGAGGGGTTCAGAAAATTACTTCAAAATATAGGCA
TAGATAAGATTAAAAGAATCCATATACACCACGACCACGACCATCACTCAGACCACGAGCATCACTCAGACCA
TGAGCGTCACTCAGACCATGAGCATCACTCAGACCACGAGCATCACTCTGACCATGATCATCACTCTCACCAT
AATCATGCTGCTTCTGGTAAAAATAAGCGAAAAGCTCTTTGCCCAGACCATGACTCAGATAGTTCAGGTAAAG
ATCCTAGAAACAGCCAGGGGAAAGGAGCTCACCGACCAGAACATGCCAGTGGTAGAAGGAATGTCAAGGACAG
TGTTAGTGCTAGTGAAGTGACCTCAACTGTGTACAACACTGTCTCTGAAGGAACTCACTTTCTAGAGACAATA
GAGACTCCAAGACCTGGAAAACTCTTCCCCAAAGATGTAAGCAGCTCCACTCCACCCAGTGTCACATCAAAGA
GCCGGGTGAGCCGGCTGGCTGGTAGGAAAACAAATGAATCTGTGAGTGAGCCCCGAAAAGGCTTTATGTATTC
CAGAAACACAAATGAAAATCCTCAGGAGTGTTTCAATGCATCAAAGCTACTGACATCTCATGGCATGGGCATC
CAGGTTCCGCTGAATGCAACAGAGTTCAACTATCTCTGTCCAGCCATCATCAACCAAATTGATGCTAGATCTT
GTCTGATTCATACAAGTGAAAAGAAGGCTGAAATCCCTCCAAAGACCTATTCATTACAAATAGCCTGGGTTGG
TGGTTTTATAGCCATTTCCATCATCAGTTTCCTGTCTCTGCTGGGGGTTATCTTAGTGCCTCTCATGAATCGG
GTGTTTTTCAAATTTCTCCTGAGTTTCCTTGTGGCACTGGCCGTTGGGACTTTGAGTGGTGATGCTTTTTTAC
ACCTTCTTCCACATTCTCATGCAAGTCACCACCATAGTCATAGCCATGAAGAACCAGCAATGGAAATGAAAAG
AGGACCACTTTTTAGTCATCTGTCTTCTCAAAACATAGAAGAAAGTGCCTATTTTGATTCCACGTGGAAGGGT
CTAACAGCTCTAGGAGGCCTGTATTTCATGTTTCTTGTTGAACATGTCCTCACATTGATCAAACAATTTAAAG
ATAAGAAGAAAAAGAATCAGAAGAAACCTGAAAATGATGATGATGTGGAGATTAAGAAGCAGTTGTCCAAGTA
TGAATCTCAACTTTCAACAAATGAGGAGAAAGTAGATACAGATGATCGAACTGAAGGCTATTTACGAGCAGAC
TCACAAGAGCCCTCCCACTTTGATTCTCAGCAGCCTGCAGTCTTGGAAGAAGAAGAGGTCATGATAGCTCATG
CTCATCCACAGGAAGTCTACAATGAATATGTACCCAGAGGGTGCAAGAATAAATGCCATTCACATTTCCACGA
TACACTCGGCCAGTCAGACGATCTCATTCACCACCATCATGACTACCATCATATTCTCCATCATCACCACCAC
CAAAACCACCATCCTCACAGTCACAGCCAGCGCTACTCTCGGGAGGAGCTGAAAGATGCCGGCGTCGCCACTC
TGGCCTGGATGGTGATAATGGGTGATGGCCTGCACAATTTCAGCGATGGCCTAGCAATTGGTGCTGCTTTTAC
TGAAGGCTTATCAAGTGGTTTAAGTACTTCTGTTGCTGTGTTCTGTCATGAGTTGCCTCATGAATTAGGTGAC
TTTGCTGTTCTACTAAAGGCTGGCATGACCGTTAAGCAGGCTGTCCTTTATAATGCATTGTCAGCCATGCTGG
CGTATCTTGGAATGGCAACAGGAATTTTCATTGGTCATTATGCTGAAAATGTTTCTATGTGGATATTTGCACT
TACTGCTGGCTTATTCATGTATGTTGCTCTGGTTGATATGGTACCTGAAATGCTGCACAATGATGCTAGTGAC
CATGGATGTAGCCGCTGGGGGTATTTCTTTTTACAGAATGCTGGGATGCTTTTGGGTTTTGGAATTATGTTAC
TTATTTCCATATTTGAACATAAAATCGTGTTTCGTATAAATTTCTAG

| | | | |
|---|---|---|---|
| NOV9f, CG56008-06 | +TL,15 | | |
| Protein Sequence | SEQ ID NO: 108 | 769 aa | MW at 86435.6kD |

MGKPIPNPLLGLDSTARKLSVILILTFALSVTNPLHELKAAAFPQTTEKISPNWESGINVDLAISTRQYHLQQ
LFYRYGENNSLSVEGFRKLLQNIGIDKIKRIHIHHDHDHHSDHEHHSDHERHSDHEHHSDHEHHSDHDHHSHH
NHAASGKNKRKALCPDHDSDSSGKDPRNSQGKGAHRPEHASGRRNVKDSVSASEVTSTVYNTVSEGTHFLETI
ETPRPGKLFPKDVSSSTPPSVTSKSRVSRLAGRKTNESVSEPRKGFMYSRNTNENPQECFNASKLLTSHGMGI
QVPLNATEFNYLCPAIINQIDARSCLIHTSEKKAEIPPKTYSLQIAWVGGFIAISIISFLSLLGVILVPLMNR
VFFKFLLSFLVALAVGTLSGDAFLHLLPHSHASHHHSHSHEEPAMEMKRGPLFSHLSSQNIEESAYFDSTWKG
LTALGGLYFMFLVEHVLTLIKQFKDKKKKNQKKPENDDDVEIKKQLSKYESQLSTNEEKVDTDDRTEGYLRAD
SQEPSHFDSQQPAVLEEEEVMIAHAHPQEVYNEYVPRGCKNKCHSHFHDTLGQSDDLIHHHHDYHHILHHHHH
QNHHPHSHSQRYSREELKDAGVATLAWMVIMGDGLHNFSDGLAIGAAFTEGLSSGLSTSVAVFCHELPHELGD
FAVLLKAGMTVKQAVLYNALSAMLAYLGMATGIFIGHYAENVSMWIFALTAGLFMYVALVDMVPEMLHNDASD
HGCSRWGYFFLQNAGMLLGFGIMLLISIFEHKIVFRINF

| | | |
|---|---|---|
| NOV9g, 311531751 | SEQ ID NO: 109 | 2211 bp |
| DNA Sequence | ORF Start: at 1 | ORF Stop: end of sequence |

AATCCCCTTCATGAACTAAAAGCAGCTGCTTTCCCCCAGACCACTGAGAAAATTAGTCCGAATTGGGAATCTG
GCATTAATGTTGACTTGGCAATTTCCACACGGCAATATCATCTACAACAGCTTTTCTACCGCTATGGAGAAAA
TAATTCTTTGTCAGTTGAGGGGTTCAGAAAATTACTTCAAAATATAGGCATAGATAAGATTAAAAGAATCCAT
ATACACCACGACCACGACCATCACTCAGACCACGAGCATCACTCAGACCATGAGCGTCACTCAGACCATGAGC
ATCACTCAGACCACGAGCATCACTCTGACCATGATCATCACTCTCACCATAATCATGCTGCTTCTGGTAAAAA
TAAGCGAAAAGCTCTTTGCCCAGACCATGACTCAGATAGTTCAGGTAAAGATCCTAGAAACAGCCAGGGGAAA
GGAGCTCACCGACCAGACATGCCAGTGGTAGAGGAATGTCAAGGACAGTGTTAGTGCTAGTGAAAAGTGACCT
CAACTGTGTACAACACTGTCTCTGAAGGAACTCACTTTCTAGAGACAATAGAGACTCCAAGACCTGGAAAACT
CTTCCCCAAAGATGTAAGCAGCTCCACTCCACCCAGTGTCACATCAAAGAGCCGGGTGAGCCGGCTGGCTGGT
AGGAAAACAAATGAATCTGTGAGTGAGCCCCGAAAAGGCTTTATGTATTCCAGAAACACAAATGAAAATCCTC
AGGAGTGTTTCAATGCATCAAAGCTACTGACATCTCATGGCATGGGCATCCAGGTTCCGCTGAATGCAACAGA
GTTCAACTATCTCTGTCCAGCCATCATCAACCAAATTGATGCTAGATCTTGTCTGATTCATACAAGTGAAAAG
AAGGCTGAAATCCCTCCAAAGACCTATTCATTACAAATAGCCTGGGTTGGTGGTTTTATAGCCATTTCCATCA
TCAGTTTCCTGTCTCTGCTGGGGGTTATCTTAGTGCCTCTCATGAATCGGGTGTTTTTCAAATTTCTCCTGAG
TTTCCTTGTGGCACTGGCCGTTGGGACTTTGAGTGGTGATGCTTTTTTACACCTTCTTCCACATTCTCATGCA
AGTCACCACCATAGTCATAGCCATGAAGAACCAGCAATGGAAATGAAAAGAGGACCACTTTTTAGTCATCTGT
CTTCTCAAAACATAGAAGAAAGTGCCTATTTTGATTCCACGTGGAAGGGTCTAACAGCTCTAGGAGGCCTGTA
TTTCATGTTTCTTGTTGAACATGTCCTCACATTGATCAAACAATTTAAAGATAAGAAGAAAAAGAATCAGAAG
AAACCTGAAAATGATGATGATGTGGAGATTAAGAAGCAGTTGTCCAAGTATGAATCTCAACTTTCAACAAATG
AGGAGAAAGTAGATACAGATGATCGAACTGAAGGCTATTTACGAGCAGACTCACAAGAGCCCTCCCACTTTGA
TTCTCAGCAGCCTGCAGTCTTGGAAGAAGAAGAGGTCATGATAGCTCATGCTCATCCACAGGAAGTCTACAAT
GAATATGTACCCAGAGGGTGCAAGAATAAATGCCATTCACATTTCCACGATACACTCGGCCAGTCAGACGATC
TCATTCACCACCATCATGACTACCATCATATTCTCCATCATCACCACCACCAAAACCACCATCCTCACAGTCA
CAGCCAGCGCTACTCTCGGGAGGAGCTGAAAGATGCCGGCGTCGCCACTCTGGCCTGGATGGTGATAATGGGT
GATGGCCAGCACAATTTCAGCGATGGCCTAGCAATTGGTGATGCTTTTACTGAAGGCTTATCAAGTGGTTTAA
GTACTTCTGTTGCTGTGTTCTGTCATGAGTTGCCTCATGAATTAGGTGACTTTGCTGTTCTACTAAAGGCTGG
CATGACCGTTAAGCAGGCTGTCCTTTATAATGCATTGTCAGCCATGCTGGCGTATCTTGGAATGGCAACAGGA
ATTTTCATTGGTCATTATGCTGAAAATGTTTCTATGTGGATATTTGCACTTACTGCTGGCTTATTCATGTATG
TTGCTCTGGTTGATATGGTACCTGAAATGCTGCACAATGATGCTAGTGACCATGGATGTAGCCGCTGGGGGTA
TTTCTTTTTACAGAATGCTGGGATGCTTTTGGGTTTTGGAATTATGTTACTTATTTCCATATTTGAACATAAA
ATCGTGTTTCGTATAAATTTC

| | | | |
|---|---|---|---|
| NOV9g, 311531751 | | | |
| Protein Sequence | SEQ ID NO: 110 | 737 aa | MW at 83133.4kD |

NPLHELKAAAFPQTTEKISPNWESGINVDLAISTRQYHLQQLFYRYGENNSLSVEGFRKLLQNIGIDKIKRIH
THHDHDHHSDHEHHSDHERHSDHEHHSDHEHHSDHDHHSHHNHAASGKNKRKALCPDHDSDSSGKDPRNSQGK
GAHRPEHASGRRNVKDSVSASEVTSTVYNTVSEGTHFLETIETPRPGKLFPKDVSSSTPPSVTSKSRVSRLAG
RKTNESVSEPRKGFMYSRNTNENPQECFNASKLLTSHGMGIQVPLNATEFNYLCPAIINQIDARSCLIHTSEK
KAEIPPKTYSLQIAWVGGFIAISIISFLSLLGVTLVPLMNRVFFKFLLSFLVALAVGTLSGDAFLHLLPHSHA
SHHHSHSHEEPAMEMKRGPLFSHLSSQNIEESAYFDSTWKGLTALGGLYFMFLVEHVLTLIKQFKDKKKKNQK
KPENDDDVEIKKQLSKYESQLSTNEEKVDTDDRTEGYLRADSQEPSHFDSQQPAVLEEEEVMIAHAHPQEVYN
EYVPRGCKNKCHSHFHDTLGQSDDLIHHHHDYHHILHHHHHQNNHPHSHSQRYSREELKDAGVATLAWMVIMG
DGQHNFSDGLAIGDAFTEGLSSGLSTSVAVFCHELPHELGDFAVLLKAGMTVKQAVLYNALSAMLAYLGMATG
IFIGHYAENVSMWIFALTAGLFMYVALVDMVPEMLHNDASDHGCSRWGYFFLQNAGMLLGFGIMLLISIFEHK
IVFRINF

| | | |
|---|---|---|
| NOV9h, SNP 13376562 | SEQ ID NO: 111 | 3445 bp |
| DNA Sequence | ORF Start: ATG at 117 | ORF Stop: TAG at 2382 |

CACCGCGTGTTCGCGCCTGGTAGAGATTTCTCGAAGACACCAGTGGGCCCGTGTGGAACCAAACCTGCGCGCG
TGGCCGGGCCGTGGGACAACGAGGCCGCGGAGACGAAGGCGCAATGGCGAGGAAGTTATCTGTAATCTTGATC
CTGACCTTTGCCCCCTCTGTCACAAATCCCCTTCATGAACTAAAAGCAGCTGCTTTCCCCCAGACCACTGAGA
AAATTAGTCCGAATTGGGAATCTGGCATTAATGTTGACTTGGCAATTTCCACACGGCAATATCATCTACAACA
GCTTTTCTACCGCTATGGAGAAAATAATTCTTTGTCAGTTGAAGGGTTCAGAAAATTACTTCAAAATATAGGC
ATAGATAAGATTAAAAGAATCCATATACACCATGACCACGACCATCACTCAGACCACGAGCATCACTCAGACC
ATGAGCGTCACTCAGACCATGAGCATCACTCAGAGCACGAGCATCACTCTGACCATGATCATCACTCTCACCA
TAATCATGCTGCTTCTGGTAAAAATAAGCGAAAAGCTCTTTGCCCAGACCATGACTCAGATAGTTCAGGTAAA
GATCCTAGAAACAGCCAGGGGAAAGGAGCTCACCGACCAGAACATGCCAGTGGTAGAAGGAATGTCAAGGACA
GTGTTAGTGCTAGTGAAGTGACCTCAACTGTGTACAACACTGTCTCTGAAGGAACTCACTTTCTAGAGACAAT
AGAGACTCCAAGACCTGGAAAACTCTTCCCCAAAGATGTAAGCAGCTCCACTCCACCCAGTGTCACATCAAAG
AGCCGGGTGAGCCGGCTGGCTGGTAGGAAAACAAATGAATCTGTGAGTGAGCCCCGAAAAGGCTTTATGTATT
CCAGAAACACAAATGAAAATCCTCAGGAGTGTTTCAATGCATCAAAGCTACTGACATCTCATGGCATGGGCAT
CCAGGTTCCGCTGAATGCAACAGAGTTCAACTATCTCTGTCCAGCCATCATCAACCAAATTGATGCTAGATCT
TGTCTGATTCATACAAGTGAAAAGAAGGCTGAAATCCCTCCAAAGACCTATTCATTACAAATAGCCTGGGTTG
GTGGTTTTATAGCCATTTCCATCATCAGTTTCCTGTCTCTGCTGGGGGTTATCTTAGTGCCTCTCATGAATCG
GGTGTTTTTCAAATTTCTCCTGAGTTTCCTTGTGGCACTGGCCGTTGGGACTTTGAGTGGTGATGCTTTTTTA
CACCTTCTTCCACATTCTCATGCAAGTCACCACCATAGTCATAGCCATGAAGAACCAGCAATGGAAATGAAAA
GAGGACCACTTTTCAGTCATCTGTCTTCTCAAAACATAGAAGAAAGTGCCTATTTTGATTCCACGTGGAAGGG
TCTAACAGCTCTAGGAGGCCTGTATTTCATGTTTCTTGTTGAACATGTCCTCACATTGATCAAACAATTTAAA
GATAAGAAGAAAAAGAATCAGAAGAAACCTGAAAATGATGATGATGTGGAGATTAAGAAGCAGTTGTCCAAGT
ATGAATCTCAACTTTCAACAAATGAGGAGAAAGTAGATACAGATGATCGAACTGAAGGCTATTTACGAGCAGA
CTCACAAGAGCCCTCCCACTTTGATTCTCAGCAGCCTGCAGTCTTGGAAGAAGAAGAGGTCATGATAGCTCAT
GCTCATCCACAGGAAGTCTACAATGAATATGTACCCAGAGGGTGCAAGAATAAATGCCATTCACATTTCCACG
ATACACTCGGCCAGTCAGACGATCTCATTCACCACCATCATGACTACCATCATATTCTCCATCATCACCACCA
CCAAAACCACCATCCTCACAGTCACAGCCAGCGCTACTCTCGGGAGGAGCTGAAAGATGCCGGCGTCGCCACT
CTGGCCTGGATGGTGATAATGGGTGATGGCCTGCACAATTTCAGCGATGGCCTAGCAATTGGTGCTGCTTTTA
CTGAAGGCTTATCAAGTGGTTTAAGTACTTCTGTTGCTGTGTTCTGTCATGAGTTGCCTCATGAATTAGGTGA
CTTTGCTGTTCTACTAAAGGCTGGCATGACCGTTAAGCAGGCTGTCCTTTATAATGCATTGTCAGCCATGCTG
GCGTATCTTGGAATGGCAACAGGAATTTTCATTGGTCATTATGCTGAAAATGTTTCTATGTGGATATTTGCAC
TTACTGCTGGCTTATTCATGTATGTTGCTCTGGTTGATATGGTACCTGAAATGCTGCACAATGATGCTAGTGA
CCATGGATGTAGCCGCTGGGGGTATTTCTTTTTACAGAATGCTGGGATGCTTTTGGGTTTTGGAATTATGTTA
CTTATTTCCATATTTGAACATAAAATCGTGTTTCGTATAAATTTCTAGTTAAGGTTTAAATGCTAGAGTAGCT
TAAAAAGTTGTCATAGTTTCAGTAGGTCATAGGGAGATGAGTTTGTATGCTGTACTATGCAGCGTTTAAAGTT
AGTGGGTTTTGTGATTTTTGTATTGAATATTGCTGTCTGTTACAAAGTCAGTTAAAGGTACGTTTTAATATTT
AAGTTATTCTATCTTGGAGATAAAATCTGTATGTGCAATTCACCGGTATTACCAGTTTATTATGTAAACAAGA
GATTTGGCATGACATGTTCTGTATGTTTCAGGGAAAAATGTCTTTAATGCTTTTTCAAGAACTAACACAGTTA
TTCCTATACTGGATTTTAGGTCTCTGAAGAACTGCTGGTGTTTAGGAATAAGAATGTGCATGAAGCCTAAAAT
ACCAAGAAAGCTTATACTGAATTTAAGCAAAGAAATAAAGGAGAAAAGAGAAGAATCTGAGAATTGGGGAGGC
ATAGATTCTTATAAAAATCACAAAATTTGTTGTAAATTAGAGGGGAGAAATTTAGAATTAAGTATAAAAAGGC
AGAATTAGTATAGAGTACATTCATTAAACATTTTTGTCAGGATTATTTCCCGTAAAAACGTAGTGAGCACTTT
TCATATACTAATTTAGTTGTACATTTAACTTTGTATAATACAGAAATCTAATATATTTAAATGAATTCAAGCA
ATATATCACTTGACCAAGAAATTGGAATTTCAAAATGTTCGTGCGGGTATATACCAGATGAGTACAGTGAGTA
GTTTTATGTATCACCAGACTGGGTTATTGCCAAGTTATATATCACCAAAAGCTGTATGACTGGATGTTCTGGT
TACCTGGTTTACAAAATTATCAGAGTAGTAAAACTTTGATATATATGAGGATATTAAAACTACACTAAGTATC
ATTTGATTCGATTCAGAAAGTACTTTGATATCTCTCAGTGCTTCAGTGCTATCATTGTGAGCAATTGTCTTTT
ATATACGGTACTGTAGCCATACTAGGCCTGTCTGTGGCATTCTCTAGATGTTTCTTTTTTACACAATAAATTC
CTTATATCAGCTTG

| | | | |
|---|---|---|---|
| NOV9h, SNP 13376562 | | | |
| Protein Sequence | SEQ ID NO: 112 | 755 aa | MW at 85030.0kD |

MARKLSVILILTFAPSVTNPLHELKAAAFPQTTEKISPNWESGINVDLAISTRQYHLQQLFYRYGENNSLSVE
GFRKLLQNIGIDKIKRIHIHHDHDHHSDHEHHSDHERHSDHEHHSEHEHHSDHDHHSHHNHAASGKNKRKALC
PDHDSDSSGKDPRNSQGKGAHRPEHASGRRNVKDSVSASEVTSTVYNTVSEGTHFLETIETPRPGKLFPKDVS
SSTPPSVTSKSRVSRLAGRKTNESVSEPRKGFMYSRNTNENPQECFNASKLLTSHGMGIQVPLNATEFNYLCP
AIINQIDARSCLIHTSEKKAEIPPKTYSLQIAWVGGFIAISIISFLSLLGVILVPLMNRVFFKFLLSFLVALA
VGTLSGDAFLHLLPHSHASHHHSHSHEEPANEMKRGPLFSHLSSQNIEESAYFDSTWKGLTALGGLYFMFLVE
HVLTLIKQFKDKKKKNQKKPENDDDVEIKKQLSKYESQLSTNEEKVDTDDRTEGYLRADSQEPSHFDSQQPAV
LEEEEVMIAHAHPQEVYNEYVPRGCKNKCHSHFHDTLGQSDDLIHHHHDYHHILHHHHHQNHHPHSHSQRYSR
EELKDAGVATLAWMVIMGDGLHNFSDGLAIGAAFTEGLSSGLSTSVAVFCHELPHELGDFAVLLKAGMTVKQA
VLYNALSAMLAYLGMATGIFIGHYAENVSMWIFALTAGLFMYVALVDMVPEMLHNDASDHGCSRWGYFFLQNA
GMLLGFGIMLLISIFEHKIVFRINF

| | | |
|---|---|---|
| NOV9i, CG56008 | SEQ ID NO: 113 | 3445 bp |
| DNA Sequence | ORF Start: ATG at | ORF Stop TAG at 2382 |

CACCGCGTGTTCGCGCCTGGTAGAGATTTCTCGAAGACACCAGTGGGCCCGTGTGGAACCAAACCTGCGCGCG
TGGCCGGGCCGTGGGACAACGAGGCCGCGGAGACGAAGGCGCAATGGCGAGGAAGTTATCTGTAATCTTGATC
CTGACCTTTGCCCX₁CTCTGTCACAAATCCCCTTCATGAACTAAAAGCAGCTGCTTTCCCCCAGACCACTGAG
AAAATTAGTCCGAATTGGGAATCTGGCATTAATGTTGACTTGGCAATTTCCACACGGCAATATCATCTACAAC
AGCTTTTCTACCGCTATGGAGAAAATAATTCTTTGTCAGTTGAAGGGTTCAGAAAATTACTTCAAAATATAGG
CATAGATAAGATTAAAAGAATCCATATACACCATGACCACGACCATCACTCAGACCACGAGCATCACTCAGAC
CATGAGCGTCACTCAGACCATGAGCATCACTCAGAGCACGAGCATCACTCTGACCATGATCATCACTCTCACC
ATAATCATGCTGCTTCTGGTAAAAATAAGCGAAAAGCTCTTTGCCCAGACCATGACTCAGATAGTTCAGGTAA
AGATCCTAGAAACAGCCAGGGGAAAGGAGCTCACCGACCAGAACATGCCAGTGGTAGAAGGAATGTCAAGGAC
AGTGTTAGTGCTAGTGAAGTGACCTCAACTGTGTACAACACTGTCTCTGAAGGAACTCACTTTCTAGAGACAA
TAGAGACTCCAAGACCTGGAAAACTCTTCCCCAAAGATGTAAGCAGCTCCACTCCACCCAGTGTCACATCAAA
GAGCCGGGTGAGCCGGCTGGCTGGTAGGAAAACAAATGAATCTGTGAGTGAGCCCCGAAAAGGCTTTATGTAT
TCCAGAAACACAAATGAAAATCCTCAGGAGTGTTTCAATGCATCAAAGCTACTGACATCTCATGGCATGGGCA
TCCAGGTTCCGCTGAATGCAACAGAGTTCAACTATCTCTGTCCAGCCATCATCAACCAAATTGATGCTAGATC
TTGTCTGATTCATACAAGTGAAAAGAAGGCTGAAATCCCTCCAAAGACCTATTCATTACAAATAGCCTGGGTT
GGTGGTTTTATAGCCATTTCCATCATCAGTTTCCTGTCTCTGCTGGGGGTTATCTTAGTGCCTCTCATGAATC
GGGTGTTTTTCAAATTTCTCCTGAGTTTCCTTGTGGCACTGGCCGTTGGGACTTTGAGTGGTGATGCTTTTTT
ACACCTTCTTCCACATTCTCATGCAAGTCACCACCATAGTCATAGCCATGAAGAACCAGCAATGGAAATGAAA
AGAGGACCACTTTTCAGTCATCTGTCTTCTCAAAACATAGAAGAAAGTGCCTATTTTGATTCCACGTGGAAGG
GTCTAACAGCTCTAGGAGGCCTGTATTTCATGTTTCTTGTTGAACATGTCCTCACATTGATCAAACAATTTAA
AGATAAGAAGAAAAAGAATCAGAAGAAACCTGAAAATGATGATGATGTGGAGATTAAGAAGCAGTTGTCCAAG
TATGAATCTCAACTTTCAACAAATGAGGAGAAAGTAGATACAGATGATCGAACTGAAGGCTATTTACGAGCAG
ACTCACAAGAGCCCTCCCACTTTGATTCTCAGCAGCCTGCAGTCTTGGAAGAAGAAGAGGTCATGATAGCTCA
TGCTCATCCACAGGAAGTCTACAATGAATATGTACCCAGAGGGTGCAAGAATAAATGCCATTCACATTTCCAC
GATACACTCGGCCAGTCAGACGATCTCATTCACCACCATCATGACTACCATCATATTCTCCATCATCACCACC
ACCAAAACCACCATCCTCACAGTCACAGCCAGCGCTACTCTCGGGAGGAGCTGAAAGATGCCGGCGTCGCCAC
TCTGGCCTGGATGGTGATAATGGGTGATGGCCTGCACAATTTCAGCGATGGCCTAGCAATTGGTGCTGCTTTT
ACTGAAGGCTTATCAAGTGGTTTAAGTACTTCTGTTGCTGTGTTCTGTCATGAGTTGCCTCATGAATTAGGTG
ACTTTGCTGTTCTACTAAAGGCTGGCATGACCGTTAAGCAGGCTGTCCTTTATAATGCATTGTCAGCCATGCT
GGCGTATCTTGGAATGGCAACAGGAATTTTCATTGGTCATTATGCTGAAAATGTTTCTATGTGGATATTTGCA
CTTACTGCTGGCTTATTCATGTATGTTGCTCTGGTTGATATGGTACCTGAAATGCTGCACAATGATGCTAGTG
ACCATGGATGTAGCCGCTGGGGGTATTTCTTTTTACAGAATGCTGGGATGCTTTTGGGTTTTGGAATTATGTT
ACTTATTTCCATATTTGAACATAAAATCGTGTTTCGTATAAATTTCTAGTTAAGGTTTAAATGCTAGAGTAGC
TTAAAAAGTTGTCATAGTTTCAGTAGGTCATAGGGAGATGAGTTTGTATGCTGTACTATGCAGCGTTTAAAGT
TAGTGGGTTTTGTGATTTTTGTATTGAATATTGCTGTCTGTTACAAAGTCAGTTAAAGGTACGTTTTAATATT
TAAGTTATTCTATCTTGGAGATAAAATCTGTATGTGCAATTCACCGGTATTACCAGTTTATTATGTAAACAAG
AGATTTGGCATGACATGTTCTGTATGTTTCAGGGAAAAATGTCTTTAATGCTTTTTCAAGAACTAACACAGTT
ATTCCTATACTGGATTTTAGGTCTCTGAAGAACTGCTGGTGTTTAGGAATAAGAATGTGCATGAAGCCTAAAA
TACCAAGAAAGCTTATACTGAATTTAAGCAAAGAAATAAAGGAGAAAAGAGAAGAATCTGAGAATTGGGGAGG
CATAGATTCTTATAAAAATCACAAAATTTGTTGTAAATTAGAGGGGAGAAATTTAGAATTAAGTATAAAAAGG
CAGAATTAGTATAGAGTACATTCATTAAACATTTTTGTCAGGATTATTTCCCGTAAAAACGTAGTGAGCACTT
TTCATATACTAATTTAGTTGTACATTTAACTTTGTATAATACAGAAATCTAAATATATTTAATGAATTCAAGC
AATATATCACTTGACCAAGAAATTGGAATTTCAAAATGTTCGTGCGGGTATATACCAGATGAGTACAGTGAGT
AGTTTTATGTATCACCAGACTGGGTTATTGCCAAGTTATATATCACCAAAAGCTGTATGACTGGATGTTCTGG
TTACCTGGTTTACAAAATTATCAGAGTAGTAAAACTTTGATATATATGAGGATATTAAAACTACACTAAGTAT
CATTTGATTCGATTCAGAAAGTACTTTGATATCTCTCAGTGCTTCAGTGCTATCATTGTGAGCAATTGTCTTT
TATATACGGTACGTAGCCATACTAGGCCTGTCTGTGGCATTCTCTAGATGTTTCTTTTTTACACAATAAATTC
CTTATATCAGCTTGT
[Wherein residue X₁ is T or C.]

| | | | |
|---|---|---|---|
| NOV9i, CG56008 | | | |
| Protein Sequence | SEQ ID NO: 114 | 755 aa | MW at 85046.0kD |

MARKLSVILILTFAZ₁SVTNPLHELKAAAFPQTTEKISPNWESGINVDLAISTRQYHLQQLFYRYGENNSLSV
EGFRKLLQNIGIDKIKRIHIHHDHDHHSDHEHHSDHERHSDHEHHSEHEHHSDHDHHSHHNHAASGKNKRKAL
CPDHDSDSSGKDPRNSQGKGAHRPEHASGRRNVKDSVSASEVTSTVYNTVSEGTHFLETIETPRPGKLFPKDV
SSSTPPSVTSKSRVSRLAGRKTNESVSEPRKGFMYSRNTNENPQECFNASKLLTSHGMGIQVPLNATEFNYLC
PAIINQIDARSCLIHTSEKKAEIPPKTYSLQIAWVGGFIAISIISFLSLLGVILVPLMNRVFFKFLLSFLVAL
AVGTLSGDAFLHLLPHSHASHHHSHSHEEPAMEMKRGPLFSHLSSQNIEESAYFDSTWKGLTALGGLYFMFLV
EHVLTLIKQFKDKKKKNQKKPENDDDVEIKKQLSKYESQLSTNEEKVDTDDRTEGYLRADSQEPSHFDSQQPA
VLEEEEVMIAHAHPQEVYNEYVPRGCKNKCHSHFHDTLGQSDDLIHHHHDYHHILHHHHHQNHHPHSHSQRYS
REELKDAGVATLAWMVIMGDGLHNFSDGLAIGAAFTEGLSSGLSTSVAVFCHELPHELGDFAVLLKAGMTVKQ
AVLYNALSANLAYLGMATGIFIGHYAENVSMWIFALTAGLFMYVALVDMVFEMLHNDASDHGCSRWGYFFLQN
AGMLLGFGIMLLISIFEHKIVFRINF
[Wherein residue Z₁ is L or P.]

A ClustalW comparison of the above protein sequences yields the following sequence alignment shown in Table 9B.

TABLE 9B
Comparison of the NOV9 protein sequences.

| | |
|---|---|
| NOV9a | |
| NOV9b | ------------------------------------------------------------ |
| NOV9c | MGAAAGWLRGAAPGPRGSQSNETTACSRLVEISRRHQWARSEPSGPPVWNQTCARGRAVG |
| NOV9d | ------------------------------------------------------------ |
| NOV9e | ------------------------------------------------------------ |
| NOV9f | -------------------------------------------------------MGKPI |
| NOV9g | ------------------------------------------------------------ |
| NOV9a | ---------MARKLSVILILTFALSVTNPLHELKAAAFPQTTEKISPNWESGINVDLAIS |
| NOV9b | ---------------------------NPLYELKAAAFPQTTEKISPNWESGINVDLAIS |
| NOV9c | QRGRGDEGAMARKLSVILILTFALSVTNPLHELKAAAFPQTTEKISPNWESGINVDLAIS |
| NOV9d | ---------MARKLSVILILTFALSVTNPLHELKAAAFPQTTEKISPNWESGINVDLAIS |
| NOV9e | ---------MARKLSVILILTFALSVTNPLHELKAAAFPQTTEKISPNWESGINVDLAIS |
| NOV9f | PNPLLGLDSTARKLSVILILTFALSVTNPLHELKAAAFPQTTEKISPNWESGINVDLAIS |
| NOV9g | ---------------------------NPLHELKAAAFPQTTEKISPNWESGINVDLAIS |
| NOV9a | TRQYHLQQLFYRYGENNSLSVEGFRKLLQNIGIDKIKRIHIHHDHDHHSDHEHHSDHERH |
| NOV9b | TRQYHLQQLFYRYGENNSLSVEGFRKLLQNIGIDKIKRIHIHHDHDHHSDHEHHSDHERH |
| NOV9c | TRQYHLQQLFYRYGENNSLSVEGFRKLLQNIGIDKIKRIHIHHDHDHHSDHEHHSDHERH |
| NOV9d | TRQYHLQQLFYRYGENNSLSVEGFRKLLQNIGIDKIKRIHIHHDHDHHSDHEHHSDHERH |
| NOV9e | TRQYHLQQLFYRYGENNSLSVEGFRKLLQNIGIDKIKRIHIHHDHDHHSDHEHHSDHERH |
| NOV9f | TRQYHLQQLFYRYGENNSLSVEGFRKLLQNIGIDKIKRIHIHHDHDHHSDHEHHSDHERH |
| NOV9g | TRQYHLQQLFYRYGENNSLSVEGFRKLLQNIGIDKIKRIHIHHDHDHHSDHEHHSDHERH |
| NOV9a | SDHEHHSEHEHHSDHDHHSHHNHAASGKNKRKALCPDHDSDSSGKDPRNSQGKGAHRPEH |
| NOV9b | SDHEHHSDHEHHSDHDHHSHHNHAASGKNKRKALCPDHDSDSSGKDPRNSQGKGAHRPEH |
| NOV9c | SDHEHHSDHEHHSDHDHHSHHNHAAFTEG-----------LSSGLST--SVAVFCHELPH |
| NOV9d | SDHEHHSDHHPHSHSQRYSREELKDAGVATLAWMVIMGDGLHNFSDG---LAIGAAFTEG |
| NOV9e | SDHEHHSDHEHHSDHDHHSHHNHAASGKNKRKALCPDHDSDSSGKDPRNSQGKGAHRPEH |
| NOV9f | SDHEHHSDHEHHSDHDHHSHHNHAASGKNKRKALCPDHDSDSSGKDPRNSQGKGAHRPEH |
| NOV9g | SDHEHHSDHEHHSDHDHHSHHNHAASGKNKRKALCPDHDSDSSGKDPRNSQGKGAHRPEH |
| NOV9a | ASGRRNVKDSVSASEVTSTVYNTVSEGTHFLETIETPRPG---KLFPKDVSSSTPPSVTS |
| NOV9b | ASGRRNVKDSVSASEVTSTVYNTVSEGTHFLETIETPRPG---KLFPKDVSSSTPPSVTS |
| NOV9c | ELGDFAVLLKAGMTVKQAVLYNALSAMLAYLGMATGIFIGHYAENVSMWIFALTAGLFMY |
| NOV9d | LSSG----LSTSVAVFCHELPHELGDFAVLLKAGMTVKQA---VLYNALSANLAYLGMAT |
| NOV9e | ASGRRNVKDSVSASEVTSTVYNTVSEGTHFLETIETPRPG---KLFPKDVSSSTPPSVTS |
| NOV9f | ASGRRNVKDSVSASEVTSTVYNTVSEGTHFLETIETPRPG---KLFPKDVSSSTPPSVTS |
| NOV9g | ASGRRNVKDSVSASEVTSTVYNTVSEGTHFLETIETPRPG---KLFPKDVSSSTPPSVTS |
| NOV9a | KSRVSRLAGRKTNESVSEPRKGFMYSRNTNENPQECFNASKLLTSHGMGIQVPLNATEFN |
| NOV9b | KSRVSRLAGRKTNESVSEPRKGFMYSRNTNENPQECFNASKLLTSHGMGIQVPLNATEFN |
| NOV9c | VALVDMVPEMLHNDASDHGCSHWGYFFLQNAGMLLGFGIMLLISIFEHKIVFRINFNSPS |
| NOV9d | GIFIGHYAENVSMWIFALTAGLFMHVALVDMVPEMLHNDASDHGCSRWGYFFLQNAGMLL |
| NOV9e | KSRVSRLAGRKTNESVSEPRKGFMYSRNTNENPQECFNASKLLTSHGMGIQVPLNATEFN |
| NOV9f | KSRVSRLAGRKTNESVSEPRKGFMYSRNTNENPQECFNASKLLTSHGMGIQVPLNATEFN |
| NOV9g | KSRVSRLAGRKTNESVSEPRKGFMYSRNTNENPQECFNASKLLTSHGMGIQVPLNATEFN |
| NOV9a | YLCPAIINQIDARSCLIHTSEKKAEIPPKTYSLQIAWVGGFIAISIISFLSLLGVILVPL |
| NOV9b | YLCPAIINQIDARSCLIHTSEKKAEIPPKTYSLQ-------------------------- |
| NOV9c | SPPPKPPSSQSQPALLSGGAERCRRRHSGLDGDNG------------------------- |
| NOV9d | GFGIMLLISIFEHKIVFRINF--------------------------------------- |
| NOV9e | YLCPAIINQIDARSCLIHTSEKKAEIPPKTYSLQIAWVGGFIAISIISFLSLLGVILVPL |
| NOV9f | YLCPAIINQIDARSCLIHTSEKKAEIPPKTYSLQIAWVGGFIAISIISFLSLLGVILVPL |
| NOV9g | YLCPAIINQIDARSCLIHTSEKKAEIPPKTYSLQIAWVGGFIAISIISFLSLLGVILVPL |
| NOV9a | MNRVFFKFLLSFLVALAVGTLSGDAFLHLLPHSHASHHHSHSHEEPAMEMKRGPLFSHLS |
| NOV9b | ------------------------------------------------------------ |
| NOV9c | ------------------------------------------------------------ |
| NOV9d | ------------------------------------------------------------ |
| NOV9e | MNRVFFKFLLSFLVALAVGTLSGDAFLHLLPHSHASHHHSHSHEEPAMEMKRGPLFSHLS |
| NOV9f | MNRVFFKFLLSFLVALAVGTLSGDAFLHLLPHSHASHHHSHSHEEPAMEMKRGPLFSHLS |
| NOV9g | MNRVFFKFLLSFLVALAVGTLSGDAFLHLLPHSHASHHHSHSHEEPAMEMKRGPLFSHLS |
| NOV9a | SQNIEESAYFDSTWKGLTALGGLYFMFLVEHVLTLIKQFKDKKKKNQKKPENDDDVEIKK |
| NOV9b | ------------------------------------------------------------ |
| NOV9c | ------------------------------------------------------------ |
| NOV9d | ------------------------------------------------------------ |
| NOV9e | SQNIEESAYFDSTWKGLTALGGLYFMFLVEHVLTLIKQFKDKKKKNQKKPENDDDVEIKK |
| NOV9f | SQNIEESAYFDSTWKGLTALGGLYFMFLVEHVLTLIKQFKDKKKKNQKKPENDDDVEIKK |
| NOV9g | SQNIEESAYFDSTWKGLTALGGLYFMFLVEHVLTLIKQFKDKKKKNQKKPENDDDVEIKK |
| NOV9a | QLSKYESQLSTNEEKVDTDDRTEGYLRADSQEPSHFDSQQPAVLEEEEVMIAHAHPQEVY |
| NOV9b | ------------------------------------------------------------ |
| NOV9c | ------------------------------------------------------------ |
| NOV9d | ------------------------------------------------------------ |
| NOV9e | QLSKYESQLSTNEEKVDTDDRTEGYLRADSQEPSHFDSQQPAVLEEEEVMIAHAHPQEVY |
| NOV9f | QLSKYESQLSTNEEKVDTDDRTEGYLRADSQEPSHFDSQQPAVLEEEEVMIAHAHPQEVY |
| NOV9g | QLSKYESQLSTNEEKVDTDDRTEGYLRADSQEPSHFDSQQPAVLEEEEVMIAHAHPQEVY |
| NOV9a | NEYVPRGCKNKCHSHFHDTLGQSDDLIHHHHDYHHILHHHHHQNHHPHSHSQRYSREELK |
| NOV9b | ------------------------------------------------------------ |
| NOV9c | ------------------------------------------------------------ |
| NOV9d | ------------------------------------------------------------ |
| NOV9e | NEYVPRGCKNKCHSHFHDTLGQSDDLIHHHHDYHHILHHHHHQNHHPHSHSQRYSREELK |
| NOV9f | NEYVPRGCKNKCHSHFHDTLGQSDDLIHHHHDYHHILHHHHHQNHHPHSHSQRYSREELK |
| NOV9g | NEYVPRGCKNKCHSHFHDTLGQSDDLIHHHHDYHHTLHHHHHQNHHPHSHSQRYSREELK |
| NOV9a | DAGVATLAWMVIMGDGLHNFSDGLAIGAAFTEGLSSGLSTSVAVFCHELPHELGDFAVLL |
| NOV9b | ------------------------------------------------------------ |
| NOV9c | ------------------------------------------------------------ |
| NOV9d | ------------------------------------------------------------ |
| NOV9e | DAGVATLAWMVIMGDGLHNFSDGLAIGAAFTEGLSSGLSTSVAVFCHELPHELGDFAVLL |
| NOV9f | DAGVATLAWMVIMGDGLHNFSDGLAIGAAFTEGLSSGLSTSVAVFCHELPHELGDFAVLL |
| NOV9g | DAGVATLAWMVIMGDGQHNFSDGLAIGDAFTEGLSSGLSTSVAVFCHELPHELGDFAVLL |
| NOV9a | KAGMTVKQAVLYNALSAMLAYLGMATGIFIGHYAENVSMWIFALTAGLFMYVALVDMVPE |
| NOV9b | ------------------------------------------------------------ |
| NOV9c | ------------------------------------------------------------ |
| NOV9d | ------------------------------------------------------------ |
| NOV9e | KAGMTVKQAVLYNALSAMLAYLGMATGIFIGHYAENVSMWIFALTAGLFMYVALVDMVPE |
| NOV9f | KAGMTVKQAVLYNALSAMLAYLGMATGIFIGHYAENVSMWIFALTAGLFMYVALVDMVPE |
| NOV9g | KAGMTVKQAVLYNALSAMLAYLGMATGIFIGHYAENVSMWIFALTAGLFMYVALVDMVPE |
| NOV9a | MLHNDASDHGCSRWGYFFLQNAGMLLGFGIMLLISIFEHKIVFRINF------------- |
| NOV9b | ------------------------------------------------------------ |
| NOV9c | ------------------------------------------------------------ |
| NOV9d | ------------------------------------------------------------ |
| NOV9e | MLHNDASDHGCSRWGYFFLQNAGMLLGFGIMLLISIFEHKIVFRINF------------- |
| NOV9f | MLHNDASDHGCSRWGYFFLQNAGMLLGFGIMLLISIFEHKIVFRINF------------- |
| NOV9g | MLHNDASDHGCSRWGYFFLQNAGMLLGFGIMLLISIFEHKIVFRINF------------- |
| NOV9a | (SEQ ID NO: 98) |
| NOV9b | (SEQ ID NO: 100) |
| NOV9c | (SEQ ID NO: 102) |
| NOV9d | (SEQ ID NO: 104) |
| NOV9e | (SEQ ID NO: 106) |
| NOV9f | (SEQ ID NO: 108) |
| NOV9g | (SEQ ID NO: 110) |

Further analysis of the NOV9g protein yielded the following properties shown in Table 9C.

TABLE 9C
Protein Sequence Properties NOV9g

| SignalP | No cleavage site detected | | | |
|---|---|---|---|---|
| analysis: | | | | |
| PSORT II | PSG: a new signal peptide prediction method | | | |
| analysis: | N-region: length 7; pos. chg 1; neg. chg 1 | | | |
| | H-region: length 8; peak value 3.45 | | | |
| | PSG score: −0.95 | | | |
| | GvH: von Heijne's method for signal seq. recognition | | | |
| | GvH score (threshold: −2.1): −10.58 | | | |
| | possible cleavage site: between 14 and 15 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 6 | | | |
| | INTEGRAL | Likelihood = | −11.15 | Transmembrane |
| | | | | 314-330 |
| | INTEGRAL | Likelihood = | −5.26 | Transmembrane |
| | | | | 336-352 |
| | INTEGRAL | Likelihood = | −1.59 | Transmembrane |
| | | | | 412-428 |
| | INTEGRAL | Likelihood = | −1.97 | Transmembrane |
| | | | | 646-662 |
| | INTEGRAL | Likelihood = | −4.73 | Transmembrane |
| | | | | 671-687 |
| | INTEGRAL | Likelihood = | −3.98 | Transmembrane |
| | | | | 713-729 |
| | PERIPHERAL | Likelihood = | 3.45 (at 628) | |
| | ALOM score: −11.15 (number of TMSs: 6) | | | |
| | MTOP: Prediction of membrane topology (Hartmann et al.) | | | |
| | Center position for calculation: 321 | | | |
| | Charge difference: 0.5 C(2.0)-N(1.5) | | | |
| | C > N: C-terminal side will be inside | | | |
| | >>> membrane topology: type 3b | | | |
| | MITDISC: discrimination of mitochondrial targeting seq | | | |
| | R content: | 0 | Hyd Moment(75): | 6.50 |
| | Hyd Moment(95): | 9.58 | G content: | 1 |
| | D/E content: | 2 | S/T content: | 3 |

Score: −6.16
Gavel: prediction of cleavage sites for mitochondrial preseq
cleavage site motif not found
NUCDISC: discrimination of nuclear localization signals
pat4: KKKK (5) at 432
pat7: none
bipartite: none
content of basic residues: 9.1%
NLS Score: −0.16
NNCN: Reinhardt's method for Cytplasmic/Nuclear discrimination
Prediction: cytoplasmic
Reliability: 55.5
Psort Results (see Details):
60.0%: plasma membrane
40.0%: Golgi body
30.0%: endoplasmic reticulum (membrane)
30.0%: microbody (peroxisome)
Psort II Results (see Details):
33.3%: endoplasmic reticulum
22.2%: vacuolar
11.1%: Golgi
11.1%: nuclear
11.1%: vesicles of secretory system
11.1%: mitochondrial

A search of the NOV9g protein against the Geneseq database, a proprietary database that contains sequences published in patents and patent publication, yielded several homologous proteins shown in Table 9D.

TABLE 9D
Geneseq Results for NOV9g

| | | | Identities/ | |
|---|---|---|---|---|
| | | Residues/ | Similarities for | |
| | | Match | the Matched | |
| Identifier | Date] | Residues | Region | Value |
| ABG76949 | Human protein, homologous to LIV-1, | 1 . . . 733 | 733/733 (99%) | 0.0 |
| | designated NOV1 - Homo sapiens, 755 | 1 . . . 753 | 736/738 (99%) | |
| | aa. [WO200255705-A2, 18 JUL. 2002] | | | |
| ABR48228 | Human bladder cancer associated protein | 1 . . . 733 | 733/733 (99%) | 0.0 |
| | sequence SEQ ID NO: 177 - Homo | 1 . . . 753 | 736/738 (99%) | |
| | sapiens, 755 aa. [WO2003003906-A2, | | | |
| | 16 JAN. 2003] | | | |
| ABU56608 | Lung cancer-associated polypeptide #201 - | 1 . . . 733 | 733/733 (99%) | 0.0 |
| | Unidentified, 755 aa. | 1 . . . 753 | 736/738 (99%) | |
| | [WO200286443-A2, 31 OCT. 2002] | | | |
| AAM51198 | Human breast cancer 4 gene | 1 . . . 733 | 733/733 (99%) | 0.0 |
| | (BCR4)-encoded protein - Homo sapiens, | 1 . . . 753 | 736/738 (99%) | |
| | 755 aa. [WO200216939-A2, | | | |
| | 28 FEB. 2002] | | | |
| ABG61889 | Prostate cancer-associated protein #90 - | 1 . . . 733 | 733/733 (99%) | 0.0 |
| | Mammalia, 755 aa. [WO200230268-A2, | 1 . . . 753 | 736/738 (99%) | |
| | 18 APR. 2002] | | | |

In a BLAST search of public sequence databases, the NOV9g protein was found to have homology to the proteins shown in the BLASTP data in Table 9E.

TABLE 9E
Public BLASTP Results for NOV9g

| | | NOV9g | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD42374 | Sequence 1 from Patent | 1 . . . 733 | 752/753 (99%) | 0.0 |
| | WO0216939 - Homo sapiens | 1 . . . 753 | 753/753 (99%) | |
| | (Human), 755 aa. | | | |
| Q13433 | Estrogen regulated LIV-1 protein - | 1 . . . 733 | 727/735 (98%) | 0.0 |
| | Homo sapiens (Human), 749 aa. | 19 . . . 747 | 730/736 (98%) | |
| G02273 | LIV-1 protein - human, 752 aa. | 1 . . . 733 | 729/736 (98%) | 0.0 |
| | | 19 . . . 747 | 730/736 (98%) | |

PFam analysis predicts that the NOV9g protein contains the domains shown in the Table 9F. Specific amino acid residues of NOV9g for each domain is shown in column 2, equivalent domains in the other NOV9 proteins of the invention are also encompassed herein.

TABLE 9F
Domain Analysis of NOV9g

| | NOV11g Match Region | | |
|---|---|---|---|
| Pfam Domain | Amino Acid Residues: | Score | Expect Value |
| Zip | 301−725 | 443.7 | 1.6e−129 |

### Example 10

### NOV10, CG59356, Nuclear Receptor Subfamily 4

The NOV10 clone was analyzed, and the nucleotide and encoded polypeptide sequences are shown in Table 10A.

TABLE 10A

NOV10 Sequence Analysis

| | | |
|---|---|---|
| NOV10a, CG59356-01 | SEQ ID NO: 115 | 3802 bp |
| DNA Sequence | ORF Start: ATG at 732 | ORF Stop: TAA at 2610 |

ATAAATGACGTGCCGAGAGAGCGAGCGAACGCGCAGCCGGGAGAGCGGAGTCTCCTGCCTCCCGCCCCCCACC
CCTCCAGCTCCTGCTCCTCCTCCGCTCCCCATACACAGACGCGCTCACACCCGCTCCCTCACTCGAACACACA
GACACAAGCGCGCACACAGGCTCCGCACACACACACTTCGCTCTCCCGCGCGCTCACACCCCTCTTGCCCTGA
GCCCTTGCCGGTGCAGCGCGGCGCCGCAGCTGGACGCCCCTCCCGGGCTCACTTTGCAACGCTGACGGTGCCG
GCAGTGGCCGTGGAGGTGGGAACAGCGGCGGCATCCTCCCCCCTGGTCACAGCCCAAGCCAGGACGCCCGCGG
AACCTCTCGGCTGTGCTCTCCCATGAGTCGGGATCGCAGCATCCCCCACCAGCCGCTCACCGCCTCCGGGAGC
CGCTGGGCTTGTACACCGCAGCCCTTCCGGGACAGCAGCTGTGACTCCCCCCCAGTGCAGATTTCGGGACAGC
TCTCTAGAAACTCGCTCTAAAGACGGAACCGCCACAGCACTCAAAGCCCACTGCGGAAGAGGGCAGCCCGGCA
AGCCCGGGCCCTGAGCCTGGACCCTTAGCGGTGCCGGGCAGCACTGCCGGCGCTTCGCCTCGCCGGACGTCCG
CTCCTCCTACACTCTCAGCCTCCGCTGGAGAGACCCCCAGCCCCACCATTCAGCGCGCAAGATACCCTCCAGA
TATGCCCTGCGTCCAAGCCCAATATAGCCCTTCCCCTCCAGGTTCCAGTTATGCGGCGCAGACATACAGCTCG
GAATACACCACGGAGATCATGAACCCCGACTACACCAAGCTGACCATGGACCTTGGCAGCACTGAGATCACGG
CTACAGCCACCACGTCCCTGCCCAGCATCAGTACCTTCGTGGAGGGCTACTCGAGCAACTACGAACTCAAGCC
TTCCTGCGTGTACCAAATGCAGCGGCCCTTGATCAAAGTGGAGGAGGGGCGGGCGCCCAGCTACCATCACCAT
CACCACCACCACCACCACCACCACCACCATCACCAGCAGCAGCATCAGCAGCCATCCATTCCTCCAGCCTCCA
GCCCGGAGGACGAGGTGCTGCCCAGCACCTCCATGTACTTCAAGCAGTCCCCACCGTCCACCCCCACCACGCC
GGCCTTCCCCCCGCAGGCGGGGGCGTTATGGGACGAGGCACTGCCCTCGGCGCCCGGCTGCATCGCACCCGGC
CCGCTGCTGGACCCGCCGATGAAGGCGGTCCCCACGGTGGCCGGCGCGCGCTTCCCGCTCTTCCACTTCAAGC
CCTCGCCGCCGCATCCCCCCGCGCCCAGCCCGGCCGGCGGCCACCACCTCGGCTACGACCCGACGGCCGCTGC
CGCGCTCAGCCTGCCGCTGGGAGCCGCAGCCGCCGCGGGCAGCCAGGCCGCCGCGCTTGAGGGCCACCCGTAC
GGGCTGCCGCTGGCCAAGAGGGCGGCCCCGCTGGCCTTCCCGCCTCTCGGCCTCACGCCCTCCCCTACCGCGT
CCAGCCTGCTGGGCGAGAGTCCCAGCCTGCCGTCGCCGCCCAGCAGGAGCTCGTCGTCTGGCGAGGGCACGTG
TGCCGTGTGCGGGGACAACGCCGCCTGCCAGCACTACGGCGTGCGAACCTGCGAGGGCTGCAAGGGCTTTTTC
AAGAGAACAGTGCAGAAAAATGCAAAATATGTTTGCCTGGCAAATAAAAACTGCCCAGTAGACAAGAGACGTC
GAAACCGATGTCAGTACTGTCGATTTCAGAAGTGTCTCAGTGTTGGAATGGTAAAAGAAGTTGTCCGTACAGA
TAGTCTGAAAGGGAGGAGAGGTCGTCTGCCTTCCAAACCAAAGAGCCCATTACAACAGGAACCTTCTCAGCCC
TCTCCACCTTCTCCTCCAATCTGCATGATGAATGCTCTTGTCCGAGCTTTAACAGACTCAACACCCAGAGATC
TTGATTATTCCAGATACTGTCCCACTGACCAGGCTGCTGCAGGCACAGATGCTGAGCATGTGCAACAATTCTA
CAACCTCCTGACAGCCTCCATTGATGTATCCAGAAGCTGGGCAGAAAGGATTCCGGGATTTACTGATCTCCCC
AAAGAAGATCAGACATTACTTATTGAATCAGCCTTTTTGGAGCTGTTTGTCCTCAGACTTTCCATCAGGTCAA
ACACTGCTGAAGATAAGTTTGTGTTCTGCAATGGACTTGTCCTGCATCGACTTCAGTGCCTTCGTGGATTTGG
GGAGTGGCTCGACTCTATTAAAGACTTTTCCTTAAATTTGCAGAGCCTGAACCTTGATATCCAAGCCTTAGCC
TGCCTGTCAGCACTGAGCATGATCACAGAAAGACATGGGTTAAAAGAACCAAAGAGAGTCGAAGAGCTATGCA
ACAAGATCACAAGCAGTTTAAAAGACCACCAGAGTAAGGGACAGGCTCTGGAACCCAACGAGTCCAAGGTCCT
GGTTGCCCTGGTAGAACTGAGGAAGATCTGCACCCTGGGCCTCCAGCGCATCTTCTACCTGAAGCTGGAAGAC
TTGGTGTCTCCACCTTCCATCATTGACAAGCTCTTCCTGGACACCCTACCTTTCTAATCAGGAGCAGTGGAGC
AGTGAGCTGCCTCCTCTCCTAGCACCCTGCTTCTACGCAGCAAAGGGATAGGTTTGGAAACCTATCATTTCCT
GTCCTTCCTTAAGAGGAAAAGCAGCTCCTGTAGAAAGCAAAGACTTTCTTTTTTTTCTGGCTCTTTTCCTTAC
AACCTAAAGCCAGAAAACTTGCAGAGTATTGTGTTGGGGTTGTGTTTTATATTTAGGCATTGGGGGATGGGGT
GGGAGGGGGTTATAGTTCATGAGGGTTTTCTAAGAAATTGCTAACAAAGCACTTTTGGACAATGCTATCCCAG
CAGGAAAAAAAAGGATAATATAACTGTTTTAAAACTCTTTCTGGGGAATCCAATTATAGTTGCTTTGTATTTA
AAAACAAGAACAGCCAAGGGTTGTTCGCCAGGGTAGGATGTGTCTTAAAGATTGGTCCCTTGAAAATATGCTT
CCTGTATCAAAGGTACGTATGTGGTGCAAACAAGGCAGAAACTTCCTTTTAATTTCCTTCTTCCTTTATTTTA
ACAAATGGTGAAAGATGGAGGATTACCTACAAATCAGACATGGCAAAACAATAATGGCTGTTTGCTTCCATAA
ACAAGTGCAATTTTTTAAAGTGCTGTCTTACTAAGTCTTGTTTATTAACTCTCCTTTATTCTATATGGAAATA
AAAAGGAGGCAGTCATGTTAGCAAATGACACGTTAATATCCCTAGCAGAGGCTGTGTTCACCTTCCCTGTCGA
TCCCTTCTGAGGTATGGCCCATCCAAGACTTTTAGGCCATTCTTGATGGAACCAGATCCCTGCCCTGACTGTC
CAGCTATCCTGAAAGTGGATCAGATTATAAACTGGATTACATGTAACTGTTTTGGTTGTGTTCTATCAACCCC
ACCAGAGTTCCCTAAACTTGCTTCAGTTATAGTAACTGACTGGTATATTCATTCAGAAGCGCCATAAGTCAGT
TGAGTATTTGATCCCTAGATAAGAACATGCAAATCAGCAGGAACTGGTCATACAGGGTAAGCACCAGGGACAA
TAAGGATTTTTATAGATATAATTTAATTTTTGGTAATTGGGTTAAGGAGACCAATTTTGGAGAGCAAGCAAAT
CTTCTTTTTAAAAAATAGTATGAATGTGAATACTAGAAAAGATTTAAGAAATAGTATGAGTGTGAGTACTAGG
AAGGAT

| | | | |
|---|---|---|---|
| NOV10a, CG59356-01 | | | |
| Protein Sequence | SEQ ID NO: 116 | 626 aa | MW at 68281.9kD |

| | |
|---|---|
| MPCVQAQYSPSPPGSSYAAQTYSSEYTTEIMNPDYTKLTMDLGSTEITATATTSLPSISTFVEGYSSMYELKP | +TL,46 |
| SCVYQMQRPLIKVEEGRAPSYHHHHHHHHHHHHHHQQQHQQPSIPPASSPEDEVLPSTSMYFKQSPPSTPTTP | |
| AFPPQAGALWDEALPSAPGCIAPGPLLDPPMKAVPTVAGARFPLFHFKPSPPHPPAPSPAGGHHLGYDPTAAA | |
| ALSLPLGAAAAAGSQAAALEGHPYGLPLAKRAAPLAFPPLGLTPSPTASSLLGESPSLPSPPSRSSSSGEGTC | |
| AVCGDNAACQHYGVRTCEGCKGFFKRTVQKNAKYVCLANKNCPVDKRRRNRCQYCRFQKCLSVGMVKEVVRTD | |
| SLKGRRGRLPSKPKSPLQQEPSQPSPPSPPICMMNALVRALTDSTPRDLDYSRYCPTDQAAAGTDAEHVQQFY | |
| NLLTASIDVSRSWAERIPGFTDLPKEDQTLLIESAFLELFVLRLSIRSNTAEDKFVFCNGLVLHRLQCLRGFG | |
| EWLDSIKDFSLNLQSLNLDIQALACLSALSMITERHGLKEPKRVEELCNKITSSLKDHQSKGQALEPNESKVL | |
| VALVELRKICTLGLQRIFYLKLEDLVSPPSIIDKLFLDTLPF | |

Further analysis of the NOV10a protein yielded the following properties shown in Table 10B.

TABLE 10B
Protein Sequence Properties NOV10a

| SignalP | No Known Signal Sequence Predicted | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 24; peak value 2.26 | | | |
| | PSG score: −2.14 | | | |
| | GvH: von Heijne's method for signal seq. recognition | | | |
| | GvH score (threshold: −2.1): −6.85 | | | |
| | possible cleavage site: between 60 and 61 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 0.63 (at 527) | | | |
| | ALOM score: −1.81 (number of TMSs: 0) | | | |
| | MITDISC: discrimination of mitochondrial targeting seq | | | |
| | R content: | 0 | Hyd Moment(75): | 0.70 |
| | Hyd Moment(95): | 0.43 | G content: | 1 |
| | D/E content: | 1 | S/T content: | 7 |

```
Score: −4.77
Gavel: prediction of cleavage sites for mitochondrial preseq
cleavage site motif not found
NUCDISC: discrimination of nuclear localization signals
pat4: KRRR (5) at 338
pat7: PVDKRRR (5) at 335
bipartite: none
content of basic residues: 9.4%
NLS Score: 0.27
checking 63 PROSITE DNA binding motifs:
Nuclear hormones receptors DNA-binding region signature (PS00031):
*** found ***
CAVCGDNAACQHYGVRTCEGCKGFFKR at 292
Leucine zipper pattern (PS00029): *** found ***
LPKEDQTLLIESAFLELFVLRL at 461
NNCN: Reinhardt's method for Cytoplasmic/Nuclear discrimination
Prediction: nuclear
Reliability: 94.1
Final Results (k = 9/23):
87.0%: nuclear
  4.3%: peroxisomal
  4.3%: cytoplasmic
  4.3%: mitochondrial
>> prediction for CG59356-01 is nuc (k = 23)
```

A search of the NOV10a protein against the Geneseq database, a proprietary database that contains sequences published in patents and patent publication, yielded several homologous proteins shown in Table 10C.

TABLE 10C
Geneseq Results for NOV10a

| | | NOV10a | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| AAW16398 | Human neuron-derived orphan receptor | 1 . . . 626 | 623/626 (99%) | 0.0 |
| | NOR-1 protein - Homo sapiens, 626 aa. | 1 . . . 626 | 624/626 (99%) | |
| | [JP09084585-A, 31 MAR. 1997] | | | |
| AAU96995 | Human nuclear receptor NOR1 protein | 1 . . . 626 | 625/626 (99%) | 0.0 |
| | sequence - Homo sapiens, 625 aa. | 1 . . . 625 | 625/626 (99%) | |
| | [WO200187923-A1, 22 NOV. 2001] | | | |
| ABB98438 | Murine Neural Orphan Receptor 1, | 1 . . . 626 | 579/631 (91%) | 0.0 |
| | NOR1, #2 - Mus musculus, 628 aa. | 1 . . . 628 | 592/631 (93%) | |
| | [WO200246391-A2, 13 JUN. 2002] | | | |
| AAR92057 | Apoptopic cerebral neuron nuclear | 1 . . . 626 | 579/631 (91%) | 0.0 |
| | receptor protein - Rattus norvegicus, | 1 . . . 628 | 592/631 (93%) | |
| | 628 aa. [JP08023980-A, 30 JAN. 1996] | | | |
| ABB98437 | Murine Neural Orphan Receptor 1, | 1 . . . 626 | 577/631 (91%) | 0.0 |
| | NOR1, #1 - Mus musculus, 627 aa. | 1 . . . 627 | 591/631 (93%) | |
| | [WO200246391-A2, 13 JUN. 2002] | | | |

In a BLAST search of public sequence databases, the NOV12a protein was found to have homology to the proteins shown in the BLASTP data in Table 12D.

TABLE 10D
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q92570 | Nuclear hormone receptor NOR-1 | 1 . . . 626 | 623/626 (99%) | 0.0 |
| | (Neuron-derived orphan receptor 1) | 1 . . . 626 | 624/626 (99%) | |
| | (Mitogen induced nuclear orphan | | | |
| | receptor) - Homo sapiens (Human), 626 | | | |
| | aa. | | | |
| S71930 | neuron-derived receptor NOR-1 - human, | 1 . . . 626 | 625/626 (99%) | 0.0 |
| | 625 aa. | 1 . . . 625 | 625/626 (99%) | |
| O97726 | Neuron-derived orphan receptor-1 alfa - | 1 . . . 626 | 593/643 (92%) | 0.0 |
| | Sus scrofa (Pig), 643 aa. | 1 . . . 643 | 604/643 (93%) | |
| P51179 | Nuclear hormone receptor NOR-1 | 1 . . . 626 | 579/631 (91%) | 0.0 |
| | (Neuron-derived orphan receptor 1) - | 1 . . . 628 | 592/631 (93%) | |
| | Rattus norvegicus (Rat), 628 aa. | | | |
| Q9QZB6 | Orphan nuclear receptor TEC long | 1 . . . 626 | 577/631 (91%) | 0.0 |
| | isoform - Mus musculus (Mouse), 627 aa. | 1 . . . 627 | 591/631 (93%) | |

PFam analysis predicts that the NOV10a protein contains the domains shown in the Table 10E.

TABLE 10E
Domain Analysis of NOV10a

| | | Identities/ | |
|---|---|---|---|
| | | Similarities | |
| | NOV12a Match Region | for the | Expect |
| Pfam Domain | Amino Acid Residues: | Matched Region | Value |
| zf-C4 | 290 . . . 365 | 49/77 (64%) | 2.2e−51 |
| | | 70/77 (91%) | |
| hormone_rec | 442 . . . 620 | 53/206 (26%) | 2.4e−33 |
| | | 142/206 (69%) | |

### Example 11

### NOV11 CG59889, KIAA1199 and KIAA1199 Extension

The NOV11 clone was analyzed, and the nucleotide and encoded polypeptide sequences are shown in Table 11A.

TABLE 11A
NOV11 Sequence Analysis

| | | |
|---|---|---|
| NOV11a, CG59889-04 | SEQ ID NO: 117 | 3864 bp |
| DNA Sequence | ORF Start: at 2 | ORF Stop: TGA at 3815 |

GTGCCCTGACCAGAGCCCTGAGTTGCAACCCTGGAACCCTGGCCATGACCAAGACCACCATGTGCATATCGGC
CAGGGCAAGACACTGCTGCTCACCTCTTCTGCCACGGTCTATTCCATCCACATCTCAGAGGGAGGCAAGCTGG
TCATTAAAGACCACGACGAGCCGATTGTTTTGCGAACCCGGCACATCCTGATTGACAACGGAGGAGAGCTGCA
TGCTGGGAGTGCCCTCTGCCCTTTCCAGGGCAATTTCACCATCATTTTGTATGGAAGGGCTGATGAAGGTATT
CAGCCGGATCCTTACTATGGTCTGAAGTACATTGGGGTTGGTAAAGGAGGCGCTCTTGAGTTGCATGGACAGA
AAAAGCTCTCCTGGACATTTCTGAACAAGACCCTTCACCCAGGTGGCATGGCAGAAGGAGGCTATTTTTTTGA
AAGGAGCTGGGGCCACCGTGGAGTTATTGTTCATGTCATCGACCCCAAATCAGGCACAGTCATCCATTCTGAC
CGGTTTGACACCTATAGATCCAAGAAAGAGAGTGAACGTCTGGTCCAGTATTTGAACGCGGTGCCCGATGGCA
GGATCCTTTCTGTTGCAGTGAATGATGAAGGTTCTCGAAATCTGGATGACATGGCCAGGAAGGCGATGACCAA
ATTGGGAAGCAAACACTTCCTGCACCTTGGATTTAGGGTGGAGTGGACGGAGTGGTTCGATCATGATAAAGTA
TCTCAGACTAAAGGTGGGGAGAAAATTTCAGACCTCTGGAAAGCTCACCCAGGAAAAATATGCAATCGTCCCA
TTGATATACAGCAGGCCACTACAATGGATGGAGTTAACCTCAGCACCGAGGTTGTCTACAAAAAAGGCCAGGA
TTATAGGTTTGCTTGCTACGACCGGGGCAGAGCCTGCCGGAGCTACCGTGTACGGTTCCTCTGTGGGAAGCCT
GTGAGGCCCAAACTCACAGTCACCATTGACACCAATGTGAACAGCACCATTCTGAACTTGGAGGATAATGTAC
AGTCATGGAAACCTGGAGATACCCTGGTCATTGCCAGTACTGATTACTCCATGTACCAGGCAGAAGAGTTCCA
GGTGCTTCCCTGCAGATCCTGCGCCCCCAACCAGGTCAAAGTGGCAGGGAAACCAATGTACCTGCACATCGGG
GAGGAGATAGACGGCGTGGACATGCGGGCGGAGGTTGGGCTTCTGAGCCGGAACATCATAGTGATGGGGGAGA
TGGAGGACAAATGCTACCCCTACAGAAACCACATCTGCAATTTCTTTGACTTCGATACCTTTGGGGGCCACAT
CAAGTTTGCTCTGGGATTTAAGGCAGCACACTTGGAGGGCACGGAGCTGAAGCATATGGGACAGCAGCTGGTG
GGTCAGTACCCGATTCACTTCCACCTGGCCGGTGATGTAGACGAAAGGGGAGGTTATGACCCACCCACATACA
TCAGGGACCTCTCCATCCATCATACATTCTCTCGCTGCGTCACAGTCCATGGCTCCAATGGCTTGTTGATCAA
GGACGTTGTGGGCTATAACTCTTTGGGCCACTGCTTCTTCACGGAAGATGGGCCGGAGGAACGCAACACTTTT
GACCACTGTCTTGGCCTCCTTGTCAAGTCTGGAACCCTCCTCCCCTCGGACCGTGACAGCAAGATGTGCAAGA
TGATCACAGAGGACTCCTACCCAGGGTACATCCCCAAGCCCAGGCAAGACTGCAATGCTGTGTCCACCTTCTG
GATGGCCAATCCCAACAACAACCTCATCAACTGTGCCGCTGCAGGATCTGAGGAAACTGGATTTTGGTTTATT
TTTCACCACGTACCAACGGGCCCCTCCGTGGGAATGTACTCCCCAGGTTATTCAGAGCACATTCCACTGGGAA
AATTCTATAACAACCGAGCACATTCCAACTACCGGGCTGGCATGATCATAGACAACGGAGTCAAAACCACCGA
GGCCTCTGCCAAGGACAAGCGGCCGTTCCTCTCAATCATCTCTGCCAGATACAGCCCTCACCAGGACGCCGAC
CCGCTGAAGCCCCGGGAGCCGGCCATCATCAGACACTTCATTGCCTACAAGAACCAGGACCACGGGGCCTGGC
TGCGCGGCGGGGATGTGTGGCTGGACAGCTGCCGGTTTGCTGACAATGGCATTGGCCTGACCCTGGCCAGTGG
TGGAACCTTCCCGTATGACGACGGCTCCAAGCAAGAGATAAAGAACAGCTTGTTTGTTGGCGAGAGTGGCAAC
GTGGGGACGGAAATGATGGACAATAGGATCTGGGGCCCTGGCGGCTTGGACCATAGCGGAAGGACCCTCCCTA
TAGGCCAGAATTTTCCAATTAGAGGAATTCAGTTATATGATGGCCCCATCAACATCCAAAACTGCACTTTCCG
AAAGTTTGTGGCCCTGGAGGGCCGGCACACCAGCGCCCTGGCCTTCCGCCTGAATAATGCCTGGCAGAGCTGC
CCCCATAACAACGTGACCGGCATTGCCTTTGAGGACGTTCCGATTACTTCCAGAGTGTTCTTCGGAGAGCCTG
GGCCCTGGTTCAACCAGCTGGACATGGATGGGGATAAGACATCTGTGTTCCATGACGTCGACGGCTCCGTGTC
CGAGTACCCTGGCTCCTACCTCACGAAGAATGACAACTGGCTGGTCCGGCACCCAGACTGCATCAATGTTCCC
GACTGGAGAGGGGCCATTTGCAGTGGGTGCTATGCACAGATGTACATTCAAGCCTACAAGACCAGTAACCTGC
GAATGAAGATCATCAAGAATGACTTCCCCAGCCACCCTCTTTACCTGGAGGGGGCGCTCACCAGGAGCACCCA
TTACCAGCAATACCAACCGGTTGTCACCCTGCAGAAGGGCTACACCATCCACTGGGACCAGACGGCCCCCGCC
GAACTCGCCATCTGGCTCATCAACTTCAACAAGGGCGACTGGATCCGAGTGGGGCTCTGCTACCCGCGAGGCA
CCACATTCTCCATCCTCTCGGATGTTCACAATCGCCTGCTGAAGCAAACGTCCAAGACGGGCGTCTTCGTGAG
GACCTTGCAGATGGACAAAGTGGAGCAGAGCTACCCTGGCAGGAGCCACTACTACTGGGACGAGGACTCAGGG
CTGTTGTTCCTGAAGCTGAAAGCTCAGAACGAGAGAGAGAAGTTTGCTTTCTGCTCCATGAAAGGCTGTGAGA
GGATAAAGATTAAAGCTCTGATTCCAAAGAACGCAGGCGTCAGTGACTGCACAGCCACAGCTTACCCCAAGTT
CACCGAGAGGGCTGTCGTAGACGTGCCGATGCCCAAGAAGCTCTTTGGTTCTCAGCTGAAAACAAAGGACCAT
TTCTTGGAGGTGAAGATGGAGAGTTCCAAGCAGCACTTCTTCCACCTCTGGAACGACTTCGCTTACATTGAAG
TGGATGGGAAGAAGTACCCCAGTTCGGAGGATGGCATCCAGGTGGTGGTGATTGACGGGAACCAAGGGCGCGT
GGTGAGCCACACGAGCTTCAGGAACTCCATTCTGCAAGGCATACCATGGCAGCTTTTCAACTATGTGGCGACC
ATCCCTGACAATTCCATAGTGCTTATGGCATCAAAGGGAAGATACGTCTCCAGAGGCCCATGGACCAGAGTGC
TGGAAAAGCTTGGGGCAGACAGGGGTCTCAAGTTGAAAGAGCAAATGGCATTCGTTGGCTTCAAAGGCAGCTT
CCGGCCCATCTGGGTGACACTGGACACTGAGGATCACAAAGCCAAAATCTTCCAAGTTGTGCCCATCCCTGTG
GTGAAGAAGAAGAAGTTGTGAGGACAGCTGCCGCCCGGTGCCACCTCGTGGTAGACTATGACGGTGAC

| | | | |
|---|---|---|---|
| NOV11a, CG59889-04 | | | |
| Protein Sequence | SEQ ID NO: 118 | 1271 aa | MW at 143122.4kD |

CPDQSPELQPWNPGHDQDHHVHIGQGKTLLLTSSATVYSIHISEGGKLVIKDHDEPIVLRTRHILIDNGGELH
AGSALCPFQGNFTIILYGRADEGIQPDPYYGLKYIGVGKGGALELHGQKKLSWTFLNKTLEPGGMAEGGYFFE
RSWGHRGVIVHVIDPKSGTVIHSDRFDTYRSKKESERLVQYLNAVPDGRILSVAVNDEGSRNLDDMARKAMTK
LGSKHFLHLGFRVEWTEWFDHDKVSQTKGGEKISDLWKAHPGKICNRPIDIQQATTMDGVNLSTEVVYKKGQD
YRFACYDRGRACRSYRVRFLCGKPVRPKLTVTIDTNVNSTILNLEDNVQSWKPGDTLVIASTDYSMYQAEEFQ
VLPCRSCAPNQVKVAGKPMYLHIGEEIDGVDMRAEVGLLSRNIIVMGEMEDKCYPYRNHICNFFDFDTFGGHI
KFALGFKAAHLEGTELKHMGQQLVGQYPIHFHLAGDVDERGGYDPPTYIRDLSIHHTFSRCVTVHGSNGLLIK
DVVGYNSLGHCFFTEDGPEERNTFDHCLGLLVKSGTLLPSDRDSKMCKMITEDSYPGYIPKPRQDCNAVSTFW
MANPNNNLINCAAAGSEETGFWFIFHHVPTGPSVGMYSPGYSEHIPLGKFYNNRAHSNYRAGMIIDNGVKTTE
ASAKDKRPFLSIISARYSPHQDADPLKPREPAIIRHFIAYKNQDHGAWLRGGDVWLDSCRFADNGIGLTLASG
GTFPYDDGSKQEIKNSLFVGESGNVGTEMMDNRIWGPGGLDHSGRTLPIGQNFPIRGIQLYDGPINIQNCTFR
KFVALEGRHTSALAFRLNNAWQSCPHNNVTGIAFEDVPITSRVFFGEPGPWFNQLDMDGDKTSVFHDVDGSVS
EYPGSYLTKNDNWLVRHPDCINVPDWRGAICSGCYAQMYIQAYKTSNLRMKIIKNDFPSHPLYLEGALTRSTH
YQQYQPVVTLQKGYTIHWDQTAPAELAIWLINFNKGDWIRVGLCYPRGTTFSILSDVHNRLLKQTSKTGVFVR
TLQMDKVEQSYPGRSHYYWDEDSGLLFLKLKAQNEREKFAFCSMKGCERIKIKALIPKNAGVSDCTATAYPKF
TERAVVDVPMPKKLFGSQLKTKDHFLEVKMESSKQHFFHLWNDFAYIEVDGKKYPSSEDGIQVVVIDGNQGRV
VSHTSFRNSILQGIPWQLFNYVATIPDNSIVLMASKGRYVSRGPWTRVLEKLGADRGLKLKEQMAFVGFKGSF
RPIWVTLDTEDHKAKIFQVVPIPVVKKKKL

| | | |
|---|---|---|
| NOV11b, CG59889-01 | SEQ ID NO: 119 | 4205 bp |
| DNA Sequence | ORF Start: ATG at 22 | ORF Stop: TGA at 4156 |

ATTAATGAATATAAAATTATTATGTACTACACAATTAGTAGAAAGCATATTTTAGAGACACACCTGCCGCAAA
ATACTCAGTCAAGGGAAGGGGCGGGTCCGAATCCAGGGGCGACGCCGCCGCCTCCGCCAGTGCCCCGGGCGTC
CCGCCGCCTCACTAAGCGCCTGGAGCGCGAGGATCGCTCCACTGCACTCCAGCCTGGGCAACAGAGCGAGACT
CTGTCTCAAAAAAAAAAAAGAAGTAAAAATAATTATGCAGTATGTTTAGACATTTTAATATTTGTTTTGATTT
CATTTTTTCTTCCCTTAAAAACACCCCTTGGGGAGACTTCGGCTGCTGGGTGCCCTGACCAGAGCCCTGAGTT
GCAACCCTGGAACCCTGGCCATGACCAAGACCACCATGTGCATATCGGCCAGGGCAAGACACTGCTGCTCACC
TCTTCTGCCACGGTCTATTCCATCCACATCTCAGAGGGAGGCAAGCTGGTCATTAAAGACCACGACGAGCCGA
TTGTTTTGCGAACCCGGCACATCCTGATTGACAACGGAGGAGAGCTGCATGCTGGGAGTGCCCTCTGCCCTTT
CCAGGGCAATTTCACCATCATTTTGTATGGAAGGGCTGATGAAGGTATTCAGCCGGATCCTTACTATGGTCTG
AAGTACATTGGGGTTGGTAAAGGAGGCGCTCTTGAGTTGCATGGACAGAAAAAGCTCTCCTGGACATTTCTGA
ACAAGACCCTTCACCCAGGTGGCATGGCAGAAGGAGGCTATTTTTTTGAAAGGAGCTGGGGCCACCGTGGAGT
TATTGTTCATGTCATCGACCCCAAATCAGGCACAGTCATCCATTCTGACCGGTTTGACACCTATAGATCCAAG
AAAGAGAGTGAACGTCTGGTCCAGTATTTGAACGCGGTGCCCGATGGCAGGATCCTTTCTGTTGCAGTGAATG
ATGAAGGTTCTCGAAATCTGGATGACATGGCCAGGAAGGCGATGACCAAATTGGGAAGCAAACACTTCCTGCA
CCTTGGATTTAGGGTGGAGTGGACGGAGTGGTTCGATCATGATAAAGTATCTCAGACTAAAGGTGGGGAGAAA
ATTTCAGACCTCTGGAAAGCTCACCCAGGAAAAATATGCAATCGTCCCATTGATATACAGCAGGCCACTACAA
TGGATGGAGTTAACCTCAGCACCGAGGTTGTCTACAAAAAAGGCCAGGATTATAGGTTTGCTTGCTACGACCG
GGGCAGAGCCTGCCGGAGCTACCGTGTACGGTTCCTCTGTGGGAAGCCTGTGAGGCCCAAACTCACAGTCACC
ATTGACACCAATGTGAACAGCACCATTCTGAACTTGGAGGATAATGTACAGTCATGGAAACCTGGAGATACCC
TGGTCATTGCCAGTACTGATTACTCCATGTACCAGGCAGAAGAGTTCCAGGTGCTTCCCTGCAGATCCTGCGC
CCCCAACCAGGTCAAAGTGGCAGGGAAACCAATGTACCTGCACATCGGGGAGGAGATAGACGGCGTGGACATG
CGGGCGGAGGTTGGGCTTCTGAGCCGGAACATCATAGTGATGGGGGAGATGGAGGACAAATGCTACCCCTACA
GAAACCACATCTGCAATTTCTTTGACTTCGATACCTTTGGGGGCCACATCAAGTTTGCTCTGGGATTTAAGGC
AGCACACTTGGAGGGCACGGAGCTGAAGCATATGGGACAGCAGCTGGTGGGTCAGTACCCGATTCACTTCCAC
CTGGCCGGTGATGTAGACGAAAGGGGAGGTTATGACCCACCCACATACATCAGGGACCTCTCCATCCATCATA
CATTCTCTCGCTGCGTCACAGTCCATGGCTCCAATGGCTTGTTGATCAAGGACGTTGTGGGCTATAACTCTTT
GGGCCACTGCTTCTTCACGGAAGATGGGCCGGAGGAACGCAACACTTTTGACCACTGTCTTGGCCTCCTTGTC
AAGTCTGGAACCCTCCTCCCCTCGGACCGTGACAGCAAGATGTGCAAGATGATCACAGAGGACTCCTACCCAG
GGTACATCCCCAAGCCCAGGCAAGACTGCAATGCTGTGTCCACCTTCTGGATGGCCAATCCCAACAACAACCT
CATCAACTGTGCCGCTGCAGGATCTGAGGAAACTGGATTTTGGTTTATTTTTCACCACGTACCAACGGGCCCC
TCCGTGGGAATGTACTCCCCAGGTTATTCAGAGCACATTCCACTGGGAAAATTCTATAACAACCGAGCACATT
CCAACTACCGGGCTGGCATGATCATAGACAACGGAGTCAAAACCACCGAGGCCTCTGCCAAGGACAAGCGGCC
GTTCCTCTCAATCATCTCTGCCAGATACAGCCCTCACCAGGACGCCGACCCGCTGAAGCCCCGGGAGCCGGCC
ATCATCAGACACTTCATTGCCTACAAGAACCAGGACCACGGGGCCTGGCTGCGCGGCGGGGATGTGTGGCTGG
ACAGCTGCCGGTTTGCTGACAATGGCATTGGCCTGACCCTGGCCAGTGGTGGAACCTTCCCGTATGACGACGG
CTCCAAGCAAGAGATAAAGAACAGCTTGTTTGTTGGCGAGAGTGGCAACGTGGGGACGGAAATGATGGACAAT
AGGATCTGGGGCCCTGGCGGCTTGGACCATAGCGGAAGGACCCTCCCTATAGGCCAGAATTTTCCAATTAGAG
GAATTCAGTTATATGATGGCCCCATCAACATCCAAAACTGCACTTTCCGAAAGTTTGTGGCCCTGGAGGGCCG
GCACACCAGCGCCCTGGCCTTCCGCCTGAATAATGCCTGGCAGAGCTGCCCCCATAACAACGTGACCGGCATT
GCCTTTGAGGACGTTCCGATTACTTCCAGAGTGTTCTTCGGAGAGCCTGGGCCCTGGTTCAACCAGCTGGACA
TGGATGGGGATAAGACATCTGTGTTCCATGACGTCGACGGCTCCGTGTCCGAGTACCCTGGCTCCTACCTCAC
GAAGAATGACAACTGGCTGGTCCGGCACCCAGACTGCATCAATGTTCCCGACTGGAGAGGGGCCATTTGCAGT
GGGTGCTATGCACAGATGTACATTCAAGCCTACAAGACCAGTAACCTGCGAATGAAGATCATCAAGAATGACT
TCCCCAGCCACCCTCTTTACCTGGAGGGGGCGCTCACCAGGAGCACCCATTACCAGCAATACCAACCGGTTGT
CACCCTGCAGAAGGGCTACACCATCCACTGGGACCAGACGGCCCCCGCCGAACTCGCCATCTGGCTCATCAAC
TTCAACAAGGGCGACTGGATCCGAGTGGGGCTCTGCTACCCGCGAGGCACCACATTCTCCATCCTCTCGGATG
TTCACAATCGCCTGCTGAAGCAAACGTCCAAGACGGGCGTCTTCGTGAGGACCTTGCAGATGGACAAAGTGGA
GCAGAGCTACCCTGGCAGGAGCCACTACTACTGGGACGAGGACTCAGGGCTGTTGTTCCTGAAGCTGAAAGCT
CAGAACGAGAGAGAGAAGTTTGCTTTCTGCTCCATGAAAGGCTGTGAGAGGATAAAGATTAAAGCTCTGATTC
CAAAGAACGCAGGCGTCAGTGACTGCACAGCCACAGCTTACCCCAAGTTCACCGAGAGGGCTGTCGTAGACGT
GCCGATGCCCAAGAAGCTCTTTGGTTCTCAGCTGAAAACAAAGGACCATTTCTTGGAGGTGAAGATGGAGAGT
TCCAAGCAGCACTTCTTCCACCTCTGGAACGACTTCGCTTACATTGAAGTGGATGGGAAGAAGTACCCCAGTT
CGGAGGATGGCATCCAGGTGGTGGTGATTGACGGGAACCAAGGGCGCGTGGTGAGCCACACGAGCTTCAGGAA
CTCCATTCTGCAAGGCATACCATGGCAGCTTTTCAACTATGTGGCGACCATCCCTGACAATTCCATAGTGCTT
ATGGCATCAAAGGGAAGATACGTCTCCAGAGGCCCATGGACCAGAGTGCTGGAAAAGCTTGGGGCAGACAGGG
GTCTCAAGTTGAAAGAGCAAATGGCATTCGTTGGCTTCAAAGGCAGCTTCCGGCCCATCTGGGTGACACTGGA
CACTGAGGATCACAAAGCCAAAATCTTCCAAGTTGTGCCCATCCCTGTGGTGAAGAAGAAGAAGTTGTGAGGA
CAGCTGCCGCCCGGTGCCACCTCGTGGTAGACTATGACGGTGAC

| | | | |
|---|---|---|---|
| NOV11b, CG59889-01 | | | |
| Protein Sequence | SEQ ID NO: 120 | 1378 aa | MW at 155014.9kD |

MYYTISRKHILETHLPQNTQSREGAGPNPGATPPPPPVPRASRRLTKRLEREDRSTALQPGQQSETLSQKKKR
SKNNYAVCLDILIFVLISFFLPLKTPLGETSAAGCPDQSPELQPWNPGHDQDHHVHIGQGKTLLLTSSATVYS
IHISEGGKLVTKDHDEPIVLRTRHILIDNGGELHAGSALCPFQGNFTIILYGRADEGIQPDPYYGLKYIGVGK
GGALELHGQKKLSWTFLNKTLHPGGMAEGGYFFERSWGHRGVIVHVIDPKSGTVIHSDRFDTYRSKKESERLV
QYLNAVPDGRILSVAVNDEGSRNLDDMARKAMTKLGSKHFLHLGFRVEWTEWFDHDKVSQTKGGEKISDLWKA
HPGKICNRPIDIQQATTMDGVNLSTEVVYKKGQDYRFACYDRGRACRSYRVRFLCGKPVRPKLTVTIDTNVNS
TILNLEDNVQSWKPGDTLVIASTDYSMYQAEEFQVLPCRSCAPNQVKVAGKPMYLHIGEEIDGVDMRAEVGLL
SRNIIVMGEMEDKCYPYRNHICNFFDFDTFGGHIKFALGFKAAHLEGTELKHMGQQLVGQYPIHFHLAGDVDE
RGGYDPPTYIRDLSIHHTFSRCVTVHGSNGLLIKDVVGYNSLGHCFFTEDGPEERNTFDHCLGLLVKSGTLLP
SDRDSKMCKMITEDSYPGYIPKPRQDCNAVSTFWMANPNNNLINCAAAGSEETGFWFIFHHVPTGPSVGMYSP
GYSEHIPLGKFYNNRAHSNYRAGMIIDNGVKTTEASAKDKRPFLSIISARYSPHQDADPLKPREPAIIRHFIA
YKNQDHGAWLRGGDVWLDSCRFADNGIGLTLASGGTFPYDDGSKQEIKNSLFVGESGNVGTEMMDNRIWGPGG
LDHSGRTLPIGQNFPIRGIQLYDGPINIQNCTFRKFVALEGRHTSALAFRLNNAWQSCPHNNVTGIAFEDVPI
TSRVFFGEPGPWFNQLDMDGDKTSVFHDVDGSVSEYPGSYLTKNDNWLVRHPDCINVPDWRGAICSGCYAQMY
IQAYKTSNLRMKIIKNDFPSHPLYLEGALTRSTHYQQYQPVVTLQKGYTIHWDQTAPAELAIWLINFNKGDWI
RVGLCYPRGTTFSILSDVHNRLLKQTSKTGVFVRTLQMDKVEQSYPGRSHYYWDEDSGLLFLKLKAQNEREKF
AFCSMKGCERIKIKALIPKNAGVSDCTATAYPKFTERAVVDVPMPKKLFGSQLKTKDHFLEVKMESSKQHFFH
LWNDFAYIEVDGKKYPSSEDGIQVVVIDGNQGRVVSHTSFRNSILQGIPWQLFNYVATIPDNSIVLMASKGRY
VSRGPWTRVLEKLGADRGLKLKEQMAFVGFKGSFRPIWVTLDTEDHKAKIFQVVPIPVVKKKKL

| | | |
|---|---|---|
| NOV11c, CG59889-07 | SEQ ID NO: 121 | 610 bp |
| DNA Sequence | ORF Start: at 11 | ORF Stop: end of sequence |

CACCAGATCTTGCCCTGACCAGAGCCCTGAGTTGCAACCCTGGAACCCTGGCCATGACCAAGACCACCATGTG
CATATCGGCCAGGGCAAGACACTGCTGCTCACCTCTTCTGCCACGGTCTATTCCATCCACATCTCAGAGGGAG
GCAAGCTGGTCATTAAAGACCACGACGAGCCGATTGTTTTGCGAACCCGGCACATCCTGATTGACAACGGAGG
AGAGCTGCATGCTGGGAGTGCCCTCTGCCCTTTCCAGGGCAATTTCACCATCATTTTGTATGGAAGGGCTGAT
GAAGGTATTCAGCCGGATCCTTACTATGGTCTGAAGTACATTGGGGTTGGTAAAGGAGGCGCTCTTGAGTTGC
ATGGACAGAAAAAGCTCTCCTGGACATTTCTGAACAAGACCCTTCACCCAGGTGGCATGGCAGAAGGAGGCTA
TTTTTTTGAAAGGAGCTGGGGCCACCGTGGAGTTATTGTTCATGTCATCGACCCCAAATCAGGCACAGTCATC
CATTCTGACCGGTTTGACACCTATAGATCCAAGAAAGAGAGTGAACGTCTGGTCCAGTATTTGAACGCGGTGC
CCGATGGCAGGATCCTTTCTGTTGCA

| | | | |
|---|---|---|---|
| NOV11c, CG59889-07 | | | |
| Protein Sequence | SEQ ID NO: 122 | 200 aa | MW at 22110.8kD |

CPDQSPELQPWNPGHDQDHHVHIGQGKTLLLTSSATVYSIHISEGGKLVIKDHDEPIVLRTRHILIDNGGELH
AGSALCPFQGNFTIILYGRADEGIQPDPYYGLKYIGVGKGGALELHGQKKLSWTFLNKTLHPGGMAEGGYFFE
RSWGHRGVIVHVIDPKSGTVIHSDRFDTYRSKKESERLVQYLNAVPDGRILSVA

| | | |
|---|---|---|
| NOV11d, CG59889-09 | SEQ ID NO: 123 | 366 bp |
| DNA Sequence | ORF Start: at 1 | ORF Stop: end of sequence |

GATGGGAAGAAGTACCCCAGTTCGGAGGATGGCATCCAGGTGGTGGTGATTGACGGGAACCAAGGGCGCGTGG
TGAGCCACACGAGCTTCAGGAACTCCATTCTGCAAGGCATACCATGGCAGCTTTTCAACTATGTGGCGACCAT
CCCTGACAATTCCATAGTGCTTATGGCATCAAAGGGAAGATACGTCTCCAGAGGCCCATGGACCAGAGTGCTG
GAAAAGCTTGGGGCAGACAGGGGTCTCAAGTTGAAAGAGCAAATGGCATTCGTTGGCTTCAAAGGCAGCTTCC
GGCCCATCTGGGTGACACTGGACACTGAGGATCACAAAGCCAAAATCTTCCAAGTTGTGCCCATCCCTGTGGT
G

| | | | |
|---|---|---|---|
| NOV11d, CG59889-09 | | | |
| Protein Sequence | SEQ ID NO: 124 | 122 aa | MW at 13642.7kD |

DGKKYPSSEDGIQVVVIDGNQGRVVSHTSFRNSILQGIPWQLFNYVATIPDNSIVLMASKGRYVSRGPWTRVL
EKLGADRGLKLKEQMAFVGFKGSFRPIWVTLDTEDHKAKIFQVVPIPVV

| | | |
|---|---|---|
| NOV11e, CG59889-10 | SEQ ID NO: 125 | 772 bp |
| DNA Sequence | ORF Start: at 11 | ORF Stop: at 764 |

CACCAGATCTCATGTGCATATCGGCCAGGGCAAGACACTGCTGCTCACCTCTTCTGCCACGGTCTATTCCATC
CACATCTCAGAGGGAGGCAAGCTGGTCATTAAAGACCACGACGAGCCGATTGTTTTGCGAACCCGGCACATCC
TGATTGACAACGGAGGAGAGCTGCATGCTGGGAGTGCCCTCTGCCCTTTCCAGGGCAATTTCACCATCATTTT
GTATGGAAGGGCTGATGAAGGTATTCAGCCGGATCCTTACTATGGTCTGAAGTACATTGGGGTTGGTAAAGGA
GGCGCTCTTGAGTTGCATGGACAGAAAAAGCTCTCCTGGACATTTCTGAACAAGACCCTTCACCCAGGTGGCA
TGGCAGAAGGAGGCTATTTTTTTGAAAGGAGCTGGGGCCACCGTGGAGTTATTGTTCATGTCATCGACCCCAA
ATCAGGCACAGTCATCCATTCTGACCGGTTTGACACCTATAGATCCAAGAAAGAGAGTGAACGTCTGGTCCAG
TATTTGAACGCGGTGCCCGATGGCAGGATCCTTTCTGTTGCAGTGAATGATGAAGGTTCTCGAAATCTGGATG
ACATGGCCAGGAAGGCGATGACCAAATTGGGAAGCAAACACTTCCTGCACCTTGGATTTAGACACCCTTGGAG
TTTTCTAACTGTGAAAGGAAATCCATCATCTTCAGTGGAAGACCATATTGAATATCATGGACATCGAGGCTCT
GCTGCTGCCCGGGTATTCAAATTGTTCCAGACACTCGAGGGC

| | | | |
|---|---|---|---|
| NOV11e, CG59889-10 | | | |
| Protein Sequence | SEQ ID NO: 126 | 251 aa | MW at 27832.4kD |

HVHIGQGKTLLLTSSATVYSIHISEGGKLVIKDHDEPIVLRTRHILIDNGGELHAGSALCPFQGNFTIILYGR
ADEGIQPDPYYGLKYIGVGKGGALELHGQKKLSWTFLNKTLHPGGMAEGGYFFERSWGHRGVIVHVIDPKSGT
VIHSDRFDTYRSKKESERLVQYLNAVPDGRILSVAVNDEGSRNLDDMARKAMTKLGSKHFLHLGFRHPWSFLT
VKGNPSSSVEDHIEYHGHRGSAAARVFKLFQT

| | | |
|---|---|---|
| NOV11f, CG59889-11 | SEQ ID NO: 127 | 1309 bp |
| DNA Sequence | ORF Start: at 11 | ORF Stop: at 1301 |

CACCAGATCTGATCATGATAAAGTATCTCAGACTAAAGGTGGGGAGAAAATTTCAGACCTCTGGAAAGCTCAC
CCAGGAAAAATATGCAATCGTCCCATTGATATACAGGCCACTACAATGGATGGAGTTAACCTCAGCACCGAGG
TTGTCTACAAAAAAGGCCAGGATTATAGGTTTGCTTGCTACGACCGGGGCAGAGCCTGCCGGAGCTACCGTGT
ACGGTTCCTCTGTGGGAAGCCTGTGAGGCCCAAACTCACAGTCACCATTGACACCAATGTGAACAGCACCATT
CTGAACTTGGAGGATAATGTACAGTCATGGAAACCTGGAGATACCCTGGTCATTGCCAGTACTGATTACTCCA
TGTACCAGGCAGAAGAGTTCCAGGTGCTTCCCTGCAGATCCTGCGCCCCCAACCAGGTCAAAGTGGCAGGGAA
ACCAATGTACCTGCACATCGGGGAGGAGATAGACGGCGTGGACATGCGGGCGGAGGTTGGGCTTCTGAGCCGG
AACATCATAGTGATGGGGGAGATGGAGGACAAATGCTACCCCTACAGAAACCACATCTGCAATTTCTTTGACT
TCGATACCTTTGGGGGCCACATCAAGTTTGCTCTGGGATTTAAGGCAGCACACTTGGAGGGCACGGAGCTGAA
GCATATGGGACAGCAGCTGGTGGGTCAGTACCCGATTCACTTCCACCTGGCCGGTGATGTAGACGAAAGGGGA
GGTTATGACCCACCCACATACATCAGGGACCTCTCCATCCATCATACATTCTCTCGCTGCGTCACAGTCCATG
GCTCCAATGGCTTGTTGATCAAGGACGTTGTGGGCTATAACTCTTTGGGCCACTGCTTCTTCACGGAAGATGG
GCCGGAGGAACGCAACACTTTTGACCACTGCCTTGGCCTCCTTGTCAAGTCTGGAACCCTCCTCCCCTCGGAC
CGTGACAGCAAGATGTGCAAGATGATCACAGAGGACTCCTACCCAGGGTACATCCCCAAGCCCAGGCAAGACT
GCAATGCTGTGTCCACCTTCTGGATGGCCAATCCCAACAACAACCTCATCAACTGTGCCGCTGCAGGATCTGA
GGAAACTGGATTTTGGTTTATTTTTCACCACGTACCAACGGGCCCCTCCGTGGGAATGTACTCCCCAGGTTAT
TCAGAGCACATTCCACTGGGAAAATTCTATAACAACCGAGCACATTCCAACTACCGGGCTGGCATGATCATAG
ACAACGGAGTCAAAACCACCGAGGCCTCTGCCAAGGACAAGCGGCCGTTCCTCTCAATCCTCGAGGGC

| | | | |
|---|---|---|---|
| NOV11f, CG59889-11 | | | |
| Protein Sequence | SEQ ID NO: 128 | 430 aa | MW at 48190.2kD |

DHDKVSQTKGGEKISDLWKAHPGKICNRPIDIQATTMDGVNLSTEVVYKKGQDYRFACYDRGRACRSYRVRFL
CGKPVRPKLTVTIDTNVNSTILNLEDNVQSWKPGDTLVIASTDYSMYQAEEFQVLPCRSCAPNQVKVAGKPMY
LHIGEEIDGVDMRAEVGLLSRNIIVMGEMEDKCYPYRNHICNFFDFDTFGGHIKFALGFKAAHLEGTELKHMG
QQLVGQYPIHFHLAGDVDERGGYDPPTYIRDLSIHHTFSRCVTVHGSNGLLIKDVVGYNSLGHCFFTEDGPEE
RNTFDHCLGLLVKSGTLLPSDRDSKMCKMITEDSYPGYIPKPRQDCNAVSTFWMANPNNNLINCAAAGSEETG
FWFIFHHVPTGPSVGMYSPGYSEHIPLGKFYNNRAHSNYRAGMIIDNGVKTTEASAKDKRPFLSI

| | | |
|---|---|---|
| NOV11g, CG59889-12 | SEQ ID NO: 129 | 1081 bp |
| DNA Sequence | ORF Start: at 11 | ORF Stop: at 1073 |

CACCAGATCTGCCTACAAGACCAGTAACCTGCGAATGAAGATCATCAAGAATGACTTCCCCAGCCACCCTCTT
TACCTGGAGGGGGCGCTCACCAGGAGCACCCATTACCAGCAATACCAACCGGTTGTCACCCTGCAGAAGGGCT
ACACCATCCACTGGGACCAGACGGCCCCCGCCGAACTCGCCATCTGGCTCATCAACTTCAACAAGGGCGACTG
GATCCGAGTGGGGCTCTGCTACCCGCGAGGCACCACATTCTCCATCCTCTCGGATGTTCACAATCGCCTGCTG
AAGCAAACGTCCAAGACGGGCGTCTTCGTGAGGACCTTGCAGATGGACAAAGTGGAGCAGAGCTACCCTGGCA
GGAGCCACTACTACTGGGACGAGGACTCAGGGCTGTTGTTCCTGAAGCTGAAAGCTCAGAACGAGAGAGAGAA
GTTTGCTTTCTGCTCCATGAAAGGCTGTGAGAGGATAAAGATTAAAGCTCTGATTCCAAAGAACGCAGGCGTC
AGTGACTGCACAGCCACAGCTTACCCCAAGTTCACCGAGAGGGCTGTCGTAGACGTGCCGATGCCCAAGAAGC
TCTTTGGTTCTCAGCTGAAAACAAAGGACCATTTCTTGGAGGTGAAGATGGAGAGTTCCAAGCAGCACTTCTT
CCACCTCTGGAACGACTTCGCTTACATTGAAGTGGATGGGAAGAAGTACCCCAGTTCGGAGGATGGCATCCAG
GTGGTGGTGATTGACGGGAACCAAGGGCGCGTGGTGAGCCACACGAGCTTCAGGAACTCCATTCTGCAAGGCA
TACCATGGCAGCTTTTCAACTATGTGGCGACCATCCCTGACAATTCCATAGTGCTTATGGCATCAAAGGGAAG
ATACGTCTCCAGAGGCCCATGGACCAGAGTGCTGGAAAAGCTTGGGGCAGACAGGGGTCTCAAGTTGAAAGAG
CAAATGGCATTCGTTGGCTTCAAAGGCAGCTTCCGGCCCATCTGGGTGACACTGGACACTGAGGATCACAAAG
CCAAAATCTTCCAAGTTGTGCCCATCCCTGTGGTGAAGAAGAAGAAGTTGCTCGAGGGC

| | | | |
|---|---|---|---|
| NOV11g, CG59889-12 | | | |
| Protein Sequence | SEQ ID NO: 130 | 354 aa | MW at 40631.7kD |

AYKTSNIRMKIIKNDFPSHPLYLEGALTRSTHYQQYQPVVTLQKGYTIHWDQTAPAELAIWLINFNKGDWIRV
GLCYPRGTTFSILSDVHNRLLKQTSKTGVFVRTLQMDKVEQSYPGRSHYYWDEDSGLLFLKLKAQNEREKFAF
CSMKGCERIKIKALIPKNAGVSDCTATAYPKFTERAVVDVPMPKKLFGSQLKTKDHFLEVKMESSKQHFFHLW
NDFAYIEVDGKKYPSSEDGIQVVVIDGNQGRVVSHTSFRNSILQGIPWQLFNYVATIPDNSIVLMASKGRYVS
RGPWTRVLEKLGADRGLKLKEQMAFVGFKGSFRPIWVTLDTEDHKAKIFQVVPIPVVKKKKL

| | | |
|---|---|---|
| NOV11h, CG59889-13 | SEQ ID NO: 131 | 4108 bp |
| DNA Sequence | ORF Start: ATG at 17 | ORF Stop: at 4100 |

CACCTCGCGAGCCAGGATGGGAGCTGCTGGGAGGCAGGACTTCCTCTTCAAGGCCATGCTGACCATCAGCTGG
CTCACTCTGACCTGCTTCCCTGGGGCCACATCCACAGTGGCTGCTGGGTGCCCTGACCAGAGCCCTGAGTTGC
AACCCTGGAACCCTGGCCATGACCAAGACCACCATGTGCATATCGGCCAGGGCAAGACACTGCTGCTCACCTC
TTCTGCCACGGTCTATTCCATCCACATCTCAGAGGGAGGCAAGCTGGTCATTAAAGACCACGACGAGCCGATT
GTTTTGCGAACCCGGCACATCCTGATTGACAACGGAGGAGAGCTGCATGCTGGGAGTGCCCTCTGCCCTTTCC
AGGGCAATTTCACCATCATTTTGTATGGAAGGGCTGATGAAGGTATTCAGCCGGATCCTTACTATGGTCTGAA
GTACATTGGGGTTGGTAAAGGAGGCGCTCTTGAGTTGCATGGACAGAAAAAACTCTCCTGGACATTTCTGAAC
AAGACCCTTCACCCAGGTGGCATGGCAGAAGGAGGCTATTTTTTTGAAAGGAGCTGGGGCCACCGTGGAGTTA
TTGTTCATGTCATCGACCCCAAATCAGGCACAGTCATCCATTCTGACCGGTTTGACACCTATAGATCCAAGAA
AGAGAGTGAACGTCTGGTCCAGTATTTGAACGCGGTGCCCGATGGCAGGATCCTTTCTGTTGCAGTGAATGAT
GAAGGTTCTCGAAATCTGGATGACATGGCCAGGAAGGCGATGACCAAATTGGGAAGCAAACACTTCCTGCACC
TTGGATTTAGACACCCTTGGAGTTTTCTAACTGTGAAAGGAAATCCATCATCTTCAGTGGAAGACCATATTGA
ATATCATGGACATCGAGGCTCTGCTGCTGCCCGGGTATTCAAATTGTTCCAGACAGAGCATGGCGAATATTTC
AATGTTTCTTTGTCCAGTGAGTGGGTTCAAGACGTGGAGTGGACGGAGTGGTTCGATCATGATAAAGTATCTC
AGACTAAAGGTGGGGAGAAAATTTCAGACCTCTGGAAAGCTCACCCAGGAAAAATATGCAATCGTCCCATTGA
TATACAGGCCACTACAATGGATGGAGTTAACCTCAGCACCGAGGTTGTCTACAAAAAAGGCCAGGATTATAGG
TTTGCTTGCTACGACCGGGGCAGAGCCTGCCGGAGCTACCGTGTACGGTTCCTCTGTGGGAAGCCTGTGAGGC
CCAAACTCACAGTCACCATTGACACCAATGTGAACAGCACCATTCTGAACTTGGAGGATAATGTACAGTCATG
GAAACCTGGAGATACCCTGGTCATTGCCAGTACTGATTACTCCATGTACCAGGCAGAAGAGTTCCAGGTGCTT
CCCTGCAGATCCTGCGCCCCCAACCAGGTCAAAGTGGCAGGGAAACCAATGTACCTGCACATCGGGGAGGAGA
TAGACGGCGTGGACATGCGGGCGGAGGTTGGGCTTCTGAGCCGGAACATCATAGTGATGGGGGAGATGGAGGA
CAAATGCTACCCCTACAGAAACCACATCTGCAATTTCTTTGACTTCGATACCTTTGGGGGCCACATCAAGTTT
GCTCTGGGATTTAAGGCAGCACACTTGGAGGGCACGGAGCTGAAGCATATGGGACAGCAGCTGGTGGGTCAGT
ACCCGATTCACTTCCACCTGGCCGGTGATGTAGACGAAAGGGGAGGTTATGACCCACCCACATACATCAGGGA
CCTCTCCATCCATCATACATTCTCTCGCTGCGTCACAGTCCATGGCTCCAATGGCTTGTTGATCAAGGACGTT
GTGGGCTATAACTCTTTGGGCCACTGCTTCTTCACGGAAGATGGGCCGGAGGAACGCAACACTTTTGACCACT
GTCTTGGCCTCCTTGTCAAGTCTGGAACCCTCCTCCCCTCGGACCGTGACAGCAAGATGTGCAAGATGATCAC
AGAGGACTCCTACCCAGGGTACATCCCCAAGCCCAGGCAAGACTGCAATGCTGTGTCCACCTTCTGGATGGCC
AATCCCAACAACAACCTCATCAACTGTGCCGCTGCAGGATCTGAGGAAACTGGATTTTGGTTTATTTTTCACC
ACGTACCAACGGGCCCCTCCGTGGGAATGTACTCCCCAGGTTATTCAGAGCACATTCCACTGGGAAAATTCTA
TAACAACCGAGCACATTCCAACTACCGGGCTGGCATGATCATAGACAACGGAGTCAAAACCACCGAGGCCTCT
GCCAAGGACAAGCGGCCGTTCCTCTCAATCATCTCTGCCAGATACAGCCCTCACCAGGACGCCGACCCGCTGA
AGCCCCGGGAGCCGGCCATCATCAGACACTTCATTGCCTACAAGAACCAGGACCGCGGGGCCTGGCTGCGCGG
CGGGGATGTGTGGCTGGACAGCTGCCGGTTTGCTGACAATGGCATTGGCCTGACCCTGGCCAGTGGTGGAACC
TTCCCGTATGACGACGGCTCCAAGCAAGAGATAAAGAACAGCTTGTTTGTTGGCGAGAGTGGCAACGTGGGGA
CGGAAATGATGGACAATAGGATCTGGGGCCCTGGCGGCTTGGACCATAGCGGAAGGACCCTCCCTATAGGCCA
GAATTTTCCAATTAGAGGAATTCAGTTATATGATGGCCCCATCAACATCCTAAACTGCACTTTCCGAAAGTTT
GTGGCCCTGGAGGGCCGGCACACCAGCGCCCTGGCCTTCCGCCTGAATAATGCCTGGCAGAGCTGCCCCCATA
ACAACGTGACCGGCATTGCCTTTGAGGACGTTCCGATTACTTCCAGAGTGTTCTTCGGAGAGCCTGGGCCCTG
GTTCAACCAGCTGGACATGGATGGGGATAAGACATCTGTGTTCCATGACGTCGACGGCTCCGTGTCCGAGTAC
CCTGGCTCCTACCTCACGAAGAATGACAACTGGCTGGTCCGGCACCCAGACTGCATCAATGTTCCCGACTGGA
GAGGGGCCATTTGCAGTGGGTGCTATGCACAGATGTACATTCAAGCCTACAAGACCAGTAACCTGCGAATGAA
GATCATCAAGAATGACTTCCCCAGCCACCCTCTTTACCTGGAGGGGGCGCTCACCAGGAGCACCCATTACCAG
CAATACCAACCGGTTGTCACCCTGCAGAAGGGCTACACCATCCACTGGGACCAGACGGCCCCCGCCGAACTCG
CCATCTGGCTCATCAACTTCAACAAGGGCGACTGGATCCGAGTGGGGCTCTGCTACCCGCGAGGCACCACATT
CTCCATCCTCTCGGATGTTCACAATCGCCTGCTGAAGCAAACGTCCAAGACGGGCGTCTTCGTGAGGACCTTG
CAGATGGACAAAGTGGAGCAGAGCTACCCTGGCAGGAGCCACTACTACTGGGACGAGGACTCAGGGCTGTTGT
TCCTGAAGCTGAAAGCTCAGAACGAGAGAGAGAAGTTTGCTTTCTGCTCCATGAAAGGCTGTGAGAGGATAAA
GATTAAAGCTCTGATTCCAAAGAACGCAGGCGTCAGTGACTGCACAGCCACAGCTTACCCCAAGTTCACCGAG
AGGGCTGTCGTAGACGTGCCGATGCCCAAGAAGCTCTTTGGTTCTCAGCTGAAAACAAAGGACCATTTCTTGG
AGGTGAAGATGGAGAGTTCCAAGCAGCACTTCTTCCACCTCTGGAACGACTTCGCTTACATTGAAGTGGATGG
GAAGAAGTACCCCAGTTCGGAGGATGGCATCCAGGTGGTGGTGATTGACGGGAACCAAGGGCGCGTGGTGAGC
CACACGAGCTTCAGGAACTCCATTCTGCAAGGCATACCATGGCAGCTTTTCAACTATGTGGCGACCATCCCTG
ACAATTCCATAGTGCTTATGGCATCAAAGGGAAGATACGTCTCCAGAGGCCCATGGACCAGAGTGCTGGAAAA
GCTTGGGGCAGACAGGGGTCTCAAGTTGAAAGAGCAAATGGCATTCGTTGGCTTCAAAGGCAGCTTCCGGCCC
ATCTGGGTGACACTGGACACTGAGGATCACAAAGCCAAAATCTTCCAAGTTGTGCCCATCCCTGTGGTGAAGA
AGAAGAAGTTGCTCGAGGGC

| | | | |
|---|---|---|---|
| NOV11h, CG59889-13 | | | |
| Protein Sequence | SEQ ID NO: 132 | 1361 aa | MW at 153000.5kD |

MGAAGRQDFLFKAMLTISWLTLTCFPGATSTVAAGCPDQSPELQPWNPGHDQDHHVHIGQGKTLLLTSSATVY
SIHISEGGKLVIKDHDEPIVLRTRHILIDNGGELHAGSALCPFQGNFTIILYGRADEGIQPDPYYGLKYIGVG
KGGALELHGQKKLSWTFLNKTLHPGGMAEGGYFFERSWGHRGVIVHVIDPKSGTVIHSDRFDTYRSKKESERL
VQYLNAVPDGRILSVAVNDEGSRNLDDMARKAMTKIGSKMFLHLGFRHPWSFLTVKGNPSSSVEDHIEYHGHR
GSAAARVFKLFQTEHGEYFNVSLSSEWVQDVEWTEWFDHDKVSQTKGGEKISDLWKAHPGKICNRPIDIQATT
MDGVNLSTEVVYKKGQDYRFACYDRGRACRSYRVRFLCGKPVRPKLTVTIDTNVNSTILNLEDNVQSWKPGDT
LVIASTDYSMYQAEEFQVLPCRSCAPNQVKVAGKPMYLHIGEEIDGVDMRAEVGLLSRNIIVMGEMEDKCYPY
RNHICNFFDFDTFGGHIKFALGFKAAHLEGTELKHMGQQLVGQYPIHFHLAGDVDERGGYDPPTYIRDLSIHH
TFSRCVTVHGSNGLLIKDVVGYNSLGHCFFTEDGPEERNTFDHCLGLLVKSGTLLPSDRDSKMCKMITEDSYP
GYIPKPRQDCNAVSTFWMANPNNNLINCAAAGSEETGFWFIFHHVPTGPSVGMYSPGYSEHIPLGKFYNNRAH
SNYRAGMIIDNGVKTTEASAKDKRPFLSIISARYSPHQDADPLKPREPAIIRHFIAYKNQDRGAWLRGGDVWL
DSCRFADNGIGLTLASGGTFPYDDGSKQEIKNSLFVGESGNVGTEMMDNRIWGPGGLDHSGRTLPIGQNFPIR
GIQLYDGPINILNCTFRKFVALEGRHTSALAFRLNNAWQSCPHNNVTGIAFEDVPITSRVFFGEPGPWFNQLD
MDGDKTSVFHDVDGSVSEYPGSYLTKNDNWLVRHPDCINVPDWRGAICSGCYAQMYIQAYKTSNLRMKIIKND
FPSHPLYLEGALTRSTHYQQYQPVVTLQKGYTIHWDQTAPAELAIWLINFNKGDWIRVGLCYPRGTTFSILSD
VHNRLLKQTSKTGVFVRTLQMDKVEQSYPGRSHYYWDEDSGLLFLKLKAQNEREKFAFCSMKGCERIKIKALI
PKNAGVSDCTATAYPKFTERAVVDVPMPKKLFGSQLKTKDHFLEVKMESSKQHFFHLWNDFAYIEVDGKKYPS
SEDGIQVVVIDGNQGRVVSHTSFRNSILQGIPWQLFNYVATIPDNSIVLMASKGRYVSRGPWTRVLEKLGADR
GLKLKEQMAFVGFKGSFRPIWVTLDTEDHKAKIFQVVPIPVVKKKKL

| | | |
|---|---|---|
| NOV11i, 311979177 | SEQ ID NO: 133 | 3058 bp |
| DNA Sequence | ORF Start: at 11 | ORF Stop: at 3053 |

CACCGGTACCGCTCACCCAGGAAAAATATGCAATCGTCCCATTGATATACAGGCCACTACAATGGATGGAGTT
AACCTCAGCACCGAGGTTGTCTACAAAAAAGGCCAGGATTATAGGTTTGCTTGCTACGACCGGGGCAGAGCCT
GCCGGAGCTACCGTGTACGGTTCCTCTGTGGGAAGCCTGTGAGGCCCAAACTCACAGTCACCATTGACACCAA
TGTGAACAGCACCATTCTGAACTTGGAGGATAATGTACAGTCATGGAAACCTGGAGATACCCTGGTCATTGCC
AGTACTGATTACTCCATGTACCAGGCAGAAGAGTTCCAGGTGCTTCCCTGCAGATCCTGCGCCCCCAACCAGG
TCAAAGTGGCAGGGAAACCAATGTACCTGCACATCGGGGAGGAGATAGACGGCGTGGACATGCGGGCGGAGGT
TGGGCTTCTGAGCCGGAACATCATAGTGATGGGGGAGATGGAGGACAAATGCTACCCCTACAGAAACCACATC
TGCAATTTCTTTGACTTCGATACCTTTGGGGGCCACATCAAGTTTGCTCTGGGATTTAAGGCAGCACACTTGG
AGGGCACGGAGCTGAAGCATATGGGACAGCAGCTGGTGGGTCAGTACCCGATTCACTTCCACCTGGCCGGTGA
TGTAGACGAAAGGGGAGGTTATGACCCACCCACATACATCAGGGACCTCTCCATCCATCATACATTCTCTCGC
TGCGTCACAGTCCATGGCTCCAATGGCTTGTTGATCAAGGACGTTGTGGGCTATAACTCTTTGGGCCACTGCT
TCTTCACGGAAGATGGGCCGGAGGAACGCAACACTTTTGACCACTGTCTTGGCCTCCTTGTCAAGTCTGGAAC
CCTCCTCCCCTCGGACCGTGACAGCAAGATGTGCAAGATGATCACAGAGGACTCCTACCCAGGGTACATCCCC
AAGCCCAGGCAAGACTGCAATGCTGTGTCCACCTTCTGGATGGCCAATCCCAACAACAACCTCATCAACTGTG
CCGCTGCAGGATCTGAGGAAACTGGATTTTGGTTTATTTTTCACCACGTACCAACGGGCCCCTCCGTGGGAAT
GTACTCCCCAGGTTATTCAGAGCACATTCCACTGGGAAAATTCTATAACAACCGAGCACATTCCAACTACCGG
GCTGGCATGATCATAGACAACGGAGTCAAAACCACCGAGGCCTCTGCCAAGGACAAGCGGCCGTTCCTCTCAA
TCATCTCTGCCAGATACAGCCCTCACCAGGACGCCGACCCGCTGAAGCCCCGGGAGCCGGCCATCATCAGACA
CTTCATTGCCTACAAGAACCAGGACCACGGGGCCTGGCTGCGCGGCGGGGATGTGTGGCTGGACAGCTGCCGG
TTTGCTGACAATGGCATTGGCCTGACCCTGGCCAGTGGTGGAACCTTCCCGTATGACGACGGCTCCAAGCAAG
AGATAAAGAACAGCTTGTTTGTTGGCGAGAGTGGCAACGTGGGGACGGAAATGATGGACAATAGGATCTGGGG
CCCTGGCGGCTTGGACCATAGCGGAAGGACCCTCCCTATAGGCCAGAATTTTCCAATTAGAGGAATTCAGTTA
TATGATGGCCCCATCAACATCCAAAACTGCACTTTCCGAAAGTTTGTGGCCCTGGAGGGCCGGCACACCAGCG
CCCTGGCCTTCCGCCTGAATAATGCCTGGCAGAGCTGCCCCCATAACAACGTGACCGGCATTGCCTTTGAGGA
CGTTCCGATTACTTCCAGAGTGTTCTTCGGAGAGCCTGGGCCCTGGTTCAACCAGCTGGACATGGATGGGGAT
AAGACATCTGTGTTCCATGACGTCGACGGCTCCGTGTCCGAGTACCCTGGCTCCTACCTCACGAAGAATGACA
ACTGGCTGGTCCGGCACCCAGACTGCATCAATGTTCCCGACTGGAGAGGGGCCATTTGCAGTGGGTGCTATGC
ACAGATGTACATTCAAGCCTACAAGACCAGTAACCTGCGAATGAAGATCATCAAGAATGACTTCCCCAGCCAC
CCTCTTTACCTGGAGGGGGCGCTCACCAGGAGCACCCATTACCAGCAATACCAACCGGTTGTCACCCTGCAGA
AGGGCTACACCATCCACTGGGACCAGACGGCCCCCGCCGAACTCGCCATCTGGCTCATCAACTTCAACAAGGG
CGACTGGATCCGAGTGGGGCTCTGCTACCCGCGAGGCACCACATTCTCCATCCTCTCGGATGTTCACAATCGC
CTGCTGAAGCAAACGTCCAAGACGGGCGTCTTCGTGAGGACCTTGCAGATGGACAAAGTGGAGCAGAGCTACC
CTGGCAGGAGCCACTACTACTGGGACGAGGACTCAGGGCTGTTGTTCCTGAAGCTGAAAGCTCAGAACGAGAG
AGAGAAGTTTGCTTTCTGCTCCATGAAAGGCTGTGAGAGGATAAAGATTAAAGCTCTGATTCCAAAGAACGCA
GGCGTCAGTGACTGCACAGCCACAGCTTACCCCAAGTTCACCGAGAGGGCTGTCGTAGACGTGCCGATGCCCA
AGAAGCTCTTTGGTTCTCAGCTGAAAACAAAGGACCATTTCTTGGAGGTGAAGATGGAGAGTTCCAAGCAGCA
CTTCTTCCACCTCTGGAACGACTTCGCTTACATTGAAGTGGATGGGAAGAAGTACCCCAGTTCGGAGGATGGC
ATCCAGGTGGTGGTGATTGACGGGAACCAAGGGCGCGTGGTGAGCCACACGAGCTTCAGGAACTCCATTCTGC
AAGGCATACCATGGCAGCTTTTCAACTATGTGGCGACCATCCCTGACAATTCCATAGTGCTTATGGCATCAAA
GGGAAGATACGTCTCCAGAGGCCCATGGACCAGAGTGCTGGAAAAGCTTGGGGCAGACAGGGGTCTCAAGTTG
AAAGAGCAAATGGCATTCGTTGGCTTCAAAGGCAGCTTCCGGCCCATCTGGGTGACACTGGACACTGAGGATC
ACAAAGCCAAAATCTTCCAAGTTGTGCCCATCCCTGTGGTGAAGAAGAAGAAGTTGCTCGAGGGC

| | | | |
|---|---|---|---|
| NOV11i, 311979177 | | | |
| Protein Sequence | SEQ ID NO: 134 | 1014 aa | MW at 114357.5kD |

AHPGKICNRPIDIQATTMDGVNLSTEVVYKKGQDYRFACYDRGRACRSYRVRFLCGKPVRPKLTVTIDTNVNS
TILNLEDNVQSWKPGDTLVIASTDYSMYQAEEFQVLPCRSCAPNQVKVAGKPMYLHIGEEIDGVDMRAEVGLL
SRNIIVMGEMEDKCYPYRNHICNFFDFDTFGGHIKFALGFKAAHLEGTELKHMGQQLVGQYPIHFHLAGDVDE
RGGYDPPTYIRDLSIHHTFSRCVTVHGSNGLLIKDVVGYNSLGHCFFTEDGPEERNTFDHCLGLLVKSGTLLP
SDRDSKMCKMITEDSYPGYIPKPRQDCNAVSTFWMANPNNNLINCAAAGSEETGFWFIFHHVPTGPSVGMYSP
GYSEHIPLGKFYNNPAHSNYRAGMIIDNGVKTTEASAKDKRPFLSIISARYSPHQDADPLKPREPAIIRHFIA
YKNQDHGAWLRGGDVWLDSCRFADNGIGLTLASGGTFPYDDGSKQEIKNSLFVGESGNVGTEMMDNRTWGPGG
LDHSGRTLPIGQNFPIRGIQLYDGPINIQNCTFRKFVALEGRHTSALAFRLNNAWQSCPHNNVTGIAFEDVPI
TSRVFFGEPGPWFNQLDMDGDKTSVFHDVDGSVSEYPGSYLTKNDNWLVRHPDCINVPDWRGAICSGCYAQMY
IQAYKTSNLRMKIIKNDFPSHPLYLEGALTRSTHYQQYQPVVTLQKGYTIHWDQTAPAELAIWLINFNKGDWI
RVGLCYPRGTTFSILSDVHNRLLKQTSKTGVFVRTLQMDKVEQSYPGRSHYYWDEDSGLLFLKLKAQNEREKF
AFCSMKGCERIKIKALIPKNAGVSDCTATAYPKFTERAVVDVPMPKKLFGSQLKTKDHFLEVKMESSKQHFFH
LWNDFAYIEVDGKKYPSSEDGIQVVVIDGNQGRVVSHTSFRNSILQGIPWQLFNYVATIPDNSIVLMASKGRY
VSRGPWTRVLEKLGADRGLKLKEQMAFVGFKGSFRPIWVTLDTEDHKAKIFQVVPIPVVKKKKLL

| | | |
|---|---|---|
| NOV11j, 314361479 | SEQ ID NO: 135 | 3997 bp |
| DNA Sequence | ORF Start: at 11 | ORF Stop: at 3992 |

CACCAGATCTTGCCCTGACCAGAGCCCTGAGTTGCAACCCTGGAACCCTGGCCATGACCAAGACCACCATGTG
CATATCGGCCAGGGCAAGACACTGCTGCTCACCTCTTCTGCCACGGTCTATTCCATCCACATCTCAGAGGGAG
GCAAGCTGGTCATTAAAGACCACGACGAGCCGATTGTTTTGCGAACCCGGCACATCCTGATTGACAACGGAGG
AGAGCTGCATGCTGGGAGTGCCCTCTGCCCTTTCCAGGGCAATTTCACCATCATTTTGTATGGAAGGGCTGAT
GAAGGTATTCAGCCGGATCCTTACTATGGTCTGAAGTACATTGGGGTTGGTAAAGGAGGCGCTCTTGAGTTGC
ATGGACAGAAAAAGCTCTCCTGGACATTTCTGAACAAGACCCTTCACCCAGGTGGCATGGCAGAAGGAGGCTA
TTTTTTTGAAAGGAGCTGGGGCCACCGTGGAGTTATTGTTCATGTCATCGACCCCAAATCAGGCACAGTCATC
CATTCTGACCGGTTTGACACCTATAGATCCAAGAAAGAGAGTGAACGTCTGGTCCAGTATTTGAACGCGGTGC
CCGATGGCAGGATCCTTTCTGTTGCAGTGAATGATGAAGGTTCTCGAAATCTGGATGACATGGCCAGGAAGGC
GATGACCAAATTGGGAAGCAAACACTTCCTGCACCTTGGATTTAGACACCCTTGGAGTTTTCTAACTGTGAAA
GGAAATCCATCATCTTCAGTGGAAGACCATATTGAATATCATGGACATCGAGGCTCTGCTGCTGCCCGGGTAT
TCAAATTGTTCCAGACAGAGCATGGCGAATATTTCAATGTTTCTTTGTCCAGTGAGTGGGTTCAAGACGTGGA
GTGGACGGAGTGGTTCGATCATGATAAAGTATCTCAGACTAAAGGTGGGGAGAAAATTTCAGACCTCTGGAAA
GCTCACCCAGGAAAAATATGCAATCGTCCCATTGATATACAGGCCACTACAATGGATGGAGTTAACCTCAGCA
CCGAGGTTGTCTACAAAAAAGGCCAGGATTATAGGTTTGCTTGCTACGACCGGGGCAGAGCCTGCCGGAGCTA
CCGTGTACGGTTCCTCTGTGGGAAGCCTGTGAGGCCCAAACTCACAGTCACCATTGACACCAATGTGAACAGC
ACCATTCTGAACTTGGAGGATAATGTACAGTCATGGAAACCTGGAGATACCCTGGTCATTGCCAGTACTGATT
ACTCCATGTACCAGGCAGAAGAGTTCCAGGTGCTTCCCTGCAGATCCTGCGCCCCCAACCAGGTCAAAGTGGC
AGGGAAACCAATGTACCTGCACATCGGGGAGGAGATAGACGGCGTGGACATGCGGGCGGAGGTTGGGCTTCTG
AGCCGGAACATCATAGTGATGGGGGAGATGGAGGACAAATGCTACCCCTACAGAAACCACATCTGCAATTTCT
TTGACTTCGATACCTTTGGGGGCCACATCAAGTTTGCTCTGGGATTTAAGGCAGCACACTTGGAGGGCACGGA
GCTGAAGCATATGGGACAGCAGCTGGTGGGTCAGTACCCGATTCACTTCCACCTGGCCGGTGATGTAGACGAA
AGGGGAGGTTATGACCCACCCACATACATCAGGGACCTCTCCATCCATCATACATTCTCTCGCTGCGTCACAG
TCCATGGCTCCAATGGCTTGTTGATCAAGGACGTTGTGGGCTATAACTCTTTGGGCCACTGCTTCTTCACGGA
AGATGGGCCGGAGGAACGCAACACTTTTGACCACTGCCTTGGCCTCCTTGTCAAGTCTGGAACCCTCCTCCCC
TCGGACCGTGACAGCAAGATGTGCAAGATGATCACAGAGGACTCCTACCCAGGGTACATCCCCAAGCCCAGGC
AAGACTGCAATGCTGTGTCCACCTTCTGGATGGCCAATCCCAACAACAACCTCATCAACTGTGCCGCTGCAGG
ATCTGAGGAAACTGGATTTTGGTTTATTTTTCACCACGTACCAACGGGCCCCTCCGTGGGAATGTACTCCCCA
GGTTATTCAGAGCACATTCCACTGGGAAAATTCTATAACAACCGAGCACATTCCAACTACCGGGCTGGCATGA
TCATAGACAACGGAGTCAAAACCACCGAGGCCTCTGCCAAGGACAAGCGGCCGTTCCTCTCAATCATCTCTGC
CAGATACAGCCCTCACCAGGACGCCGACCCGCTGAAGCCCCGGGAGCCGGCCATCATCAGACACTTCATTGCC
TACAAGAACCAGGACCACGGGGCCTGGCTGCGCGGCGGGGATGTGTGGCTGGACAGCTGCCGGTTTGCTGACA
ATGGCATTGGCCTGACCCTGGCCAGTGGTGGAACCTTCCCGTATGACGACGGCTCCAAGCAAGAGATAAAGAA
CAGCTTGTTTGTTGGCGAGAGTGGCAACGTGGGGACGGAAATGATGGACAATAGGATCTGGGGCCCTGGCGGC
TTGGACCATAGCGGAAGGACCCTCCCTATAGGCCAGAATTTTCCAATTAGAGGAATTCAGTTATATGATGGCC
CCATCAACATCCAAAACTGCACTTTCCGAAAGTTTGTGGCCCTGGAGGGCCGGCACACCAGCGCCCTGGCCTT
CCGCCTGAATAATGCCTGGCAGAGCTGCCCCCATAACAACGTGACCGGCATTGCCTTTGAGGACGTTCCGATT
ACTTCCAGAGTGTTCTTCGGAGAGCCTGGGCCCTGGTTCAACCAGCTGGACATGGATGGGGATAAGACATCTG
TGTTCCATGACGTCGACGGCTCCGTGTCCGAGTACCCTGGCTCCTACCTCACGAAGAATGGCAACTGGCTGGT
CCGGCACCCAGACTGCATCAATGTTCCCGACTGGAGAGGGGCCATTTGCAGTGGGTGCTATGCACAGATGTAC
ATTCAAGCCTACAAGACCAGTAACCTGCGAATGAAGATCATCAAGAATGACTTCCCCAGCCACCCTCTTTACC
TGGAGGGGGCGCTCACCAGGAGCACCCATTACCAGCAATACCAACCGGTTGTCACCCTGCAGAAGGGCTACAC
CATCCACTGGGACCAGACGGCCCCCGCCGAACTCGCCATCTGGCTCATCAACTTCAACAAGGGCGACTGGATC
CGAGTGGGGCTCTGCTACCCGCGAGGCACCACATTCTCCATCCTCTCGGATGTTCACAATCGCCTGCTGAAGC
AAACGTCCAAGACGGGCGTCTTCGTGAGGACCTTGCAGATGGACAAAGTGGAGCAGAGCTACCCTGGCAGGAG
CCACTACTACTGGGACGAGGACTCAGGGCTGTTGTTCCTGAAGCTGAAAGCTCAGAACGAGAGAGAGAAGTTT
GCTTTCTGCTCCATGAAAGGCTGTGAGAGGATAAAGATTAAAGCTCTGATTCCAAAGAACGCAGGCGTCAGTG
ACTGCACAGCCACAGCTTACCCCAAGTTCACCGAGAGGGCTGTCGTAGACGTGCCGATGCCCAAGAAGCTCTT
TGGTTCTCAGCTGAAAACAAAGGACCATTTCTTGGAGGTGAAGATGGAGAGTTCCAAGCAGCACTTCTTCCAC
CTCTGGAACGACTTCGCTTACATTGAAGTGGATGGGAAGAAGTACCCCAGTTCGGAGGATGGCATCCAGGTGG
TGGTGATTGACGGGAACCAAGGGCGCGTGGTGAGCCACACGAGCTTCAGGAACTCCATTCTGCAAGGCATACC
ATGGCAGCTTTTCAACTATGTGGCGACCATCCCTGACAATTCCATAGTGCTTATGGCATCAAAGGGAAGATAC
GTCTCCAGAGGCCCATGGACCAGAGTGCTGGAAAAGCTTGGGGCAGACAGGGGTCTCAAGTTGAAAGAGCAAA
TGGCATTCGTTGGCTTCAAAGGCAGCTTCCGGCCCATCTGGGTGACACTGGACACTGAGGATCACAAAGCCAA
AATCTTCCAAGTTGTGCCCATCCCTGTGGTGAAGAAGAAGAAGTTGCTCGAGGGC

| | | | |
|---|---|---|---|
| NOV11j, 314361479 | | | |
| Protein Sequence | SEQ ID NO: 136 | 1327 aa | MW at 149436.0kD |

CPDQSPELQPWNPGHDQDHHVHIGQGKTLLLTSSATVYSIHISEGGKLVIKDHDEPIVLRTRHILIDNGGELH
AGSALCPFQGNFTIILYGRADEGIQPDPYYGLKYIGVGKGGALELHGQKKLSWTFLNKTLHPGGMAEGGYFFE
RSWGHRGVIVHVIDPKSGTVIHSDRFDTYRSKKESERLVQYLNAVPDGRILSVAVNDEGSRNLDDMARKAMTK
LGSKHFLHLGFRHPWSFLTVKGNPSSSVEDHTEYHGHRGSAAARVFKLFQTEHGEYFNVSLSSEWVQDVEWTE
WFDHDKVSQTKGGEKISDLWKAHPGKICNRPIDIQATTMDGVNLSTEVVYKKGQDYRFACYDRGRACRSYRVR
FLCGKPVRPKLTVTIDTNVNSTILNLEDNVQSWKPGDTLVIASTDYSMYQAEEFQVLPCRSCAPNQVKVAGKP
MYLHIGEEIDGVDMRAEVGLLSRNIIVMGEMEDKCYPYRNHICNFFDFDTFGGHIKFALGFKAAHLEGTELKH
MGQQLVGQYPIHFHLAGDVDERGGYDPPTYIRDLSIHHTFSRCVTVHGSNGLLIKDVVGYNSLGHCFFTEDGP
EERNTFDHCLGLLVKSGTLLPSDRDSKMCKMITEDSYPGYIPKPRQDCNAVSTFWMANPNNNLINCAAAGSEE
TGFWFIFHHVPTGPSVGMYSPGYSEHIPLGKFYNNRAHSNYRAGMIIDNGVKTTEASAKDKRPFLSIISARYS
PHQDADPLKPREPAIIRHFIAYKNQDHGAWLRGGDVWLDSCRFADNGIGLTLASGGTFPYDDGSKQEIKNSLF
VGESGNVGTEMMDNRIWGPGGLDHSGRTLPIGQNFPIRGIQLYDGPINIQNCTFRKFVALEGRHTSALAFRLN
NAWQSCPHNNVTGIAFEDVPITSRVFFGEPGPWFNQLDMDGDKTSVFHDVDGSVSEYPGSYLTKNGNWLVRHP
DCINVPDWRGAICSGCYAQMYIQAYKTSNLRMKIIKNDFPSHPLYLEGALTRSTHYQQYQPVVTLQKGYTIHW
DQTAPAELAIWLINFNKGDWIRVGLCYPRGTTFSILSDVHNRLLKQTSKTGVFVRTLQMDKVEQSYPGRSHYY
WDEDSGLLFLKLKAQNEREKFAFCSMKGCERIKIKALIPKNAGVSDCTATAYPKFTERAVVDVPMPKKLFGSQ
LKTKDHFLEVKMESSKQHFFHLWNDFAYIEVDGKKYPSSEDGIQVVVIDGNQGRVVSHTSFRNSILQGIPWQL
FNYVATIPDNSIVLMASKGRYVSRGPWTRVLEKLGADRGLKLKEQMAFVGFKGSFRPIWVTLDTEDHKAKIFQ
VVPIPVVKKKKLL

A ClustalW comparison of the above protein sequences yields the following sequence alignment shown in Table 11B.

TABLE 11B
Comparison of the NOV11 protein sequences.

| | |
|---|---|
| NOV11a | CPDQSPELQPWNPGHDQDHHVHIGQGKTLLLTSSATVYSIHISEGGKLVIKDHDEPIVLR |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | ------------------------------------------------------------ |
| NOV11j | ------------------------------------------------------------ |
| NOV11a | TRHILIDNGGELHAGSALCPFQGNFTIILYGRADEGIQPDPYYGLKYIGVGKGGALELHG |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | ------------------------------------------------------------ |
| NOV11j | ------------------------------------------------------------ |
| NOV11a | QKKLSWTFLNKTLHPGGMAEGGYFFERSWGHRGVIVHVIDPKSGTVIHSDRFDTYRSKKE |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | ------------------------------------------------------------ |
| NOV11j | ------------------------------------------------------------ |
| NOV11a | SERLVQYLNAVPDGRILSVAVNDEGSRNLDDMARKAMTKLGSKMFLHLGFRVEWTEWFDH |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | ------------------------------------------------------------ |
| NOV11j | ------------------------------------------------------------ |
| NOV11a | DKVSQTKGGEKISDLWKAHPGKICNRPIDIQQATTMDGVNLSTEVVYKKGQDYRFACYDR |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | ---------------TGTAHPGKICNRPIDIQATTMDGVNLSTEVVYKKGQDYRFACYDR |
| NOV11j | ------------------------------------------------------------ |
| NOV11a | GRACRSYRVRFLCGKPVRPKLTVTIDTNVNSTILNLEDNVQSWKPGDTLVIASTDYSMYQ |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | GRACRSYRVRFLCGKPVRPKLTVTIDTNVNSTILNLEDNVQSWKPGDTLVIASTDYSMYQ |
| NOV11j | ------------------------------------------------------------ |
| NOV11a | AEEFQVLPCRSCAPNQVKVAGKPMYLHIGEEIDGVDMRAEVGLLSRNIIVMGEMEDKCYP |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | AEEFQVLPCRSCAPNQVKVAGKPMYLHIGEEIDGVDMRAEVGLLSRNIIVMGEMEDKCYP |
| NOV11j | ------------------------------------------------------------ |
| NOV11a | YRNHICNFFDFDTFGGHIKFALGFKAAHLEGTELKHMGQQLVGQYPIHFHLAGDVDERGG |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | YRNHICNFFDFDTFGGHIKFALGFKAAHLEGTELKHMGQQLVGQYPIHFHLAGDVDERGG |
| NOV11j | ------------------------------------------------------------ |
| NOV11a | YDPPTYIRDLSIHHTFSRCVTVHGSNGLLIKDVVGYNSLGHCFFTEDGPEERNTFDHCLG |
| NOV11b | ------------------------------------------------------------ |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ------------------------------------------------------------ |
| NOV11i | YDPPTYIRDLSIHHTFSRCVTVHGSNGLLIKDVVGYNSLGHCFFTEDGPEERNTFDHCLG |
| NOV11j | ------------------------------------------------------------ |
| NOV11a | LLVKSGTLLPSDRDSKMCKMITEDSYPGYIPKPRQDCNAVSTFWMANPNNNLINCAAAGS |
| NOV11b | -----------------------MYYTISRKHILETHLPQNTQSREGAGPNPGATPPPPP |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ---------------------------------------MGAAGRQDFLFKANLTISWLT |
| NOV11i | LLVKSGTLLPSDRDSKMCKMITEDSYPGYIPKPRQDCNAVSTFWMANPNNNLINCAAAGS |
| NOV11j | ------------------------------------------------------------ |
| NOV11a | EETGFWFIFHHVPTGPSVGMYSPGYSEHIPLGKFYNNRAHSNYRAGMIIDNGVKTTEASA |
| NOV11b | VPRASRRLTKRLEREDRSTALQPGQQSETLSQKKKRSKNNYAVCLDILIFVLISFFLPLK |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | LTCFPGATSTVAAGCPDQSPELQPWNPGHDQDHHVHIGQGKTLLLTSSATVYSIHISEGG |
| NOV11i | EETGFWFIFHHVPTGPSVGMYSPGYSEHIPLGKFYNNRAHSNYRAGMIIDNGVKTTEASA |
| NOV11j | -----------TRSCPDQSPELQPWNPGHDQDHHVHIGQGKTLLLTSSATVYSIHISEGG |
| NOV11a | KDKRPFLSIISARYSPHQDADPLKPREPAIIRHFIAYKNQDHGAWLRGGDVWLDSCRFAD |
| NOV11b | TPLGETSAAGCPDQSPELQPWNPGHDQDHHVHIGQGKTLLLTSSATVYSIHISEGGKLVI |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | KLVIKDHDEPIVLRTRHILIDNGGELHAGSALCPFQGNFTIILYGRADEGIQPDPYYGLK |
| NOV11i | KDKRPFLSIISARYSPHQDADPLKPREPAIIRHFIAYKNQDHGAWLRGGDVWLDSCRFAD |
| NOV11j | KLVIKDHDEPIVLRTRHILIDNGGELHAGSALCPFQGNFTIILYGRADEGIQPDPYYGLK |
| NOV11a | NGIGLTLASGGTFPYDDGSKQEIKNSLFVGESGNVGTEMMDNRIWGPGGLDHSGRTLPIG |
| NOV11b | KDHDEPIVLRTRHILIDNGGELHAGSALCPFQGNFTIILYGRADEGIQPDPYYGLKYIGV |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | YIGVGKGGALELHGQKKLSWTFLNKTLHPGGMAEGGYFFERSWGHRGVIVHVIDPKSGTV |
| NOV11i | NGIGLTLASGGTFPYDDGSKQEIKNSLFVGESGNVGTEMMDNRIWGPGGLDHSGRTLPIG |
| NOV11j | YIGVGKGGALELHGQKKLSWTFLNKTLHPGGMAEGGYFFERSWGHRGVIVHVIDPKSGTV |
| NOV11a | QNFPTRGTQLYDGPINIQNCTFRKFVALEGRHTSALAFRLNNAWQSCPHNNVTGIAFEDV |
| NOV11b | GKGGALELHGQKKLSWTFLNKTLHPGGMAEGGYFFERSWGHRGVIVHVIDPKSGTVIHSD |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | IHSDRFDTYRSKKESERLVQYLNAVPDGRILSVAVNDEGSRNLDDMARKAMTKLGSKHFL |
| NOV11i | QNFPIRGIQLYDGPINIQNCTFRKFVALEGRHTSALAFRLNNAWQSCPHNNVTGIAFEDV |
| NOV11j | IHSDRFDTYRSKKESERLVQYLNAVPDGRILSVAVNDEGSRNLDDMARKAMTKLGSKHFL |
| NOV11a | PTTSRVFFGEPGPWFNQLDMDGDKTSVFHDVDGSVSEYPGSYLTKNDNWLVRHPDCINVP |
| NOV11b | RFDTYRSKKESERLVQYLNAVPDGRILSVAVNDEGSRNLDDMARKAMTKLGSKHFLHLGF |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | HLGFRHPWSFLTVKGNPSSSVEDHIEYHGHRGSAAARVFKLFQTEHGEYFNVSLSSEWVQ |
| NOV11i | PITSRVFFGEPGPWFNQLDMDGDKTSVFHDVDGSVSEYPGSYLTKNDNWLVRHPDCINVP |
| NOV11j | HLGFRHPWSFLTVKGNPSSSVEDHIEYHGHRGSAAARVFKLFQTEHGEYFNVSLSSEWVQ |
| NOV11a | DWRGAICSGCYAQMYIQAYKTSNLRMKIIKNDFPSHPLYLEGALTRSTHYQQYQPVVTLQ |
| NOV11b | RVEWTEWFDHDKVSQTKGGEKISDLWKAHPGKICNRPIDIQQATTMDGVNLSTEVVYKKG |
| NOV11c | ------------------------------------------------------------ |
| NOV11d | ------------------------------------------------------------ |
| NOV11e | ------------------------------------------------------------ |
| NOV11f | --------DHDKVSQTKGGEKISDLWKAHPGKICNRPIDIQ-ATTMDGVNLSTEVVYKKG |
| NOV11g | -----------------AYKTSNLRMKIIKNDFPSHPLYLEGALTRSTHYQQYQPVVTLQ |
| NOV11h | DVEWTEWFDHDKVSQTKGGEKISDLWKAHPGKICNRPIDIQ-ATTMDGVNLSTEVVYKKG |
| NOV11i | DWRGAICSGCYAQMYIQAYKTSNLRMKIIKNDFPSHPLYLEGALTRSTHYQQYQPVVTLQ |
| NOV11j | DVEWTEWFDHDKVSQTKGGEKISDLWKAHPGKICNRPIDIQ-ATTMDGVNLSTEVVYKKG |
| NOV11a | KGYTIHWDQTAPAELAIWLINFN-KGDWIRVGLCYPRGTTFSILSDVHNRLLKQTSKTGV |
| NOV11b | QDYRFACYDRGRACRSYRVRFLCGKPVRPKLTVTIDTNVNSTILNLEDNVQSWKPGDTLV |
| NOV11c | ------------------------------------------------------CPDQSP |
| NOV11d | ------------------------------------------------------------ |
| NOV11e | ------------------------------------------------------------ |
| NOV11f | QDYRFACYDRGRACRSYRVRFLCGKPVRPKLTVTIDTNVNSTILNLEDNVQSWKPGDTLV |
| NOV11g | KGYTIHWDQTAPAELAIWLINFN-KGDWIRVGLCYPRGTTFSILSDVHNRLLKQTSKTGV |
| NOV11h | QDYRFACYDRGRACRSYRVRFLCGKPVRPKLTVTIDTNVNSTILNLEDNVQSWKPGDTLV |
| NOV11i | KGYTIHWDQTAPAELAIWLINFN-KGDWIRVGLCYPRGTTFSILSDVHNRLLKQTSKTGV |
| NOV11j | QDYRFACYDRGRACRSYRVRFLCGKPVRPKLTVTIDTNVNSTILNLEDNVQSWKPGDTLV |
| NOV11a | FVRTLQMDKVEQSYPGRSHYYWDEDSGLLFLKLKAQNEREKFAFCSMKGCERIKIKALIP |
| NOV11b | IASTDYSMYQAEEFQVLPCRSCAPNQVKVAGKPMYLHIGEEIDGVDMRAEVGLLSRNIIV |
| NOV11c | ELQPWNPGHDQDHHVHIGQGKTLLLTSSATVYSIHISEGGKLVIKDHDEPIVLRTRHILI |
| NOV11d | ------------------------------------------------------------ |
| NOV11e | -------------HVHIGQGKTLLLTSSATVYSIHISEGGKLVIKDHDEPIVLRTRHILI |
| NOV11f | IASTDYSMYQAEEFQVLPCRSCAPNQVKVAGKPMYLHIGEEIDGVDMRAEVGLLSRNIIV |
| NOV11g | FVRTLQMDKVEQSYPGRSHYYWDEDSGLLFLKLKAQNEREKFAFCSMKGCERIKIKALIP |
| NOV11h | IASTDYSMYQAEEFQVLPCRSCAPNQVKVAGKPMYLHIGEEIDGVDMRAEVGLLSRNIIV |
| NOV11i | FVRTLQMDKVEQSYPGRSHYYWDEDSGLLFLKLKAQNEREKFAFCSMKGCERIKIKALIP |
| NOV11j | IASTDYSMYQAEEFQVLPCRSCAPNQVKVAGKPMYLHIGEEIDGVDMRAEVGLLSRNIIV |
| NOV11a | KNAGVSDCTATAYPKFTERAVVDVPMPKKLFGSQLKTKDHFLEVKME-SSKQHFFHLWND |
| NOV11b | MGEMEDKCYPYRNHICNFFDFDTFGGHIKFALGFKAAHLEGTELKHM-GQQLVGQYPIHF |
| NOV11c | DNGGELHAGSALCPFQGNFTIILYGRADEGIQPDPYYGLKYIGVGKGGALELHGQKKLSW |
| NOV11d | ------------------------------------------------------------ |
| NOV11e | DNGGELHAGSALCPFQGNFTIILYGRADEGIQPDPYYGLKYIGVGKGGALELHGQKKLSW |
| NOV11f | MGEMEDKCYPYRNHICNFFDFDTFGGHIKFALGFKAAHLEGTELKHM-GQQLVGQYPIHF |
| NOV11g | KNAGVSDCTATAYPKFTERAVVDVPMPKKLFGSQLKTKDHFLEVKME-SSKQHFFHLWND |
| NOV11h | MGEMEDKCYPYRNHICNFFDFDTFGGHIKFALGFKAAHLEGTELKHM-GQQLVGQYPIHF |
| NOV11i | KNAGVSDCTATAYPKFTERAVVDVPMPKKLFGSQLKTKDHFLEVKME-SSKQHFFHLWND |
| NOV11j | MGEMEDKCYPYRNHICNFFDFDTFGGHIKFALGFKAAHLEGTELKHM-GQQLVGQYPIHF |
| NOV11a | FAYIEVDGK----------KYPSSEDGIQVVVIDGNQGRVVSHTSFRNSILQGIPWQ--- |
| NOV11b | HLAGDVDERGGYDPPTYIRDLSIHHTFSRCVTVHGSNGLLIKDVVGYNSLGHCFFTEDGP |
| NOV11c | TFLNKTLHPGGMAEGGYFFERSWGHRGVIVHVIDPKSGTVIHSDRFDTYRSKKESER--- |
| NOV11d | ------DGK----------KYPSSEDGIQVVVIDGNQGRVVSHTSFRNSILQGIPWQ--- |
| NOV11e | TFLNKTLHPGGMAEGGYFFERSWGHRGVIVHVIDPKSGTVIHSDRFDTYRSKKESER--- |
| NOV11f | HLAGDVDERGGYDPPTYIRDLSIHHTFSRCVTVHGSNGLLIKDVVGYNSLGHCFFTEDGP |
| NOV11g | FAYIEVDGK----------KYPSSEDGIQVVVIDGNQGRVVSHTSFRNSILQGIPWQ--- |
| NOV11h | HLAGDVDERGGYDPPTYIRDLSIHHTFSRCVTVHGSNGLLIKDVVGYNSLGHCFFTEDGP |
| NOV11i | FAYIEVDGK----------KYPSSEDGIQVVVIDGNQGRVVSHTSFRNSILQGIPWQ--- |
| NOV11j | HLAGDVDERGGYDPPTYIRDLSIHHTFSRCVTVHGSNGLLIKDVVGYNSLGHCFFTEDGP |
| NOV11a | ----LFNYVATIPDNSIVLMASKG-----RYVSRGPWTRVLEKLGADRGLKLKEQMA--- |
| NOV11b | EERNTFDHCLGLLVKSGTLLPSDRDSKMCKMITEDSYPGYIPKPRQDCNAVSTFWMANPN |
| NOV11c | ----LVQYLNAVPDGRILSVA--------------------------------------- |
| NOV11d | ----LFNYVATIPDNSIVLMASKG-----RYVSRGPWTRVLEKLGADRGLKLKEQMA--- |
| NOV11e | ----LVQYLNAVPDGRILSVAVNDEG---SRNLDDMARKAMTKLGSKHFLHLGFRHP--- |
| NOV11f | EERNTFDHCLGLLVKSGTLLPSDRDSKMCKMITEDSYPGYIPKPRQDCNAVSTFWMANPN |
| NOV11g | ----LFNYVATIPDNSIVLMASKG-----RYVSRGPWTRVLEKLGADRGLKLKEQMA--- |
| NOV11h | EERNTFDHCLGLLVKSGTLLPSDRDSKMCKMITEDSYPGYIPKPRQDCNAVSTFWMANPN |
| NOV11i | ----LFNYVATIPDNSIVLMASKG-----RYVSRGPWTRVLEKLGADRGLKLKEQMA--- |
| NOV11j | EERNTFDHCLGLLVKSGTLLPSDRDSKMCKMITEDSYPGYIPKPRQDCNAVSTFWMANPN |
| NOV11a | ------------FVGFKGSFRPIWVTLDTEDHKAKIFQVVPIPVVKKKKL---------- |
| NOV11b | NNLINCAAAGSEETGFWFIFHHVPTGPSVGMYSPGYSEHIPLGKFYNNRAHSNYRAGMII |
| NOV11c | ------------------------------------------------------------ |
| NOV11d | ------------FVGFKGSFRPIWVTLDTEDHKAKIFQVVPIPVV--------------- |
| NOV11e | -------------WSFLTVKGNPSSSVEDHIEYHGHRGSAAARVFKLFQT---------- |
| NOV11f | NNLINCAAAGSEETGFWFIFHHVPTGPSVGMYSPGYSEHIPLGKFYNNRAHSNYRAGMII |
| NOV11g | ------------FVGFKGSFRPIWVTLDTEDHKAKIFQVVPIPVVKKKKL---------- |
| NOV11h | NNLINCAAAGSEETGFWFIFHHVPTGPSVGMYSPGYSEHIPLGKFYNNRAHSNYRAGMII |
| NOV11i | ------------FVGFKGSFRPIWVTLDTEDHKAKIFQVVPIPVVKKKKLLEG------- |
| NOV11j | NNLINCAAAGSEETGFWFIFHHVPTGPSVGMYSPGYSEHIPLGKFYNNRAHSNYRAGMII |
| NOV11a | ------------------------------------------------------------ |
| NOV11b | DNGVKTTEASAKDKRPFLSIISARYSPHQDADPLKPREPAIIRHFIAYKNQDHGAWLRGG |
| NOV11c | ------------------------------------------------------------ |
| NOV11d | ------------------------------------------------------------ |
| NOV11e | ------------------------------------------------------------ |
| NOV11f | DNGVKTTEASAKDKRPFLSI---------------------------------------- |
| NOV11g | ------------------------------------------------------------ |
| NOV11h | DNGVKTTEASAKDKRPFLSIISARYSPHQDADPLKPREPAIIRHFIAYKNQDRGAWLRGG |
| NOV11i | ------------------------------------------------------------ |
| NOV11j | DNGVKTTEASAKDKRPFLSIISARYSPHQDADPLKPREPAIIRHFIAYKNQDHGAWLRGG |
| NOV11a | ------------------------------------------------------------ |
| NOV11b | DVWLDSCRFADNGIGLTLASGGTFPYDDGSKQEIKNSLFVGESGNVGTEMMDNRIWGPGG |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | DVWLDSCRFADNGIGLTLASGGTFPYDDGSKQEIKNSLFVGESGNVGTEMMDNRIWGPGG |
| NOV11i | ------------------------------------------------------------ |
| NOV11j | DVWLDSCRFADNGIGLTLASGGTFPYDDGSKQEIKNSLFVGESGNVGTEMMDNRIWGPGG |
| NOV11a | ------------------------------------------------------------ |
| NOV11b | LDHSGRTLPIGQNFPIRGIQLYDGPINIQNCTFRKFVALEGRHTSALAFRLNNAWQSCPH |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | LDHSGRTLPIGQNFPIRGIQLYDGPINILNCTFRKFVALEGRHTSALAFRLNNAWQSCPH |
| NOV11i | ------------------------------------------------------------ |
| NOV11j | LDHSGRTLPIGQNFPIRGIQLYDGPINIQNCTFRKFVALEGRHTSALAFRLNNAWQSCPH |
| NOV11a | ------------------------------------------------------------ |
| NOV11b | NNVTGIAFEDVPITSRVFFGEPGPWFNQLDMDGDKTSVFHDVDGSVSEYPGSYLTKNDNW |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | NNVTGIAFEDVPITSRVFFGEPGPWFNQLDMDGDKTSVFHDVDGSVSEYPGSYLTKNDNW |
| NOV11i | ------------------------------------------------------------ |
| NOV11j | NNVTGIAFEDVPITSRVFFGEPGPWFNQLDMDGDKTSVFHDVDGSVSEYPGSYLTKNGNW |
| NOV11a | ------------------------------------------------------------ |
| NOV11b | LVRHPDCINVPDWRGAICSGCYAQMYIQAYKTSNLRMKIIKNDFPSHPLYLEGALTRSTH |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | LVRHPDCINVPDWRGAICSGCYAQMYIQAYKTSNLRMKIIKNDFPSHPLYLEGALTRSTH |
| NOV11i | ------------------------------------------------------------ |
| NOV11j | LVRHPDCINVPDWRGAICSGCYAQMYIQAYKTSNLRMKIIKNDFPSHPLYLEGALTRSTH |
| NOV11a | ------------------------------------------------------------ |
| NOV11b | YQQYQPVVTLQKGYTIHWDQTAPAELAIWLINFNKGDWIRVGLCYPRGTTFSILSDVHNR |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | YQQYQPVVTLQKGYTIHWDQTAPAELAIWLINFNKGDWIRVGLCYPRGTTFSILSDVHNR |
| NOV11i | ------------------------------------------------------------ |
| NOV11j | YQQYQPVVTLQKGYTIHWDQTAPAELAIWLINFNKGDWIRVGLCYPRGTTFSILSDVHNR |
| NOV11a | ------------------------------------------------------------ |
| NOV11b | LLKQTSKTGVFVRTLQMDKVEQSYPGRSHYYWDEDSGLLFLKLKAQNEREKFAFCSMKGC |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | LLKQTSKTGVFVRTLQMDKVEQSYPGRSHYYWDEDSGLLFLKLKAQNEREKFAFCSMKGC |
| NOV11i | ------------------------------------------------------------ |
| NOV11j | LLKQTSKTGVFVRTLQMDKVEQSYPGRSHYYWDEDSGLLFLKLKAQNEREKFAFCSMKGC |
| NOV11a | ------------------------------------------------------------ |
| NOV11b | ERIKIKALIPKNAGVSDCTATAYPKFTERAVVDVPMPKKLFGSQLKTKDHFLEVKMESSK |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | ERIKIKALIPKNAGVSDCTATAYPKFTERAVVDVPMPKKLFGSQLKTKDHFLEVKMESSK |
| NOV11i | ------------------------------------------------------------ |
| NOV11j | ERIKIKALIPKNAGVSDCTATAYPKFTERAVVDVPMPKKLFGSQLKTKDNFLEVKMESSK |
| NOV11a | ------------------------------------------------------------ |
| NOV11b | QHFFHLWNDFAYIEVDGKKYPSSEDGIQVVVIDGNQGRVVSHTSFRNSILQGIPWQLFNY |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | QHFFHLWNDFAYIEVDGKKYPSSEDGIQVVVIDGNQGRVVSHTSFRNSILQGIPWQLFNY |
| NOV11i | ------------------------------------------------------------ |
| NOV11j | QHFFHLWNDFAYIEVDGKKYPSSEDGIQVVVIDGNQGRVVSHTSFRNSILQGIPWQLFNY |
| NOV11a | ------------------------------------------------------------ |
| NOV11b | VATIPDNSIVLMASKGRYVSRGPWTRVLEKLGADRGLKLKEQMAFVGFKGSFRPIWVTLD |
| NOV11c | ------------------------------------------------------------ |
| NOV11d | ------------------------------------------------------------ |
| NOV11e | ------------------------------------------------------------ |
| NOV11f | ------------------------------------------------------------ |
| NOV11g | ------------------------------------------------------------ |
| NOV11h | VATIPDNSIVLMASKGRYVSRGPWTRVLEKLGADRGLKLKEQMAFVGFKGSFRPIWVTLD |
| NOV11i | ------------------------------------------------------------ |
| NOV11j | VATIPDNSIVLMASKGRYVSRGPWTRVLEKLGADRGLKLKEQMAFVGFKGSFRPIWVTLD |
| NOV11a | ------------------------- |
| NOV11b | TEDHKAKIFQVVPIPVVKKKKL--- |
| NOV11c | ------------------------- |
| NOV11d | ------------------------- |
| NOV11e | ------------------------- |
| NOV11f | ------------------------- |
| NOV11g | ------------------------- |
| NOV11h | TEDHKAKIFQVVPIPVVKKKKL--- |
| NOV11i | ------------------------- |
| NOV11j | TEDHKAKIFQVVPIPVVKKKKLLEG |
| NOV11a | (SEQ ID NO: 118) |
| NOV11b | (SEQ ID NO: 120) |
| NOV11c | (SEQ ID NO: 122) |
| NOV11d | (SEQ ID NO: 124) |
| NOV11e | (SEQ ID NO: 126) |
| NOV11f | (SEQ ID NO: 128) |
| NOV11g | (SEQ ID NO: 130) |
| NOV11h | (SEQ ID NO: 132) |
| NOV11i | (SEQ ID NO: 134) |
| NOV11j | (SEQ ID NO: 136) |

Further analysis of the NOV11j protein yielded the following properties shown in Table 11C.

TABLE 11C
Protein Sequence Properties NOV11j

| | |
|---|---|
| SignalP | No Known Signal Sequence Predicted |
| analysis: | |
| PSORT II | Psort Results (see Details): |
| analysis: | 74.5%: microbody (peroxisome) |
| | 30.0%: nucleus |
| | 17.2%: lysosome (lumen) |
| | 10.0%: mitochondrial matrix space |
| | Details of Psort Prediction |
| | >>> MUS belongs to the animal class |
| | *** Reasoning Step: 2 |
| | SRCFLG: 1 |
| | Prelim. Calc. of ALOM (thresh: 0.5) count: 0 |
| | McG: Length of UR: 7 |
| | Peak Value of UR: −1.04 |
| | Net Charge of CR: −1 |
| | McG: Discrim Score: −23.99 |
| | GvH: Signal Score (−3.5): 1.65 |
| | Possible site: 39 |
| | >>> Seems to have no N-terminal signal seq. |
| | Amino Acid Composition: calculated from 1 |
| | new cnt: 0 ** thrshld changed to −2 |
| | involving clv. sig in the ALOMREC or not: 0B |
| | ALOM program count: 0 value: 4.51 threshold: −2.0 |
| | PERIPHERAL Likelihood = 4.51 |
| | modified ALOM score: −1.80 |
| | Gavel: Bound. Mitoch. Preseq. R-2 motif: 4 TRSCPD |
| | mtdisc (mit) Status: negative (−8.24) |
| | *** Reasoning Step: 3 |
| | KDEL Count: 0 |
| | Goal mtmx modified Score: 0.10 |
| | SKL motif: pos: 505(1332), count: 1 AHL |
| | pox modified by SKL scr: 0.3 |
| | Poxaac Score: 4.27 |
| | >>> POX Status: positive |
| | pox modified by aac scr: 0.636 |
| | >>> lys: 0.22 Status: notclr |
| | Goal lys: modified. Score: 0.172 |
| | Nuc-4 pos: 1324 (5) KKKK |
| | nuc modified. Score: 0.60 |
| | >>> Nuclear Signal. Status: notclr (0.30) |

A search of the NOV11j protein against the Geneseq database, a proprietary database that contains sequences published in patents and patent publication, yielded several homologous proteins shown in Table 11D.

TABLE 11D
Geneseq Results for NOV11j

| | | NOV11j | | |
|---|---|---|---|---|
| | | Residues/ | Identities/ | |
| Geneseq | Protein/Organism/Length [Patent #, | Match | Similarities for the | Expect |
| Identifier | Date] | Residues | Matched Region | Value |
| ABR58552 | Human cancer related protein SEQ ID | 1 . . . 1326 | 1322/1323 (99%) | 0.0 |
| | NO: 209 - Homo sapiens, 1361 aa. | 33 . . . 1358 | 1322/1323 (99%) | |
| | [WO2003025138-A2, 27 MAR. 2003] | | | |
| ABU52404 | Human GPCR related protein NOV42b - | 1 . . . 1326 | 1322/1323 (99%) | 0.0 |
| | Homo sapiens, 1361 aa. | 33 . . . 1358 | 1322/1323 (99%) | |
| | [WO200279398-A2, 10 OCT. 2002] | | | |
| ABP54684 | Metastatic colorectal cancer-associated | 1 . . . 1326 | 1322/1323 (99%) | 0.0 |
| | polypeptide - Homo sapiens, 1361 aa. | 33 . . . 1358 | 1322/1323 (99%) | |
| | [WO200268677-A2, 06 SEP. 2002] | | | |

In a BLAST search of public sequence databases, the NOV11j protein was found to have homology to the proteins shown in the BLASTP data in Table 11E.

TABLE 11E
Public BLASTP Results for NOV11j

| | | NOV11j | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8BI06y | Hypothetical 110.4 kDa protein | 1..1079 | 998/1075 (92%) | 0.0 |
| | homolog-Mus musculus (Mouse), | 53...1130 | 1039/1075 (96%) | |
| | 1142 aa. | | | |
| Q9ULM1 | Hypothetical protein KIAA1199- | 314..1326 | 1009/1010 (99%) | |
| | Homo sapiens (Human), 1013 aa | 1..1010 | 1009/1010 (99%) | |
| | (fragment). | | | |
| Q8WUL3 | Hypothetical protein-Homo | 1..944 | 939/941 (99%) | |
| | sapiens (Human), 992 aa. | 33.976 | 939/941 (99%) | |

### Example 12

### NOV12, CG88912, Beta-neoendorphin-dynorphin Precursor

The NOV12 clone was analyzed, and the nucleotide and encoded polypeptide sequences are shown in Table 12A.

TABLE 12A
NOV12 Sequence Analysis

| | | |
|---|---|---|
| NOV12a, CG88912-02 | SEQ ID NO: 137 | 619 bp |
| DNA Sequence | ORF Start: at 1 | ORF Stop: TAA at 604 |

GCTGCCTGCCTCCTCATGTTCCCCTCCACCACAGCGGACTGCCTGTCGCGGTGCTCCTTGTGTGCTGTAAAGA
CCCAGGATGGTCCCAAACCTATCAATCCCCTGATTTGCTCCCTGCAATGCCAGGCTGCCCTGCTGCCCTCTGA
GGAATGGGAGAGATGCCAGAGCTTTCTGTCTTTTTTCACCCCCTCCACCCTTGGGCTCAATGACAAGGAGGAC
TTGGGGAGCAAGTCGGTTGGGGAAGGGCCCTACAGTGAGCTGGCCAAGCTCTCTGGGTCATTCCTGAAGGAGC
TGAACGATGGTGCCATGGAGACTGGCACACTCTATCTCGCTGAGGAGGACCCCAAGGAGCAGGTCAAACGCTA
TGGGGGCTTTTTGCGCAAATACCCCAAGAGGAGCTCAGAGGTGGCTGGGGAGGGGGACGGGGATAGCATGGGC
CATGAGGACCTGTACAAACGCTATGGGGGCTTCTTGCGGCGCATTCGTCCCAAGCTCAAGTGGGACAACCAGA
AGCGCTATGGCGGTTTTCTCCGGCGCCAGTTCAAGGTGGTGACTCGGTCTCAGGAAGATCCGAATGCTTACTC
TGGAGAGCTTTTTGATGCATAAGCACTTCTTTTCA

| | | | |
|---|---|---|---|
| NOV12a, CG88912-02 | | | |
| Protein Sequence | SEQ ID NO: 138 | 201 aa | MW at 22447.1kD |

AACLLMFPSTTADCLSRCSLCAVKTQDGPKPINPLICSLQCQAALLPSEEWERCQSFLSFFTPSTLGLNDKED
LGSKSVGEGPYSELAKLSGSFLKELNDGAMETGTLYLAEEDPKEQVKRYGGFLRKYPKRSSEVAGEGDGDSMG
HEDLYKRYGGFLRRIRPKLKWDNQKRYGGFLRRQFKVVTRSQEDPNAYSGELFDA

| | | |
|---|---|---|
| NOV12b, CG88912-01 | SEQ ID NO: 139 | 758 bp |
| DNA Sequence | ORF Start: ATG at 16 | ORF Stop: TGA at 379 |

TCTGCCTGCCTCCTCATGTTCCCCTCCACCACAGCGGACTGCCTGTCGCGGTGCTCCTTGTGTGCTGTAAAGA
CCCAGGATGGTCCCAAACCTATCAATCCCCTGATTTGCTCCCTGCAATGCCAGGCTGCCCTGCTGCCCTCTGA
GGAATGGGAGAGATGCCAGAGCTTTCTGTCTTTTTTCACCCCCTCCACCCTTGGGCTCAATGACAAGGAGGAC
TTGGGGAGCAAGTCGGTTGGGGAAGGGCCCTACAGTGAGCTGGCCAAGCTCTCTGGGTCATTCCTGAAGGAGC
TGGAGAAAAGCAAGTTTTCTCCCAAGTATCTCAACAAAGGAGAACACTCTGAGCAAGAGCCTGGAGGAGAAGC
TCAGGGGTCTCTCTGACGGGTTTAGGGAGGGAGCAGAGTCTGAGCTGATGAGGGATGCCCAGCTGAACGATGG
TGCCATGGAGACTGGCACACTCTATCTCGCTGAGGAGGACCCCAAGGAGCAGGTCAAACGCTATGGGGGCTTT
TTGCGCAAATACCCCAAGAGGAGCTCAGAGGTGGCTGGGGAGGGGGACGGGGATAGCATGGGCCATGAGGACC
TGTACAAACGCTATGGGGGCTTCTTGCGGCGCATTCGTCCCAAGCTCAAGTGGGACAACCAGAAGCGCTATGG
CGGTTTTCTCCGGCGCCAGTTCAAGGTGGTGACTCGGTCTCAGGAAGATCCGAATGCTTACTCTGGAGAGCTT
TTTGATGCATAAGCACCTCTTTTCATGA

| | | | |
|---|---|---|---|
| NOV12b, CG88912-01 | | | |
| Protein Sequence | SEQ ID NO: 140 | 121 aa | MW at 13107.6kD |

MFPSTTADCLSRCSLCAVKTQDGPKPINPLICSLQCQAALLPSEEWERCQSFLSFFTPSTLGLNDKEDLGSKS
VGEGPYSELAKLSGSFLKELEKSKFSPKYLNKGEHSEQEPGGEAQGSL

| | | |
|---|---|---|
| NOV12c, 310907706 | SEQ ID NO: 141 | 603 bp |
| DNA Sequence | ORF Start: at 1 | ORF Stop: end of sequence |

GCTGCCTGCCTCCTCATGTTCCCCTCCACCACAGCGGACTGCCTGTCGCGGTGCTCCTTGTGTGCTGTAAAGA
CCCAGGATGGTCCCAAACCTATCAATCCCCTGATTTGCTCCCTGCAATGCCAGGCTGCCCTGCTGCCCTCTGA
GGAATGGGAGAGATGCCAGAGCTTTCTGTCTTTTTTCACCCCCTCCACCCTTGGGCTCAATGACAAGGAGGAC
TTGGGGAGCAAGTCGGTTGGGGAAGGGCCCTACAGTGAGCTGGCCAAGCTCTCTGGGTCATTCCTGAAGGAGC
TGAACGATGGTGCCATGGAGACTGGCACACTCTATCTCGCTGAGGAGGACCCCAAGGAGCAGGTCAAACGCTA
TGGGGGCTTTTTGCGCAAATACCCCAAGAGGAGCTCAGAGGTGGCTGGGGAGGGGGACGGGGATAGCATGGGC
CATGAGGACCTGTACAAACGCTATGGGGGCTTCTTGCGGCGCATTCGTCCCAAGCTCAAGTGGGACAACCAGA
AGCGCTATGGCGGTTTTCTCCGGCGCCAGTTCAAGGTGGTGACTCGGTCTCAGGAAGATCCGAATGCTTACTC
TGGAGAGCTTTTTGATGCA

| | | | |
|---|---|---|---|
| NOV12c, 310907706 | | | |
| Protein Sequence | SEQ ID NO: 142 | 201 aa | MW at 22447.4kD |

AACLLMFPSTTADCLSRCSLCAVKTQDGPKPINPLICSLQCQAALLPSEEWERCQSFLSFFTPSTLGLNDKED
LGSKSVGEGPYSELAKLSGSFLKELNDGAMETGTLYLAEEDPKEQVKRYGGFLRKYPKRSSEVAGEGDGDSMG
HEDLYKRYGGFLRRIRPKLKWDNQKRYGGFLRRQFKVVTRSQEDPNAYSGELFDA

A ClustalW comparison of the above protein sequences yields the following sequence alignment shown in Table 12B.

TABLE 12B
Comparison of the NOV12 protein sequences.

| | |
|---|---|
| NOV12a | ----AACLLMFPSTTADCLSRCSLCAVKTQDGPKPINPLICSLQCQAALLPSEEWERCQS |
| NOV12b | ---------MFPSTTADCLSRCSLCAVKTQDGPKPINPLICSLQCQAALLPSEEWERCQS |
| NOV12c | ----AACLLMFPSTTADCLSRCSLCAVKTQDGPKPINPLICSLQCQAALLPSEEWERCQS |
| NOV12a | FLSFFTPSTLGLNDKEDLGSKSVGEGPYSELAKLSGSFLKELNDGAMETGTLYLAEEDPK |
| NOV12b | FLSFFTPSTLGLNDKEDLGSKSVGEGPYSELAKLSGSFLKELEKSKFSPKYLNKGEHSEQ |
| NOV12c | FLSFFTPSTLGLNDKEDLGSKSVGEGPYSELAKLSGSFLKELNDGAMETGTLYLAEEDPK |
| NOV12a | EQVKRYGGFLRKYPKRSSEVAGEGDGDSMGHEDLYKRYGGFLRRIRPKLKWDNQKRYGGF |
| NOV12b | EPGGEAQGSL-------------------------------------------------- |
| NOV12c | EQVKRYGGFLRKYPKRSSEVAGEGDGDSMGHEDLYKRYGGFLRRIRPKLKWDNQKRYGGF |
| NOV12a | LRRQFKVVTRSQEDPNAYSGELFDA--- |
| NOV12b | ---------------------------- |
| NOV12c | LRRQFKVVTRSQEDPNAYSGELFDA--- |
| NOV12a | (SEQ ID NO: 138) |
| NOV12b | (SEQ ID NO: 140) |
| NOV12c | (SEQ ID NO: 142) |

Further analysis of the NOV12c protein yielded the following properties shown in Table 12C.

TABLE 12C
Protein Sequence Properties NOV12c

| SignalP | Cleavage site between residues 16 and 17 | | | |
|---|---|---|---|---|
| analysis: | | | | |
| PSORT II | PSG: a new signal peptide prediction method | | | |
| analysis: | N-region: length 0; pos. chg 0; neg. chg 0 | | | |
| | H-region: length 16; peak value 9.99 | | | |
| | PSG score: 5.59 | | | |
| | GvH: von Heijne's method for signal seq. recognition | | | |
| | GvH score (threshold: −2.1): −2.34 | | | |
| | possible cleavage site: between 16 and 17 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 4.88 (at 3) | | | |
| | ALOM score: 4.88 (number of TMSs: 0) | | | |
| | MTOP: Prediction of membrane topology (Hartmann et al.) | | | |
| | Center position for calculation: 6 | | | |
| | Charge difference: −1.0 C(0.0)-N(1.0) | | | |
| | N >= C: N-terminal side will be inside | | | |
| | MITDISC: discrimination of mitochondrial targeting seq | | | |
| | R content: | 0 | Hyd Moment(75): | 1.15 |
| | Hyd Moment(95): | 1.14 | G content: | 1 |
| | D/E content: | 1 | S/T content: | 6 |

| | |
|---|---|
| Score: | −4.93 |
| Gavel: prediction of cleavage sites for mitochondrial preseq | |
| R-2 motif at 31 SRC\|SL | |
| NUCDISC: discrimination of nuclear localization signals | |
| pat4: none | |
| pat7: none | |
| bipartite: none | |
| content of basic residues: 13.5% | |
| NLS Score: −0.47 | |
| NNCN: Reinhardt's method for Cytplasmic/Nuclear discrimination | |
| Prediction: nuclear | |
| Reliability: 76.7 | |
| Psort Results (see Details): | |
| 37.0%: outside | |
| 13.2%: microbody (peroxisome) | |
| 10.0%: endoplasmic reticulum (membrane) | |
| 10.0%: endoplasmic reticulum (lumen) | |
| Psort II Results (see Details): | |
| 44.4%: extracellular, including cell wall | |
| 33.3%: mitochondrial | |
| 22.2%: nuclear | |

A search of the NOV12c protein against the Geneseq database, a proprietary database that contains sequences published in patents and patent publication, yielded several homologous proteins shown in Table 12D.

TABLE 12D
Geneseq Results for NOV12c

| | | NOV12c | Identities/ | |
|---|---|---|---|---|
| | | Residues/ | Similarities for | |
| Geneseq | Protein/Organism/Length [Patent #, | Match | the Matched | Expect |
| Identifier | Date] | Residues | Region | Value |
| ABU99162 | Novel human GPCR related protein | 1 . . . 201 | 201/201 (100%) | 1.3e−107 |
| | NOV19a - Homo sapiens, 201 aa. | 1 . . . 201 | 201/201 (100%) | |
| | [WO200299116-A2, 12 DEC. 2002] | | | |
| AAM79544 | Human protein SEQ ID NO 3190 - | 1 . . . 201 | 119/153 (77%) | 1.2e−54 |
| | Homo sapiens, 256 aa. | 11 . . . 256 | 128/153 (83%) | |
| | [WO200157190-A2, 09 AUG. 2001] | | | |
| AAM78560 | Human protein SEQ ID NO 1222 - | 1 . . . 201 | 119/153 (77%) | 1.2e−54 |
| | Homo sapiens, 254 aa. | 9 . . . 254 | 128/153 (83%) | |
| | [WO200157190-A2, 09 AUG. 2001] | | | |

In a BLAST search of public sequence databases, the NOV12c protein was found to have homology to the proteins shown in the BLASTP data in Table 12E.

TABLE 12E
Public BLASTP Results for NOV12c

| | | NOV12c | Identities/ | |
|---|---|---|---|---|
| Protein | | Residues/ | Similarities for | |
| Accession | | Match | the Matched | Expect |
| Number | Protein/Organism/Length | Residues | Portion | Value |
| P01213 | Beta-neoendorphin-dynorphin precursor | 1 . . . 201 | 119/153 (77%) | 1.3e−54 |
| | (Proenkephalin B) (Preprodynorphin) | 9 . . . 254 | 128/153 (83%) | |
| | [Contains: Beta-neoendorphin; Dynorphin; | | | |
| | Leu- Enkephalin; Rimorphin; Leumorphin] - | | | |
| | Homo sapiens (Human), 254 aa. | | | |
| P01214 | Beta-neoendorphin-dynorphin precursor | 1 . . . 200 | 91/104 (87%) | 1.9e−44 |
| | (Proenkephalin B) (Preprodynorphin) | 9 . . . 255 | 93/104 (89%) | |
| | [Contains: Beta-neoendorphin; Dynorphin; | | | |
| | Leu- Enkephalin; Rimorphin; Leumorphin] - | | | |
| | Sus scrofa (Pig), 256 aa. | | | |
| Q95104 | Beta-neoendorphin-dynorphin precursor | 1 . . . 200 | 94/125 (75%) | 5.2e−42 |
| | (Proenkephalin B) (Preprodynorphin) | 9 . . . 257 | 101/125 (80%) | |
| | [Contains: Beta-neoendorphin; Dynorphin; | | | |
| | Leu- Enkephalin; Rimorphin; Leumorphin] - | | | |
| | Bos taurus (Bovine), 258 aa. | | | |

PFam analysis predicts that the NOV12c protein contains the domains shown in the Table 12F. Specific amino acid residues of NOV12c for each domain is shown in column 2, equivalent domains in the other NOV12 proteins of the invention are also encompassed herein.

TABLE 12F
Domain Analysis of NOV12c

| | NOV12c Match Region | | |
|---|---|---|---|
| Pfam Domain | Amino acid residues: | Score | Expect Value |
| Opiods_neuropep | 1 . . . 205 | 399.8 | 2.7e−116 |

### Example B

### Sequencing Methodology and Identification of NOVX Clones

1. GeneCalling™ Technology: A method of differential gene expression profiling between two or more samples (Nature Biotechnology 17:198-803 1999) was used to identify NOVX genes. Briefly cDNA was derived from various human samples of whole tissue, primary cells or tissue cultured primary cells or cell lines representing multiple tissue types, normal and diseased states, physiological states, and developmental states from different donors. Samples were obtained as. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.
2. SeqCalling™ Technology: The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.
3. PathCalling™ Technology: The NOVX nucleic acid sequences are derived by laboratory screening of cDNA library by the two-hybrid approach by methods previously described (Nature 403: 623-627, 2000; U.S. Pat. Nos. 6,057,101 and 6,083,693).
4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.
5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.
6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 pi or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 590 C primer optimal Tm; 20 C maximum primer difference (if probe does not have 5′ G, probe Tm must be 100 C greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 950 C for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1×TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_screening_panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDR1), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1 D included 2 control wells and 94 test samples of RNA (Panel 4R) or cDNA (Panels 4D and 4.1 D) from human cell lines or tissues related to inflammatory conditions. Controls included total RNA from normal tissues such as colon, lung (Stratagene, La. Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDR1, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta˜1-5 ng/ml, TNF alpha ˜5-10 ng/ml, IFN gamma ˜20-50 ng/ml, IL-4˜5-10 ng/ml, IL-9˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of −2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+ lymphocytes were isolated by depleting mononuclear cells of CD8+, CD56+, CD14+ and CD19+ cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+ RO CD4+ and CD8+ lymphocytes were cultured in culture media at 10⁶ cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10 cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+lymphocytes (Poietic Systems, German Town, Md.) were cultured at 10⁵-10⁶cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NC1-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −200 C overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μl RNAsin and 8 μl DNAse and incubated at 370 C for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

Al_Comprehensive Panel_v1.0

Autoimmunity (Al) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N.J.), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta˜1-10 ng/ml, TNF alpha˜5-50 ng/ml, or prostaglandin E2 for Nhost cells) for 1, 6, 18 or 24 h. All these cells were starved for at least 5 h and cultured in their corresponding basal medium with ˜0.1 to 1% FBS.

Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDRI (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

Al.05 Chondrosarcoma

Al.05 chondrosarcoma plates included SW1353 cells (ATCC) subjected to serum starvation and treated for 6 and 18 h with cytokines that are known to induce MMP (1, 3 and 13) synthesis (e.g. IL1 beta). These treatments included: IL-1 beta (10 ng/ml), IL-1 beta+TNF-alpha (50 ng/ml), IL-1 beta+Oncostatin (50 ng/ml) and PMA (100 ng/ml). Supernatants were collected and analyzed for MMP 1, 3 and 13 production. RNA was prepared from these samples using standard procedures.

Panels 5D and 51

Panel 5D and 51 included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr 2 1999:143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5 l also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (PI), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −800 C in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. NOV1, CG101729-O₂: FGFR4 Variant.

Expression of gene CG101729-02 was assessed using the primer-probe sets Ag4038, Ag4044 and Ag7932, described in Tables M, AB and AC. Results of the RTQ-PCR runs are shown in Tables AD, AE, AF and AG. CG101729-02 represents a full-length physical clone.

TABLE AA
Probe Name Ag4038

| | | | Start | SEQ |
|---|---|---|---|---|
| Primers | Sequences | Length | Position | ID No |
| Forward | 5′-ctgaagcacatcgtcatc | 21 | 866 | 143 |
| | aac-3′ | | | |
| Probe | TET-5′-cggtttcccctatg | 26 | 907 | 144 |
| | tgcaagtcctaa-3′-TAMRA | | | |
| Reverse | 5′-ctccacctctgagctatt | 22 | 943 | 145 |
| | gatg-3′ | | | |

TABLE AB
Probe Name Ag4044

| | | | Start | SEQ |
|---|---|---|---|---|
| Primers | Sequences | Length | Position | ID No |
| Forward | 5′-cgtcaagatgctcaaaga | 22 | 1480 | 146 |
| | caac-3′ | | | |
| Probe | TET-5′-ctctgacaaggacc | 24 | 1504 | 147 |
| | tggccgacct-3′-TAMRA | | | |
| Reverse | 5′-gatcagcttcatcacctc | 21 | 1538 | 148 |
| | cat-3′ | | | |

TABLE AC
Probe Name Ag7932

| | | | Start | SEQ |
|---|---|---|---|---|
| Primers | Sequences | Length | Position | ID No |
| Forward | 5′-cgtgcgtctctcctcca- | 17 | 1332 | 149 |
| | 3′ | | | |
| Probe | TET-5′-cttcccaagcacca | 21 | 1370 | 150 |
| | gcgaggc-3′-TAMRA | | | |
| Reverse | 5′-cacgtactacctggccaa | 20 | 1408 | 151 |
| | ag-3′ | | | |

TABLE AD
AI comprehensive panel v1.0

| Tissue Name | A | B |
|---|---|---|
| 110967 COPD-F | 2.6 | 0.5 |
| 110980 COPD-F | 0.5 | 2.8 |
| 110968 COPD-M | 2.2 | 0.3 |
| 110977 COPD-M | 11.3 | 7.5 |
| 110989 Emphysema-F | 5.1 | 3.7 |
| 110992 Emphysema-F | 11.4 | 2.3 |
| 110993 Emphysema-F | 0.9 | 1.2 |
| 110994 Emphysema-F | 0.0 | 0.9 |
| 110995 Emphysema-F | 19.1 | 8.3 |
| 110996 Emphysema-F | 3.7 | 2.9 |
| 110997 Asthma-M | 1.3 | 0.0 |
| 111001 Asthma-F | 2.7 | 2.1 |
| 111002 Asthma-F | 6.9 | 2.3 |
| 111003 Atopic Asthma-F | 10.9 | 5.1 |
| 111004 Atopic Asthma-F | 23.8 | 19.3 |
| 111005 Atopic Asthma-F | 15.9 | 13.3 |
| 111006 Atopic Asthma-F | 1.9 | 1.1 |
| 111417 Allergy-M | 5.7 | 2.2 |
| 112347 Allergy-M | 0.0 | 0.3 |
| 112349 Normal Lung-F | 0.0 | 0.1 |
| 112357 Normal Lung-F | 62.4 | 50.0 |
| 112354 Normal Lung-M | 23.7 | 24.5 |
| 112374 Crohns-F | 0.8 | 0.0 |
| 112389 Match Control Crohns-F | 2.5 | 2.1 |
| 112375 Crohns-F | 0.0 | 0.3 |
| 112732 Match Control Crohns-F | 1.7 | 0.9 |
| 112725 Crohns-M | 0.0 | 0.0 |
| 112387 Match Control Crohns-M | 2.0 | 0.9 |
| 112378 Crohns-M | 0.0 | 0.0 |
| 112390 Match Control Crohns-M | 8.7 | 6.2 |
| 112726 Crohns-M | 14.9 | 13.2 |
| 112731 Match Control Crohns-M | 4.4 | 10.2 |
| 112380 Ulcer Col-F | 5.4 | 8.8 |
| 112734 Match Control Ulcer Col-F | 4.3 | 4.0 |
| 112384 Ulcer Col-F | 2.3 | 2.4 |
| 112737 Match Control Ulcer Col-F | 6.1 | 4.9 |
| 112386 Ulcer Col-F | 0.0 | 0.0 |
| 112738 Match Control Ulcer Col-F | 40.6 | 28.3 |
| 112381 Ulcer Col-M | 0.0 | 0.0 |
| 112735 Match Control Ulcer Col-M | 0.0 | 0.0 |
| 112382 Ulcer Col-M | 4.5 | 4.5 |
| 112394 Match Control Ulcer Col-M | 0.0 | 0.0 |
| 112383 Ulcer Col-M | 6.9 | 3.4 |
| 112736 Match Control Ulcer Col-M | 1.2 | 0.6 |
| 112423 Psoriasis-F | 4.4 | 1.5 |
| 112427 Match Control Psoriasis-F | 6.1 | 6.3 |
| 112418 Psoriasis-M | 0.8 | 0.3 |
| 112723 Match Control Psoriasis-M | 54.7 | 51.1 |
| 112419 Psoriasis-M | 1.5 | 1.2 |
| 112424 Match Control Psoriasis-M | 2.0 | 0.8 |
| 112420 Psoriasis-M | 6.4 | 6.9 |
| 112425 Match Control Psoriasis-M | 9.9 | 4.5 |
| 104689 (MF) OA Bone-Backus | 0.0 | 0.0 |
| 104690 (MF) Adj “Normal” Bone-Backus | 2.2 | 0.0 |
| 104691 (MF) OA Synovium-Backus | 1.7 | 0.3 |
| 104692 (BA) OA Cartilage-Backus | 23.3 | 11.3 |
| 104694 (BA) OA Bone-Backus | 0.0 | 0.0 |
| 104695 (BA) Adj “Normal” Bone-Backus | 3.1 | 0.6 |
| 104696 (BA) OA Synovium-Backus | 0.0 | 0.4 |
| 104700 (SS) OA Bone-Backus | 1.0 | 0.4 |
| 104701 (SS) Adj “Normal” Bone-Backus | 0.0 | 0.3 |
| 104702 (SS) OA Synovium-Backus | 0.9 | 0.3 |
| 117093 OA Cartilage Rep7 | 1.6 | 1.9 |
| 112672 OA Bone5 | 0.0 | 1.6 |
| 112673 OA Synovium5 | 0.0 | 1.0 |
| 112674 OA Synovial Fluid cells5 | 1.8 | 0.0 |
| 117100 OA Cartilage Rep14 | 1.7 | 2.0 |
| 112756 OA Bone9 | 2.6 | 0.0 |
| 112757 OA Synovium9 | 17.7 | 11.3 |
| 112758 OA Synovial Fluid Cells9 | 1.3 | 0.3 |
| 117125 RA Cartilage Rep2 | 3.2 | 1.2 |
| 113492 Bone2 RA | 68.8 | 84.7 |
| 113493 Synovium2 RA | 22.5 | 25.2 |
| 113494 Syn Fluid Cells RA | 47.6 | 50.7 |
| 113499 Cartilage4 RA | 48.6 | 74.2 |
| 113500 Bone4 RA | 54.0 | 89.5 |
| 113501 Synovium4 RA | 30.8 | 59.9 |
| 113502 Syn Fluid Cells4 RA | 20.4 | 34.6 |
| 113495 Cartilage3 RA | 54.7 | 63.3 |
| 113496 Bone3 RA | 77.4 | 68.8 |
| 113497 Synovium3 RA | 43.2 | 36.3 |
| 113498 Syn Fluid Cells3 RA | 100.0 | 100.0 |
| 117106 Normal Cartilage Rep20 | 0.9 | 2.2 |
| 113663 Bone3 Normal | 0.0 | 0.3 |
| 113664 Synovium3 Normal | 0.0 | 0.0 |
| 113665 Syn Fluid Cells3 Normal | 0.0 | 0.0 |
| 117107 Normal Cartilage Rep22 | 1.4 | 0.1 |
| 113667 Bone4 Normal | 0.0 | 1.8 |
| 113668 Synovium4 Normal | 1.4 | 0.5 |
| 113669 Syn Fluid Cells4 Normal | 4.5 | 2.9 |

Column A - Rel. Ex. (%) Ag4038, Run 257315330
Column B - Rel. Exp. (%) Ag4044, Run 257315364

TABLE AE
General screening panel v1.7

| Tissue Name | A |
|---|---|
| Adipose | 0.8 |
| HUVEC | 1.7 |
| Melanoma* Hs688(A).T | 0.0 |
| Melanoma* Hs688(B).T | 2.8 |
| Melanoma (met) SK-MEL-5 | 0.8 |
| Testis | 1.2 |
| Prostate ca. (bone met) PC-3 | 0.0 |
| Prostate ca. DU145 | 19.9 |
| Prostate pool | 0.8 |
| Uterus pool | 1.1 |
| Ovarian ca. OVCAR-3 | 13.5 |
| Ovarian ca. (ascites) SK-OV-3 | 2.9 |
| Ovarian ca. OVCAR-4 | 15.5 |
| Ovarian ca. OVCAR-5 | 19.1 |
| Ovarian ca. IGROV-1 | 88.9 |
| Ovarian ca. OVCAR-8 | 61.6 |
| Ovary | 4.5 |
| Breast ca. MCF-7 | |
| Breast ca. MDA-MB-231 | 0.6 |
| Breast ca. BT 549 | 1.6 |
| Breast ca. T47D | 17.4 |
| 113452 mammary gland | 0.9 |
| Trachea | 1.4 |
| Lung | 32.3 |
| Fetal Lung | 68.3 |
| Lung ca. NCI-N417 | 0.0 |
| Lung ca. LX-1 | 41.5 |
| Lung ca. NCI-H146 | 0.1 |
| Lung ca. SHP-77 | 0.3 |
| Lung ca. NCI-H23 | 33.7 |
| Lung ca. NCI-H460 | 0.1 |
| Lung ca. HOP-62 | 1.3 |
| Lung ca. NCI-H522 | 0.9 |
| Lung ca. DMS-114 | 1.4 |
| Liver | 28.7 |
| Fetal Liver | 31.2 |
| Kidney pool | 34.4 |
| Fetal Kidney | 2.3 |
| Renal ca. 786-0 | 13.6 |
| Renal ca. A498 | 20.4 |
| Renal ca. ACHN | 23.0 |
| Renal ca. UO-31 | 0.7 |
| Renal ca. TK-10 | 29.3 |
| Bladder | 1.6 |
| Gastric ca. (liver met.) NCI-N87 | 1.8 |
| Stomach | 0.0 |
| Colon ca. SW-948 | 19.6 |
| Colon ca. SW480 | 0.3 |
| Colon ca. (SW480 met) SW620 | 100.0 |
| Colon ca. HT29 | 9.2 |
| Colon ca. HCT-116 | 73.7 |
| Colon cancer tissue | 0.3 |
| Colon ca. SW1116 | 8.4 |
| Colon ca. Colo-205 | 42.9 |
| Colon ca. SW-48 | 59.0 |
| Colon | 21.9 |
| Small Intestine | 0.8 |
| Fetal Heart | 0.2 |
| Heart | 0.0 |
| Lymph Node Pool | 1.5 |
| Lymph Node pool 2 | 5.2 |
| Fetal Skeletal Muscle | 2.7 |
| Skeletal Muscle pool | 0.0 |
| Skeletal Muscle | 1.3 |
| Spleen | 4.6 |
| Thymus | 0.0 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.1 |
| CNS cancer (astro) SNB-75 | 0.3 |
| CNS cancer (glio) SNB-19 | 1.1 |
| CNS cancer (glio) SF-295 | 0.3 |
| Brain (Amygdala) | 0.4 |
| Brain (Cerebellum) | 0.7 |
| Brain (Fetal) | 4.7 |
| Brain (Hippocampus) | 0.4 |
| Cerebral Cortex pool | 0.4 |
| Brain (Substantia nigra) | 0.0 |
| Brain (Thalamus) | 0.0 |
| Brain (Whole) | 0.0 |
| Spinal Cord | 0.9 |
| Adrenal Gland | 15.9 |
| Pituitary Gland | 0.6 |
| Salivary Gland | 0.5 |
| Thyroid | 1.7 |
| Pancreatic ca. PANC-1 | 0.0 |
| Pancreas pool | 6.0 |

Column A - Rel. Ex. (%) Ag7932, Run 318010162

TABLE AF
PGI1.0

| Tissue Name | A |
|---|---|
| 162191 Normal Lung 1 (IBS) | 2.9 |
| 160468 MD lung | 7.3 |
| 156629 MD Lung 13 | 2.8 |
| 162570 Normal Lung 4 (Aastrand) | 5.4 |
| 162571 Normal Lung 3 (Aastrand) | 1.7 |
| 162187 Fibrosis Lung 2 (Genomic Collaborative) | 92.7 |
| 151281 Fibrosis lung 11 (Ardais) | 62.0 |
| 162186 Fibrosis Lung 1 (Genomic Collaborative) | 100.0 |
| 162190 Asthma Lung 4 (Genomic Collaborative) | 45.1 |
| 160467 Asthma Lung 13 (MD) | 5.9 |
| 137027 Emphysema Lung 1 (Ardais) | 8.4 |
| 137028 Emphysema Lung 2 (Ardais) | 18.2 |
| 137040 Emphysema Lung 3 (Ardais) | 24.5 |
| 137041 Emphysema Lung 4 (Ardais) | 9.8 |
| 137043 Emphysema Lung 5 (Ardais) | 16.2 |
| 142817 Emphysema Lung 6 (Ardais) | 22.2 |
| 142818 Emphysema Lung 7 (Ardais) | 2.3 |
| 142819 Emphysema Lung 8 (Ardais) | 17.2 |
| 142820 Emphysema Lung 9 (Ardais) | 4.1 |
| 142821 Emphysema Lung 10 (Ardais) | 16.2 |
| 162185 Emphysema Lung 12 (Ardais) | 42.9 |
| 162184 Emphysema Lung 13 (Ardais) | 13.6 |
| 162183 Emphysema Lung 14 (Ardais) | 38.7 |
| 162188 Emphysema Lung 15 (Genomic Collaborative) | 93.3 |
| 162177 NAT UC Colon 1 (Ardais) | 9.7 |
| 162176 UC Colon 1 (Ardais) | 7.0 |
| 162179 NAT UC Colon 2 (Ardais) | 5.0 |
| 162178 UC Colon 2 (Ardais) | 2.4 |
| 162181 NAT UC Colon 3 (Ardais) | 15.3 |
| 162180 UC Colon 3 (Ardais) | 4.0 |
| 162182 NAT UC Colon 4 (Ardais) | 18.2 |
| 137042 UC Colon 1108 | 1.4 |
| 137029 UC Colon 8215 | 1.6 |
| 137031 UC Colon 8217 | 1.2 |
| 137036 UC Colon 1137 | 3.9 |
| 137038 UC Colon 1491 | 3.0 |
| 137039 UC Colon 1546 | 9.4 |
| 162593 Crohn's 47751 (NDRI) | 0.3 |
| 162594 NAT Crohn's 47751 (NDRI) | 1.3 |

Column A - Rel. Exp. (%) Ag4044, Run 429319809

TABLE AG
general oncology screening panel v 2.4

| Tissue Name | A | B |
|---|---|---|
| Colon cancer 1 | 35.8 | 19.9 |
| CC Margin (ODO3921) | 9.2 | 3.9 |
| Colon cancer 2 | 9.5 | 6.0 |
| Colon NAT 2 | 9.3 | 2.9 |
| Colon cancer 3 | 62.0 | 40.3 |
| Colon NAT 3 | 9.9 | 4.3 |
| Colon malignant cancer 4 | 25.9 | 13.0 |
| Colon NAT 4 | 3.5 | 2.0 |
| Lung cancer 1 | 0.7 | 0.5 |
| Lung NAT 1 | 2.2 | 0.7 |
| Lung cancer 2 | 100.0 | 100.0 |
| Lung NAT 2 | 1.6 | 3.4 |
| Squamous cell carcinoma 3 | 12.3 | 5.4 |
| Lung NAT 3 | 0.5 | 0.6 |
| Metastatic melanoma 1 | 3.4 | 1.6 |
| Melanoma 2 | 0.1 | 0.1 |
| Melanoma 3 | 0.0 | 0.1 |
| Metastatic melanoma 4 | 23.7 | 11.2 |
| Metastatic melanoma 5 | 17.4 | 9.1 |
| Bladder cancer 1 | 0.0 | 0.0 |
| Bladder NAT 1 | 0.0 | 0.0 |
| Bladder cancer 2 | 1.6 | 0.5 |
| Bladder NAT 2 | 0.0 | 0.0 |
| Bladder NAT 3 | 0.1 | 0.0 |
| Bladder NAT 4 | 0.9 | 1.5 |
| Prostate adenocarcinoma 1 | 4.0 | 3.1 |
| Prostate adenocarcinoma 2 | 0.0 | 0.2 |
| Prostate adenocarcinoma 3 | 0.5 | 0.5 |
| Prostate adenocarcinoma 4 | 25.5 | 18.2 |
| Prostate NAT 5 | 0.0 | 0.1 |
| Prostate adenocarcinoma 6 | 0.0 | 0.2 |
| Prostate adenocarcinoma 7 | 1.2 | 0.3 |
| Prostate adenocarcinoma 8 | 0.0 | 0.0 |
| Prostate adenocarcinoma 9 | 6.3 | 5.6 |
| Prostate NAT 10 | 0.0 | 0.0 |
| Kidney cancer 1 | 7.5 | 5.0 |
| Kidney NAT 1 | 6.5 | 6.0 |
| Kidney cancer 2 | 69.7 | 58.6 |
| Kidney NAT 2 | 7.7 | 12.9 |
| Kidney cancer 3 | 12.8 | 16.3 |
| Kidney NAT 3 | 2.4 | 6.0 |
| Kidney cancer 4 | 61.6 | 21.6 |
| Kidney NAT 4 | 29.1 | 13.2 |

Column A - Rel. Exp. (%) Ag408, Run 268362923
Column B - Rel. Exp. (%) Ag4044, Run 268362934

Al_comprehensive panel_v1.0 Summary: Ag4044/Ag4038 Moderate levels of expression of this gene were detected in all the samples derived from rheumatoid arthritis bone and adjacent bone, cartilage, synovium and synovial fluid samples, while no expression could be seen in normal control samples. Therefore, modulation of this gene, encoded protein and/or use of antibodies or small molecule targeting this gene or gene product is useful in the treatment of inflammatory and autoimmune diseases such as rheumatoid arthritis.

General_screening_panel_v1.7 Summary: Ag7932 and Ag7932 are specific to the deletion splice variant of FGFR4, CG101729-02. The expression of this soluble FGFR4 variant was elevated in a number of ovarian cancer cell lines. The gene's expression is useful in differentiating ovarian cancer from normal ovarian tissue. Therapeutic modulation of this soluble orm of FGFR4, expressed protein and/or use of antibodies or small molecule drugs targeting the ene or gene product would be useful in the treatment of ovarian cancer.

PGI1.0 Summary: Ag4044 Elevated expression levels of this gene were detected in diseased lung tissues with Fibrosis, Asthma, and Emphysema as compared with normal lung tissues. Therapeutic modulation of this gene, expressed protein and/or use of antibodies or small molecule drugs targeting the gene or gene product would be useful in the treatment of Fibrosis, Asthma, and Emphysema.

general oncology screening panel_V_{—}2.4 Summary: Ag4044/Ag4038 Elevated expression levels of this gene were detected in colon cancer samples as compared to normal adjacent tissues. The gene's expression is useful in differentiating colon cancer tissue from normal colon tissue. Therapeutic modulation of this gene, expressed protein and/or use of antibodies or small molecule drugs targeting the gene or gene product are useful in the treatment of colon cancer.

B. NOV3, CG185793-02: MMP15.

Expression of gene CG185793-02 was assessed using the primer-probe sets Ag3682 and Ag7951, described in Tables BA and BB. Results of the RTQ-PCR runs are shown in Tables BC and BD.

TABLE BA
Probe Name Ag3682

| | | | | |
|---|---|---|---|---|
| | | | Start | SEQ |
| Primers | Sequences | Length | Position | ID No |

| | | | | |
|---|---|---|---|---|
| Forward | 5′-gctactggctctttcgag | 21 | 990 | 152 |
| | aag-3′ | | | |
| Probe | TET-5′-ctacccacagccgc | 25 | 1027 | 153 |
| | tgaccagctat-3′-TAMRA | | | |
| Reverse | 5′-cgtgtcaatgcggtcata | 19 | 1066 | 154 |
| | g-3′ | | | |

TABLE BB
Probe Name Ag7951

| | | | Start | SEQ |
|---|---|---|---|---|
| Primers | Sequences | Length | Position | ID No |
| Forward | 5′-gtggaaggacgttgacaa | 21 | 393 | 155 |
| | ctt-3′ | | | |
| Probe | TET-5′-atctccgtggcatc | 26 | 431 | 156 |
| | cagcagctctac-3′-TAMRA | | | |
| Reverse | 5′-tggactctgcatttccaa | 21 | 459 | 157 |
| | gtt-3′ | | | |

TABLE BC
General screening panel v1.7

| Tissue Name | A |
|---|---|
| Adipose | 1.5 |
| HUVEC | 0.0 |
| Melanoma* Hs688(A).T | 0.0 |
| Melanoma* Hs688(B).T | 0.2 |
| Melanoma (met) SK-MEL-5 | 0.2 |
| Testis | 2.5 |
| Prostate ca. (bone met) PC-3 | 0.0 |
| Prostate ca. DU145 | 1.2 |
| Prostate pool | 0.2 |
| Uterus pool | 0.1 |
| Ovarian ca. OVCAR-3 | 0.8 |
| Ovarian ca. (ascites) SK-OV-3 | 0.0 |
| Ovarian ca. OVCAR-4 | 0.0 |
| Ovarian ca. OVCAR-5 | 6.0 |
| Ovarian ca. IGROV-1 | 0.3 |
| Ovarian ca. OVCAR-8 | 0.0 |
| Ovary | 1.2 |
| Breast ca. MCF-7 | 1.8 |
| Breast ca. MDA-MB-231 | 0.0 |
| Breast ca. BT 549 | 0.0 |
| Breast ca. T47D | 0.0 |
| 113452 mammary gland | |
| Trachea | 2.7 |
| Lung | 9.8 |
| Fetal Lung | 2.7 |
| Lung ca. NCI-N417 | 0.0 |
| Lung ca. LX-1 | 0.2 |
| Lung ca. NCI-H146 | 0.0 |
| Lung ca. SHP-77 | 1.7 |
| Lung ca. NCI-H23 | 0.0 |
| Lung ca. NCI-H460 | 0.0 |
| Lung ca. HOP-62 | 0.0 |
| Lung ca. NCI-H522 | 1.6 |
| Lung ca. DMS-114 | 0.0 |
| Liver | 10.0 |
| Fetal Liver | 1.7 |
| Kidney pool | 11.4 |
| Fetal Kidney | 1.2 |
| Renal ca. 786-0 | 0.0 |
| Renal ca. A498 | 0.0 |
| Renal ca. ACHN | 0.0 |
| Renal ca. UO-31 | 3.5 |
| Renal ca. TK-10 | 0.9 |
| Bladder | 0.3 |
| Gastric ca. (liver met.) NCI-N87 | 0.0 |
| Stomach | 0.0 |
| Colon ca. SW-948 | 0.0 |
| Colon ca. SW480 | 0.0 |
| Colon ca. (SW480 met) SW620 | 0.0 |
| Colon ca. HT29 | 2.2 |
| Colon ca. HCT-116 | 9.7 |
| Colon cancer tissue | 0.0 |
| Colon ca. SW1116 | 0.2 |
| Colon ca. Colo-205 | 0.0 |
| Colon ca. SW-48 | 1.1 |
| Colon | 13.4 |
| Small Intestine | 0.0 |
| Fetal Heart | 13.9 |
| Heart | 0.6 |
| Lymph Node Pool | 0.3 |
| Lymph Node pool 2 | 0.4 |
| Fetal Skeletal Muscle | 0.0 |
| Skeletal Muscle pool | 0.7 |
| Skeletal Muscle | 15.5 |
| Spleen | 7.5 |
| Thymus | 0.0 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.0 |
| Brain (Cerebellum) | 1.9 |
| Brain (Fetal) | 10.4 |
| Brain (Hippocampus) | 0.0 |
| Cerebral Cortex pool | 0.0 |
| Brain (Substantia nigra) | 0.0 |
| Brain (Thalamus) | 0.0 |
| Brain (Whole) | 1.7 |
| Spinal Cord | 0.1 |
| Adrenal Gland | 0.1 |
| Pituitary Gland | 1.5 |
| Salivary Gland | 0.0 |
| Thyroid | 100.0 |
| Pancreatic ca. PANC-1 | 0.0 |
| Pancreas pool | 0.9 |

Column A - Rel. Ex. (%) Ag7951, Run 319261585

TABLE BD
general oncology screening panel v 2.4

| Tissue Name | A |
|---|---|
| Colon cancer 1 | 33.9 |
| CC Margin (ODO3921) | 17.3 |
| Colon cancer 2 | 23.0 |
| Colon NAT 2 | 26.6 |
| Colon cancer 3 | 43.2 |
| Colon NAT 3 | 19.6 |
| Colon malignant cancer 4 | 100.0 |
| Colon NAT 4 | 9.8 |
| Lung cancer 1 | 7.6 |
| Lung NAT 1 | 1.2 |
| Lung cancer 2 | 27.9 |
| Lung NAT 2 | 1.7 |
| Squamous cell carcinoma 3 | 17.1 |
| Lung NAT 3 | 0.6 |
| Metastatic melanoma 1 | 4.4 |
| Melanoma 2 | 0.9 |
| Melanoma 3 | 0.9 |
| Metastatic melanoma 4 | 9.5 |
| Metastatic melanoma 5 | 11.0 |
| Bladder cancer 1 | 0.6 |
| Bladder NAT 1 | 0.0 |
| Bladder cancer 2 | 1.3 |
| Bladder NAT 2 | 0.1 |
| Bladder NAT 3 | 0.1 |
| Bladder NAT 4 | 2.0 |
| Prostate adenocarcinoma 1 | 2.1 |
| Prostate adenocarcinoma 2 | 0.5 |
| Prostate adenocarcinoma 3 | 1.4 |
| Prostate adenocarcinoma 4 | 16.7 |
| Prostate NAT 5 | 1.0 |
| Prostate adenocarcinoma 6 | 1.7 |
| Prostate adenocarcinoma 7 | 2.0 |
| Prostate adenocarcinoma 8 | 1.1 |
| Prostate adenocarcinoma 9 | 3.3 |
| Prostate NAT 10 | 0.6 |
| Kidney cancer 1 | 12.6 |
| Kidney NAT 1 | 4.6 |
| Kidney cancer 2 | 10.2 |
| Kidney NAT 2 | 7.1 |
| Kidney cancer 3 | 4.7 |
| Kidney NAT 3 | 3.8 |
| Kidney cancer 4 | 9.3 |
| Kidney NAT 4 | 7.1 |

Column A - Rel. Exp. (%) Ag362, Run 267742159

General_screening_panel_v1.7 Summary: Ag7951 Highest gene expression was detected in Thyroid (CT=9.5). Moderate gene expression was seen in spleen, brain, kidney, skeletal muscle, liver, colon, and lung. This ubiquitous pattern of expression indicates that this gene product is involved in homeostatic processes for these and other cell types and tissues. This gene was expressed at much higher level in fetal (CT=32.3) when compared to adult heart (CT=35). This observation indicates that the protein product may enhance heart growth or development in the fetus and thus act in a regenerative capacity in the adult. This gene's expression is useful in distinguishing fetal heart tissue from adult heart tissue. Therapeutic modulation of this gene, expressed protein and/or use of antibodies or small molecule drugs targeting the gene or gene product are useful in treatment of heart related diseases.

general oncology screening panel_V_{—}2.4 Summary: Ag3682 Highest gene expression was detected in a malignant colon cancer sample (CT=27.96). Expression of this gene was upregulated in all lung cancer and prostate cancer samples when compared to the matched control margins. Moderate expression of this gene was seen in all melanoma samples. Therefore, expression of this gene is useful to differentiate lung, prostate and melanoma cancerous tissues from corresponding normal tissue. Therapeutic modulation of this gene, expressed protein and/or use of antibodies or small molecule drugs targeting the gene or gene product would be useful in the treatment of melanoma, prostate, and lung cancers.

C. NOV6 CG54470: FGF19-X.

Expression of gene CG54470 was assessed using the primer-probe sets Ag78b and Ag78, described in Tables CA and CB. Results of the RTQ-PCR runs are shown in Tables CC and CD.

TABLE CA
Probe Name Ag78b

| | | | Start | SEQ |
|---|---|---|---|---|
| Primers | Sequences | Length | Position | ID No |
| Forward | 5′-gaccagccagcacagaaa | 20 | 93 | 158 |
| | cc-3′ | | | |
| Probe | TET-5′-agtgctcgaacccg | 23 | 60 | 159 |
| | gtctcgtcc-3′-TAMRA | | | |
| Reverse | 5′-ggacccgagccattgat | 18 | 37 | 160 |
| | g-3′ | | | |

TABLE CB
Probe Name Ag78

| | | | Start | SEQ |
|---|---|---|---|---|
| Primers | Sequences | Length | Position | ID No |
| Forward | 5′-gaccagccagcacagaaa | 20 | 93 | 161 |
| | cc-3′ | | | |
| Probe | TET-5′-tcctgagtgctcga | 24 | 64 | 162 |
| | acccggtctc-3′-TAMRA | | | |
| Reverse | 5′-ggacccgagccattgat | 18 | 37 | 163 |
| | g-3′ | | | |

TABLE CC
Panel 1.3D

| Tissue Name | A |
|---|---|
| Liver adenocarcinoma | 0.0 |
| Pancreas | 0.0 |
| Pancreatic ca. CAPAN 2 | 0.4 |
| Adrenal gland | 0.0 |
| Thyroid | 0.0 |
| Salivary gland | 0.0 |
| Pituitary gland | 0.0 |
| Brain (fetal) | 0.0 |
| Brain (whole) | 0.0 |
| Brain (amygdala) | 0.0 |
| Brain (cerebellum) | 0.2 |
| Brain (hippocampus) | 0.2 |
| Brain (substantia nigra) | 0.0 |
| Brain (thalamus) | 0.0 |
| Cerebral Cortex | 0.0 |
| Spinal cord | 0.0 |
| glio/astro U87-MG | 0.0 |
| glio/astro U-118-MG | 0.0 |
| astrocytoma SW1783 | 0.0 |
| neuro*; met SK-N-AS | 0.9 |
| astrocytoma SF-539 | 0.0 |
| astrocytoma SNB-75 | 0.0 |
| glioma SNB-19 | 0.0 |
| glioma U251 | 0.0 |
| glioma SF-295 | 8.7 |
| Heart (Fetal) | 2.2 |
| Heart | 0.0 |
| Skeletal muscle (Fetal) | 3.4 |
| Skeletal muscle | 2.4 |
| Bone marrow | 0.5 |
| Thymus | 0.2 |
| Spleen | 0.0 |
| Lymph node | 0.0 |
| Colorectal | 0.4 |
| Stomach | 0.0 |
| Small intestine | 0.0 |
| Colon ca. SW480 | 1.0 |
| Colon ca.* SW620 (SW480 met) | 3.6 |
| Colon ca. HT29 | 1.4 |
| Colon ca. HCT-116 | 0.0 |
| Colon ca. CaCo-2 | 0.0 |
| CC Well to Mod Diff (ODO3866) | 0.0 |
| Colon ca. HCC-2998 | 0.0 |
| Gastric ca. (liver met) NCI-N87 | 0.0 |
| Bladder | 0.2 |
| Trachea | 1.5 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.1 |
| Renal ca. UO-31 | 0.0 |
| Renal ca. TK-10 | 0.3 |
| Liver | 17.2 |
| Liver (fetal) | 19.2 |
| Liver ca. (hepatoblast) HepG2 | 0.0 |
| Lung | 0.6 |
| Lung (fetal) | 0.0 |
| Lung ca. (small cell) LX-1 | 5.6 |
| Lung ca. (small cell) NCI-H69 | 100.0 |
| Lung ca. (s. cell var.) SHP-77 | 7.9 |
| Lung ca. (large cell)NCI-H460 | 1.8 |
| Lung ca. (non-sm. cell) A549 | 0.4 |
| Lung ca. (non-s. cell) NCI-H23 | 1.9 |
| Lung ca. (non-s. cell) HOP-62 | 0.0 |
| Lung ca. (non-s. cl) NCI-H522 | 3.0 |
| Lung ca. (squam.) SW 900 | 0.0 |
| Lung ca. (squam.) NCI-H596 | 1.6 |
| Mammary gland | 0.0 |
| Breast ca.* (pl. ef) MCF-7 | 0.3 |
| Breast ca.* (pl. ef) MDA-MB-231 | 0.9 |
| Breast ca.* (pl. ef) T47D | 0.0 |
| Breast ca. BT-549 | 0.7 |
| Breast ca. MDA-N | 0.0 |
| Ovary | 0.3 |
| Ovarian ca. OVCAR-3 | 0.0 |
| Ovarian ca. OVCAR-4 | 0.0 |
| Ovarian ca. OVCAR-5 | 0.7 |
| Ovarian ca. OVCAR-8 | 0.4 |
| Ovarian ca. IGROV-1 | 2.2 |
| Ovarian ca. (ascites) SK-OV-3 | 0.9 |
| Uterus | 0.0 |
| Placenta | 0.7 |
| Prostate | 0.0 |
| Prostate ca.* (bone met) PC-3 | 0.0 |
| Testis | 0.3 |
| Melanoma Hs688(A).T | 0.0 |
| Melanoma* (met) Hs688(B).T | 0.0 |
| Melanoma UACC-62 | 0.3 |
| Melanoma M14 | 0.0 |
| Melanoma LOX IMVI | 0.0 |
| Melanoma* (met) SK-MEL-5 | 0.0 |
| Adipose | 0.0 |

Column A - Rel. Exp. (%) Ag78b, Run 152827429

TABLE CD
Panel 2D

| Tissue Name | A | B |
|---|---|---|
| Normal Colon | 0.6 | 0.0 |
| CC Well to Mod Diff (ODO3866) | 0.0 | 0.6 |
| CC Margin (ODO3866) | 0.0 | 0.1 |
| CC Gr. 2 rectosigmoid (ODO3868) | 0.0 | 0.2 |
| CC Margin (ODO3868) | 0.0 | 0.0 |
| CC Mod Diff (ODO3920) | 0.0 | 0.0 |
| CC Margin (ODO3920) | 0.0 | 0.0 |
| CC Gr. 2 ascend colon (ODO3921) | 0.0 | 0.0 |
| CC Margin (ODO3921) | 0.0 | 0.0 |
| CC from Partial Hepatectomy | 5.3 | 12.5 |
| (ODO4309) Mets | | |
| Liver Margin (ODO4309) | 100.0 | 100.0 |
| Colon mets to lung (OD04451-01) | 0.0 | 0.0 |
| Lung Margin (OD04451-02) | 0.0 | 0.0 |
| Normal Prostate 6546-1 | 0.0 | 0.0 |
| Prostate Cancer (OD04410) | 0.0 | 0.0 |
| Prostate Margin (OD04410) | 0.0 | 0.2 |
| Prostate Cancer (OD04720-01) | 0.0 | 0.1 |
| Prostate Margin (OD04720-02) | 0.3 | 0.0 |
| Normal Lung | 0.1 | 0.2 |
| Lung Met to Muscle (ODO4286) | 0.0 | 0.0 |
| Muscle Margin (ODO4286) | 0.0 | 0.0 |
| Lung Malignant Cancer (OD03126) | 0.0 | 0.0 |
| Lung Margin (OD03126) | 0.0 | 0.0 |
| Lung Cancer (OD04404) | 0.0 | 0.0 |
| Lung Margin (OD04404) | 0.0 | 0.0 |
| Lung Cancer (OD04565) | 0.0 | 0.0 |
| Lung Margin (OD04565) | 0.0 | 0.0 |
| Lung Cancer (OD04237-01) | 0.0 | 0.4 |
| Lung Margin (OD04237-02) | 0.0 | 0.0 |
| Ocular Mel Met to Liver (ODO4310) | 0.0 | 0.6 |
| Liver Margin (ODO4310) | 44.8 | 60.7 |
| Melanoma Metastasis | 0.0 | 0.0 |
| Lung Margin (OD04321) | 0.0 | 0.0 |
| Normal Kidney | 0.0 | 0.4 |
| Kidney Ca, Nuclear grade 2 (OD04338) | 0.0 | 0.0 |
| Kidney Margin (OD04338) | 0.0 | 0.0 |
| Kidney Ca Nuclear grade 1/2 (OD04339) | 0.0 | 0.0 |
| Kidney Margin (OD04339) | 0.0 | 0.0 |
| Kidney Ca, Clear cell type (OD04340) | 0.0 | 0.0 |
| Kidney Margin (OD04340) | 0.6 | 0.0 |
| Kidney Ca, Nuclear grade 3 (OD04348) | 0.0 | 0.0 |
| Kidney Margin (OD04348) | 0.0 | 0.0 |
| Kidney Cancer (OD04622-01) | 0.0 | 0.0 |
| Kidney Margin (OD04622-03) | 0.0 | 0.0 |
| Kidney Cancer (OD04450-01) | 0.0 | 0.0 |
| Kidney Margin (OD04450-03) | 0.0 | 0.0 |
| Kidney Cancer 8120607 | 0.0 | 0.8 |
| Kidney Margin 8120608 | 0.0 | 0.0 |
| Kidney Cancer 8120613 | 0.0 | 0.0 |
| Kidney Margin 8120614 | 0.0 | 0.0 |
| Kidney Cancer 9010320 | 0.2 | 0.0 |
| Kidney Margin 9010321 | 0.0 | 0.4 |
| Normal Uterus | 0.0 | 0.0 |
| Uterine Cancer 064011 | 0.0 | 0.0 |
| Normal Thyroid | 0.0 | 0.0 |
| Thyroid Cancer | 0.0 | 0.4 |
| Thyroid Cancer A302152 | 0.3 | 0.4 |
| Thyroid Margin A302153 | 0.0 | 0.0 |
| Normal Breast | 0.0 | 0.0 |
| Breast Cancer | 0.0 | 0.0 |
| Breast Cancer (OD04590-01) | 0.0 | 0.0 |
| Breast Cancer Mets (OD04590-03) | 0.0 | 0.0 |
| Breast Cancer Metastasis | 0.0 | 0.0 |
| Breast Cancer | 0.0 | 0.0 |
| Breast Cancer | 0.0 | 0.1 |
| Breast Cancer 9100266 | 0.0 | 0.0 |
| Breast Margin 9100265 | 0.0 | 0.0 |
| Breast Cancer A209073 | 0.0 | 0.0 |
| Breast Margin A209073 | 0.0 | 0.0 |
| Normal Liver | 1.2 | 1.0 |
| Liver Cancer | 13.9 | 18.0 |
| Liver Cancer 1025 | 8.1 | 6.4 |
| Liver Cancer 1026 | 13.2 | 13.8 |
| Liver Cancer 6004-T | 33.9 | 16.6 |
| Liver Tissue 6004-N | 0.6 | 0.7 |
| Liver Cancer 6005-T | 34.2 | 28.1 |
| Liver Tissue 6005-N | 15.8 | 14.1 |
| Normal Bladder | 0.3 | 0.0 |
| Bladder Cancer | 0.0 | 0.0 |
| Bladder Cancer | 0.9 | 1.7 |
| Bladder Cancer (OD04718-01) | 0.0 | 0.3 |
| Bladder Normal Adjacent (OD04718-03) | 0.5 | 0.3 |
| Normal Ovary | 0.0 | 0.0 |
| Ovarian Cancer | 0.0 | 0.4 |
| Ovarian Cancer (OD04768-07) | 5.6 | 7.4 |
| Ovary Margin (OD04768-08) | 0.0 | 0.0 |
| Normal Stomach | 0.0 | 0.0 |
| Gastric Cancer 9060358 | 0.0 | 0.0 |
| Stomach Margin 9060359 | 0.0 | 0.2 |
| Gastric Cancer 9060395 | 0.0 | 0.0 |
| Stomach Margin 9060394 | 0.0 | 0.0 |
| Gastric Cancer 9060397 | 0.0 | 0.0 |
| Stomach Margin 9060396 | 0.0 | 0.0 |
| Gastric Cancer 064005 | 0.0 | 0.0 |

Column A - Rel. Exp. (%) Ag78, Run 158135898
Column B - Rel. Exp. (%) Ag78b, Run 152827454

Panel 1.3D Summary: Ag78b Moderate gene expression was detected in cancer cell lines derived from lung, while no expression was seen in normal lung tissue. Thus, the gene's expression is useful in differentiating lung cancer from normal lung tissue. Therapeutic modulation of this gene, expressed protein and/or use of antibodies or small molecule drugs targeting the gene or gene product are useful in the treatment of lung cancer.

Panel 2D Summary: Ag78 Gene expression was highest in a sample derived from normal liver tissue adjacent to a colon cancer metastasis. In addition, there was substantial expression in samples derived from normal liver and liver cancers as well as a sample derived from liver tissue adjacent to an ocular melanoma metastasis. Of particular interest is the difference in expression of this gene between liver cancers and their adjacent normal tissues. There was a 20-fold and 2-fold difference in expression between liver cancer samples when compared to matched margins (6004-T vs 6004-N and 6005-T vs 6005-N, respectively). Gene expression is useful in differentiating liver cancer tissue from normal liver tissue. Therapeutic modulation of this gene, expressed protein and/or use of antibodies or small molecule drugs targeting the gene or gene product are useful in the treatment of liver cancer.

D. NOV7, CG55051: Alpha-2-macroglobulin like.

Expression of gene CG55051 was assessed using the primer-probe sets Agl 180 and Ag1312, described in Tables DA and DB. Results of the RTQ-PCR runs are shown in Tables DC, DD, DE and DF.

TABLE DA
Probe Name Ag1180

| | | | Start | SEQ |
|---|---|---|---|---|
| Primers | Sequences | Length | Position | ID No |
| Forward | 5′-cctggaaatagggtacca | 22 | 3027 | 164 |
| | gaag-3′ | | | |
| Probe | TET-5′-acacagcaatggct | 26 | 3063 | 165 |
| | catacagtgcct-3′-TAMRA | | | |
| Reverse | 5′-tcagccatgtgtttccat | 20 | 3105 | 166 |
| | tt-3′ | | | |

TABLE DB
Probe Name Ag1312

| | | | Start | SEQ |
|---|---|---|---|---|
| Primers | Sequences | Length | Position | ID No |
| Forward | 5′-cctggaaatagggtacca | 22 | 3027 | 167 |
| | gaag-3′ | | | |
| Probe | TET-5′-acacagcaatggct | 26 | 3063 | 168 |
| | catacagtgcct-3′-TAMRA | | | |
| Reverse | 5′-tcagccatgtgtttccat | 20 | 3105 | 169 |
| | tt-3′ | | | |

TABLE DC
AI comprehensive panel v1.0

| Tissue Name | A |
|---|---|
| 110967 COPD-F | 0.0 |
| 110980 COPD-F | 0.0 |
| 110968 COPD-M | 0.0 |
| 110977 COPD-M | 0.0 |
| 110989 Emphysema-F | 0.0 |
| 110992 Emphysema-F | 0.0 |
| 110993 Emphysema-F | 0.0 |
| 110994 Emphysema-F | 0.0 |
| 110995 Emphysema-F | 0.0 |
| 110996 Emphysema-F | 0.0 |
| 110997 Asthma-M | 0.0 |
| 111001 Asthma-F | 0.0 |
| 111002 Asthma-F | 0.0 |
| 111003 Atopic Asthma-F | 0.1 |
| 111004 Atopic Asthma-F | 0.0 |
| 111005 Atopic Asthma-F | 0.0 |
| 111006 Atopic Asthma-F | 0.0 |
| 111417 Allergy-M | 0.0 |
| 112347 Allergy-M | 0.0 |
| 112349 Normal Lung-F | 0.0 |
| 112357 Normal Lung-F | 0.1 |
| 112354 Normal Lung-M | 0.0 |
| 112374 Crohns-F | 0.0 |
| 112389 Match Control Crohns-F | 49.7 |
| 112375 Crohns-F | 0.1 |
| 112732 Match Control Crohns-F | 53.6 |
| 112725 Crohns-M | 0.0 |
| 112387 Match Control Crohns-M | 0.1 |
| 112378 Crohns-M | 0.0 |
| 112390 Match Control Crohns-M | 0.1 |
| 112726 Crohns-M | 0.1 |
| 112731 Match Control Crohns-M | 0.1 |
| 112380 Ulcer Col-F | 0.0 |
| 112734 Match Control Ulcer Col-F | 100.0 |
| 112384 Ulcer Col-F | 0.2 |
| 112737 Match Control Ulcer Col-F | 0.0 |
| 112386 Ulcer Col-F | 0.0 |
| 112738 Match Control Ulcer Col-F | 0.0 |
| 112381 Ulcer Col-M | 0.0 |
| 112735 Match Control Ulcer Col-M | 0.0 |
| 112382 Ulcer Col-M | 61.6 |
| 112394 Match Control Ulcer Col-M | 0.1 |
| 112383 Ulcer Col-M | 0.1 |
| 112736 Match Control Ulcer Col-M | 46.0 |
| 112423 Psoriasis-F | 0.0 |
| 112427 Match Control Psoriasis-F | 0.5 |
| 112418 Psoriasis-M | 0.1 |
| 112723 Match Control Psoriasis-M | 0.0 |
| 112419 Psoriasis-M | 0.2 |
| 112424 Match Control Psoriasis-M | 0.0 |
| 112420 Psoriasis-M | 0.2 |
| 112425 Match Control Psoriasis-M | 0.1 |
| 104689 (MF) OA Bone-Backus | 0.1 |
| 104690 (MF) Adj “Normal” Bone-Backus | 0.0 |
| 104691 (MF) OA Synovium-Backus | 0.0 |
| 104692 (BA) OA Cartilage-Backus | 0.0 |
| 104694 (BA) OA Bone-Backus | 0.0 |
| 104695 (BA) Adj “Normal” Bone-Backus | 0.0 |
| 104696 (BA) OA Synovium-Backus | 0.0 |
| 104700 (SS) OA Bone-Backus | 0.0 |
| 104701 (SS) Adj “Normal” Bone-Backus | 0.1 |
| 104702 (SS) OA Synovium-Backus | 0.1 |
| 117093 OA Cartilage Rep7 | 0.0 |
| 112672 OA Bone5 | 0.6 |
| 112673 OA Synovium5 | 0.7 |
| 112674 OA Synovial Fluid cells5 | 0.4 |
| 117100 OA Cartilage Rep14 | 0.0 |
| 112756 OA Bone9 | 0.0 |
| 112757 OA Synovium9 | 0.0 |
| 112758 OA Synovial Fluid Cells9 | 0.0 |
| 117125 RA Cartilage Rep2 | 0.0 |
| 113492 Bone2 RA | 0.0 |
| 113493 Synovium2 RA | 0.0 |
| 113494 Syn Fluid Cells RA | 0.0 |
| 113499 Cartilage4 RA | 0.0 |
| 113500 Bone4 RA | 0.0 |
| 113501 Synovium4 RA | 0.0 |
| 113502 Syn Fluid Cells4 RA | 0.0 |
| 113495 Cartilage3 RA | 0.0 |
| 113496 Bone3 RA | 0.0 |
| 113497 Synovium3 RA | 0.0 |
| 113498 Syn Fluid Cells3 RA | 0.0 |
| 117106 Normal Cartilage Rep20 | 0.0 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.0 |
| 113667 Bone4 Normal | 0.1 |
| 113668 Synovium4 Normal | 0.0 |
| 113669 Syn Fluid Cells4 Normal | 0.0 |

Column A - Rel. Ex. (%) Ag1180, Run 228061003

TABLE DD
Panel 1.3D

| Tissue Name | A |
|---|---|
| Liver adenocarcinoma | 0.0 |
| Pancreas | 0.0 |
| Pancreatic ca. CAPAN 2 | 11.3 |
| Adrenal gland | 0.0 |
| Thyroid | 1.6 |
| Salivary gland | 7.5 |
| Pituitary gland | 0.8 |
| Brain (fetal) | 4.5 |
| Brain (whole) | 8.9 |
| Brain (amygdala) | 22.7 |
| Brain (cerebellum) | 8.0 |
| Brain (hippocampus) | 4.9 |
| Brain (substantia nigra) | 1.9 |
| Brain (thalamus) | 6.7 |
| Cerebral Cortex | 6.8 |
| Spinal cord | 47.6 |
| glio/astro U87-MG | 0.0 |
| glio/astro U-118-MG | 0.0 |
| astrocytoma SW1783 | 0.0 |
| neuro*; met SK-N-AS | 0.0 |
| astrocytoma SF-539 | 0.0 |
| astrocytoma SNB-75 | 0.0 |
| glioma SNB-19 | 0.0 |
| glioma U251 | 0.0 |
| glioma SF-295 | 0.0 |
| Heart (Fetal) | 0.0 |
| Heart | 0.0 |
| Skeletal muscle (Fetal) | 0.0 |
| Skeletal muscle | 0.9 |
| Bone marrow | 0.0 |
| Thymus | 14.1 |
| Spleen | 1.4 |
| Lymph node | 0.0 |
| Colorectal | 0.0 |
| Stomach | 17.8 |
| Small intestine | 0.0 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| CC Well to Mod Diff (ODO3866) | 0.0 |
| Colon ca. HCC-2998 | 0.0 |
| Gastric ca. (liver met) NCI-N87 | 100.0 |
| Bladder | 0.0 |
| Trachea | 7.5 |
| Kidney | 0.4 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.9 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 3.0 |
| Breast ca.* (pl. ef) MCF-7 | 0.9 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 2.5 |
| Ovarian ca. OVCAR-3 | 0.2 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca. (ascites) SK-OV-3 | 0.3 |
| Uterus | 0.0 |
| Placenta | 11.3 |
| Prostate | 0.0 |
| Prostate ca.* (bone met) PC-3 | 0.0 |
| Testis | 17.1 |
| Melanoma Hs688(A).T | 0.0 |
| Melanoma* (met) Hs688(B).T | 0.0 |
| Melanoma UACC-62 | 0.9 |
| Melanoma M14 | 0.0 |
| Melanoma LOX IMVI | 0.0 |
| Melanoma* (met) SK-MEL-5 | 0.0 |
| Adipose | 0.0 |

Column A - Rel. Exp. (%) Ag1180, Run 165920069

TABLE DE
Panel 2D

| Tissue Name | A |
|---|---|
| Normal Colon | 0.1 |
| CC Well to Mod Diff (ODO3866) | 0.1 |
| CC Margin (ODO3866) | 0.0 |
| CC Gr. 2 rectosigmoid (ODO3868) | 0.0 |
| CC Margin (ODO3868) | 0.0 |
| CC Mod Diff (ODO3920) | 0.1 |
| CC Margin (ODO3920) | 0.1 |
| CC Gr. 2 ascend colon (ODO3921) | 0.0 |
| CC Margin (ODO3921) | 0.0 |
| CC from Partial Hepatectomy (ODO4309) Mets | 0.0 |
| Liver Margin (ODO4309) | 0.0 |
| Colon mets to lung (OD04451-01) | 0.0 |
| Lung Margin (OD04451-02) | 0.0 |
| Normal Prostate 6546-1 | 3.3 |
| Prostate Cancer (OD04410) | 0.0 |
| Prostate Margin (OD04410) | 0.6 |
| Prostate Cancer (OD04720-01) | 0.5 |
| Prostate Margin (OD04720-02) | 0.8 |
| Normal Lung | 0.1 |
| Lung Met to Muscle (ODO4286) | 0.0 |
| Muscle Margin (ODO4286) | 0.0 |
| Lung Malignant Cancer (OD03126) | 0.0 |
| Lung Margin (OD03126) | 0.0 |
| Lung Cancer (OD04404) | 18.4 |
| Lung Margin (OD04404) | 0.0 |
| Lung Cancer (OD04565) | 0.1 |
| Lung Margin (OD04565) | 0.0 |
| Lung Cancer (OD04237-01) | 0.0 |
| Lung Margin (OD04237-02) | 0.0 |
| Ocular Mel Met to Liver (ODO4310) | 0.1 |
| Liver Margin (ODO4310) | 0.0 |
| Melanoma Metastasis | 0.0 |
| Lung Margin (OD04321) | 0.0 |
| Normal Kidney | 0.1 |
| Kidney Ca, Nuclear grade 2 (OD04338) | 0.0 |
| Kidney Margin (OD04338) | 0.0 |
| Kidney Ca Nuclear grade 1/2 (OD04339) | 0.0 |
| Kidney Margin (OD04339) | 0.0 |
| Kidney Ca, Clear cell type (OD04340) | 0.0 |
| Kidney Margin (OD04340) | 0.0 |
| Kidney Ca, Nuclear grade 3 (OD04348) | 0.0 |
| Kidney Margin (OD04348) | 0.0 |
| Kidney Cancer (OD04622-01) | 0.0 |
| Kidney Margin (OD04622-03) | 0.1 |
| Kidney Cancer (OD04450-01) | 0.0 |
| Kidney Margin (OD04450-03) | 0.0 |
| Kidney Cancer 8120607 | 0.0 |
| Kidney Margin 8120608 | 0.0 |
| Kidney Cancer 8120613 | 0.0 |
| Kidney Margin 8120614 | 0.0 |
| Kidney Cancer 9010320 | 0.0 |
| Kidney Margin 9010321 | 0.0 |
| Normal Uterus | 0.0 |
| Uterine Cancer 064011 | 0.4 |
| Normal Thyroid | 0.4 |
| Thyroid Cancer | 0.0 |
| Thyroid Cancer A302152 | 0.0 |
| Thyroid Margin A302153 | 0.0 |
| Normal Breast | 0.1 |
| Breast Cancer | 0.0 |
| Breast Cancer (OD04590-01) | 0.0 |
| Breast Cancer Mets (OD04590-03) | 0.0 |
| Breast Cancer Metastasis | 0.0 |
| Breast Cancer | 1.2 |
| Breast Cancer | 0.0 |
| Breast Cancer 9100266 | 0.0 |
| Breast Margin 9100265 | 0.0 |
| Breast Cancer A209073 | 0.2 |
| Breast Margin A209073 | 0.1 |
| Normal Liver | 0.0 |
| Liver Cancer | 0.0 |
| Liver Cancer 1025 | 0.0 |
| Liver Cancer 1026 | 0.0 |
| Liver Cancer 6004-T | 0.0 |
| Liver Tissue 6004-N | 0.0 |
| Liver Cancer 6005-T | 0.0 |
| Liver Tissue 6005-N | 0.0 |
| Normal Bladder | 0.0 |
| Bladder Cancer | 0.0 |
| Bladder Cancer | 12.9 |
| Bladder Cancer (OD04718-01) | 0.6 |
| Bladder Normal Adjacent (OD04718-03) | 0.0 |
| Normal Ovary | 0.1 |
| Ovarian Cancer | 0.8 |
| Ovarian Cancer (OD04768-07) | 100.0 |
| Ovary Margin (OD04768-08) | 0.1 |
| Normal Stomach | 0.0 |
| Gastric Cancer 9060358 | 0.0 |
| Stomach Margin 9060359 | 0.0 |
| Gastric Cancer 9060395 | 0.2 |
| Stomach Margin 9060394 | 0.0 |
| Gastric Cancer 9060397 | 0.1 |
| Stomach Margin 9060396 | 0.0 |
| Gastric Cancer 064005 | 0.1 |

Column A - Rel. Exp. (%) Ag1180, Run 162599404

TABLE DEF
Panel 4D

| Tissue Name | A | B |
|---|---|---|
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.1 | 0.1 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 0.0 |
| CD45RO CD4 lymphocyte act | 0.0 | 0.0 |
| CD8 lymphocyte act | 0.0 | 0.0 |
| Secondary CD8 lymphocyte rest | 0.0 | 0.0 |
| Secondary CD8 lymphocyte act | 0.0 | 0.1 |
| CD4 lymphocyte none | 0.0 | 0.0 |
| 2ry Th1/Th2/Tr1 anti-CD95 CH11 | 0.0 | 0.0 |
| LAK cells rest | 0.0 | 0.0 |
| LAK cells IL-2 | 0.0 | 0.0 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.2 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 0.0 | 0.0 |
| Dendritic cells LPS | 0.0 | 0.0 |
| Dendritic cells anti-CD40 | 0.1 | 0.0 |
| Monocytes rest | 0.0 | 0.0 |
| Monocytes LPS | 0.1 | 0.1 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 5.3 | 5.7 |
| Small airway epithelium none | 28.7 | 38.7 |
| Small airway epithelium TNFalpha + IL-1beta | 100.0 | 100.0 |
| Coronery artery SMC rest | 0.0 | 0.0 |
| Coronery artery SMC TNFalpha + IL-1beta | 0.0 | 0.0 |
| Astrocytes rest | 0.0 | 0.0 |
| Astrocytes TNFalpha + IL-1beta | 0.0 | 0.1 |
| KU-812 (Basophil) rest | 0.1 | 0.1 |
| KU-812 (Basophil) PMA/ionomycin | 0.1 | 0.0 |
| CCD1106 (Keratinocytes) none | 1.7 | 1.7 |
| 93580 CCD1106 (Keratinocytes) TNFa and IFNg | 15.3 | 14.8 |
| Liver cirrhosis | 0.0 | 0.0 |
| Lupus kidney | 0.0 | 0.0 |
| NCI-H292 none | 0.3 | 0.1 |
| NCI-H292 IL-4 | 0.3 | 0.1 |
| NCI-H292 IL-9 | 0.0 | 0.1 |
| NCI-H292 IL-13 | 0.1 | 0.1 |
| NCI-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 0.0 | 0.0 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 0.0 |
| IBD Colitis 2 | 0.0 | 0.0 |
| IBD Crohn's | 0.0 | 0.0 |
| Colon | 0.0 | 0.0 |
| Lung | 0.0 | 0.0 |
| Thymus | 0.1 | 0.2 |
| Kidney | 1.8 | 3.0 |

Column A - Rel. Exp. (%) Ag1180, Run 139410602
Column B - Rel. Exp. (%) Ag1312, Run 138968169

Al_comprehensive panel_v1.0 Summary: Ag1180 High gene expression was detected in Crohns tissues from female patients, while no expression was detected in Crohns samples from male patients. The gene's expression is useful in differentiating Crohns disease colon tissue from normal colon tissue in female patients. Therapeutic modulation of this gene, expressed protein and/or use of antibodies or small molecule drugs targeting the gene or gene product are useful in the treatment of Crohns disease and other inflammatory disorders including psoriasis, allergy, asthma, inflammatory bowel disease, rheumatoid arthritis and osteoarthritis.

Panel 1.3D Summary: Agl 180 Moderate levels of gene expression were detected in gastric cancer cell lines (CT=30.4) and lower levels in pancreatic cancer cell lines (CT=33.5). Gene expression is useful for differentiating gastric and pancreatic cancerous tissue from normal tissue. Therapeutic modulation of this gene, expressed protein and/or use of antibodies or small molecule drugs targeting the gene or gene product are useful in the treatment of gastric and pancreatic cancers. Low levels of gene expression was detected in brain. Among tissues involved in central nervous system function, this gene is specifically expressed at low to moderate levels in the amygdala, cerebellum, cortex, hippocampus and thalamus, and expressed highly in the spinal cord and cerebral cortex. Alpha-2-macroglobulin has been implicated in Alzheimer's disease, both genetically and biochemically in the clearance of beta amyloid. The high similarity of this gene's protein product to alpha-2-macroglobulin suggests indicates its involvement in Alzheimer's. Therapeutic modulation of this gene, expressed protein and/or use of antibodies or small molecule drugs targeting the gene or gene product are useful in the treatment of Alzheimer's disease. Agents that increase expression, concentration, or activity of this gene will aid in the clearance of A-beta, which is a hallmark of Alzheimer's disease histopathology.

Panel 2D Summary: Agl 180 Highest gene expression was detected in ovarian cancer tissue (CT=25.67) and it is overexpressed in ovarian cancer samples when compared to the normal margins. There was low but significant expression of this gene in some breast, bladder, and lung cancer samples. Expression of this gene can be used to differentiate ovarian breast, bladder, and lung cancerous tissue from normal specimens. Therapeutic modulation of this gene, expressed protein and/or use of antibodies or small molecule drugs targeting the gene or gene product would be useful in the treatment of bladder, ovarian, breast, and lung cancer.

Panel 4D Summary: Ag1180/Ag1312 Expression of this gene was detected at moderate levels in small airway epithelium (CT=28) and is slightly upregulated when treated with TNF-alpha+IL-1beta (CT=26-27). This gene encodes a protein that is a macroglobulin-like molecule belonging to a class of proteinase inhibitor that can behave as a potent modulator of the inflammatory reaction and tissue repair mechanism. Therapeutic modulation of this gene, expressed protein and/or use of antibodies or small molecule drugs targeting the gene or gene product are useful in the treatment of asthma and emphysema. Expression of this gene was detected in keratinocytes stimulated with the inflammatory cytokines TNF-alpha+IL-1 beta (CT=29). The gene's expression is useful in differentiating keratinocytes stimulated with the inflammatory cytokines TNF-alpha+IL-1 beta from unstimulated keratinocytes. Therapeutic modulation of this gene, expressed protein and/or use of antibodies or small molecule drugs targeting the gene or gene product would be useful in the treatment of skin related disease such as psoriasis, eczema, and contact dermatitis.

E. NOV 8, CG55060: SLPI.

Expression of gene CG55060 was assessed using the primer-probe set Ag588, described in Table EA. Results of the RTQ-PCR runs are shown in Tables EB, EC, ED, EE, EF and EG.

TABLE EA
Probe Name Ag588

| | | | | |
|---|---|---|---|---|
| | | | Start | SEQ |
| Primers | Sequences | Length | Position | ID No |

| | | | | |
|---|---|---|---|---|
| Forward | 5′-tgccttcaccatgaagtc | 20 | 9 | 170 |
| | ca-3′ | | | |
| Probe | TET-5′-cttcctggtgctgc | 23 | 42 | 171 |
| | ttgccctgg-3′-TAMRA | | | |
| Reverse | 5′-agcccaaggtgccagagt | 19 | 66 | 172 |
| | t-3′ | | | |

TABLE EB
Ardais Kidney 1.0

| Tissue Name | A |
|---|---|
| Kidney cancer(10A8) | 5.5 |
| Kidney NAT(10A9) | 0.2 |
| Kidney cancer(10AA) | 0.0 |
| Kidney NAT(10AB) | 0.2 |
| Kidney cancer(10AC) | 0.3 |
| Kidney NAT(10AD) | 10.2 |
| Kidney cancer(10B6) | 0.1 |
| Kidney NAT(10B7) | 0.4 |
| Kidney cancer(10B8) | 2.2 |
| Kidney NAT(10B9) | 0.4 |
| Kidney cancer(10BC) | 30.6 |
| Kidney NAT(10BD) | 2.1 |
| Kidney cancer(10BE) | 0.0 |
| Kidney NAT(10BF) | 0.1 |
| Kidney cancer(10C2) | 1.1 |
| Kidney NAT(10C3) | 0.5 |
| Kidney cancer(10C4) | 0.9 |
| Kidney NAT(10C5) | 0.2 |
| Kidney cancer(10B4) | 4.1 |
| Kidney cancer(10C8) | 0.0 |
| Kidney cancer(10D0) | 0.0 |
| Kidney cancer(10C0) | 92.7 |
| Kidney cancer(10C6) | 0.5 |
| Kidney cancer(10C9) | 0.1 |
| Kidney cancer(10D1) | 0.0 |
| Kidney cancer(10CA) | 100.0 |
| Kidney cancer(10D2) | 0.0 |
| Kidney cancer(10CB) | 3.0 |
| Kidney cancer(10D4) | 2.9 |
| Kidney cancer(10CD) | 0.1 |
| Kidney cancer(10D5) | 0.0 |
| Kidney cancer(10CE) | 0.0 |
| Kidney cancer(10D6) | 0.2 |
| Kidney cancer(10CF) | 0.0 |
| Kidney cancer(10D8) | 0.5 |
| Kidney cancer(10CC) | 1.0 |
| Kidney cancer(10D3) | 3.3 |
| Kidney NAT(10D9) | 0.6 |
| Kidney NAT(10DB) | 5.6 |
| Kidney NAT(10DC) | 0.1 |
| Kidney NAT(10DD) | 1.1 |
| Kidney NAT(10DE) | 1.7 |
| Kidney NAT(10B1) | 3.5 |
| Kidney NAT(10DA) | 0.1 |

Column A - Rel. Exp. (%) Ag588, Run 369943434

TABLE EC
CNS neurodegeneration v1.0

| Tissue Name | A |
|---|---|
| AD 1 Hippo | 6.1 |
| AD 2 Hippo | 33.7 |
| AD 3 Hippo | 100.0 |
| AD 4 Hippo | 14.3 |
| AD 5 Hippo | 3.8 |
| AD 6 Hippo | 3.1 |
| Control 2 Hippo | 1.1 |
| Control 4 Hippo | 7.9 |
| Control (Path) 3 Hippo | 26.8 |
| AD 1 Temporal Ctx | 10.6 |
| AD 2 Temporal Ctx | 9.9 |
| AD 3 Temporal Ctx | 15.8 |
| AD 4 Temporal Ctx | 3.6 |
| AD 5 Inf Temporal Ctx | 0.5 |
| AD 5 Sup Temporal Ctx | 4.4 |
| AD 6 Inf Temporal Ctx | 2.8 |
| AD 6 Sup Temporal Ctx | 5.2 |
| Control 1 Temporal Ctx | 16.0 |
| Control 2 Temporal Ctx | 0.4 |
| Control 3 Temporal Ctx | 3.1 |
| Control 3 Temporal Ctx | 2.7 |
| AH3 3975 | 1.3 |
| AH3 3954 | 3.3 |
| AH3 4624 | 6.1 |
| AH3 4640 | 0.9 |
| AD 1 Occipital Ctx | 5.1 |
| AD 2 Occipital Ctx (Missing) | 0.4 |
| AD 3 Occipital Ctx | 14.1 |
| AD 4 Occipital Ctx | 3.4 |
| AD 5 Occipital Ctx | 4.4 |
| AD 5 Occipital Ctx | 4.9 |
| Control 1 Occipital Ctx | 15.2 |
| Control 2 Occipital Ctx | 0.6 |
| Control 3 Occipital Ctx | 1.6 |
| Control 4 Occipital Ctx | 2.0 |
| Control (Path) 1 Occipital Ctx | 0.8 |
| Control (Path) 2 Occipital Ctx | 2.3 |
| Control (Path) 3 Occipital Ctx | 17.0 |
| Control (Path) 4 Occipital Ctx | 1.2 |
| Control 1 Parietal Ctx | 15.4 |
| Control 2 Parietal Ctx | 3.0 |
| Control 3 Parietal Ctx | 2.3 |
| Control (Path) 1 Parietal Ctx | 2.8 |
| Control (Path) 2 Parietal Ctx | 7.1 |
| Control (Path) 3 Parietal Ctx | 10.4 |
| Control (Path) 4 Parietal Ctx | 4.2 |

Column A - Rel. Ep. (%) Ag588, Run 224758452

TABLE ED
General screening panel v1.5

| Tissue Name | A |
|---|---|
| Adipose | 0.9 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 2.6 |
| Testis Pool | 0.2 |
| Prostate ca.* (bone met) PC-3 | 0.6 |
| Prostate Pool | 0.1 |
| Placenta | 0.0 |
| Uterus Pool | 0.4 |
| Ovarian ca. OVCAR-3 | 6.5 |
| Ovarian ca. SK-OV-3 | 11.3 |
| Ovarian ca. OVCAR-4 | 6.4 |
| Ovarian ca. OVCAR-5 | 4.4 |
| Ovarian ca. IGROV-1 | 4.5 |
| Ovarian ca. OVCAR-8 | 0.1 |
| Ovary | 0.9 |
| Breast ca. MCF-7 | 0.1 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.4 |
| Trachea | 100.0 |
| Lung | 0.0 |
| Fetal Lung | 3.6 |
| Lung ca. NCI-N417 | 0.0 |
| Lung ca. LX-1 | 1.9 |
| Lung ca. NCI-H146 | 0.0 |
| Lung ca. SHP-77 | 0.0 |
| Lung ca. A549 | 0.4 |
| Lung ca. NCI-H526 | 0.0 |
| Lung ca. NCI-H23 | 0.3 |
| Lung ca. NCI-H460 | 2.2 |
| Lung ca. HOP-62 | 0.3 |
| Lung ca. NCI-H522 | 0.0 |
| Liver | 0.3 |
| Fetal Liver | 0.0 |
| Liver ca. HepG2 | 0.2 |
| Kidney Pool | 0.1 |
| Fetal Kidney | 0.0 |
| Renal ca. 786-0 | 0.0 |
| Renal ca. A498 | 0.5 |
| Renal ca. ACHN | 0.0 |
| Renal ca. UO-31 | 0.3 |
| Renal ca. TK-10 | 0.0 |
| Bladder | 1.0 |
| Gastric ca. (liver met.) NCI-N87 | 6.3 |
| Gastric ca. KATO III | 0.2 |
| Colon ca. SW-948 | 0.7 |
| Colon ca. SW480 | 0.2 |
| Colon ca.* (SW480 met) SW620 | 0.0 |
| Colon ca. HT29 | 0.0 |
| Colon ca. HCT-116 | 0.0 |
| Colon ca. CaCo-2 | 0.2 |
| Colon cancer tissue | 0.8 |
| Colon ca. SW1116 | 0.0 |
| Colon ca. Colo-205 | 0.3 |
| Colon ca. SW-48 | 1.4 |
| Colon Pool | 0.1 |
| Small Intestine Pool | 1.0 |
| Stomach Pool | 0.2 |
| Bone Marrow Pool | 3.3 |
| Fetal Heart | 0.0 |
| Heart Pool | 0.0 |
| Lymph Node Pool | 0.1 |
| Fetal Skeletal Muscle | 0.0 |
| Skeletal Muscle Pool | 0.2 |
| Spleen Pool | 0.0 |
| Thymus Pool | 0.3 |
| CNS cancer (glio/astro) U87-MG | 0.0 |
| CNS cancer (glio/astro) U-118-MG | 0.1 |
| CNS cancer (neuro; met) SK-N-AS | 0.0 |
| CNS cancer (astro) SF-539 | 0.0 |
| CNS cancer (astro) SNB-75 | 2.0 |
| CNS cancer (glio) SNB-19 | 2.8 |
| CNS cancer (glio) SF-295 | 36.6 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.3 |
| Adrenal Gland | 0.1 |
| Pituitary gland Pool | 0.7 |
| Salivary Gland | 20.4 |
| Thyroid (female) | 0.1 |
| Pancreatic ca. CAPAN2 | 5.1 |
| Pancreas Pool | 2.4 |

Column A - Rel. Ex. (%) Ag588, Run 248445830

TABLE EE
Panel 2D

| Tissue Name | A |
|---|---|
| Normal Colon | 4.8 |
| CC Well to Mod Diff (ODO3866) | 1.3 |
| CC Margin (ODO3866) | 0.9 |
| CC Gr. 2 rectosigmoid (ODO3868) | 1.8 |
| CC Margin (ODO3868) | 0.0 |
| CC Mod Diff (ODO3920) | 3.1 |
| CC Margin (ODO3920) | 0.5 |
| CC Gr. 2 ascend colon (ODO3921) | 2.3 |
| CC Margin (ODO3921) | 0.4 |
| CC from Partial Hepatectomy (ODO4309) Mets | 1.8 |
| Liver Margin (ODO4309) | 2.4 |
| Colon mets to lung (OD04451-01) | 5.3 |
| Lung Margin (OD04451-02) | 32.8 |
| Normal Prostate 6546-1 | 5.0 |
| Prostate Cancer (OD04410) | 0.3 |
| Prostate Margin (OD04410) | 0.2 |
| Prostate Cancer (OD04720-01) | 0.7 |
| Prostate Margin (OD04720-02) | 1.8 |
| Normal Lung | 56.3 |
| Lung Met to Muscle (ODO4286) | 0.0 |
| Muscle Margin (ODO4286) | 24.5 |
| Lung Malignant Cancer (OD03126) | 42.0 |
| Lung Margin (OD03126) | 40.3 |
| Lung Cancer (OD04404) | 27.4 |
| Lung Margin (OD04404) | 42.6 |
| Lung Cancer (OD04565) | 13.7 |
| Lung Margin (OD04565) | 18.3 |
| Lung Cancer (OD04237-01) | 6.4 |
| Lung Margin (OD04237-02) | 12.8 |
| Ocular Mel Met to Liver (ODO4310) | 0.0 |
| Liver Margin (ODO4310) | 3.6 |
| Melanoma Metastasis | 0.4 |
| Lung Margin (OD04321) | 77.9 |
| Normal Kidney | 1.6 |
| Kidney Ca, Nuclear grade 2 (OD04338) | 3.3 |
| Kidney Margin (OD04338) | 3.0 |
| Kidney Ca Nuclear grade 1/2 (OD04339) | 6.7 |
| Kidney Margin (OD04339) | 0.7 |
| Kidney Ca, Clear cell type (OD04340) | 0.0 |
| Kidney Margin (OD04340) | 2.5 |
| Kidney Ca, Nuclear grade 3 (OD04348) | 7.1 |
| Kidney Margin (OD04348) | 1.8 |
| Kidney Cancer (OD04622-01) | 0.3 |
| Kidney Margin (OD04622-03) | 2.3 |
| Kidney Cancer (OD04450-01) | 9.2 |
| Kidney Margin (OD04450-03) | 1.5 |
| Kidney Cancer 8120607 | 33.2 |
| Kidney Margin 8120608 | 1.7 |
| Kidney Cancer 8120613 | 0.0 |
| Kidney Margin 8120614 | 0.9 |
| Kidney Cancer 9010320 | 27.4 |
| Kidney Margin 9010321 | 2.4 |
| Normal Uterus | 0.1 |
| Uterine Cancer 064011 | 63.3 |
| Normal Thyroid | 1.7 |
| Thyroid Cancer | 13.8 |
| Thyroid Cancer A302152 | 1.3 |
| Thyroid Margin A302153 | 0.5 |
| Normal Breast | 5.5 |
| Breast Cancer | 0.0 |
| Breast Cancer (OD04590-01) | 0.9 |
| Breast Cancer Mets (OD04590-03) | 0.7 |
| Breast Cancer Metastasis | 0.1 |
| Breast Cancer | 1.2 |
| Breast Cancer | 4.1 |
| Breast Cancer 9100266 | 1.7 |
| Breast Margin 9100265 | 1.6 |
| Breast Cancer A209073 | 12.9 |
| Breast Margin A209073 | 6.1 |
| Normal Liver | 1.0 |
| Liver Cancer | 14.4 |
| Liver Cancer 1025 | 2.5 |
| Liver Cancer 1026 | 4.2 |
| Liver Cancer 6004-T | 5.3 |
| Liver Tissue 6004-N | 0.1 |
| Liver Cancer 6005-T | 5.1 |
| Liver Tissue 6005-N | 1.4 |
| Normal Bladder | 2.7 |
| Bladder Cancer | 2.7 |
| Bladder Cancer | 8.2 |
| Bladder Cancer (OD04718-01) | 2.0 |
| Bladder Normal Adjacent (OD04718-03) | 0.9 |
| Normal Ovary | 0.6 |
| Ovarian Cancer | 100.0 |
| Ovarian Cancer (OD04768-07) | 21.9 |
| Ovary Margin (OD04768-08) | 4.1 |
| Normal Stomach | 2.3 |
| Gastric Cancer 9060358 | 0.5 |
| Stomach Margin 9060359 | 2.6 |
| Gastric Cancer 9060395 | 5.4 |
| Stomach Margin 9060394 | 4.9 |
| Gastric Cancer 9060397 | 14.1 |
| Stomach Margin 9060396 | 5.1 |
| Gastric Cancer 064005 | 0.2 |

Column A - Rel. Exp. (%) Ag588, Run 144773993

TABLE EF
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 1.4 |
| Secondary CD8 lymphocyte rest | 0.0 |
| Secondary CD8 lymphocyte act | 0.0 |
| CD4 lymphocyte none | 0.0 |
| 2ry Th1/Th2/Tr1 anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.2 |
| PBMC PHA-L | 0.0 |
| Ramos (B cell) none | 0.0 |
| Ramos (B cell) ionomycin | 0.0 |
| B lymphocytes PWM | 0.2 |
| B lymphocytes CD40L and IL-4 | 0.0 |
| EOL-1 dbcAMP | 0.2 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.1 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 3.7 |
| Small airway epithelium none | 53.6 |
| Small airway epithelium TNFalpha + IL-1beta | 100.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.9 |
| KU-812 (Basophil) rest | 0.0 |
| KU-812 (Basophil) PMA/ionomycin | 0.0 |
| CCD1106 (Keratinocytes) none | 0.7 |
| CCD1106 (Keratinocytes) TNFalpha + IL-1beta | 0.6 |
| Liver cirrhosis | 1.7 |
| Lupus kidney | 9.9 |
| NCI-H292 none | 49.0 |
| NCI-H292 IL-4 | 61.6 |
| NCI-H292 IL-9 | 83.5 |
| NCI-H292 IL-13 | 37.4 |
| NCI-H292 IFN gamma | 43.2 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.1 |
| Colon | 0.7 |
| Lung | 36.3 |
| Thymus | 1.4 |
| Kidney | 3.9 |

Column A - Rel. Exp. (%) Ag588, Run 163588119

TABLE EG
Panel 5D

| Tissue Name | A |
|---|---|
| 97457 Patient-02go adipose | 100.0 |
| 97476 Patient-07sk skeletal muscle | 7.8 |
| 97477 Patient-07ut uterus | 0.3 |
| 97478 Patient-07pl placenta | 1.8 |
| 97481 Patient-08sk skeletal muscle | 9.0 |
| 97482 Patient-08ut uterus | 0.4 |
| 97483 Patient-08pl placenta | 1.1 |
| 97486 Patient-09sk skeletal muscle | 7.6 |
| 97487 Patient-09ut uterus | 1.5 |
| 97488 Patient-09pl placenta | 0.4 |
| 97492 Patient-10ut uterus | 7.1 |
| 97493 Patient-10pl placenta | 0.3 |
| 97495 Patient-11go adipose | 63.3 |
| 97496 Patient-11sk skeletal muscle | 6.9 |
| 97497 Patient-11ut uterus | 1.0 |
| 97498 Patient-11pl placenta | 0.5 |
| 97500 Patient-12go adipose | 52.5 |
| 97501 Patient-12sk skeletal muscle | 3.1 |
| 97502 Patient-12ut uterus | 0.2 |
| 97503 Patient-12pl placenta | 0.1 |
| 94721 Donor 2 U - A Mesenchymal Stem Cells | 0.0 |
| 94722 Donor 2 U - B Mesenchymal Stem Cells | 0.1 |
| 94723 Donor 2 U - C Mesenchymal Stem Cells | 0.0 |
| 94709 Donor 2 AM - A adipose | 1.8 |
| 94710 Donor 2 AM - B adipose | 1.2 |
| 94711 Donor 2 AM - C adipose | 1.0 |
| 94712 Donor 2 AD - A adipose | 2.6 |
| 94713 Donor 2 AD - B adipose | 3.6 |
| 94714 Donor 2 AD - C adipose | 3.0 |
| 94742 Donor 3 U - A Mesenchymal Stem Cells | 0.0 |
| 94743 Donor 3 U - B Mesenchymal Stem Cells | 0.2 |
| 94730 Donor 3 AM - A adipose | 2.4 |
| 94731 Donor 3 AM - B adipose | 1.0 |
| 94732 Donor 3 AM - C adipose | 1.4 |
| 94733 Donor 3 AD - A adipose | 2.8 |
| 94734 Donor 3 AD - B adipose | 1.1 |
| 94735 Donor 3 AD - C adipose | 2.8 |
| 77138 Liver HepG2untreated | 0.1 |
| 73556 Heart Cardiac stromal cells (primary) | 0.0 |
| 81735 Small Intestine | 58.2 |
| 72409 Kidney Proximal Convoluted Tubule | 13.0 |
| 82685 Small intestine Duodenum | 0.2 |
| 90650 Adrenal Adrenocortical adenoma | 0.1 |
| 72410 Kidney HRCE | 15.4 |
| 72411 Kidney HRE | 3.9 |
| 73139 Uterus Uterine smooth muscle cells | 0.0 |

Column A - Rel. Exp. (%) Ag588, Run 248989995

Ardais Kidney 1.0 Summary: Ag588 High gene expression was detected in kidney cancer samples. The gene's expression is useful in differentiating kidney cancer tissue from normal kidney tissue. Therapeutic modulation of this gene, expressed protein and/or use of antibodies or small molecule drugs targeting the gene or gene product are useful in the treatment of kidney cancer.

CNS_neurodegeneration_v1.0 Summary: Ag588 Moderate expression levels of this gene were detected in brain in an independent group of individuals. This gene was slightly upregulated in the temporal cortex of Alzheimer's disease patients. The gene's expression is useful in differentiating temporal cortex tissue of Alzheimer's disease patients from normal temporal cortex tissue. Therapeutic modulation of this gene, expressed protein and/or use of antibodies or small molecule drugs targeting the gene or gene product are useful in the treatment of Alzheimer's disease.

General_screening_panel_v1.5 Summary: Ag588 Highest expression of this gene was seen in the trachea (CT=18). High levels of expression were also seen in ovarian, pancreatic, brain, colon, gastric, and squamous cell carcinoma cell lines. Therapeutic modulation of this gene, expressed protein and/or use of antibodies or small molecule drugs targeting the gene or gene product are useful in the treatment of ovarian, pancreatic, brain, colon, gastric, and squamous cell cancers.

Panel 2D Summary: Ag588 Highest expression was detected in an ovarian cancer sample (CTs=22). Gene overexpression was detected ovarian, uterine, thyroid and kidney cancer samples when compared to the expression in normal adjacent tissue. Gene expression is useful for differentiating these cancer samples from other samples on this panel and as a marker of these cancers. This gene encodes secretory leucocyte protease inhibitor (SLPI), a potent inhibitor of granulocyte elastase and cathepsin G, as well as pancreatic enzymes like trypsin, chymotrypsin and pancreatic elastase. SLPI has also been shown to inhibit HIV-1 infections by blocking viral DNA synthesis. Therapeutic modulation of this gene, expressed protein and/or use of antibodies or small molecule drugs targeting the gene or gene product are useful in the treatment of ovarian, uterine, thyroid, and kidney cancers.

Panel 4D Summary: Ag588 Highest gene expression was detected in TNF-a/IL1-b treated small airway epithelium. High gene expression were also seen in untreated small airway epithelium, normal lung, and a cluster of treated and untreated samples derived from the NCI-H292 cell line, a human airway epithelial cell line that produces mucins. Mucus overproduction is a feature of bronchial asthma and chronic obstructive pulmonary disease samples. The expression of this gene in the mucoepidermoid cell line NCI-H292 and in small airway epithelium indicates that this gene is involved in the proliferation or activation of airway epithelium. Therapeutic modulation of this gene, expressed protein and/or use of antibodies or small molecule drugs targeting the gene or gene product are useful in the treatment of symptoms caused by inflammation in lung epithelia in chronic obstructive pulmonary disease, asthma, allergy, and emphysema.

Panel 5D Summary: Ag588 Prominent expression of this gene was detected in adipose (CTs=26-27). Therapeutic modulation of this gene, expressed protein and/or use of antibodies or small molecule drugs targeting the gene or gene product are useful in the treatment of obesity and diabetes.

F. NOV 9, CG56008-01: LIV-1 Protein, Estrogen Regulated.

Expression of gene CG56008-01 was assessed using the primer-probe set Ag2169, described in Table FA. Results of the RTQ-PCR runs are shown in Tables FB, FC, FD, FE, FF, FG and FH.

TABLE FA
Probe Name Ag2169

| | | | Start | SEQ |
|---|---|---|---|---|
| Primers | Sequences | Length | Position | ID No |
| Forward | 5′-cccgaaaaggctttatgt | 22 | 856 | 173 |
| | attc-3′ | | | |
| Probe | TET-5′-cagaaacacaaatg | 27 | 878 | 174 |
| | aaaatcctcagga-3′- | | | |
| | TAMRA | | | |
| Reverse | 5′-tgtcagtagctttgatgc | 22 | 911 | 175 |
| | attg-3′ | | | |

TABLE FB
Ardais Panel 1.1

| Tissue Name | A |
|---|---|
| 136803 Lung cancer(368) | 35.4 |
| 136804 Lung cancer(369) | 62.0 |
| 136805 Lung NAT(36A) | 11.9 |
| 136787 lung cancer(356) | 1.6 |
| 136788 lung NAT(357) | 15.4 |
| 136806 Lung cancer(36B) | 19.8 |
| 136807 Lung NAT(36C) | 11.0 |
| 136810 Lung NAT(36F) | 37.1 |
| 136789 lung cancer(358) | 10.4 |
| 136802 Lung cancer(365) | 16.5 |
| 136811 Lung cancer(370) | 81.8 |
| 136791 Lung cancer(35A) | 11.7 |
| 136794 lung NAT(35D) | 15.5 |
| 136815 Lung cancer(374) | 5.4 |
| 136816 Lung NAT(375) | 42.9 |
| 136813 Lung cancer(372) | 100.0 |
| 136814 Lung NAT(373) | 2.9 |
| 136795 Lung cancer(35E) | 35.8 |
| 136797 Lung cancer(360) | 4.5 |
| 136799 Lung cancer(362) | 4.3 |
| 136800 Lung NAT(363) | 6.6 |

Column A - Rel. Exp. (%) Ag2169, Run 306368466

TABLE FC
Panel 1.3D

| Tissue Name | A | B |
|---|---|---|
| Liver adenocarcinoma | 1.8 | 2.0 |
| Pancreas | 1.0 | 0.4 |
| Pancreatic ca. CAPAN 2 | 1.0 | 1.0 |
| Adrenal gland | 0.8 | 0.6 |
| Thyroid | 2.0 | 0.9 |
| Salivary gland | 1.2 | 0.8 |
| Pituitary gland | 3.1 | 2.2 |
| Brain (fetal) | 2.2 | 1.7 |
| Brain (whole) | 2.6 | 2.1 |
| Brain (amygdala) | 2.0 | 1.1 |
| Brain (cerebellum) | 1.4 | 0.9 |
| Brain (hippocampus) | 6.1 | 4.5 |
| Brain (substantia nigra) | 0.5 | 0.8 |
| Brain (thalamus) | 2.5 | 2.0 |
| Cerebral Cortex | 2.8 | 3.1 |
| Spinal cord | 1.6 | 1.4 |
| glio/astro U87-MG | 1.2 | 0.8 |
| glio/astro U-118-MG | 12.0 | 9.3 |
| astrocytoma SW1783 | 2.8 | 3.0 |
| neuro*; met SK-N-AS | 10.7 | 6.7 |
| astrocytoma SF-539 | 1.7 | 1.5 |
| astrocytoma SNB-75 | 2.8 | 3.8 |
| glioma SNB-19 | 1.0 | 0.9 |
| glioma U251 | 0.8 | 0.8 |
| glioma SF-295 | 3.4 | 3.0 |
| Heart (Fetal) | 0.4 | 0.5 |
| Heart | 0.2 | 0.1 |
| Skeletal muscle (Fetal) | 1.2 | 1.4 |
| Skeletal muscle | 0.2 | 0.2 |
| Bone marrow | 0.4 | 0.2 |
| Thymus | 0.3 | 0.3 |
| Spleen | 1.1 | 0.8 |
| Lymph node | 0.8 | 0.5 |
| Colorectal | 0.3 | 0.2 |
| Stomach | 1.5 | 0.8 |
| Small intestine | 0.9 | 0.5 |
| Colon ca. SW480 | 1.6 | 1.2 |
| Colon ca.* SW620 (SW480 met) | 0.7 | 0.5 |
| Colon ca. HT29 | 0.8 | 0.6 |
| Colon ca. HCT-116 | 4.2 | 3.1 |
| Colon ca. CaCo-2 | 0.9 | 1.1 |
| CC Well to Mod Diff (ODO3866) | 1.3 | 1.2 |
| Colon ca. HCC-2998 | 2.1 | 1.6 |
| Gastric ca. (liver met) NCI-N87 | 2.0 | 1.6 |
| Bladder | 1.0 | 0.6 |
| Trachea | 1.6 | 1.6 |
| Kidney | 0.5 | 0.5 |
| Kidney (fetal) | 1.1 | 0.8 |
| Renal ca. 786-0 | 2.6 | 1.7 |
| Renal ca. A498 | 4.2 | 3.2 |
| Renal ca. RXF 393 | 1.2 | 0.8 |
| Renal ca. ACHN | 2.6 | 2.7 |
| Renal ca. UO-31 | 3.3 | 2.4 |
| Renal ca. TK-10 | 2.0 | 1.5 |
| Liver | 0.1 | 0.1 |
| Liver (fetal) | 0.5 | 0.3 |
| Liver ca. (hepatoblast) HepG2 | 1.5 | 1.3 |
| Lung | 0.8 | 0.6 |
| Lung (fetal) | 1.5 | 1.5 |
| Lung ca. (small cell) LX-1 | 1.0 | 0.7 |
| Lung ca. (small cell) NCI-H69 | 10.0 | 6.3 |
| Lung ca. (s. cell var.) SHP-77 | 3.9 | 4.9 |
| Lung ca. (large cell)NCI-H460 | 1.3 | 1.2 |
| Lung ca. (non-sm. cell) A549 | 0.9 | 0.6 |
| Lung ca. (non-s. cell) NCI-H23 | 5.4 | 0.0 |
| Lung ca. (non-s. cell) HOP-62 | 1.8 | 2.0 |
| Lung ca. (non-s. cl) NCI-H522 | 1.8 | 1.2 |
| Lung ca. (squam.) SW 900 | 1.2 | 0.8 |
| Lung ca. (squam.) NCI-H596 | 3.1 | 3.0 |
| Mammary gland | 11.7 | 10.4 |
| Breast ca.* (pl. ef) MCF-7 | 100.0 | 100.0 |
| Breast ca.* (pl. ef) MDA-MB-231 | 2.5 | 2.1 |
| Breast ca.* (pl. ef) T47D | 5.7 | 3.3 |
| Breast ca. BT-549 | 4.5 | 3.6 |
| Breast ca. MDA-N | 2.6 | 2.8 |
| Ovary | 2.0 | 1.3 |
| Ovarian ca. OVCAR-3 | 2.2 | 2.0 |
| Ovarian ca. OVCAR-4 | 0.3 | 0.2 |
| Ovarian ca. OVCAR-5 | 0.6 | 0.5 |
| Ovarian ca. OVCAR-8 | 1.6 | 0.9 |
| Ovarian ca. IGROV-1 | 0.8 | 0.5 |
| Ovarian ca. (ascites) SK-OV-3 | 4.0 | 3.2 |
| Uterus | 1.1 | 0.8 |
| Placenta | 3.4 | 2.1 |
| Prostate | 5.5 | 4.6 |
| Prostate ca.* (bone met) PC-3 | 2.0 | 1.3 |
| Testis | 1.9 | 1.6 |
| Melanoma Hs688(A).T | 4.8 | 4.8 |
| Melanoma* (met) Hs688(B).T | 6.2 | 5.2 |
| Melanoma UACC-62 | 0.3 | 0.3 |
| Melanoma M14 | 2.8 | 2.6 |
| Melanoma LOX IMVI | 6.6 | 0.4 |
| Melanoma* (met) SK-MEL-5 | 7.1 | 5.1 |
| Adipose | 1.2 | 0.8 |

Column A - Rel. Exp. (%) Ag2169, Run 149923246
Column B - Rel. Exp. (%) Ag2169, Run 151268473

TABLE FD
Panel 2.2

| Tissue Name | A |
|---|---|
| Normal Colon | 1.6 |
| Colon cancer (OD06064) | 4.8 |
| Colon Margin (OD06064) | 0.4 |
| Colon cancer (OD06159) | 0.2 |
| Colon Margin (OD06159) | 0.3 |
| Colon cancer (OD06297-04) | 0.4 |
| Colon Margin (OD06297-05) | 1.2 |
| CC Gr. 2 ascend colon (ODO3921) | 0.5 |
| CC Margin (ODO3921) | 0.4 |
| Colon cancer metastasis (OD06104) | 0.4 |
| Lung Margin (OD06104) | 0.5 |
| Colon mets to lung (OD04451-01) | 0.7 |
| Lung Margin (OD04451-02) | 1.1 |
| Normal Prostate | 4.5 |
| Prostate Cancer (OD04410) | 3.4 |
| Prostate Margin (OD04410) | 2.3 |
| Normal Ovary | 0.6 |
| Ovarian cancer (OD06283-03) | 0.4 |
| Ovarian Margin (OD06283-07) | 0.3 |
| Ovarian Cancer | 0.7 |
| Ovarian cancer (OD06145) | 0.7 |
| Ovarian Margin (OD06145) | 1.8 |
| Ovarian cancer (OD06455-03) | 1.2 |
| Ovarian Margin (OD06455-07) | 0.5 |
| Normal Lung | 0.6 |
| Invasive poor diff. lung adeno (ODO4945-01 | 1.1 |
| Lung Margin (ODO4945-03) | 0.5 |
| Lung Malignant Cancer (OD03126) | 0.9 |
| Lung Margin (OD03126) | 0.4 |
| Lung Cancer (OD05014A) | 0.7 |
| Lung Margin (OD05014B) | 1.4 |
| Lung cancer (OD06081) | 0.2 |
| Lung Margin (OD06081) | 0.2 |
| Lung Cancer (OD04237-01) | 0.9 |
| Lung Margin (OD04237-02) | 1.6 |
| Ocular Mel Met to Liver (ODO4310) | 3.5 |
| Liver Margin (ODO4310) | 0.4 |
| Melanoma Metastasis | 2.0 |
| Lung Margin (OD04321) | 1.2 |
| Normal Kidney | 0.6 |
| Kidney Ca, Nuclear grade 2 (OD04338) | 3.9 |
| Kidney Margin (OD04338) | 1.3 |
| Kidney Ca Nuclear grade 1/2 (OD04339) | 1.2 |
| Kidney Margin (OD04339) | 0.8 |
| Kidney Ca, Clear cell type (OD04340) | 0.8 |
| Kidney Margin (OD04340) | 1.4 |
| Kidney Ca, Nuclear grade 3 (OD04348) | 0.9 |
| Kidney Margin (OD04348) | 5.3 |
| Kidney malignant cancer (OD06204B) | 1.9 |
| Kidney normal adjacent tissue (OD06204E) | 1.2 |
| Kidney Cancer (OD04450-01) | 2.4 |
| Kidney Margin (OD04450-03) | 1.9 |
| Kidney Cancer 8120613 | 0.1 |
| Kidney Margin 8120614 | 0.3 |
| Kidney Cancer 9010320 | 0.5 |
| Kidney Margin 9010321 | 0.3 |
| Kidney Cancer 8120607 | 0.8 |
| Kidney Margin 8120608 | 0.2 |
| Normal Uterus | 1.4 |
| Uterine Cancer 064011 | 1.1 |
| Normal Thyroid | 0.2 |
| Thyroid Cancer | 0.6 |
| Thyroid Cancer A302152 | 1.6 |
| Thyroid Margin A302153 | 0.5 |
| Normal Breast | 7.6 |
| Breast Cancer | 7.6 |
| Breast Cancer | 0.0 |
| Breast Cancer (OD04590-01) | 30.6 |
| Breast Cancer Mets (OD04590-03) | 34.4 |
| Breast Cancer Metastasis | 100.0 |
| Breast Cancer | 2.3 |
| Breast Cancer 9100266 | 25.2 |
| Breast Margin 9100265 | 3.2 |
| Breast Cancer A209073 | 1.1 |
| Breast Margin A209073 | 5.1 |
| Breast cancer (OD06083) | 8.1 |
| Breast cancer node metastasis (OD06083) | 1.2 |
| Normal Liver | 0.5 |
| Liver Cancer 1026 | 0.1 |
| Liver Cancer 1025 | 0.8 |
| Liver Cancer 6004-T | 0.7 |
| Liver Tissue 6004-N | 0.2 |
| Liver Cancer 6005-T | 0.4 |
| Liver Tissue 6005-N | 0.4 |
| Liver Cancer | 0.7 |
| Normal Bladder | 0.7 |
| Bladder Cancer | 0.4 |
| Bladder Cancer | 1.5 |
| Normal Stomach | 1.3 |
| Gastric Cancer 9060397 | 0.2 |
| Stomach Margin 9060396 | 0.5 |
| Gastric Cancer 9060395 | 0.4 |
| Stomach Margin 9060394 | 1.1 |
| Gastric Cancer 064005 | 0.4 |

Column A - Rel. Exp. (%) Ag2169, Run 176282877

TABLE FE
Panel 2D

| Tissue Name | A |
|---|---|
| Normal Colon | 3.2 |
| CC Well to Mod Diff (ODO3866) | 0.6 |
| CC Margin (ODO3866) | 0.2 |
| CC Gr. 2 rectosigmoid (ODO3868) | 0.1 |
| CC Margin (ODO3868) | 0.2 |
| CC Mod Diff (ODO3920) | 0.2 |
| CC Margin (ODO3920) | 0.3 |
| CC Gr. 2 ascend colon (ODO3921) | 1.0 |
| CC Margin (ODO3921) | 0.3 |
| CC from Partial Hepatectomy (ODO4309) Mets | 1.6 |
| Liver Margin (ODO4309) | 0.5 |
| Colon mets to lung (OD04451-01) | 0.2 |
| Lung Margin (OD04451-02) | 0.4 |
| Normal Prostate 6546-1 | 7.7 |
| Prostate Cancer (OD04410) | 15.1 |
| Prostate Margin (OD04410) | 7.4 |
| Prostate Cancer (OD04720-01) | 3.4 |
| Prostate Margin (OD04720-02) | 6.7 |
| Normal Lung | 1.4 |
| Lung Met to Muscle (ODO4286) | 1.4 |
| Muscle Margin (ODO4286) | 0.7 |
| Lung Malignant Cancer (OD03126) | 1.7 |
| Lung Margin (OD03126) | 1.1 |
| Lung Cancer (OD04404) | 2.0 |
| Lung Margin (OD04404) | 1.0 |
| Lung Cancer (OD04565) | 1.0 |
| Lung Margin (OD04565) | 0.5 |
| Lung Cancer (OD04237-01) | 3.1 |
| Lung Margin (OD04237-02) | 0.9 |
| Ocular Mel Met to Liver (ODO4310) | 3.7 |
| Liver Margin (ODO4310) | 0.2 |
| Melanoma Metastasis | 3.5 |
| Lung Margin (OD04321) | 0.9 |
| Normal Kidney | 2.5 |
| Kidney Ca, Nuclear grade 2 (OD04338) | 2.8 |
| Kidney Margin (OD04338) | 1.8 |
| Kidney Ca Nuclear grade 1/2 (OD04339) | 0.7 |
| Kidney Margin (OD04339) | 1.4 |
| Kidney Ca, Clear cell type (OD04340) | 2.5 |
| Kidney Margin (OD04340) | 1.8 |
| Kidney Ca, Nuclear grade 3 (OD04348) | 1.0 |
| Kidney Margin (OD04348) | 1.5 |
| Kidney Cancer (OD04622-01) | 0.9 |
| Kidney Margin (OD04622-03) | 0.2 |
| Kidney Cancer (OD04450-01) | 1.1 |
| Kidney Margin (OD04450-03) | 1.4 |
| Kidney Cancer 8120607 | 0.5 |
| Kidney Margin 8120608 | 0.3 |
| Kidney Cancer 8120613 | 0.4 |
| Kidney Margin 8120614 | 0.2 |
| Kidney Cancer 9010320 | 0.8 |
| Kidney Margin 9010321 | 0.5 |
| Normal Uterus | 0.0 |
| Uterine Cancer 064011 | 1.8 |
| Normal Thyroid | 1.4 |
| Thyroid Cancer | 1.7 |
| Thyroid Cancer A302152 | 0.9 |
| Thyroid Margin A302153 | 1.5 |
| Normal Breast | 3.9 |
| Breast Cancer | 19.8 |
| Breast Cancer (OD04590-01) | 46.7 |
| Breast Cancer Mets (OD04590-03) | 43.2 |
| Breast Cancer Metastasis | 100.0 |
| Breast Cancer | 2.4 |
| Breast Cancer | 2.5 |
| Breast Cancer 9100266 | 41.2 |
| Breast Margin 9100265 | 5.0 |
| Breast Cancer A209073 | 4.0 |
| Breast Margin A209073 | 4.1 |
| Normal Liver | 0.2 |
| Liver Cancer | 0.2 |
| Liver Cancer 1025 | 0.2 |
| Liver Cancer 1026 | 0.3 |
| Liver Cancer 6004-T | 0.2 |
| Liver Tissue 6004-N | 0.5 |
| Liver Cancer 6005-T | 0.2 |
| Liver Tissue 6005-N | 0.1 |
| Normal Bladder | 1.5 |
| Bladder Cancer | 0.3 |
| Bladder Cancer | 1.7 |
| Bladder Cancer (OD04718-01) | 3.0 |
| Bladder Normal Adjacent (OD04718-03) | 2.9 |
| Normal Ovary | 0.3 |
| Ovarian Cancer | 3.3 |
| Ovarian Cancer (OD04768-07) | 3.1 |
| Ovary Margin (OD04768-08) | 0.4 |
| Normal Stomach | 0.5 |
| Gastric Cancer 9060358 | 0.2 |
| Stomach Margin 9060359 | 0.4 |
| Gastric Cancer 9060395 | 0.8 |
| Stomach Margin 9060394 | 0.5 |
| Gastric Cancer 9060397 | 1.0 |
| Stomach Margin 9060396 | 0.1 |
| Gastric Cancer 064005 | 1.0 |

Column A - Rel. Exp. (%) Ag2169, Run 148722818

TABLE FF
Panel 3D

| Tissue Name | A |
|---|---|
| 94905 Daoy Medulloblastoma/Cerebellum | 3.2 |
| 94906 TE671 Medulloblastom/Cerebellum | 1.2 |
| 94907 D283 Med Medulloblastoma/Cerebellum | 19.2 |
| 94908 PFSK-1 Primitive Neuroectodermal/Cerebellum | 16.4 |
| 94909 XF-498 CNS | 15.5 |
| 94910 SNB-78 CNS/glioma | 20.3 |
| 94911 SF-268 CNS/glioblastoma | 2.5 |
| 94912 T98G Glioblastoma | 5.4 |
| 96776 SK-N-SH Neuroblastoma (metastasis) | 16.5 |
| 94913 SF-295 CNS/glioblastoma | 7.2 |
| 94914 Cerebellum | 6.1 |
| 96777 Cerebellum | 2.5 |
| 94916 NCI-H292 Mucoepidermoid lung carcinoma | 32.8 |
| 94917 DMS-114 Small cell lung cancer | 9.1 |
| 94918 DMS-79 Small cell lung cancer/neuroendocrine | 100.0 |
| 94919 NCI-H146 Small cell lung cancer/neuroendocrine | 31.6 |
| 94920 NCI-H526 Small cell lung cancer/neuroendocrine | 25.0 |
| 94921 NCI-N417 Small cell lung cancer/neuroendocrine | 5.0 |
| 94923 NCI-H82 Small cell lung cancer/neuroendocrine | 10.1 |
| 94924 NCI-H157 Squamous cell lung cancer (metastasis) | 12.9 |
| 94925 NCI-H1155 Large cell lung cancer/neuroendocrine | 17.7 |
| 94926 NCI-H1299 Large cell lung cancer/neuroendocrine | 15.0 |
| 94927 NCI-H727 Lung carcinoid | 4.0 |
| 94928 NCI-UMC-11 Lung carcinoid | 21.3 |
| 94929 LX-1 Small cell lung cancer | 6.1 |
| 94930 Colo-205 Colon cancer | 3.9 |
| 94931 KM12 Colon cancer | 6.1 |
| 94932 KM20L2 Colon cancer | 3.5 |
| 94933 NCI-H716 Colon cancer | 8.8 |
| 94935 SW-48 Colon adenocarcinoma | 4.2 |
| 94936 SW1116 Colon adenocarcinoma | 6.3 |
| 94937 LS 174T Colon adenocarcinoma | 3.4 |
| 94938 SW-948 Colon adenocarcinoma | 0.8 |
| 94939 SW-480 Colon adenocarcinoma | 3.2 |
| 94940 NCI-SNU-5 Gastric carcinoma | 1.4 |
| KATO III- Gastric carcinoma | 11.0 |
| 94943 NCI-SNU-16 Gastric carcinoma | 7.2 |
| 94944 NCI-SNU-1 Gastric carcinoma | 9.2 |
| 94946 RF-1 Gastric adenocarcinoma | 6.1 |
| 94947 RF-48 Gastric adenocarcinoma | 9.5 |
| 96778 MKN-45 Gastric carcinoma | 12.8 |
| 94949 NCI-N87 Gastric carcinoma | 4.4 |
| 94951 OVCAR-5 Ovarian carcinoma | 0.5 |
| 94952 RL95-2 Uterine carcinoma | 5.4 |
| 94953 HelaS3 Cervical adenocarcinoma | 11.7 |
| 94954 Ca Ski Cervical epidermoid carcinoma (metastasis | 11.6 |
| 94955 ES-2 Ovarian clear cell carcinoma | 4.4 |
| 94957 Ramos Stimulated with PMA/ionomycin 6 h | 5.0 |
| 94958 Ramos Stimulated with PMA/ionomycin 14 h | 6.2 |
| 94962 MEG-01 Chronic myelogenous leukemia (megokaryoblast) | 3.3 |
| 94963 Raji Burkitt's lymphoma | 1.2 |
| 94964 Daudi Burkitt's lymphoma | 4.6 |
| 94965 U266 B-cell plasmacytoma/myeloma | 11.4 |
| 94968 CA46 Burkitt's lymphoma | 2.1 |
| 94970 RL non-Hodgkin's B-cell lymphoma | 0.6 |
| 94972 JM1 pre-B-cell lymphoma/leukemia | 3.0 |
| 94973 Jurkat T cell leukemia | 11.7 |
| 94974 TF-1 Erythroleukemia | 2.9 |
| 94975 HUT 78 T-cell lymphoma | 2.9 |
| 94977 U937 Histiocytic lymphoma | 4.2 |
| 94980 KU-812 Myelogenous leukemia | 1.3 |
| 769-P- Clear cell renal carcinoma | 11.3 |
| 94983 Caki-2 Clear cell renal carcinoma | 8.0 |
| 94984 SW 839 Clear cell renal carcinoma | 2.6 |
| 94986 G401 Wilms' tumor | 4.1 |
| 94987 Hs766T Pancreatic carcinoma (LN metastasis) | 12.3 |
| 94988 CAPAN-1 Pancreatic adenocarcinoma (liver metastasis) | 2.2 |
| 94989 SU86.86 Pancreatic carcinoma (liver metastasis) | 3.2 |
| 94990 BxPC-3 Pancreatic adenocarcinoma | 4.8 |
| 94991 HPAC Pancreatic adenocarcinoma | 10.4 |
| 94992 MIA PaCa-2 Pancreatic carcinoma | 3.4 |
| 94993 CFPAC-1 Pancreatic ductal adenocarcinoma | 22.1 |
| 94994 PANC-1 Pancreatic epithelioid ductal carcinoma | 14.1 |
| 94996 T24 Bladder carcinma (transitional cell | 10.7 |
| 5637- Bladder carcinoma | 11.8 |
| 94998 HT-1197 Bladder carcinoma | 4.2 |
| 94999 UM-UC-3 Bladder carcinma (transitional cell) | 2.5 |
| 95000 A204 Rhabdomyosarcoma | 4.3 |
| 95001 HT-1080 Fibrosarcoma | 15.3 |
| 95002 MG-63 Osteosarcoma (bone) | 3.5 |
| 95003 SK-LMS-1 Leiomyosarcoma (vulva) | 10.2 |
| 95004 SJRH30 Rhabdomyosarcoma (met to bone marrow) | 3.1 |
| 95005 A431 Epidermoid carcinoma | 4.8 |
| 95007 WM266-4 Melanoma | 11.0 |
| DU 145- Prostate carcinoma (brain metastasis) | 0.0 |
| 95012 MDA-MB-468 Breast adenocarcinoma | 5.5 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| 95017 CAL 27 Squamous cell carcinoma of tongue | 7.3 |

Column A - Rel. Exp. (%) Ag2169, Run 170745433

TABLE FG
Panel 4D

| Tissue Name | A |
|---|---|
| Secondary Th1 act | 12.9 |
| Secondary Th2 act | 15.3 |
| Secondary Tr1 act | 17.6 |
| Secondary Th1 rest | 2.2 |
| Secondary Th2 rest | 2.9 |
| Secondary Tr1 rest | 3.7 |
| Primary Th1 act | 18.7 |
| Primary Th2 act | 23.8 |
| Primary Tr1 act | 24.3 |
| Primary Th1 rest | 17.4 |
| Primary Th2 rest | 6.0 |
| Primary Tr1 rest | 6.2 |
| CD45RA CD4 lymphocyte act | 12.9 |
| CD45RO CD4 lymphocyte act | 21.2 |
| CD8 lymphocyte act | 8.9 |
| Secondary CD8 lymphocyte rest | 9.5 |
| Secondary CD8 lymphocyte act | 5.4 |
| CD4 lymphocyte none | 1.6 |
| 2ry Th1/Th2/Tr1 anti-CD95 CH11 | 3.8 |
| LAK cells rest | 8.4 |
| LAK cells IL-2 | 8.2 |
| LAK cells IL-2 + IL-12 | 13.3 |
| LAK cells IL-2 + IFN gamma | 17.1 |
| LAK cells IL-2 + IL-18 | 14.7 |
| LAK cells PMA/ionomycin | 9.2 |
| NK Cells IL-2 rest | 7.0 |
| Two Way MLR 3 day | 7.3 |
| Two Way MLR 5 day | 7.3 |
| Two Way MLR 7 day | 6.2 |
| PBMC rest | 1.9 |
| PBMC PWM | 41.2 |
| PBMC PHA-L | 14.8 |
| Ramos (B cell) none | 9.7 |
| Ramos (B cell) ionomycin | 47.6 |
| B lymphocytes PWM | 71.2 |
| B lymphocytes CD40L and IL-4 | 9.1 |
| EOL-1 dbcAMP | 9.8 |
| EOL-1 dbcAMP PMA/ionomycin | 7.2 |
| Dendritic cells none | 9.6 |
| Dendritic cells LPS | 18.3 |
| Dendritic cells anti-CD40 | 12.2 |
| Monocytes rest | 5.7 |
| Monocytes LPS | 8.0 |
| Macrophages rest | 12.3 |
| Macrophages LPS | 4.8 |
| HUVEC none | 3.9 |
| HUVEC starved | 8.8 |
| HUVEC IL-1beta | 4.1 |
| HUVEC IFN gamma | 3.5 |
| HUVEC TNF alpha + IFN gamma | 9.3 |
| HUVEC TNF alpha + IL4 | 4.8 |
| HUVEC IL-11 | 1.2 |
| Lung Microvascular EC none | 4.2 |
| Lung Microvascular EC TNFalpha + IL-1beta | 7.3 |
| Microvascular Dermal EC none | 4.3 |
| Microsvasular Dermal EC TNFalpha + IL-1beta | 7.0 |
| Bronchial epithelium TNFalpha + IL1beta | 24.1 |
| Small airway epithelium none | 15.7 |
| Small airway epithelium TNFalpha + IL-1beta | 100.0 |
| Coronery artery SMC rest | 18.9 |
| Coronery artery SMC TNFalpha + IL-1beta | 13.9 |
| Astrocytes rest | 16.7 |
| Astrocytes TNFalpha + IL-1beta | 15.2 |
| KU-812 (Basophil) rest | 1.1 |
| KU-812 (Basophil) PMA/ionomycin | 5.5 |
| CCD1106 (Keratinocytes) none | 14.8 |
| CCD1106 (Keratinocytes) TNFalpha + IL-1beta | 2.9 |
| Liver cirrhosis | 0.9 |
| Lupus kidney | 1.5 |
| NCI-H292 none | 30.8 |
| NCI-H292 IL-4 | 40.6 |
| NCI-H292 IL-9 | 35.8 |
| NCI-H292 IL-13 | 17.7 |
| NCI-H292 IFN gamma | 23.8 |
| HPAEC none | 2.0 |
| HPAEC TNF alpha + IL-1 beta | 9.6 |
| Lung fibroblast none | 15.2 |
| Lung fibroblast TNF alpha + IL-1 beta | 15.3 |
| Lung fibroblast IL-4 | 37.4 |
| Lung fibroblast IL-9 | 23.2 |
| Lung fibroblast IL-13 | 23.5 |
| Lung fibroblast IFN gamma | 38.7 |
| Dermal fibroblast CCD1070 rest | 36.3 |
| Dermal fibroblast CCD1070 TNF alpha | 46.3 |
| Dermal fibroblast CCD1070 IL-1 beta | 18.6 |
| Dermal fibroblast IFN gamma | 14.5 |
| Dermal fibroblast IL-4 | 29.9 |
| IBD Colitis 2 | 0.2 |
| IBD Crohn's | 0.5 |
| Colon | 4.9 |
| Lung | 8.1 |
| Thymus | 14.8 |
| Kidney | 7.1 |

Column A - Rel. Exp. (%) Ag2169, Run 148725333

TABLE FH
general oncology screening panel v 2.4

| Tissue Name | A |
|---|---|
| Colon cancer 1 | 10.3 |
| CC Margin (ODO3921) | 5.6 |
| Colon cancer 2 | 34.4 |
| Colon NAT 2 | 4.7 |
| Colon cancer 3 | 31.2 |
| Colon NAT 3 | 9.2 |
| Colon malignant cancer 4 | 44.4 |
| Colon NAT 4 | 2.8 |
| Lung cancer 1 | 7.5 |
| Lung NAT 1 | 1.8 |
| Lung cancer 2 | 39.2 |
| Lung NAT 2 | 2.0 |
| Squamous cell carcinoma 3 | 27.9 |
| Lung NAT 3 | 0.5 |
| Metastatic melanoma 1 | 33.7 |
| Melanoma 2 | 4.2 |
| Melanoma 3 | 6.3 |
| Metastatic melanoma 4 | 56.6 |
| Metastatic melanoma 5 | 58.6 |
| Bladder cancer 1 | 2.8 |
| Bladder NAT 1 | 0.0 |
| Bladder cancer 2 | 11.7 |
| Bladder NAT 2 | 0.6 |
| Bladder NAT 3 | 1.3 |
| Bladder NAT 4 | 3.2 |
| Prostate adenocarcinoma 1 | 43.5 |
| Prostate adenocarcinoma 2 | 8.4 |
| Adenocarcinoma of the prostate | 100.0 |
| Prostate adenocarcinoma 4 | 9.7 |
| Prostate NAT 5 | 20.3 |
| Prostate adenocarcinoma 6 | 33.7 |
| Prostate adenocarcinoma 7 | 24.7 |
| Prostate adenocarcinoma 8 | 7.4 |
| Prostate adenocarcinoma 9 | 70.7 |
| Prostate NAT 10 | 11.1 |
| Kidney cancer 1 | 9.2 |
| Kidney NAT 1 | 5.7 |
| Kidney cancer 2 | 27.7 |
| Kidney NAT 2 | 19.3 |
| Kidney cancer 3 | 5.6 |
| Kidney NAT 3 | 5.6 |
| Kidney cancer 4 | 14.4 |
| Kidney NAT 4 | 7.5 |

Column A - Rel. Exp. (%) Ag219, Run 258707952

Ardais Panel 1.1 Summary: Ag2169 Highest gene expression was detected in a lung cancer samples (CT=24.2). Thus, expression of this gene can be used to differentiate between lung cancer tissue and normal lung tissue and as a marker of lung cancer. Therapeutic modulation of this gene, expressed protein and/or use of antibodies or small molecule drugs targeting the gene or gene product are useful in the treatment of lung cancer

Panel 1.3D Summary: Ag2169 The expression of this gene was highest in a sample of breast cancer cell line (MCF-7)(CTs=26). Therapeutic modulation of this gene, through the use of small molecule drugs, antibodies or protein therapeutics is useful in the treatment of breast cancer. Differential expression of this gene can be used to differentiate between breast cancer cells and normal breast cells. This gene was moderately expressed in a variety of metabolic tissues, including pancreas, adrenal, thyroid, pituitary, adult and fetal heart, fetal liver and adipose. As a zinc transporter, this gene is a potential target for the enhancement of insulin secretion and sensitivity in Type 2 diabetes. Therapeutic modulation of this gene, expressed protein and/or use of antibodies or small molecule drugs targeting the gene or gene product are useful in the treatment of metabolic and endocrine disease, including obesity and Types 1 and 2 diabetes. This gene is differentially expressed in fetal (CTs=31-32) vs adult skeletal muscle (CTs=34-35). The relative overexpression of this gene in fetal skeletal muscle suggests that the protein product may enhance muscular growth or development in the fetus and thus may also act in a regenerative capacity in the adult. Therapeutic modulation of this gene, expressed protein and/or use of antibodies or small molecule drugs targeting the gene or gene product are useful for restoring muscle mass or function in weak or dystrophic muscle. Among tissues of CNS origin, gene expression was moderate in all regions examined. This gene, a LIV-1 homolog, is involved in zinc homeostasis. Zinc is critical to brain functions as it serves as an endogenous neuromodulator in synaptic neurotransmission. Therapeutic modulation of this gene, expressed protein and/or use of antibodies or small molecule drugs targeting the gene or gene product are useful in the treatment of learning deficiencies and seizure disorders associated with improper zinc trafficking.

Panel 2.2 and 2D Summary: Ag2169 Gene expression was detected in breast cancer, while expression of this gene in other tissues was almost absent with the exception of prostate derived samples. Gene expression is useful distinguish breast cancer samples from the other samples in the panel. Therapeutic modulation of this gene, expressed protein and/or use of antibodies or small molecule drugs targeting the gene or gene product are useful in the treatment of breast cancer.

Panel 3D Summary: Ag2169 The expression of this gene was highest in a sample derived from a lung cancer cell line (DMS 79)(CT=27.8). There were significant levels of expression in other lung cancer cell lines. The expression of this gene can be used to distinguish DMS 79 cells from other samples in the panel. Therapeutic modulation of this gene, expressed protein and/or use of antibodies or small molecule drugs targeting the gene or gene product are useful in the treatment of lung cancer.

Panel 4D Summary: Ag2169 The highest expression of this gene was seen in small airway epithelium stimulated with TNF-alpha and IL-1 beta (CTs=27). Moderate expression levels were also seen in pokeweed mitogen-activated peripheral blood mononuclear cells (mainly B cells), ionomycin-activated Ramos B cell, pokeweed mitogen-activated purified peripheral blood B lymphocytes, B lymphocytes activated with CD40L and IL-4, and a number of cytokine-activated and resting cells including NCI-H292 pulmonary mucoepidermoid epithelial cells, lung fibroblasts, and dermal fibroblasts. Based expression in cytokine-activated B cells and cells in lung and skin, therapeutic modulation of this gene, expressed protein and/or use of antibodies or small molecule drugs targeting the gene or gene product are useful in the treatment of autoimmune and inflammatory diseases in which activated B cells present antigens generating aberrant immune responses, such as, but not limited to Crohn's disease, ulcerative colitis, multiple sclerosis, chronic obstructive pulmonary disease, asthma, emphysema, rheumatoid arthritis, or psoriasis.

general oncology screening panel_V_{—}2.4 Summary: Ag2169 High gene expression was seen in a prostate cancer, with prominent expression seen in melanoma (CT=28.7) and in colon cancer but not adjacent normal colon tissue. Expression of this gene is useful to differentiate colon cancer from normal colon tissue. Therapeutic modulation of this gene, expressed protein and/or use of antibodies or small molecule drugs targeting the gene or gene product are useful in the treatment of prostate, colon cancer and melanoma.

G. NOV10, CG59356-01: Nuclear Receptor Subfamily 4.

Expression of gene CG59356-01 was assessed using the primer-probe set Ag3554, described in TableGA. Results of the RTQ-PCR runs are shown in Tables GB, GC, GD, GE and GF.

TABLE GA
Probe Name Ag3554

| | | | Start | SEQ |
|---|---|---|---|---|
| Primers | Sequences | Length | Position | ID No |
| Forward | 5′-atacacagacgcgctcac | 19 | 104 | 176 |
| | a-3′ | | | |
| Probe | TET-5′-ctccctcactcgaa | 26 | 127 | 177 |
| | cacacagacaca-3′-TAMRA | | | |
| Reverse | 5′-ggagagcgaagtgtgtgt | 20 | 173 | 178 |
| | gt-3′ | | | |

TABLE GB
AI comprehensive panel v1.0

| Tissue Name | A |
|---|---|
| 110967 COPD-F | 2.0 |
| 110980 COPD-F | 1.0 |
| 110968 COPD-M | 2.2 |
| 110977 COPD-M | 13.7 |
| 110989 Emphysema-F | 0.9 |
| 110992 Emphysema-F | 0.0 |
| 110993 Emphysema-F | 0.1 |
| 110994 Emphysema-F | 0.2 |
| 110995 Emphysema-F | 0.0 |
| 110996 Emphysema-F | 0.0 |
| 110997 Asthma-M | 0.0 |
| 111001 Asthma-F | 0.1 |
| 111002 Asthma-F | 0.3 |
| 111003 Atopic Asthma-F | 0.6 |
| 111004 Atopic Asthma-F | 3.6 |
| 111005 Atopic Asthma-F | 0.2 |
| 111006 Atopic Asthma-F | 0.0 |
| 111417 Allergy-M | 0.2 |
| 112347 Allergy-M | 0.0 |
| 112349 Normal Lung-F | 0.0 |
| 112357 Normal Lung-F | 100.0 |
| 112354 Normal Lung-M | 34.4 |
| 112374 Crohns-F | 0.3 |
| 112389 Match Control Crohns-F | 1.0 |
| 112375 Crohns-F | 0.1 |
| 112732 Match Control Crohns-F | 0.0 |
| 112725 Crohns-M | 0.0 |
| 112387 Match Control Crohns-M | 0.7 |
| 112378 Crohns-M | 0.0 |
| 112390 Match Control Crohns-M | 0.3 |
| 112726 Crohns-M | 28.7 |
| 112731 Match Control Crohns-M | 7.0 |
| 112380 Ulcer Col-F | 1.5 |
| 112734 Match Control Ulcer Col-F | 9.3 |
| 112384 Ulcer Col-F | 23.0 |
| 112737 Match Control Ulcer Col-F | 6.7 |
| 112386 Ulcer Col-F | 0.0 |
| 112738 Match Control Ulcer Col-F | 1.5 |
| 112381 Ulcer Col-M | 0.0 |
| 112735 Match Control Ulcer Col-M | 0.0 |
| 112382 Ulcer Col-M | 2.3 |
| 112394 Match Control Ulcer Col-M | 0.0 |
| 112383 Ulcer Col-M | 17.8 |
| 112736 Match Control Ulcer Col-M | 0.1 |
| 112423 Psoriasis-F | 29.5 |
| 112427 Match Control Psoriasis-F | 13.9 |
| 112418 Psoriasis-M | 1.7 |
| 112723 Match Control Psoriasis-M | 0.0 |
| 112419 Psoriasis-M | 3.9 |
| 112424 Match Control Psoriasis-M | 1.1 |
| 112420 Psoriasis-M | 0.3 |
| 112425 Match Control Psoriasis-M | 1.4 |
| 104689 (MF) OA Bone-Backus | 0.0 |
| 104690 (MF) Adj “Normal” Bone-Backus | 0.0 |
| 104691 (MF) OA Synovium-Backus | 0.0 |
| 104692 (BA) OA Cartilage-Backus | 0.0 |
| 104694 (BA) OA Bone-Backus | 0.0 |
| 104695 (BA) Adj “Normal” Bone-Backus | 0.0 |
| 104696 (BA) OA Synovium-Backus | 0.9 |
| 104700 (SS) OA Bone-Backus | 0.0 |
| 104701 (SS) Adj “Normal” Bone-Backus | 0.1 |
| 104702 (SS) OA Synovium-Backus | 0.6 |
| 117093 OA Cartilage Rep7 | 0.2 |
| 112672 OA Bone5 | 1.0 |
| 112673 OA Synovium5 | 0.0 |
| 112674 OA Synovial Fluid cells5 | 0.0 |
| 117100 OA Cartilage Rep14 | 0.0 |
| 112756 OA Bone9 | 0.0 |
| 112757 OA Synovium9 | 0.0 |
| 112758 OA Synovial Fluid Cells9 | 0.0 |
| 117125 RA Cartilage Rep2 | 8.2 |
| 113492 Bone2 RA | 27.4 |
| 113493 Synovium2 RA | 24.7 |
| 113494 Syn Fluid Cells RA | 41.5 |
| 113499 Cartilage4 RA | 31.9 |
| 113500 Bone4 RA | 40.9 |
| 113501 Synovium4 RA | 22.2 |
| 113502 Syn Fluid Cells4 RA | 13.4 |
| 113495 Cartilage3 RA | 32.1 |
| 113496 Bone3 RA | 40.3 |
| 113497 Synovium3 RA | 34.2 |
| 113498 Syn Fluid Cells3 RA | 39.0 |
| 117106 Normal Cartilage Rep20 | 0.0 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.1 |
| 113667 Bone4 Normal | 7.1 |
| 113668 Synovium4 Normal | 7.6 |
| 113669 Syn Fluid Cells4 Normal | 11.2 |

Column A - Rel. Ex. (%) Ag3554, Run 244570378

TABLE GC
General screening panel v1.4

| Tissue Name | A |
|---|---|
| Adipose | 22.7 |
| Melanoma* Hs688(A).T | 0.4 |
| Melanoma* Hs688(B).T | 0.8 |
| Melanoma* M14 | 3.1 |
| Melanoma* LOXIMVI | 0.1 |
| Melanoma* SK-MEL-5 | 42.9 |
| Squamous cell carcinoma SCC-4 | 0.0 |
| Testis Pool | 1.6 |
| Prostate ca.* (bone met) PC-3 | 0.0 |
| Prostate Pool | 4.5 |
| Placenta | 0.7 |
| Uterus Pool | 0.3 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.1 |
| Ovarian ca. OVCAR-8 | 0.0 |
| Ovary | 9.7 |
| Breast ca. MCF-7 | 0.3 |
| Breast ca. MDA-MB-231 | 0.4 |
| Breast ca. BT 549 | 10.4 |
| Breast ca. T47D | 0.3 |
| Breast ca. MDA-N | 1.9 |
| Breast Pool | 5.1 |
| Trachea | 12.4 |
| Lung | 13.3 |
| Fetal Lung | 100.0 |
| Lung ca. NCI-N417 | 0.0 |
| Lung ca. LX-1 | 0.0 |
| Lung ca. NCI-H146 | 0.0 |
| Lung ca. SHP-77 | 1.0 |
| Lung ca. A549 | 2.0 |
| Lung ca. NCI-H526 | 0.1 |
| Lung ca. NCI-H23 | 6.1 |
| Lung ca. NCI-H460 | 6.1 |
| Lung ca. HOP-62 | 0.0 |
| Lung ca. NCI-H522 | 0.1 |
| Liver | 0.1 |
| Fetal Liver | 0.0 |
| Liver ca. HepG2 | 0.0 |
| Kidney Pool | 10.5 |
| Fetal Kidney | 0.7 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.3 |
| Bladder | 2.7 |
| Gastric ca. (liver met.) NCI-N87 | 0.8 |
| Gastric ca. KATO III | 0.1 |
| Colon ca. SW-948 | 0.0 |
| Colon ca. SW480 | 0.1 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 27.5 |
| Colon ca. SW1116 | 0.0 |
| Colon ca. Colo-205 | 0.0 |
| Colon ca. SW-48 | 0.0 |
| Colon Pool | 2.6 |
| Small Intestine Pool | 20.6 |
| Stomach Pool | 6.9 |
| Bone Marrow Pool | 0.5 |
| Fetal Heart | 18.2 |
| Heart Pool | 17.9 |
| Lymph Node Pool | 4.2 |
| Fetal Skeletal Muscle | 2.6 |
| Skeletal Muscle Pool | 59.9 |
| Spleen Pool | 37.6 |
| Thymus Pool | 1.9 |
| CNS cancer (glio/astro) U87-MG | 0.2 |
| CNS cancer (glio/astro) U-118-MG | 4.8 |
| CNS cancer (neuro; met) SK-N-AS | 0.0 |
| CNS cancer (astro) SF-539 | 0.0 |
| CNS cancer (astro) SNB-75 | 2.7 |
| CNS cancer (glio) SNB-19 | 0.1 |
| CNS cancer (glio) SF-295 | 0.0 |
| Brain (Amygdala) Pool | 3.6 |
| Brain (cerebellum) | 9.7 |
| Brain (fetal) | 5.0 |
| Brain (Hippocampus) Pool | 6.3 |
| Cerebral Cortex Pool | 2.0 |
| Brain (Substantia nigra) Pool | 8.5 |
| Brain (Thalamus) Pool | 4.8 |
| Brain (whole) | 3.5 |
| Spinal Cord Pool | 7.4 |
| Adrenal Gland | 39.2 |
| Pituitary gland Pool | 10.4 |
| Salivary Gland | 3.3 |
| Thyroid (female) | 26.4 |
| Pancreatic ca. CAPAN2 | 0.0 |
| Pancreas Pool | 1.1 |

Column A - Rel. Exp (%) Ag3554, Run 217049423

TABLE GD
Panel 4.1D

| Tissue Name | A |
|---|---|
| Secondary Th1 act | 8.4 |
| Secondary Th2 act | 29.3 |
| Secondary Tr1 act | 6.4 |
| Secondary Th1 rest | 0.0 |
| Secondary Th2 rest | 0.0 |
| Secondary Tr1 rest | 0.0 |
| Primary Th1 act | 0.0 |
| Primary Th2 act | 51.4 |
| Primary Tr1 act | 45.4 |
| Primary Th1 rest | 0.0 |
| Primary Th2 rest | 0.0 |
| Primary Tr1 rest | 0.0 |
| CD45RA CD4 lymphocyte act | 1.4 |
| CD45RO CD4 lymphocyte act | 6.5 |
| CD8 lymphocyte act | 0.0 |
| Secondary CD8 lymphocyte rest | 3.1 |
| Secondary CD8 lymphocyte act | 0.6 |
| CD4 lymphocyte none | 0.0 |
| 2ry Th1/Th2/Tr1 anti-CD95 CH11 | 0.0 |
| LAK cells rest | 1.6 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 100.0 |
| NK Cells IL-2 rest | 0.3 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.1 |
| PBMC PHA-L | 2.0 |
| Ramos (B cell) none | 0.0 |
| Ramos (B cell) ionomycin | 0.0 |
| B lymphocytes PWM | 11.2 |
| B lymphocytes CD40L and IL-4 | 11.7 |
| EOL-1 dbcAMP | 0.0 |
| EOL-1 dbcAMP PMA/ionomycin | 1.7 |
| Dendritic cells none | 9.7 |
| Dendritic cells LPS | 8.1 |
| Dendritic cells anti-CD40 | 2.4 |
| Monocytes rest | 0.0 |
| Monocytes LPS | 17.4 |
| Macrophages rest | 0.0 |
| Macrophages LPS | 6.0 |
| HUVEC none | 0.0 |
| HUVEC starved | 0.0 |
| HUVEC IL-1beta | 0.6 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 6.2 |
| NCI-H292 none | 0.0 |
| NCI-H292 IL-4 | 0.1 |
| NCI-H292 IL-9 | 0.3 |
| NCI-H292 IL-13 | 0.1 |
| NCI-H292 IFN gamma | 0.0 |
| HPAEC none | 0.0 |
| HPAEC TNF alpha + IL-1 beta | 1.0 |
| Lung fibroblast none | 2.2 |
| Lung fibroblast TNF alpha + IL-1 beta | 13.7 |
| Lung fibroblast IL-4 | 0.5 |
| Lung fibroblast IL-9 | 0.6 |
| Lung fibroblast IL-13 | 0.0 |
| Lung fibroblast IFN gamma | 24.1 |
| Dermal fibroblast CCD1070 rest | 0.0 |
| Dermal fibroblast CCD1070 TNF alpha | 1.5 |
| Dermal fibroblast CCD1070 IL-1 beta | 6.3 |
| Dermal fibroblast IFN gamma | 0.0 |
| Dermal fibroblast IL-4 | 0.0 |
| Dermal Fibroblasts rest | 0.0 |
| Neutrophils TNFa + LPS | 67.8 |
| Neutrophils rest | 0.0 |
| Colon | 0.0 |
| Lung | 0.0 |
| Thymus | 0.0 |
| Kidney | 0.1 |

Column A - Rel. Exp. (%) Ag3554, Run 244570242

| Tissue Name | A |
|---|---|
| 97457 Patient-02go adipose | 0.0 |
| 97476 Patient-07sk skeletal muscle | 11.2 |
| 97477 Patient-07ut uterus | 0.0 |
| 97478 Patient-07pl placenta | 0.0 |
| 99167 Bayer Patient 1 | 0.6 |
| 97482 Patient-08ut uterus | 0.0 |
| 97483 Patient-08pl placenta | 0.0 |
| 97486 Patient-09sk skeletal muscle | 0.0 |
| 97487 Patient-09ut uterus | 0.0 |
| 97488 Patient-09pl placenta | 0.0 |
| 97492 Patient-10ut uterus | 0.0 |
| 97493 Patient-10pl placenta | 0.0 |
| 97495 Patient-11go adipose | 24.0 |
| 97496 Patient-11sk skeletal muscle | 4.0 |
| 97497 Patient-11ut uterus | 0.0 |
| 97498 Patient-11pl placenta | 0.0 |
| 97500 Patient-12go adipose | 20.6 |
| 97501 Patient-12sk skeletal muscle | 5.8 |
| 97502 Patient-12ut uterus | 0.1 |
| 97503 Patient-12pl placenta | 0.0 |
| 94721 Donor 2 U - A Mesenchymal Stem Cells | 0.0 |
| 94722 Donor 2 U - B Mesenchymal Stem Cells | 0.0 |
| 94723 Donor 2 U - C Mesenchymal Stem Cells | 0.0 |
| 94709 Donor 2 AM - A adipose | 0.0 |
| 94710 Donor 2 AM - B adipose | 0.0 |
| 94711 Donor 2 AM - C adipose | 0.0 |
| 94712 Donor 2 AD - A adipose | 0.0 |
| 94713 Donor 2 AD - B adipose | 0.0 |
| 94714 Donor 2 AD - C adipose | 0.0 |
| 94742 Donor 3 U - A Mesenchymal Stem Cells | 0.0 |
| 94743 Donor 3 U - B Mesenchymal Stem Cells | 0.0 |
| 94730 Donor 3 AM - A adipose | 0.0 |
| 94731 Donor 3 AM - B adipose | 0.0 |
| 94732 Donor 3 AM - C adipose | 0.0 |
| 94733 Donor 3 AD - A adipose | 0.0 |
| 94734 Donor 3 AD - B adipose | 0.0 |
| 94735 Donor 3 AD - C adipose | 0.0 |
| 77138 Liver HepG2untreated | 0.0 |
| 73556 Heart Cardiac stromal cells (primary) | 0.0 |
| 81735 Small Intestine | 0.0 |
| 72409 Kidney Proximal Convoluted Tubule | 0.0 |
| 82685 Small intestine Duodenum | 0.0 |
| 90650 Adrenal Adrenocortical adenoma | 100.0 |
| 72410 Kidney HRCE | 0.0 |
| 72411 Kidney HRE | 0.0 |
| 73139 Uterus Uterine smooth muscle cells | 0.0 |

Column A - Rel. Exp. (%) Ag3554, Run 253329898

TABLE GF
general oncology screening panel v 2.4

| Tissue Name | A |
|---|---|
| Colon cancer 1 | 7.9 |
| CC Margin (ODO3921) | 3.9 |
| Colon cancer 2 | 0.8 |
| Colon NAT 2 | 0.3 |
| Colon cancer 3 | 2.4 |
| Colon NAT 3 | 4.0 |
| Colon malignant cancer 4 | 2.4 |
| Colon NAT 4 | 0.6 |
| Lung cancer 1 | 3.2 |
| Lung NAT 1 | 0.4 |
| Lung cancer 2 | 12.8 |
| Lung NAT 2 | 0.6 |
| Squamous cell carcinoma 3 | 2.5 |
| Lung NAT 3 | 0.0 |
| Metastatic melanoma 1 | 62.9 |
| Melanoma 2 | 0.0 |
| Melanoma 3 | 0.0 |
| Metastatic melanoma 4 | 100.0 |
| Metastatic melanoma 5 | 31.9 |
| Bladder cancer 1 | 0.4 |
| Bladder NAT 1 | 0.0 |
| Bladder cancer 2 | 0.0 |
| Bladder NAT 2 | 0.0 |
| Bladder NAT 3 | 0.0 |
| Bladder NAT 4 | 7.2 |
| Prostate adenocarcinoma 1 | 12.1 |
| Prostate adenocarcinoma 2 | 0.4 |
| Prostate adenocarcinoma 3 | 2.7 |
| Prostate adenocarcinoma 4 | 1.4 |
| Prostate NAT 5 | 0.3 |
| Prostate adenocarcinoma 6 | 1.9 |
| Prostate adenocarcinoma 7 | 4.8 |
| Prostate adenocarcinoma 8 | 0.1 |
| Prostate adenocarcinoma 9 | 9.3 |
| Prostate NAT 10 | 0.0 |
| Kidney cancer 1 | 39.0 |
| Kidney NAT 1 | 9.7 |
| Kidney cancer 2 | 22.1 |
| Kidney NAT 2 | 18.4 |
| Kidney cancer 3 | 7.0 |
| Kidney NAT 3 | 8.0 |
| Kidney cancer 4 | 5.6 |
| Kidney NAT 4 | 6.3 |

Column A - Rel. Exp. (%) Ag354, Run 259737951

Al_comprehensive panel_v1.0 Summary: Ag3554 The highest expression of this gene was detected in a normal lung sample (CT=26). This gene is downregulated in lung samples from patients suffering from COPD, emphysema or asthma. The gene's expression is useful in differentiating COPD, emphysema or asthma lung tissue from normal lung tissue. Therapeutic modulation of this gene or gene product is useful in the treatment of COPD, emphysema or asthma. This gene was upregulated in cartilage, bone, synovium and synovial fluid from rheumathoid arthritic patients and is therefore useful in differentiating these tissues from rheumathoid arthritic verses normal joints. Therapeutic modulation of this gene, expressed protein and/or use of antibodies or small molecule drugs targeting the gene or gene product are useful in the treatment of rheumathoid arthritis.

General_screening_panel_v1.4 Summary: Ag3554 Highest expression of this gene was detected in fetal lung (CT=25.2) and it was overexpressed as compared to adult lung. The gene product enhances lung growth or development in the fetus and thus can also act in a regenerative capacity in the adult. Therapeutic modulation of this gene, expressed protein and/or use of small molecule drugs targeting the gene or gene product are useful in the treatment of lung diseases. High to moderate levels of gene expression were seen in tissues with metabolic/endocrine functions including pancreas, adipose, adrenal gland, thyroid, pituitary gland, skeletal muscle, heart, liver and the gastrointestinal tract. Therapeutic modulation of this gene, expressed protein and/or use of small molecule drugs targeting the gene or gene product are useful in the treatment of endocrine/metabolically related diseases, such as obesity and diabetes. Moderate gene expression was seen in all regions of the central nervous system examined, including amygdala, hippocampus, substantia nigra, thalamus, cerebellum, cerebral cortex, and spinal cord. Therapeutic modulation of this gene, expressed protein and/or use of small molecule drugs targeting the gene or gene product are useful in the treatment of central nervous system disorders such as Alzheimer's disease, Parkinson's disease, epilepsy, multiple sclerosis, schizophrenia and depression.

Significant expression of this gene was also observed in colon cancer tissue and cell lines derived from melanoma, brain, gastric, lung and breast cancers. Gene expression is useful for differentiating these cancerous tissues from their normal counterparts. This gene encodes for nuclear receptor NOR1. In extraskeletal myxoid chondrosarcoma, chromosomal translocation creates a gene fusion between EWS and the orphan nuclear receptor NOR1, EWS/NOR1, which is believed to lead to malignant transformation by functioning as a transcriptional activator or regulator of mRNA splicing (Clark et. al., 1996 Oncogene 12: 229-235, PubMed ID: 8570200; Ohkura et al., 2002, J Biol Chem 277(1):535-43, PMID: 11673470). Therapeutic modulation of this gene, expressed protein and/or use of small molecule drugs targeting the gene or gene product are useful in the treatment of melanoma, chondrosarcoma, and brain, gastric, lung and breast cancers.

Panel 4.1 D Summary: Ag3554 The highest gene expression was detected in LAK cells treated with PMA and ionomycin (CT=25). This gene was upregulated in stimulated immune cells, including activated primary and secondary Th1 and Th2 cell, activated CD4 lymphocytes, lung fibroblasts treated with interferon gamma, lung fibroblasts treated with TNF alpha and IL-1 beta, and mononcytes and macrophages stimulated with LPS. The gene's expression is useful in differentiating these stimulated immune cell types from resting cells. Therapeutic modulation of this gene, expressed protein and/or use of antibodies or small molecule drugs targeting the gene or gene product are useful in the treatment of: immunosupressed individuals, inflammatory disorders and autoimmune diseases, such as asthma, emphysema, allergy, psoriasis, arthritis, ulcerative colitis, rheumatoid disease and inflammatory bowel disease.

Panel 5 Islet Summary: Ag3554 Highest expression of this gene was detected in adrenocortical adenoma sample (CT=27.9). Thus, this gene may play a role in tumor development. Therapeutic modulation of this gene, expressed protein and/or use of small molecule drugs targeting the gene or gene product are useful in the treatment of adrenocortical adenoma. Moderate levels of gene expression were detected in skeletal muscle and visceral adipose of obese and diabetic patients. Therapeutic modulation of this gene, expressed protein and/or use of small molecule drugs targeting the gene or gene product are useful in the treatment of obesity and diabetes.

general oncology screening panel_V_{—}2.4 Summary: Ag3554 The highest expression of this gene was detected in metastatic melanoma sample (CT=26) and this gene was overexpressed in colon, kidney, prostate and lung cancers when compared to normal adjacent tissues. Gene expression is useful in differentiating colon, kidney, prostate, lung cancer and melanoma tissues from their normal counterparts. Therapeutic modulation of this gene, expressed protein and/or use of antibodies or small molecule drugs targeting the gene or gene product are useful in the treatment of cancers of the colon, kidney, prostate, skin and lung.

H. NOV11, CG59889-01: KIAA1199, and CG59889-04: KIAA1199 Extension.

Expression of genes CG59889-01 and CG59889-04 was assessed using the primer-probe set Ag3626, described in Table HA. Results of the RTQ-PCR runs are shown in Tables HB, HC, HD and HE.

TABLE HA
Probe Name Ag3626

| | | | Start | SEQ |
|---|---|---|---|---|
| Primers | Sequences | Length | Position | ID No |
| Forward | 5′-ctgaggatcacaaagcca | 20 | 3750 | 179 |
| | aa-3′ | | | |
| Probe | TET-5′-atcttccaagttgt | 26 | 3770 | 180 |
| | gcccatccctgt-3′-TAMRA | | | |
| Reverse | 5′-cagctgtcctcacaactt | 22 | 3805 | 181 |
| | cttc-3′ | | | |

TABLE HB
AI comprehensive panel v1.0

| Tissue Name | A |
|---|---|
| 110967 COPD-F | 1.0 |
| 110980 COPD-F | 1.6 |
| 110968 COPD-M | 2.2 |
| 110977 COPD-M | 8.5 |
| 110989 Emphysema-F | 16.3 |
| 110992 Emphysema-F | 4.3 |
| 110993 Emphysema-F | 3.3 |
| 110994 Emphysema-F | 1.2 |
| 110995 Emphysema-F | 11.6 |
| 110996 Emphysema-F | 1.6 |
| 110997 Asthma-M | 0.9 |
| 111001 Asthma-F | 2.6 |
| 111002 Asthma-F | 9.2 |
| 111003 Atopic Asthma-F | 4.0 |
| 111004 Atopic Asthma-F | 7.6 |
| 111005 Atopic Asthma-F | 2.0 |
| 111006 Atopic Asthma-F | 2.4 |
| 111417 Allergy-M | 3.4 |
| 112347 Allergy-M | 0.4 |
| 112349 Normal Lung-F | 0.1 |
| 112357 Normal Lung-F | 13.8 |
| 112354 Normal Lung-M | 1.5 |
| 112374 Crohns-F | 28.9 |
| 112389 Match Control Crohns-F | 3.5 |
| 112375 Crohns-F | 43.8 |
| 112732 Match Control Crohns-F | 8.2 |
| 112725 Crohns-M | 6.1 |
| 112387 Match Control Crohns-M | 15.6 |
| 112378 Crohns-M | 0.2 |
| 112390 Match Control Crohns-M | 16.8 |
| 112726 Crohns-M | 6.5 |
| 112731 Match Control Crohns-M | 6.1 |
| 112380 Ulcer Col-F | 5.0 |
| 112734 Match Control Ulcer Col-F | 29.9 |
| 112384 Ulcer Col-F | 21.9 |
| 112737 Match Control Ulcer Col-F | 0.5 |
| 112386 Ulcer Col-F | 0.9 |
| 112738 Match Control Ulcer Col-F | 2.0 |
| 112381 Ulcer Col-M | 0.1 |
| 112735 Match Control Ulcer Col-M | 8.5 |
| 112382 Ulcer Col-M | 4.0 |
| 112394 Match Control Ulcer Col-M | 2.0 |
| 112383 Ulcer Col-M | 14.9 |
| 112736 Match Control Ulcer Col-M | 4.4 |
| 112423 Psoriasis-F | 3.3 |
| 112427 Match Control Psoriasis-F | 13.7 |
| 112418 Psoriasis-M | 1.8 |
| 112723 Match Control Psoriasis-M | 15.1 |
| 112419 Psoriasis-M | 4.6 |
| 112424 Match Control Psoriasis-M | 2.0 |
| 112420 Psoriasis-M | 12.2 |
| 112425 Match Control Psoriasis-M | 9.6 |
| 104689 (MF) OA Bone-Backus | 22.7 |
| 104690 (MF) Adj “Normal” Bone-Backus | 12.4 |
| 104691 (MF) OA Synovium-Backus | 28.5 |
| 104692 (BA) OA Cartilage-Backus | 45.1 |
| 104694 (BA) OA Bone-Backus | 39.8 |
| 104695 (BA) Adj “Normal” Bone-Backus | 26.2 |
| 104696 (BA) OA Synovium-Backus | 45.4 |
| 104700 (SS) OA Bone-Backus | 13.1 |
| 104701 (SS) Adj “Normal” Bone-Backus | 31.6 |
| 104702 (SS) OA Synovium-Backus | 13.0 |
| 117093 OA Cartilage Rep7 | 8.4 |
| 112672 OA Bone5 | 31.6 |
| 112673 OA Synovium5 | 15.7 |
| 112674 OA Synovial Fluid cells5 | 15.1 |
| 117100 OA Cartilage Rep14 | 2.3 |
| 112756 OA Bone9 | 100.0 |
| 112757 OA Synovium9 | 0.6 |
| 112758 OA Synovial Fluid Cells9 | 1.9 |
| 117125 RA Cartilage Rep2 | 1.3 |
| 113492 Bone2 RA | 4.2 |
| 113493 Synovium2 RA | 2.0 |
| 113494 Syn Fluid Cells RA | 5.5 |
| 113499 Cartilage4 RA | 4.3 |
| 113500 Bone4 RA | 9.5 |
| 113501 Synovium4 RA | 6.1 |
| 113502 Syn Fluid Cells4 RA | 3.7 |
| 113495 Cartilage3 RA | 3.9 |
| 113496 Bone3 RA | 7.4 |
| 113497 Synovium3 RA | 2.4 |
| 113498 Syn Fluid Cells3 RA | 3.3 |
| 117106 Normal Cartilage Rep20 | 1.8 |
| 113663 Bone3 Normal | 0.8 |
| 113664 Synovium3 Normal | 0.1 |
| 113665 Syn Fluid Cells3 Normal | 0.2 |
| 117107 Normal Cartilage Rep22 | 1.2 |
| 113667 Bone4 Normal | 8.1 |
| 113668 Synovium4 Normal | 6.0 |
| 113669 Syn Fluid Cells4 Normal | 17.0 |

Column A - Rel. Ex. (%) Ag3626, Run 234222205

TABLE HC
Ardais Colon1.0

| Tissue Name | A |
|---|---|
| 95318 colon (CHTN20435) | 19.9 |
| 95319 colon NAT (CHTN20435) | 0.3 |
| 95325 colon NAT (CHTN20473) | 0.4 |
| 97743 Colon cancer (CHTN20803) | 0.4 |
| 97745 Colon NAT (CHTN20867) | 1.0 |
| 97759 Colon cancer (OD06064) | 10.6 |
| 97760 Colon NAT (OD06064) | 0.3 |
| 98861 Colon cancer (OD06297-04) | 33.0 |
| 98862 Colon NAT (OD06297-015) | 0.7 |
| 98940 Colon malignant cancer (OD06205C) | 14.3 |
| 98941 Colon normal adjacent tissue (OD06205K) | 0.4 |
| 106291 colon adenocarcinoma (OD06787-02B) | 70.7 |
| 106292 colon NAT (OD06787-06F) | 0.8 |
| 106293 colon adenocarcinoma (OD06801-05E) | 19.8 |
| 108831 Colon cancer (OD06877) | 1.9 |
| 108832 Colon NAT (OD06877) | 0.3 |
| 138067 Colon cancer(CHTN 23212) | 65.1 |
| 138079 Colon cancer(CHTN 23624) | 13.9 |
| 138080 Colon NAT(CHTN 23624) | 0.3 |
| 142327 Colon cancer(8A3) | 6.4 |
| 142330 Colon cancer(8A6) | 6.9 |
| 142331 Colon cancer(8A7) | 17.3 |
| 142332 Colon NAT(8A8) | 1.3 |
| 142333 Colon cancer(8A9) | 83.5 |
| 142334 Colon NAT(8AA) | 1.1 |
| 142335 Colon cancer(8AB) | 76.8 |
| 142336 Colon cancer(8AC) | 100.0 |
| 142337 Colon NAT(8AD) | 2.1 |
| 142338 Colon cancer(8AE) | 59.5 |
| 142339 Colon NAT(8AF) | 1.8 |
| 142340 Colon cancer(8B0) | 22.5 |
| 142341 Colon cancer(8B1) | 72.7 |
| 142344 Colon cancer(8B7) | 66.0 |
| 145860 Colon NAT(9F1) | 1.6 |
| 145861 Colon cancer(9F2) | 19.3 |
| 145862 Colon NAT(A1D) | 2.0 |
| 145863 Colon cancer(9DB) | 17.0 |
| 145864 Colon NAT(A15) | 1.3 |
| 145865 Colon cancer(A14) | 49.3 |
| 145866 Colon NAT(9CC) | 1.5 |
| 145867 Colon cancer(9B9) | 74.7 |
| 148367 Colon Cancer(8677) | 11.5 |
| 148368 Colon NAT(8677) | 0.3 |
| 148372 Colon NAT(8842) | 0.2 |
| 148373 Colon Cancer(8869) | 27.4 |
| 148374 Colon NAT(8869) | 0.7 |
| 148375 Colon Cancer(8908) | 4.3 |
| 148376 Colon NAT(8908) | 0.2 |
| 148377 Colon Cancer(8688) | 9.0 |
| 148378 Colon NAT(8688) | 0.3 |
| 148379 Colon Cancer(8747) | 3.0 |
| 149748 Colon cancer(AC0) | 81.2 |
| 149752 Colon cancer(AC1) | 97.3 |
| 149754 Colon cancer(AC3) | 25.5 |
| 153791 Colon cancer(CHTN203C096) | 21.2 |
| 153792 Colon NAT(CHTN203C097) | 0.5 |
| 153797 Colon NAT(CHTN24753) | 2.9 |
| 154975 Colon NAT Pool | 0.5 |
| 152266 SW620 | 11.9 |
| 152297 47.HCT-116 | 1.8 |
| 155776 HT-29 | 55.5 |
| 155782 16. DLD-2 | 62.4 |
| 172030 Normal colon | 0.2 |

Column A - Rel. Exp. (%) Ag3626, Run 428498605

TABLE HD
Panel 4.1D

| Tissue Name | A |
|---|---|
| Secondary Th1 act | 0.4 |
| Secondary Th2 act | 0.1 |
| Secondary Tr1 act | 0.3 |
| Secondary Th1 rest | 0.0 |
| Secondary Th2 rest | 0.6 |
| Secondary Tr1 rest | 0.2 |
| Primary Th1 act | 0.3 |
| Primary Th2 act | 0.6 |
| Primary Tr1 act | 0.6 |
| Primary Th1 rest | 0.2 |
| Primary Th2 rest | 0.2 |
| Primary Tr1 rest | 0.3 |
| CD45RA CD4 lymphocyte act | 29.1 |
| CD45RO CD4 lymphocyte act | 0.3 |
| CD8 lymphocyte act | 0.1 |
| Secondary CD8 lymphocyte rest | 0.2 |
| Secondary CD8 lymphocyte act | 0.6 |
| CD4 lymphocyte none | 0.3 |
| 2ry Th1/Th2/Tr1 anti-CD95 CH11 | 0.5 |
| LAK cells rest | 0.7 |
| LAK cells IL-2 | 0.4 |
| LAK cells IL-2 + IL-12 | 0.0 |
| LAK cells IL-2 + IFN gamma | 0.6 |
| LAK cells IL-2 + IL-18 | 0.2 |
| LAK cells PMA/ionomycin | 0.3 |
| NK Cells IL-2 rest | 0.6 |
| Two Way MLR 3 day | 1.4 |
| Two Way MLR 5 day | 0.6 |
| Two Way MLR 7 day | 0.4 |
| PBMC rest | 0.2 |
| PBMC PWM | 5.1 |
| PBMC PHA-L | 8.3 |
| Ramos (B cell) none | 0.3 |
| Ramos (B cell) ionomycin | 0.8 |
| B lymphocytes PWM | 0.4 |
| B lymphocytes CD40L and IL-4 | 0.9 |
| EOL-1 dbcAMP | 0.1 |
| EOL-1 dbcAMP PMA/ionomycin | 0.6 |
| Dendritic cells none | 0.3 |
| Dendritic cells LPS | 0.2 |
| Dendritic cells anti-CD40 | 0.8 |
| Monocytes rest | 0.9 |
| Monocytes LPS | 40.6 |
| Macrophages rest | 0.1 |
| Macrophages LPS | 0.5 |
| HUVEC none | 0.4 |
| HUVEC starved | 0.1 |
| HUVEC IL-1beta | 0.2 |
| HUVEC IFN gamma | 0.2 |
| HUVEC TNF alpha + IFN gamma | 0.2 |
| HUVEC TNF alpha + IL4 | 0.1 |
| HUVEC IL-11 | 0.2 |
| Lung Microvascular EC none | 0.7 |
| Lung Microvascular EC TNFalpha + IL-1beta | 1.2 |
| Microvascular Dermal EC none | 0.1 |
| Microsvasular Dermal EC TNFalpha + IL-1beta | 0.7 |
| Bronchial epithelium TNFalpha + IL1beta | 0.5 |
| Small airway epithelium none | 1.1 |
| Small airway epithelium TNFalpha + IL-1beta | 1.1 |
| Coronery artery SMC rest | 28.5 |
| Coronery artery SMC TNFalpha + IL-1beta | 19.9 |
| Astrocytes rest | 61.6 |
| Astrocytes TNFalpha + IL-1beta | 100.0 |
| KU-812 (Basophil) rest | 0.3 |
| KU-812 (Basophil) PMA/ionomycin | 0.3 |
| CCD1106 (Keratinocytes) none | 0.6 |
| CCD1106 (Keratinocytes) TNFalpha + IL-1beta | 0.9 |
| Liver cirrhosis | 0.4 |
| NCI-H292 none | 9.5 |
| NCI-H292 IL-4 | 5.5 |
| NCI-H292 IL-9 | 4.2 |
| NCI-H292 IL-13 | 2.5 |
| NCI-H292 IFN gamma | 1.2 |
| HPAEC none | 0.1 |
| HPAEC TNF alpha + IL-1 beta | 2.2 |
| Lung fibroblast none | 75.8 |
| Lung fibroblast TNF alpha + IL-1 beta | 11.1 |
| Lung fibroblast IL-4 | 53.6 |
| Lung fibroblast IL-9 | 27.2 |
| Lung fibroblast IL-13 | 34.2 |
| Lung fibroblast IFN gamma | 20.4 |
| Dermal fibroblast CCD1070 rest | 99.3 |
| Dermal fibroblast CCD1070 TNF alpha | 64.6 |
| Dermal fibroblast CCD1070 IL-1 beta | 64.2 |
| Dermal fibroblast IFN gamma | 3.3 |
| Dermal fibroblast IL-4 | 1.4 |
| Dermal Fibroblasts rest | 66.9 |
| Neutrophils TNFa + LPS | 0.1 |
| Neutrophils rest | 0.0 |
| Colon | 0.1 |
| Lung | 8.8 |
| Thymus | 1.2 |
| Kidney | 0.3 |

Column A - Rel. Exp. (%) Ag3626, Run 169946026

TABLE HE
general oncology screening panel v 2.4

| Tissue Name | A |
|---|---|
| Colon cancer 1 | 30.6 |
| Colon NAT 1 | 0.8 |
| Colon cancer 2 | 19.9 |
| Colon NAT 2 | 0.6 |
| Colon cancer 3 | 100.0 |
| Colon NAT 3 | 1.1 |
| Colon malignant cancer 4 | 79.0 |
| Colon NAT 4 | 0.3 |
| Lung cancer 1 | 15.8 |
| Lung NAT 1 | 0.9 |
| Lung cancer 2 | 11.0 |
| Lung NAT 2 | 0.7 |
| Squamous cell carcinoma 3 | 21.3 |
| Lung NAT 3 | 0.2 |
| Metastatic melanoma 1 | 0.4 |
| Melanoma 2 | 0.5 |
| Melanoma 3 | 0.2 |
| Metastatic melanoma 4 | 8.0 |
| Metastatic melanoma 5 | 13.1 |
| Bladder cancer 1 | 0.6 |
| Bladder NAT 1 | 0.0 |
| Bladder cancer 2 | 0.2 |
| Bladder NAT 2 | 0.1 |
| Bladder NAT 3 | 0.0 |
| Bladder NAT 4 | 0.1 |
| Prostate adenocarcinoma 1 | 0.8 |
| Prostate adenocarcinoma 2 | 0.3 |
| Prostate adenocarcinoma 3 | 0.8 |
| Prostate adenocarcinoma 4 | 42.9 |
| Prostate NAT 5 | 0.0 |
| Prostate adenocarcinoma 6 | 0.1 |
| Prostate adenocarcinoma 7 | 0.5 |
| Prostate adenocarcinoma 8 | 0.0 |
| Prostate adenocarcinoma 9 | 2.6 |
| Prostate NAT 10 | 0.3 |
| Kidney cancer 1 | 1.1 |
| Kidney NAT 1 | 1.3 |
| Kidney cancer 2 | 4.1 |
| Kidney NAT 2 | 1.2 |
| Kidney cancer 3 | 2.1 |
| Kidney NAT 3 | 0.8 |
| Kidney cancer 4 | 0.7 |
| Kidney NAT 4 | 0.5 |

Column A - Rel. Exp. (%) Ag366, Run 260268656

Al_comprehensive panelv1.0 Summary: Ag3626 Transcript expression was higher in some joint tissues isolated from osteoarthritic (OA) patients as compared to normal joint tissues, with highest expression in an OA bone sample (CT=28.5). The gene's expression is useful in differentiating OA joint tissue from normal joint tissue. The transscript or the protein it encodes can be used as a marker for osteoarthritic tissues. Therapeutic modulation of this gene, expressed protein and/or use of antibodies or small molecule drugs targeting the gene or gene product are useful in the treatment of arthritis.

Ardais Colon 1.0 Summary: Ag3626 This gene was highly expressed in a colon cancer as compared to their normal adjacent tissue (NAT) counterparts. The gene's expression is useful in differentiating colon cancer tissue from normal colon tissue. Therapeutic modulation of this gene, expressed protein and/or use of antibodies or small molecule drugs targeting the gene or gene product are useful in the treatment of colon cancer.

Panel 4.1 D Summary: Ag3626 Highest gene expression was seen in TNF-alpha and IL-1 beta treated astrocytes (CT=26). Therapeutic modulation of this gene and/or use of antibodies or small molecule drugs targeting the gene or gene product are useful in the treatment of inflammatory CNS diseases such as multiple sclerosis. This gene was expressed in certain samples from lung and dermal fibroblasts. Therapeutic modulation of this gene and/or use of antibodies or small molecule drugs targeting the gene or gene product are useful in the treatment of lung inflammatory diseases such as asthma, and chronic obstructive pulmonary diseases, inflammatory skin diseases such as psoriasis, atopic dermatitis, ulcerative dermatitis, ulcerative colitis.

general oncology screening panel_V_{—}2.4 Summary: Ag3626 This gene was overexpressed in 4 out of 4 colon cancer and 3 out of 3 lung cancer samples as compared to Normal Adjacent Tissues (NATs). This gene was also expressed in melanoma, prostate adenocarcinoma and kidney cancer samples. The Gene expression is useful in differentiating skin, colon, lung, prostate and kidney cancerous tissues from normal counterparts. Therapeutic modulation of this gene and/or use of antibodies or small molecule drugs targeting the gene or gene product are useful in the treatment of cancers of the colon, lung, skin, prostate and kidney.

I. NOV12, CG88912-O₂: BETA-NEOENDORPHIN-DYNORPHIN PRECURSOR.

Expression of gene CG88912-02 was assessed using the primer-probe set Ag7210, described in Table IA. Results of the RTQ-PCR runs are shown in Table IB.

TABLE IA
Probe Name Ag7210

| | | | Start | SEQ |
|---|---|---|---|---|
| Primers | Sequences | Length | Position | ID No |
| Forward | 5′-cctgaaggagctgaacga | 20 | 282 | 182 |
| | tg-3′ | | | |
| Probe | TET-5′-ccatggagactggc | 26 | 305 | 183 |
| | acactctatctc-3′-TAMRA | | | |
| Reverse | 5′-tagcgtttgacctgctcc | 20 | 346 | 184 |
| | tt-3′ | | | |

TABLE IB
General screening panel v1.7

| Tissue Name | A |
|---|---|
| Adipose | 0.0 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 0.1 |
| Prostate ca. (bone met) PC-3 | 0.0 |
| Prostate ca. DU145 | 0.0 |
| Prostate pool | 0.0 |
| Uterus pool | 0.0 |
| Ovarian ca. OVCAR-3 | 0.0 |
| Ovarian ca. (ascites) SK-OV-3 | 0.0 |
| Ovarian ca. OVCAR-4 | 0.0 |
| Ovarian ca. OVCAR-5 | 0.0 |
| Ovarian ca. IGROV-1 | 100.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| 113452 mammary gland | 0.0 |
| Trachea | 0.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Lung ca. DMS-114 | 0.0 |
| Liver | 0.0 |
| Fetal Liver | 0.0 |
| Kidney pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Stomach | 0.0 |
| Colon ca. SW-948 | 0.0 |
| Colon ca. SW480 | 0.0 |
| Colon ca. (SW480 met) SW620 | 0.0 |
| Colon ca. HT29 | 0.0 |
| Colon ca. HCT-116 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon | 0.0 |
| Small Intestine | 0.0 |
| Fetal Heart | 0.0 |
| Heart | 0.0 |
| Lymph Node Pool | 0.0 |
| Lymph Node pool 2 | 0.0 |
| Fetal Skeletal Muscle | 0.0 |
| Skeletal Muscle pool | 0.0 |
| Skeletal Muscle | 0.0 |
| Spleen | 0.0 |
| Thymus | 0.0 |
| CNS cancer (glio/astro) SF-268 | 0.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 0.7 |
| Brain (Cerebellum) | 0.0 |
| Brain (Fetal) | 0.3 |
| Brain (Hippocampus) | 0.6 |
| Cerebral Cortex pool | 0.1 |
| Brain (Substantia nigra) | 0.1 |
| Brain (Thalamus) | 0.4 |
| Brain (Whole) | 0.6 |
| Spinal Cord | 0.1 |
| Adrenal Gland | 0.0 |
| Pituitary Gland | 24.7 |
| Salivary Gland | 0.0 |
| Thyroid | 0.0 |
| Pancreatic ca. PANC-1 | 0.0 |
| Pancreas pool | 0.0 |

Column A - Rel. Ex. (%) Ag7210, Run 318040771

General_screening_panel_v1.7 Summary: Ag7210 The highest gene expression was detected in ovarian cancer cell line IGROV-1 (CT=23). Gene expression was detected in testis and brain. The gene's expression is useful in differentiating brain and testicular tissues from the other tissues represented on this panel. Therapeutic modulation of this gene, expressed protein and/or use of antibodies or small molecule drugs targeting the gene or gene product are useful in the treatment of disorders of the central nervous system including Alzheimer's disease, Parkinson's disease, trauma, stroke, epilepsy, pain, multiple sclerosis, schizophrenia, bipolar disorder, depression, autism, drug and alcohol addiction.

### Example D

### Gene Expression Analysis Using CuraChip in Human Tissues

Background: CuraGen has developed a gene microarray (CuraChip 1.2) for target identification. It provides a high-throughput means of global mRNA expression analyses of CuraGen's collection of cDNA sequences representing the Pharmaceutically Tractable Genome (PTG). This sequence set includes genes which can be developed into protein therapeutics, or used to develop antibody or small molecule therapeutics. CuraChip 1.2 contains ˜11,000 oligos representing approximately 8,500 gene loci, including (but not restricted to) kinases, ion channels, G-protein coupled receptors (GPCRs), nuclear hormone receptors, proteases, transporters, metabolic enzymes, hormones, growth factors, chemokines, cytokines, complement and coagulation factors, and cell surface receptors.

The CuraChip cDNAs were represented as 30-mer oligodeoxyribonucleotides (oligos) on a glass microchip. Hybridization methods using the longer CuraChip oligos are more specific compared to methods using 25-mer oligos. CuraChip oligos were synthesized with a linker, purified to remove truncated oligos (which can influence hybridization strength and specificity), and spotted on a glass slide. Oligo-dT primers were used to generate cRNA probes for hybridization from samples of interest. A biotin-avidin conjugation system was used to detect hybridized probes with a fluorophore-labeled secondary antibody. Gene expression was analyzed using clustering and correlation bioinformatics tools such as Spotfire® (Spotfire, Inc., 212 Elm Street, Somerville, Mass. 02144) and statistical tools such as multivariate analysis (MVA).

A number of control spots are present on CuraChip 1.2 for efficiency calculations and to provide alternative normalization methods. For example, CuraChip 1.2 contains a number of empty or negative control spots, as well as positive control spots containing a dilution series of oligos that detect the highly-expressed genes Ubiquitin and glyceraldehyde-3-phosphate dehydrogenase (GAPD). An analysis of spot signal level was performed using raw data from 67 hybridizations using all oligos. The maximum signal intensity for each oligo across all 67 hybridizations was determined, and the fold-over-background for this maximum signal was calculated (i.e. if the background reading is 20 and the raw spot intensity is 100, then the fold-over-background for that spot is 5×). The negative control or empty spots do occasionally “fire” or give a signal over the background level; however, they do not fire very strongly, with 77.1% of empty spots firing <3×over background and 91.7%<5×. The positive control spots (Ubiquitin and GAPD) always fired at >100×background. The experimental oligos (CuraOligos) fired over the entire range of intensities, with some at low fold-over-background intensities. Since the negative control spots do fire occasionally at low levels, we have set a suggested threshhold for data analysis at >5×background.

Approximately 561 samples of RNA from tissues obtained from surgically dissected diseased- and non-diseased tissues, and treated and untreated cell lines, were used to generate labelled nucleic acid which was hybridized to PTG Chip 1.2. Oligonucleotides corresponding to specific genes under investigation were used to determine gene expression profile.

I. Expression analysis of NOV2 CG124800-02: Oligonucleotide (optg2_{—}0013773, TAAAGGTCTCCACAGAGTTTATGCCATATT) (SEQ ID NO: 185) corresponding to CG124800-02 was used to determine specific gene expression on PTG Chip 1.2. Elevated levels of gene expression were detected in Alzheimer's disease and colon cancer samples as compared to the normal samples (Table DI). The gene's expression is useful for differentiating Alzheimer's disease brain tissue and colon cancer tissue from normal brain and normal colon, respectively. Therapeutic modulation of this gene, expressed protein and/or use of antibodies or small molecule drugs targeting the gene or gene product would be useful in the treatment of Alzheimer's disease and colon cancer.

TABLE DI
CG124800-02

| | Level of expression |
|---|---|
| G1C4D21B11-39_Alzheier's disease B4951 | 1431.15 |
| G1C4D21B11-40_Alzheimer's disease B4953 | 959.87 |
| G1C4D21B11-41_Alzheimer's disease B5018 | 1123.4 |
| G1C4D21B11-43_Alzheimer's disease B5019 | 935.43 |
| G1C4D21B11-44_Alzheimer's disease B5086 | 851.64 |
| G1C4D21B11-51_Alzheimer's disease B5096 | 852.47 |
| G1C4D21B11-52_Alzheimer's disease B5098 | 1354.42 |
| G1C4D21B11-54_Alzheimer's disease B5129 | 1515.67 |
| G1C4D21B11-55_Alzheimer's disease B5210 | 369.98 |
| G1C4D21B11-56_Control B4810 | 627.86 |
| G1C4D21B11-57_Control B4825 | 212.3 |
| G1C4D21B11-58_Control B4930 | 676.9 |
| G1C4D21B11-59_Control B4932 | 131.09 |
| G1C4D21B11-60_Control B5024 | 96.44 |
| G1C4D21B11-61_Control B5113 | 651.75 |
| G1C4D21B11-62_Control B5140 | 1305.36 |
| G1C4D21B11-63_Control B5190 | 422.09 |
| G1C4D21B11-64_Control B5220 | 126.97 |
| G1C4D21B11-65_Control B5245 | 516.33 |
| G1C4E19B13-12_Colon NAT(9F1) | 433.47 |
| G1C4E19B13-13_Colon cancer(9F2) | 572.44 |
| G1C4E19B13-14_Colon NAT(A1D) | 306.05 |
| G1C4E19B13-15_Colon cancer(9DB) | 6278.14 |
| G1C4E19B13-16_Colon NAT(A15) | 305.91 |
| G1C4E19B13-17_Colon cancer(A14) | 1554.8 |
| G1C4E19B13-18_Colon NAT(ACB) | 272.53 |
| G1C4E19B13-19_Colon cancer(AC0) | 657.42 |
| G1C4E19B13-2_Colon cancer(8A4) | 762.73 |
| G1C4E19B13-20_Colon NAT(ACD) | 416.35 |
| G1C4E19B13-21_Colon cancer(AC4) | 514.59 |
| G1C4E19B13-22_Colon NAT(AC2) | 171.76 |
| G1C4E19B13-23_Colon cancer(AC1) | 1090.92 |
| G1C4E19B13-24_Colon NAT(ACC) | 330.16 |
| G1C4E19B13-25_Colon cancer(AC3) | 468.83 |

II. Expression analysis of NOV4 CG186317-02: Oligonucleotide (optg2_{—}1203115, ATGCTGTGMCGAGTGTGATATTACTGMT) (SEQ ID NO: 186) corresponding to CG186317-02 was used to determine specific gene expression on PTG Chip 1.2. Significant gene expression was detected in brain. Reduced expression was seen in Alzheimer's disease samples and in amygdala and anterior cingulate from clinically depressed patients as compared to the normal samples (Table DII). Gene expression is useful in differentiating Alzheimer's disease and depressed amygdala and anterior cingulate samples from normal brain samples. Therapeutic modulation of this gene, expressed protein and/or use of antibodies or small molecule drugs targeting the gene or gene product would be useful in the treatment of central nervous system disorders such as Alzheimer's disease and depression.

TABLE DII
CG186317-02

| | Level of expression |
|---|---|
| G1C4D21B11-39_Alzheimer's disease B4951 | 77.45 |
| G1C4D21B11-40_Alzheimer's disease B4953 | 199.38 |
| G1C4D21B11-41_Alzheimer's disease B5018 | 39.53 |
| G1C4D21B11-43_Alzheimer's disease B5019 | 16.78 |
| G1C4D21B11-44_Alzheimer's disease B5086 | 117.75 |
| G1C4D21B11-51_Alzheimer's disease B5096 | 94.01 |
| G1C4D21B11-52_Alzheimer's disease B5098 | 104.19 |
| G1C4D21B11-54_Alzheimer's disease B5129 | 43.82 |
| G1C4D21B11-55_Alzheimer's disease B5210 | 134.3 |
| G1C4D21B11-56_Control B4810 | 266.49 |
| G1C4D21B11-57_Control B4825 | 320.93 |
| G1C4D21B11-58_Control B4930 | 60.34 |
| G1C4D21B11-59_Control B4932 | 495.27 |
| G1C4D21B11-60_Control B5024 | 429.83 |
| G1C4D21B11-61_Control B5113 | 140.35 |
| G1C4D21B11-62_Control B5140 | 101.42 |
| G1C4D21B11-63_Control B5190 | 104.48 |
| G1C4D21B11-64_Control B5220 | 348.21 |
| G1C4D21B11-65_Control B5245 | 227.33 |
| G1C4E21B14-62_Schizophrenia thalamus 477 | 93.13 |
| G1C4E21B14-63_Schizophrenia thalamus 532 | 255.67 |
| G1C4E21B14-64_Schizophrenia thalamus 683 | 188.96 |
| G1C4E21B14-65_Schizophrenia thalamus 544 | 51.59 |
| G1C4E21B14-66_Schizophrenia thalamus 1671 | 0 |
| G1C4E21B14-67_Schizophrenia thalamus 1737 | 0 |
| G1C4E21B14-68_Schizophrenia thalamus 2464 | 184.62 |
| G1C4E21B14-69_Schizophrenia thalamus 2586 | 62.52 |
| G1C4E23B15-1_Depression amygdala 600 | 81.27 |
| G1C4E23B15-10_Depression amygdala 759 | 143.59 |
| G1C4E23B15-11_Depression anterior cingulate 759 | 144.24 |
| G1C4E23B15-12_Control amygdala 552 | 233.29 |
| G1C4E23B15-14_Control anterior cingulate 482 | 378.72 |
| G1C4E23B15-15_Depression anterior cingulate 721 | 129.64 |
| G1C4E23B15-16_Control amygdala 3175 | 522.18 |
| G1C4E23B15-17_Depression anterior cingulate 600 | 175.33 |
| G1C4E23B15-18_Depression anterior cingulate 588 | 135.98 |
| G1C4E23B15-19_Control anterior cingulate 3175 | 408.96 |
| G1C4E23B15-2_Control anterior cingulate 606 | 563.12 |
| G1C4E23B15-20_Depression anterior cingulate 567 | 158.03 |

G1 C4E23B15-21_Depression Amygdala 588 132.49

III. Expression analysis of NOV5, CG192920-01: Oligonucleotide (optg2_{—}1201806, ACTTATAGCGTTTCCTCCTCGAAATTCTAC) (SEQ ID NO: 187) corresponding to CG192920-01 was used to determine specific gene expression on PTG Chip 1.2. Reduced gene expression was detected in colon cancer samples as compared to the normal adjacent tissue (NAT) (Table DII). Gene expression is useful in differentiating colon cancer from normal colon tissue. Therapeutic modulation of this gene, expressed protein and/or use of antibodies or small molecule drugs targeting the gene or gene product would be useful in the treatment of colon cancer.

TABLE DIII
CG192920-01

| | Level of expression |
|---|---|
| G1C4E19B13-10_Colon NT(8B6) | 561.84 |
| G1C4E19B13-12_Colon NAT(9F1) | 461.6 |
| G1C4E19B13-13_Colon cancer(9F2) | 280 |
| G1C4E19B13-14_Colon NAT(A1D) | 182.05 |
| G1C4E19B13-15_Colon cancer(9DB) | 194.77 |
| G1C4E19B13-16_Colon NAT(A15) | 164.03 |
| G1C4E19B13-17_Colon cancer(A14) | 343.44 |
| G1C4E19B13-18_Colon NAT(ACB) | 267.87 |
| G1C4E19B13-19_Colon cancer(AC0) | 139.31 |
| G1C4E19B13-2_Colon cancer(8A4) | 159.57 |
| G1C4E19B13-20_Colon NAT(ACD) | 477.22 |
| G1C4E19B13-21_Colon cancer(AC4) | 141.46 |
| G1C4E19B13-22_Colon NAT(AC2) | 272.11 |
| G1C4E19B13-23_Colon cancer(AC1) | 124.75 |

### Other Embodiments

Although particular embodiments are disclosed herein in detail, this is done by way of example for purposes of illustration only, and is not intended to be limiting with respect to the scope of the appended claims, which follow. In particular, it is contemplated by the inventors that various substitutions, alterations, and modifications will be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

1. An isolated polypeptide comprising the mature form of an amino acid sequence selected from the group consisting of SEQ ID NO:2n, wherein n is an integer between 1 and 71 or is 94.

2. A composition comprising the polypeptide of claim 1 and a carrier.

3. A kit comprising, in one or more containers, the composition of claim 2.

4. A method for determining the presence or amount of the polypeptide of claim 1 in a sample, the method comprising:
(a) providing the sample;
(b) introducing the sample to an antibody that binds immunospecifically to the polypeptide; and
(c) determining the presence or amount of the antibody bound to the polypeptide,
wherein the presence or amount of the antibody indicates the presence or amount of the polypeptide in the sample.

5. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of the polypeptide in the sample of step (a) to the expression of the polypeptide present in a control sample from a second mammalian subject known not to have, or not to be predisposed to the disease,
wherein an alteration in the level of expression of the polypeptide in the first subject as compared to the control sample indicates the presence of or predisposition to the disease.

6. A method of identifying an agent that binds to the polypeptide of claim 1, the method comprising:
(a) introducing the polypeptide to the agent; and
(b) determining whether the agent binds to the polypeptide.

7. The method of claim 6 wherein the agent is a cellular receptor or a downstream effector.

8. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

9. A method for screening for a modulator of activity of or of latency or predisposition to a pathology associated with the polypeptide of claim 1, the method comprising:
(a) administering a test compound to a test animal at increased risk for a pathology associated with the polypeptide of claim 1, wherein the test animal recombinantly expresses the polypeptide of claim 1;
(b) measuring the activity of the polypeptide in the test animal after administering the compound of step (a); and
(c) comparing the activity of the polypeptide in the test animal with the activity of the polypeptide in a control animal not administered the compound, wherein a change in the activity of the polypeptide in the test animal relative to the control animal indicates the test compound is a modulator of activity of or latency or predisposition to, a pathology associated with the polypeptide of claim 1.

10. The method of claim 9, wherein said test animal is a recombinant test animal that expresses the polypeptide as a transgene or expresses the transgene under the control of a promoter at an increased level relative to a wild-type test animal, and wherein the promoter is not the native gene promoter of the transgene.

11. An antibody that immunospecifically binds to the polypeptide of claim 1.

12. The antibody of claim 11, wherein the antibody is a human monoclonal antibody.

13. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n-1, wherein n is an integer between 1 and 71.

14. The method of claim 13 wherein the cell is chosen from the group comprising a bacterial cell, an insect cell, a yeast cell and a mammalian cell.

15. An isolated polypeptide comprising an amino acid sequence selected from the group consisting of SEQ ID NO:2n, wherein n is an integer between 1 and 71 or is 94.

16. A method of treating a pathological state in a mammal, the method comprising administering to the mammal a polypeptide in an amount that is sufficient to alleviate the pathological state, wherein the polypeptide comprises the amino acid sequence selected from the group consisting of SEQ ID NO:2n, wherein n is an integer between 1 and 71 or is 94, or a biologically active fragment thereof.

17. An isolated nucleic acid molecule comprising a nucleic acid sequence selected from the group consisting of SEQ ID NO:2n-1, wherein n is an integer between 1 and 71.

18. An isolated nucleic acid molecule encoding the mature form of a polypeptide having an amino acid sequence selected from the group consisting of SEQ ID NO:2n, wherein n is an integer between 1 and 71 or is 94.

19. A vector comprising the nucleic acid molecule of claim 18.

20. A cell comprising the vector of claim 19.
